SEQUENCE LISTING (17 sequences)

SEQ ID NO: 1 - DNA, 864 bp, Primate
  CDS (1)..(864)
  misc_feature (1)..(864): LP231
atggcgctcgggctgctcatcgccgtgccgctgctgctgcaggcggcgccccgaggcgccgcgcactatgagatgatgggcacctgccgcatgatctgcgacccttacactgccgcgcccggcggggagcccccgggtgcaaaggcgcagccacccggacccagcaccgccgccctggaagtcatgcaggacctcagcgccaaccctcctcctcctttcatccagggacccaagggcgacccggggcgaccgggcaagccagggccgcgggggccccctggagagccgggcccgcctggacccaggggccctccgggagagaagggcgactcggggcggcccgggctgccagggctgcaactgacggcgggcacggccagcggcgtcggggtggtgggcggcggggccggggtaggtggcgattccgagggtgaagtgaccagtgcgctgagcgccaccttcagcggccccaagatcgccttctatgtgggtctcaagagcccccacgaaggctatgaggtgctgaagttcgatgacgtggtcaccaacctcggcaatcactatgaccccaccacgggcaagttcagctgccaggtacgcggcatctacttcttcacctaccacatcctcatgcgcggcggcgacggcaccagcatgtgggcggacctctgcaagaacgggcaggtccgggccagcgccattgcacaggacgccgaccagaactacgactacgccagtaacagcgtggtgctgcacttggattcaggggacgaagtgtatgtgaagctggatggcgggaaggctcacggaggcaataataacaagtacagcacgttctcgggctttcttctgtacccggattag

SEQ ID NO: 2 - protein, 287 aa, Primate
MALGLLIAVPLLLQAAPRGAAHYEMMGTCRMICDPYTAAPGGEPPGAKAQPPGPSTAALEVMQDLSANPPPPFIQGPKGDPGRPGKPGPRGPPGEPGPPGPRGPPGEKGDSGRPGLPGLQLTAGTASGVGVVGGGAGVGGDSEGEVTSALSATFSGPKIAFYVGLKSPHEGYEVLKFDDVVTNLGNHYDPTTGKFSCQVRGIYFFTYHILMRGGDGTSMWADLCKNGQVRASAIAQDADQNYDYASNSVVLHLDSGDEVYVKLDGGKAHGGNNNKYSTFSGFLLYPD

SEQ ID NO: 3 - DNA, 921 bp, primate
  CDS (1)..(921)
  misc_feature (1)..(921): LP285
atgagtctcaaaatgcttataagcaggaacaagctgattttactactaggaatagtcttttttgaacgaggtaaatctgcaactctttcgctccccaaagctcccagttgtgggcagagtctggttaaggtacagccttggaattattttaacattttcagtcgcattcttggaggaagccaagtggagaagggttcctatccctggcaggtatctctgaaacaaaggcagaagcatatttgtggaggaagcatcgtctcaccacagtgggtgatcacggcggctcactgcattgcaaacagaaacattgtgtctactttgaatgttactgctggagagtatgacttaagccagacagacccaggagagcaaactctcactattgaaactgtcatcatacatccacatttctccaccaagaaaccaatggactatgatattgcccttttgaagatggctggagccttccaatttggccactttgtggggcccatatgtcttccagagctgcgggagcaatttgaggctggttttatttgtacaactgcaggctggggccgcttaactgaaggtggcgtcctctcacaagtcttgcaggaagtgaatctgcctattttgacctgggaagagtgtgtggcagctctgttaacactaaagaggcccatcagtgggaagacctttctttgcacaggttttcctgatggagggagagacgcatgtcagggagattcaggaggttcactcatgtgccggaataagaaaggggcctggactctggctggtgtgacttcctggggtttgggctgtggtcgaggctggagaaacaatgtgaggaaaagtgatcaaggatcccctgggatcttcacagacattagtaaagtgctttcctggatccacgaacacatccaaactggtaactaa

SEQ ID NO: 4 - protein, 306 aa, primate
MSLKMLISRNKLILLLGIVFFERGKSATLSLPKAPSCGQSLVKVQPWNYFNIFSRILGGSQVEKGSYPWQVSLKQRQKHICGGSIVSPQWVITAAHCIANRNIVSTLNVTAGEYDLSQTDPGEQTLTIETVIIHPHFSTKKPMDYDIALLKMAGAFQFGHFVGPICLPELREQFEAGFICTTAGWGRLTEGGVLSQVLQEVNLPILTWEECVAALLTLKRPISGKTFLCTGFPDGGRDACQGDSGGSLMCRNKKGAWTLAGVTSWGLGCGRGWRNNVRKSDQGSPGIFTDISKVLSWIHEHIQTGN

SEQ ID NO: 5 - DNA, 930 bp, Primate
  misc_feature (1)..(930): LP272
atggaatgcatggggctcctgcgccccctcttcctccttagcggctgctgccaggccctggagatctcactggaccaggaacatattccctttggacccgtggtgtatcagacgcaagccacncgtcgcatcctcatgttgaacacaggcgatgtgggtgcaaggtttaaatgggacatcaaaaaatttgagcctcatttctccattagcccagaagaaggctatattacctcaggcatggaggtttcttttgaagtgacctaccatcccaccgaggtgggaaaggagagcctttgtaaaaacattctctgctacatccagggaggcagtcctctgagtctaaccctgtctggagtctgcgtgggaccacctgcggtaaaagaggtagtgaatttcacgtgccaggtgcgctccaagcacacgcagaccatcctgctgtcaaaccgcaccaaccagacctggaatctgcaccccatctttgagggcgagcactgggaggggcctgagttcatcaccctggaggcccaccagcaaaacaagccctatgagatcacctacaggccccgcaccatgaacttggagaaccgcaagcaccagggcaccctcttcttccccctcccagatgggaccggctggctgtatgctctgcatgggacttctgagctccccaaagctgtagccaatatctatcgtgaagtgccatgtaagaccccctacactgagcttctgccaatcaccaactggctgaacaagccccagagattccgggtcatcgtggaaatactgaaaccagagaagccggacctaagcatcactatgaagggccttgattacattgatgtactgtctggctctaagaaagactacaagctgaacttcttttcccacaaggagggaacgtacgctgcaaanngatcttgcggaagctga

SEQ ID NO: 6 - protein, 309 aa, Primate
  MISC_FEATURE (41)..(41): The ′xaa′ at location 41 stands for Thr
MECMGLLRPLFLLSGCCQALEISLDQEHIPFGPVVYQTQAXRRILMLNTGDVGARFKWDIKKFEPHFSISPEEGYITSGMEVSFEVTYHPTEVGKESLCKNILCYIQGGSPLSLTLSGVCVGPPAVKEVVNFTCQVRSKHTQTILLSNRTNQTWNLHPIFEGEHWEGPEFITLEAHQQNKPYEITYRPRTMNLENRKHQGTLFFPLPDGTGWLYALHGTSELPKAVANIYREVPCKTPYTELLPITNWLNKPQRFRVIVEILKPEKPDLSITMKGLDYIDVLSGSKKDYKLNFFSHKEGTYAAXXSCGS

SEQ ID NO: 7 - DNA, 936 bp, Primate
  CDS (1)..(936)
  misc_feature (1)..(936): LP357
atgggcctcaccctgctcttgctgctgctcctgggactagaaggtcagggcatagttggcagcctccctgaggtgctgcaggcacccgtgggaagctccattctggtgcagtgccactacaggctccaggatgtcaaagctcagaaggtgtggtgccggttcttgccggaggggtgccagcccctggtgtcctcagctgtggatcgcagagctccagcgggcaggcgtacgtttctcacagacctgggtgggggcctgctgcaggtggaaatggttaccctgcaggaagaggatgctggcgagtatggctgcatggtggatggggccagggggccccagattttgcacagagtctctctgaacatactgcccccagaggaagaagaagagacccataagattggcagtctggctgagaacgcattctcagaccctgcaggcagtgccaaccctttggaacccagccaggatgagaagagcatccccttgatctggggtgctgtgctcctggtaggtctgctggtggcagcggtggtgctgtttgctgtgatggccaagaggaaacaagggaacaggcttggtgtctgtggccgattcctgagcagcagagtttcaggcatgaatccctcctcagtggtccaccacgtcagtgactctggaccggctgctgaattgcctttggatgtaccacacattaggcttgactcaccaccttcatttgacaataccacctacaccagcctacctcttgattccccatcaggaaaaccttcactcccagctccatcctcattgccccctctacctcctaaggtcctggtctgctccaagcctgtgacatatgccacagtaatcttcccgggagggaacaagggtggagggacctcgtgtgggccagcccagaatccacctaacaatcagactccatccagctaa

SEQ ID NO: 8 - protein, 311 aa, Primate
MGLTLLLLLLLGLEGQGIVGSLPEVLQAPVGSSILVQCHYRLQDVKAQKVWCRFLPEGCQPLVSSAVDRRAPAGRRTFLTDLGGGLLQVEMVTLQEEDAGEYGCMVDGARGPQILHRVSLNILPPEEEEETHKIGSLAENAFSDPAGSANPLEPSQDEKSIPLIWGAVLLVGLLVAAVVLFAVMAKRKQGNRLGVCGRFLSSRVSGMNPSSVVHHVSDSGPAAELPLDVPHIRLDSPPSFDNTTYTSLPLDSPSGKPSLPAPSSLPPLPPKVLVCSKPVTYATVIFPGGNKGGGTSCGPAQNPPNNQTPSS

SEQ ID NO: 9 - DNA, 86 bp, Artificial Sequence
  Primer
gcgcctcgagatttccccgaaatctagatttccccgaaatgatttccccgaaatgatttccccgaaatatctgccatctcaattag

SEQ ID NO: 10 - DNA, 27 bp, Artificial Sequence
  Primer
gcggcaagctttttgcaaagcctaggc

SEQ ID NO: 11 - DNA, 270 bp, Artificial Sequence
  Primer
ctcgagatttccccgaaatctagatttccccgaaatgattccccgaaatgatttccccgaaatatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 12 - DNA, 32 bp, Artificial Sequence
  Primer
gcgctcgagggatgacagcgatagaaccccgg

SEQ ID NO: 13 - DNA, 31 bp, Artificial Sequence
  Primer
gcgaagcttcgcgactccccggatccgcctc

SEQ ID NO: 14 - DNA, 12 bp, Artificial Sequence
  Primer
ggggactttccc

SEQ ID NO: 15 - DNA, 70 bp, Artificial Sequence
  Primer
gcggcctcgaggggactttcccggggactttccggggatccgggactttccatcctgccatctcaattag

SEQ ID NO: 16 - DNA, 27 bp, Artificial Sequence
  Primer
gcggcaagctttttgcaaagcctaggc

SEQ ID NO: 17 - DNA, 256 bp, Artificial Sequence
  Primer
ctcgaggggactttcccggggactttccggggactttccgggactttccatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

(19) United States
(12) Patent Application Publication
(10) Pub. No.: US 2004/0266674 A1
(43) Pub. Date: Dec. 30, 2004

(54) Lp mammalian proteins; related reagents

(76) Inventors: Bradley Jay Mills, Fountaintown, IN (US); Santosh Kumar Mishra, The Capricorn (SG); Eric Wen Su, Carmel, IN (US); Gabor Varga, Indianapolis, IN (US); He Wang, Carmel, IN (US)

Correspondence Address: Eli Lilly and Company, Gerald P Keleher, Patent Division, P O Box 6288, Indianapolis, IN, 46206-6288, US

(21) Appl. No.: 10/487,462

(22) PCT Filed: Aug. 23, 2002
(86) PCT No.: PCT/US02/21857

Related U.S. Application Data
(60) Provisional application No. 60/317,188, filed on Sep. 5, 2001.

(51) Int. Cl. A61K 38/17; C07K 14/705; C07H 21/04

(52) U.S. Cl. 514/12; 530/350; 435/69.1; 435/320.1; 435/325; 536/23.5

(57) ABSTRACT

Isolated nucleic acid molecules encoding polypeptides from a human, reagents related thereto (including purified polypeptides specific antibodies) are provided. Methods of using said reagents and diagnostic kits are also provided.

### FIELD OF THE INVENTION

The present invention generally relates to compositions related to proteins. In particular, it provides purified genes, polynucleotide sequences, proteins, polypeptides, antibodies, binding compositions, and related reagents useful, e.g., in the diagnosis, treatment, and prevention of cell proliferative, autoimmune/inflammatory, cardiovascular, neurological, and developmental disorders, and in the assessment of the effects of exogenous compounds on the expression of nucleic acid and amino acid sequences of such proteins.

### BACKGROUND OF THE INVENTION

Protein transport and secretion are essential for cellular function. Protein transport is mediated by a signal peptide located at the amino terminus of the protein to be transported or secreted. Proteins targeted to the ER may either proceed through the secretory pathway or remain in any of the secretory organelles such as the ER, Golgi apparatus, or lysosomes. Proteins that transit through the secretory pathway are either secreted into the extracellular space or retained in the plasma membrane. Proteins that are retained in the plasma membrane contain one or more transmembrane domains, each comprised of about 20 hydrophobic amino acid residues. Secreted proteins are generally synthesized as inactive precursors that are activated by post-translational processing events during transit through the secretory pathway. Such events include glycosylation, proteolysis, and removal of the signal peptide by a signal peptidase. Examples of secreted proteins with amino terminal signal peptides are discussed below and include proteins with important roles in cell-to-cell signaling. Such proteins include transmembrane receptors and cell surface markers, extracellular matrix molecules, cytokines, hormones, growth and differentiation factors, enzymes, neuropeptides, and vasomediators (reviewed in Alberts, et al. (1994) Molecular Biology of The Cell, Garland Publishing, New York, N.Y., pp. 557-560, 582-592.). The discovery of new secreted proteins and the polynucleotides encoding them satisfies a need in the art by providing new compositions which are useful in the diagnosis, prevention, and treatment of cell proliferative, autoimmune/inflammatory, cardiovascular, neurological, and developmental disorders, and in the assessment of the effects of exogenous compounds on the expression of nucleic acid and amino acid sequences of secreted proteins.

### SUMMARY OF THE INVENTION

The present invention is based in part upon the discovery of LP (LP231, LP272, LP285, or LP357) proteins and/or polypeptides. The invention provides substantially pure, isolated, and/or recombinant LP protein or peptide (LP231, LP272, LP285, or LP357) exhibiting identity over a length of at least about 12 contiguous amino acids to a corresponding sequence of SEQ ID NO: Y; a natural sequence LP (LP231, LP272, LP285, or LP357) of SEQ ID NO: Y; a fusion protein comprising LP (LP231, LP272, LP285, or LP357) sequence. In preferred embodiments, the portion is at least about 20, 25, 30, 35, 40, 45, 50, 55, 60, 65, 70, 75, 80, 85, 90, 95, or 100 contiguous amino acid residues in length. In other embodiments, the LP (LP231, LP272, LP285, or LP357): LP231 comprises a mature sequence of Table 1; LP285 comprises a mature sequence of Table 2; LP272 comprises a mature sequence of Table 3; LP357 comprises a mature sequence of Table 4; protein or peptide: is from a warm blooded animal selected from a mammal, including a primate; comprises at least one polypeptide segment of SEQ ID NO:Y exhibits a plurality of portions exhibiting the identity; is a natural allelic variant of the LP (LP231, LP272, LP285, or LP357) has a length at least about 30 amino acids; exhibits at least two non-overlapping epitopes which are specific for a mammalian LP(LP231, LP272, LP285, or LP357) exhibits identity over a length of at least about 20 amino acids to LP(LP231, LP272, LP285, or LP357) exhibits at least two non-overlapping epitopes which are specific for a LP (LP231, LP272, LP285, or LP357) exhibits identity over a length of at least about 25 amino acids to a primate LP (LP231, LP272, LP285, or LP357) is glycosylated; is a synthetic polypeptide; is attached to a solid substrate; is conjugated to another chemical moiety; is a 5-fold or less substitution from natural LP (LP231, LP272, LP285, or LP357) sequence; or is a deletion or insertion variant from a natural LP (LP231, LP272, LP285, or LP357) sequence. Various preferred embodiments include a composition comprising: a sterile LP (LP231, LP272, LP285, or LP357) protein or peptide and a carrier, wherein the carrier is: an aqueous compound, including water, saline, and/or buffer; and/or formulated for oral, rectal, nasal, topical, or parenteral administration. The invention further provides a fusion protein, comprising: mature protein comprising sequence of Table 1, 2, 3, or 4 a detection or purification tag, including a FLAG, His6, or Ig sequence; or sequence of another LP (LP231, LP272, LP285, or LP357) protein or peptide. These reagents also make available a kit comprising such an LP (LP231, LP272, LP285, or LP357) protein or polypeptide, and: a compartment comprising the protein or polypeptide; and/or instructions for use or disposal of reagents in the kit. Providing an antigen, the invention further provides a binding compound comprising an antigen binding portion from an antibody, which specifically binds to a natural LP (LP231, LP272, LP285, or LP357) protein or polypeptide, wherein: the protein or polypeptide is a primate protein; the binding compound is an Fv, Fab, or Fab2 fragment; the binding compound is conjugated to another chemical moiety; or the antibody: is raised against a peptide sequence of a mature polypeptide comprising sequence of Table 1, 2, 3, or 4 is raised against a mature LP (LP231, LP272, LP285, or LP357) is immunoselected; is a polyclonal antibody; binds to a denatured LP, (LP231, LP272, LP285, or LP357) exhibits a Kd to antigen of at least 30 μM; is attached to a solid substrate, including a bead or plastic membrane; is in a sterile composition; or is detectably labeled, including, for example, a radioactive, enzymatic, structural, or fluorescent label. Preferred kits include those containing the binding compound, and: a compartment comprising the binding compound; and/or instructions for use or disposal of reagents in the kit. Many of the kits will be used for making a qualitative or quantitative analysis. Other preferred compositions will be those comprising: a sterile binding compound, or the binding compound and a carrier, wherein the carrier is: an aqueous compound, including water, saline, and/or buffer; and/or formulated for oral, rectal, nasal, topical, or parenteral administration. The present invention further provides an isolated or recombinant LP nucleic acid encoding a protein or peptide or fusion protein described above, wherein: the LP protein and/or polypeptide is from a mammal, including a primate; or the LP nucleic acid: encodes an antigenic peptide sequence from an LP (LP231, LP272, LP285, or LP357) of Table 1, 2, 3, or 4 encodes a plurality of antigenic peptide sequences from an LP (LP231, LP272, LP285, or LP357) of Table 1, 2, 3, or 4 exhibits identity to a natural cDNA encoding the segment; is an expression vector, further comprises an origin of replication; is from a natural source; comprises a detectable label; comprises synthetic nucleotide sequence; is less than 6 kb, preferably less than 3 kb; is from a mammal, including a primate; comprises a natural full length coding sequence; is a hybridization probe for a gene encoding an LP family protein; or is a PCR primer, PCR product, or mutagenesis primer. In certain embodiments, the invention provides a cell or tissue comprising such a recombinant LP nucleic acid. Preferred cells include: a prokaryotic cell; a eukaryotic cell; a bacterial cell; a yeast cell; an insect cell; a mammalian cell; a mouse cell; a primate cell; or a human cell. Other kit embodiments include a kit comprising the described LP nucleic acid, and: a compartment comprising the LP nucleic acid; a compartment further comprising an LP (LP231, LP272, LP285, or LP357) protein or polypeptide; and/or instructions for use or disposal of reagents in the kit. In many versions, the kit is capable of making a qualitative or quantitative analysis. Other LP nucleic acid embodiments include those which: hybridize under wash conditions of at least 42° C., 45° C., 47° C., 50° C., 55° C., 60° C., 65° C., or 70° C. and less than about 500 mM, 450 mM, 400 mM, 350 mM, 300 mM, 250 mM, 200 mM, 100 mM, to an LP of SEQ ID NO: X that exhibit identity over a stretch of at least about 30, 32, 34, 36, 38, 39, 40, 42, 44, 46, 48, 49, 50, 52, 54, 56, 58, 59, 75, or at least about 150 contiguous nucleotides to an LP (LP231, LP272, LP285, or LP357). In other embodiments: the wash conditions are at 55° C. and/or 300 mM salt; 60° C. and/or 150 mM salt; the identity is over a stretch is at least 55 or 75 nucleotides. In other embodiments, the invention provides a method of modulating physiology or development of a cell or tissue culture cells comprising introducing into such cell an agonist or antagonist of an LP (LP231, LP272, LP285, or LP357).

### DETAILED DESCRIPTION OF THE PREFERRED EMBODIMENTS

I. General

It is to be understood that this invention is not limited to the particular compositions, methods, and techniques described herein, as such compositions, methods, and techniques may, of course, vary. It is also to be understood that the terminology used herein is for the purpose of describing particular embodiments only, and is not intended to limit the scope of the present invention which is only limited by the appended claims.

As used herein, including the appended claims, singular forms of words such as “a,” “an,” and “the” include, e.g., their corresponding plural referents unless the context clearly dictates otherwise. Thus, for example, reference to “an organism” includes, e.g., one or more different organisms, reference to “a cell” includes, e.g., one or more of such cells, and reference to “a method” include, e.g., reference to equivalent steps and methods known to a person of ordinary skill in the art, and so forth. Unless otherwise defined, all technical and scientific terms used herein have the same meaning as commonly understood by a person of ordinary skill in the art to which this invention belongs. Although methods and materials similar or equivalent to those described herein can be used to practice or test the present invention, suitable methods and materials are described below. All publications, patent applications, patents, and other references discussed herein are provided solely for their disclosure before the filing date of the present application. Nothing herein is to be construed as an admission that the invention is not entitled to antedate any such disclosure by virtue of its prior invention. All publications, patent applications, patents, and other references mentioned herein are incorporated by reference in their entirety for the teachings for which they are cited (as the context clearly dictates), including all figures, drawings, pictures, graphs, hyperlinks, and other form of browser-executable code.

Polynucleotide sequences encoding an LP of the present invention are analyzed with respect to the tissue sources from which they were derived. Various cDNA library/tissue information described herein is found in the cDNA library/tissues of the LIFESEQ GOLD™ database (Incyte Genomics, Palo Alto Calif.) which corresponding information is incorporated herein by reference. Generally, in the LIFESEQ GOLD™ database a cDNA sequence is derived from a cDNA library constructed from a primate, (e.g., a human tissue). Each tissue is generally classified into an organ/tissue category (such as, e.g., cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract). Typically, the number of libraries in each category is counted and divided by the total number of libraries across all categories. Results using the LIFESEQ GOLD™ database reflect the tissue-specific expression of cDNA encoding an LP of the present invention. Additionally, each LP sequence of the invention is also searched via BLAST against the UniGene database. The UniGene database contains a non-redundant set of gene-oriented clusters. Each UniGene cluster theoretically contains sequences that represent a unique gene, as well as related information such as the tissue types in which the gene has been expressed and map location.

Particularly interesting portions, segments, or fragments of LP's of the present invention are discovered based on an analysis of hydrophobicity plots calculated via the “GREASE” application, which is a computer program implementation based on the Kyte-Doolittle algorithm (J. Mol. Biol. (1982) 157:105-132) that calculates a hydropathic index for each amino acid position in a polypeptide via a moving average of relative hydrophobicity. A hydrophilicity plot is determined based on a hydrophilicity scale derived from HPLC peptide retention times (see, e.g., Parker, et al., 1986 Biochemistry 25:5425-5431). Another hydrophobicity index is calculated based on the method of Cowan and Whittaker (Peptide Research 3:75-80; 1990). Antigenic features of LPs are calculated based on antigenicity plots (such as, e.g., via algorithms of: Welling, et al. 1985 FEBS lett. 188:215-218; the Hopp and Woods Antigenicity Prediction (Hopp & Woods, 1981 Proc. Natl. Acad. Sci., 78, 3824); the Parker Antigenicity Prediction (Parker, et al. 1986 Biochemistry, 25, 5425); the Protrusion Index (Thornton) Antigenicity Prediction (Thornton, et al. 1986 EMBO J., 5, 409); and the Welling Antigenicity Prediction (Welling, et al. 1985 FEBS Letters.188, 215)). Particularly interesting secondary LP structural features (e.g., such as a helix, a strand, or a coil) are discovered based on an application which is a computer implementation program based on the Predator (Frishman, and Argos, (1997) Proteins, 27, 329-335; and Frishman, D. and Argos, P. (1996) Prot. Eng., 9, 133-142); GOR IV (Methods in Enzymology 1996 R. F. Doolittle Ed., vol. 266, 540-553 Garnier J, Gibrat J-F, Robson B); and Simpa96 (Levin, et al., J FEBS Lett 1986 September 15;205(2):303-308) algorithms. One of skill in the art can use such programs to discover such secondary structural features without undue experimentation given the sequences supplied herein.

Features of LP NO: 1 (LP231)

LP231 is a novel secreted polypeptide (SEQ ID NO: 2) that exhibits (see Table 1 below) amino acid sequence similarity and/or identity and a domain architecture to a distinct family of proteins that are generally characterized as comprising collagenous helical structures at their amino portions and a globular domain at their carboxy portions (see, e.g., Prockop & Kivirikko 1995 Ann. Rev. Biochem 64:403-434). Examples of members of this family include: the C1q A, B, and C chains of the complement C1q complex; collagen alpha 1(X); lung surfacant proteins SP-A and SP-D; mannan binding protein; hibernation proteins HP-20, HP-25, and HP-27; AdipoQ/ACRP30; and cerebellins. The sequence characteristics of LP231 suggest that it is a newly discovered primate (e.g., human) member of this protein family. Furthermore, it has recently been shown that the three-dimensional structure of a member of this family—ACRP30—is superimposable onto the three-dimensional structure of tumor necrosis factor proteins CNF's) since, in spite of the overall low sequence similarity between these distinct sequences, they display ten-strand jelly-roll fold topology due to the conservation of key amino acid residues (Shapiro & Scherer 1997 Current Biology 8:335-338). These findings suggest that ACRP30-like proteins and TNFs also define a family—the Complement C1q/Tumor Necrosis Factor (NF) family—which have similar functions and modes of action due to the similarity of their higher-order structural features; features brought about via the conservation of key amino acid residues among the sequences of this family. LP231 also exhibits similarly conserved key amino acid residues to those key amino acid residues critical for bequeathing the superimposable higher-order structural topology that TNFs and ACRP30 share (see Table 1 below).

Like many complement proteins, TNF alpha is produced in response to infection and plays a variety of roles, such as for example, in: inflammation, cell proliferation, cell death, immunity, and energy homeostasis—where it is implicated in cachexia, obesity, and insulin resistance (Hotamisligil & Spiegelman 1994 Diabetes 43:1271-1278; Uysal, et al. 1997 Nature 389:610-614). TNF alpha also regulates the expression levels of some downstream components of the complement system. TNF alpha is also a major secretory product of adipocytes. Similar activities have been observed for C1q family proteins such as ACRP30 and Hib27. Given the presence of the key conserved amino acid residues between LP231, ACRP30, and TNFs, it is likely that LP231 is also a member of the Complement C1q/Tumor Necrosis Factor (TNF) family with similar functionalities due to the possession of a similarly conserved higher-order structural topology. Moreover, LP231's sequence similarity to the C1q-B chain of C1q reinforces this suggestion since members of the Complement C1q/Tumor Necrosis Factor (TNF) family are also known to play a role in role in inflammation, cell proliferation, cell death, immunity, and energy homeostasis processes. Specifically, LP231 exhibits a collagenous-like region at its amino portion that is comprised of a repeated number of Gly-Xaa-Yaa motifs. Such repeats are characteristic of collagenous regions of, for example, the atypical collagens. Characteristically, such Gly-Xaa-Yaa repeats are predicted to form collagen-like triple helices via multimerization with other proteins. Some proteins with Gly-Xaa-Yaa repeats multimerize (often in the form of trimers) by forming stable collagen triple-helical and coiled-coil type structures. For example, in C1q, collagen-like domains containing Gly-Xaa-Yaa repeats form triple-helical collagen-like structures that are held together by both covalent and non-covalent bonds. LP231 sequence similarity to the C1qB-chain of the C1q complex suggests that it may also form multimers. In its carboxy-portion, LP231 exhibits a C1q globular-like domain similar to the globular domain of the C1q B-chain of the C1q complex. The distinctive globular domain of the C1q family of proteins is situated at the carboxy end of a collagen ‘stalk.’ The C1q-globular-like domain is found in the C-terminal ends of secreted (or membrane-bound) vertebrate proteins, which typically are short-chain collagens and/or collagen-like molecules (Smith, et al. 1994 Biochem. J. 301:249-256; Brass, et al. 1992 FEBS Lett. 303:126-128; Petry, et al. 1992 Eur. J. Biochem. 209:129-134). C1q is part of the classical pathway of the complement component of the immune system. C1q interacts with aggregated IgGs via its globular domains and initiates complement cascade by proteolytically activating factors C2 and C4. Given the similarity of LP231 to the C1qB-chain and the possession by LP231 of both collagenous-like and globular-like domains, an LP231 as described herein is also likely to participate in complement-mediated processes or in complement-related diseases, conditions, and/or syndromes.

LP231 also exhibits similarity to mouse Gliacolin (which is expressed in glial cells) and C1q related factor, which is expressed in areas of the brain involved in motor function (Berube, et al. 1999 Brain Res Mol Brain Res 63(2):233-40). Complement-mediated conditions have been implicated in neural states or disease such as traumatic brain injury (Kaczorowski, et al. 1995J. Cereb. Blood Flow Metab. 15:860-864), myasthenia gravis (Piddlesden, et al. 1996 J. Neuroimmunol. 71:173-177), encephalomyelitis (Piddlesden, et al. 1994J. Immunol. 152:5477-5484), and Guillian-Barré syndrome (Jung, et al. 1995 Neuroscience Letters 200:167-70) suggesting that LP231 may also play a similar role in the nervous system via, for example, a complement-mediated process. It has been discovered that LP231 nucleic acid sequence (SEQ ID NO: 1) is expressed in the following number of LIFESEQ GOLD™ database tissue and cDNA libraries: Germ Cells 1/5, and the Nervous System 6/231. Using Western blot techniques, LP231 has been discovered in the following human cells and/or tissues: the glomeruli, tubular epithelial cells, and interstitium of the kidney; the epithelium and stroma of the prostate; the stroma of the ovary; in hepatocytes, kupffer cells, and biliary duct epithelium of the liver; in the myocardium and endocardium of the heart; in alveolar epithelial cells and macrophages of the lung; in the intima, media, and adventitia of blood vessels; in the trabeculae and red pulp of the spleen; in myocytes of skeletal muscle; in islet and acinar cells of the pancreas; cells of the cerebrum and cerebellum of the CNS; in villous and crypt epithelial cells of the gut; in ductal and epithelial cells of the breast; in the cortex and medulla of the thymus; in adipocytes and in neoplastic cells (e.g., neoplastic cells of the colon, prostate, ovary, and breast).

LP231 nucleic acid sequence has been localized to the 2q13 region of human chromosome number 2. Moreover, the following diseases, conditions, syndromes, disorders, or pathological states have also been mapped to this region of the human genome: hypohidrotic ectodermal dysplasia, which results in abnormal morphogenesis of teeth, hair, and sweat glands (Monreal et al. 1999 “Mutations in the human homologue of mouse dl cause autosomal recessive and dominant hypohidrotic ectodermal dysplasia” Nature Genet. 22:366-369); distal interphalangeal joint osteoarthritis, which represents a specific form of osteoarthritis (Leppavuori, et al 1999 “Genome scan for predisposing loci for distal interphalangeal joint osteoarthritis: evidence for a locus on 2q” Am. J. Hum. Genet. 65: 1060-1067), permanent congenital hypothyroidism of thyroid dysgenesis involving the PAX8 gene (Macchia, et al 1998 “PAX8 mutations associated with congenital hypothyroidism caused by thyroid dysgenesis” Nature Genet. 19:83-86), mental retardation (Kumada, et al. 1990 “Autosomal fragile site at 2q13 in a proband with mental retardation” Hiroshima J Med Sci 39(1):19-21), juvenile nephronophthisis (Hildebrandt, et al., 1996 “Physical mapping of the gene for juvenile nephronophthisis (NPH1) by construction of a complete YAC contig of 7 Mb on chromosome 2q13” Cytogenet Cell Genet ;73(3):235-9; Konrad, et al. 1996 “Large homozygous deletions of the 2q13 region are a major cause of juvenile nephronophthisis” Hum Mol Genet 5(3):367-71); psychomotor retardation (Lacbawan, et al. 1999 “Rare interstitial deletion (2) (p11.2p13) in a child with pericentric inversion (2)(p11.2q13) of paternal origin” Am J Med Genet Nov 19;87(2):139-42); and thrombophilia due to protein C dysfunction (Patracchini, et al 1989 “Sublocalization of the human protein C gene on chromosome 2q13-q14” Hum. Genet. 81: 191-192; Berdeaux, et al “Dysfunctional protein C deficiency (type II): a report of 11 cases in 3 American families and review of the literature” Am. J. Clin. Path. 99: 677-686, 1993). Accordingly, an isolated and/or recombinant molecule comprising LP231 nucleic acid sequence meets the statutory utility requirement of 35 U.S.C. §101 since such a molecule can be used, for example, to hybridize near a nucleic acid sequence associated with one or more of the above stated diseases, conditions, syndromes, disorders, or pathological states and thus serve as a marker for such a disease gene.

Table 1: Primate, e.g., human, LP231 polynucleotide sequence (SEQ ID NO: 1) and corresponding polypeptide (SEQ ID NO: 2). The ORF for LP231 is 1-864 bp (with the start (ATG) and stop codons (TAG) identified in bold typeface and underlined. In case the numbering is misidentified herein, one skilled in the art could easily determine the open reading frame without undue experimentation given the teachings herein.

LP231 DNA sequence (864 bp) (ORF=1-864):

LP231 (start (atg) and stop (tga) codons are indicated in bold typeface and underlined).

ATGCCGCTCGGGCTGCTCATCGCCGTCCCGCTGCTGCTQCAGGCGGCGCCCCGAGGCGCCGCGCACTATGAG
ATGATGGGCACCTGCCGCATGATCTGCGACCCTTACACTGCCGCGCCCGGCGGGGAGCCCCCGGGTGCAAAG
GCGCAGCCACCCGGACCCAGCACCGCCGCCCTGGAAGTCATGCAGGACCTCAGCGCCAACCCTCCTCCTCCT
TTCATCCAGGGACCCAAGGGCGACCCGGGGCGACCGGGCAAGCCAGGGCCGCGGGGGCCCCCTGGAGAGCCG
GGCCCGCCTGCACCCAGGGGCCCTCCGGGAGAGAAGGGCGACTCGGGGCGGCCCGGGCTGCCAGGGCTGCAA
CTGACGGCGGGCACGGCCAGCGGCGTCGGGGTGGTGGGCGGCGGGGCCGGGGTAGGTGGCGATTCCGAGGGT
GAAGTGACCAGTGCGCTGAGCGCCACCTTCAGCGGCCCCAAGATCGCCTTCTATGTGGGTCTCAAGAGCCCC
CACGAAGGCTATGAGGTGCTGAAGTTCGATGACGTGGTCACCAACCTCGGCAATCACTATGACCCCACCACG
GGCAAGTTCAGCTGCCAGGTACGCGGCATCTACTTCTTCACCTACCACATCCTCATGCGCGGCGGCGACGGC
ACCAGCATGTGGGCGGACCTCTGCAAGAACGGOCAGGTCCGGGCCAGCGCCATTGCACACGACGCCGACCAG
AACTACGACTACGCCAGTAACAGCGTGGTGCTGCACTTGGATTCAGGGGACGAAGTGTATGTGAAGCTGGAT
GGCGGGAAGGCTCACGGAGGCAATAATAACAAGTACAGCACGTTCTCGGGCTTTCTTCTGTACCCGGATTAG

LP231 Full-Length Sequence (287 aa):

LP231 (SEQ ID NO: 2) The underlined portion indicates a predicted signal sequence (Met-1 to Ala-15). A predicted SP cleavage site is between Ala-15 and Ala-16 indicated as follows: 1 MALGLLIAVPLLLQA\hat{ }AP 17. An LP encompassed herein includes full-length forms encoded by an ORF disclosed herein, as well as any mature forms therefrom. Such a mature LP could be formed, for example, by the removal of a signal peptide and/or by aainopeptidase modification. All forms of LP231 such as, both precursor and activated forms are encompassed herein. Further, as used herein, a “mature” LP encompasses, e.g., post-translational modifications other than proteolytic cleavages (such as, e.g., by way of a non-limiting example, glycosylations, gamma-carboxylations, beta-hydroxylations, myristylations, phosphorylations, prenylations, acylations, and sulfations). Such variants are also encompassed by an LP of the present invention. Further, an LP of the invention encompasses all fragments, analogs, homologs, and derivatives of an LP described herein, thus the invention encompasses both LP precursors and any modified versions (such as, e.g., by post-translational modification) of an LP encoded by an LP nucleic acid sequence described herein

MALGLLIAVPLLLQAAPRGAAHYEMMGTCRMICDPYTAAPGGEPPGAKAQPPGPSTAALEVMQDLSANPPPP
FIQGPKGDPGRPGKPGPRGPPGEPGPPGPRGPPGEKGDSGRPGLPGLQLTAGTASGVGVVGGGAGVGGDSEG
EVTSALSATFSGPKIAFYVGLKSPHEGYEVLKFDDVVTNLGNHYDPTTGKFSCQVRGIYFFTYHILMRGGDG
TSMWADLCKNGQVRASAIAQDADQNYDYASNSVVLHLDSGDEVYVKLDGGKAHGGNNNKYSTFSGFLLYPD

An LP231 Mature Sequence (272aa):

A predicted mature LP231 sequence is as follows below. Mature LP231 has a C1q like architecture that can be divided grossly into an amino-wards collagenous-like portion consisting of about 21 Gly-Xaa-Yaa repeats (indicated below by single underlining) and a carboxy-wards globular-like domain portion indicated below by double underling.

APRGAAHYEMMGTCRMICDPYTAAPGGEPPGAKAQPPGPSTAALEVMQDLSANPPPPFIQGPKGDPGRPGK
PGPRGPPGEPGPPGPRGPPGEKGDSGRPGLPGLQLTAGTASGVGVVGGGAGVGGDSEGEVTSALSATFSGP
KIAFYVGLKSPHEGYEVLKFDDVVTNLGNHYDPTTGKFSCQVRGIYFFTYHILMRGGDGTSMWADLCKNGQ
VRASAIAQDADQNYDYASNSVVLHLDSGDEVYVKLDGGKAHGGNNNKYSTFSGFLLYPD*

Comparison of the Collagen-like Domains of LP231 and C1q

A BLOSUM62 amino acid substitution matrix was used to conduct a PILEUP sequence alignment (Henikoff and Henikoff 1992 Proc. Natl. Acad. Sci. USA 89: 10915-10919) of the collagenous-like domains of C1q and LP231. Note the similar number of Gly-Xaa-Yaa repeats between the sequences. Collagen: domain 1 of 1, from 61 to 120: score 22.3, E=0.00022

| C1q | | *->GppGppGppGppGppGppGppGpaGapGppGppGepGpPGppGppGp |
|---|---|---|
| | | Gp+G pG+pG pGp+GppG+pGp+G++GppG +G++G+PG pG |
| LP231cpart | 61 | GPKGDPGRPGKPGPRGPPGEPGPPGPRGPPGEKGDSGRPGLPGLQLT 107 |
| C1q | | pGppGapGapGpp<-* |
| | | +G + G G |
| LP231cpart | 108 | AGTASGVGVVGGG 120 |

Comparison of the Globular-like Domains of LP231 and C1q

A BLOSUM62 amino acid substitution matrix was used to conduct a PILEUP sequence alignment (Henikoff and Henikoff 1992 Proc. Natl. Acad. Sci. USA 89: 10915-10919) of the globular-like domains of C1q and LP231.

C1q: domain 1 of 1, from 10 to 134: score 115.9, E=7.6e-31

| C1q: domain | | *->AFtvirstnrpPaEmsnpgqpViFdeVLyNqqghYdpaTGkFtCkvP | |
|---|---|---|---|
| | | AF v + + p + ++ +Fd V++N ++hYdp TGkF+C v | |
| LP231 | 10 | AFYVGLKS--PHE----GYEVLKFDDVVTNLGNHYDPTTGKFSCQVR | 50 |
| C1q: domain | | GlYyFsFhvsskg...tRqnvcVsLmrSSrngvrqkVmefcdeyakgtyq | |
| | | G+Y F++h+ +g+++t + + L + ++vr ++ + d++++ y+ | |
| LP231 | 51 | GIYFFTYHILMRGgdgT--SMWADLCK--NGQVR-ASAIAQDADQN--YD | 93 |
| C1q: domain | | vaSGGavLqLrqGDrVWLelddkqtngllggegvhSvFSGFLl<-* | |
| | | aS+++vL L GD+V+++ld ++++ g ++S+FSGFLl | |
| LP231 | 94 | YASNSVVLHLDSGDEVYVKLDGGKAH--GGNNNKYSTFSGFLL 134 | |

Camparison of LP231 with C1q-B

A BLOSUM62 amino acid substitution matrix was used to conduct a PILEUP sequence alignment (see, Henikoff and Henikoff 1992 Proc. Natl. Acad. Sci. USA 89: 1091510919) of LP231 and the human complement component 1, q subcomponent, beta polypeptide.

C1q=Complement component 1, q subcomponent, beta polypeptide.

| | |
|---|---|
| C1q | MVLLLVILIPVLVSSAG-TSAHYEMLGTCRMVCDPYGGT---KAPSTAATPDRG------ |
| LP231 | MALGLLIAVPLLLQAAPRGAAHYEMMGTCRMICDPYTAAPGGEPPGAKAQPPGPSTAALE |
| | *.* *:* :*:*:.:* :*****:*****:*** .: :.*.: * * |
| C1q | LMQSL-----PTFIQGPKGEAGRPGKAGPRGPPGEPGPPGPVGPPGEKGEPGRQGLP--- |
| LP231 | VMQDLSANPPPPFIQGPKGDPGRPGKPGPRGPPGEPGPPGPRGPPGEKCDSGRPGLPGLQ |
| | :**.* *.*******:.*****.************** *******:.** *** |
| C1q | ------------GPPGAPGLNAAGAISAATYSTV--PKIAFYAGLKRQHEGYEVLKFDDV |
| LP231 | LTAGTASGVGVVGGGAGVGGDSEGEVTSALSATFSGPKIAFYVGLKSPHEGYEVLKFDDV |
| | * .. * :: * :::* :*. ******.*** ************ |
| C1q | VTNLGNHYDPTTGKFTCSIPGIYFFTYHVLMRGGDGTSMWADLCKNNQVRASAIAQDADQ |
| LP231 | VTNLGNHYDPTTGKFSCQVRGIYFFTYHILMRGCDGTSMWADLCKNGQVRASAIAQDADQ |
| | ***************:*.: ********:*****************.************* |
| C1q | NYDYASNSVVLHLEPGDEVYIKLDGGKAHGGNNNKYSTFSGFIIYAD |
| LP231 | NYDYASNSVVLHLDSGDEVYVKLDGGKAHGGNNNKYSTFSGFLLYPD |
| | *************:.*****:*********************::*,* |

Comparison of LP231 with ACRPs

A BLOSUM62 amino acid substitution matrix was used to conduct a PILEUP sequence alignment (see, Henikoff and Henikoff 1992 Proc. Natl. Acad. Sci. USA 89: 1091510919) of LP231 and ACRP proteins.

>gs:AAB30232 Human adipocyte complement related protein homologue zacrp2.

Length=285

Score=118 bits (292), Expect=2e-26

Identities=85/272 (31%), Positives=127/272 (46%), Gaps=34/272 (12%)

| | | | |
|---|---|---|---|
| LP231: | 27 | GTCRMICD-PYTAAPGGEPPGAKAQPPGPSTA-------ALEVMQDLSANPPPPFIQGPK | 78 |
| | | G+ +++C P P G PPGA PGPS + + +GP | |
| Sbjct: | 30 | GSPQLVCSLPGPQGPPG-PPGA----PGPSGMMGRMGFPGKDGQDGHDGDRGDSGEEGPP | 84 |
| Query: | 79 | GDPGRPGKPGPRGPPGEPGPPGPRGPPGEKGDSGRPGLPGLQLTAGTASGVGVVGGGAGV | 138 |
| | | G G GKPGP+G G G GPRGP G G G+ G PG + G G+ G + | |
| Sbjct: | 85 | GRTGNRGKPGPKGKAGAIGRAGPRGPKGVNGTPGKHGTPGKKGPKGKKGEPGLPGPCSCG | 144 |
| Query: | 139 | GGDSEGEVTSALSATFSGPKIAPYVGLKSPHEGYEVLKFDDVVTNLGNHYDPTTGKFSCQ | 198 |
| | | G ++ + A++ ++ ++ +KFD ++ N G HY+ ++GKF C | |
| Sbjct: | 145 | SGHTKSAFSVAVTKSYPRERLP--------------IKFDKILMNEGGHYNASSGKPVCG | 190 |
| Query: | 199 | VRGIYFFTYHILMRGGDGTSMWADLCKNGQVRASAIAQDADQNYDYASNSVVLHLDSGDE | 258 |
| | | V GIY+FTY I + + L NGQ R + N+D AS S +L L GDE | |
| Sbjct: | 191 | VPGIYYFTYDITLA---NKHLAIGLVHNGQYRIRTFDANTG-NHDVASGSTILALKQGDE | 246 |
| Query: | 259 | VYVKLDGGKAHGGNNNKY---STFSGFLLYPD 287 | |
| | | V++++ + +G + Y S F+GFL+Y D | |
| Sbjct: | 247 | VWLQIFYSEQNGLFYDPYWTDSLFTGFLIYAD 278 | |

>gs:AAW09108 Human adipocyte complement related protein Acrp30.

Length=244

Score=116 bits (288), Expect=7e-26

Identities=84/243 (34%), Positives=118/243 (47%), Gaps=37/243 (15%)

| | | | |
|---|---|---|---|
| Query: | 52 | PGPSTAALEVMQDLSANPPPPFIQGPKGDPGRPGKPGPRGPPGEPGPPGPRGPPGEKGDS | 111 |
| | | P P A M + +P G G PGR G+ G G GE G PG GP G+ G++ | |
| Sbjct: | 30 | PLPKGACTGWMAGIPGHP------GHNGAPGRDGRDGTPGEKGEKGDPGLIGPKGDIGET | 83 |
| Query: | 112 | GRPGLPGLQLTAGTASGVGVVGGGAGVGGDSEGEVTSALSATFSGPKIAFYVGLKS---- | 167 |
| | | G PG G + G G G GA V + AF VGL++ | |
| Sbjct: | 84 | GVPGAEGPRGFPGIQGRKGEPGEGAYV------------------YRSAFSVGLETYVTI | 125 |
| Query: | 168 | PHEGYEVLKFDDVVTNLGNHYDPTTGKFSCQVRGIYFFTYHILMRGGDGTSMWADLCKNG | 227 |
| | | P+ ++F + N NHYD +TGKF C + G+Y+F YHI + D + L K | |
| Sbjct: | 126 | PN---MPIRFTKIFYNQQNHYDGSTGKFHCNIPGLYYFAYHITVYMKD---VKVSLFKKD | 179 |
| Query: | 228 | QVRASAIAQDADQNYDYASNSVVLHLDSGDEVYVKLDG-GKAHG--GNNNKYSTFSGFLL | 284 |
| | | + Q + N D AS SV+LHL+ GD+V++++ G G+ +G +N+ STF+GFLL | |
| Sbjct: | 180 | KAMLFTYDQYQENNVDQASGSVLLHLEVGDQVWLQVYGEGELNGLYADNDNDSTFTGFLL | 239 |
| Query: | 285 | YPD 287 | |
| | | Y D | |
| Sbjct: | 240 | YHD 242 | |

Structural Comparison of LP231 with TNFs and the Globular Domains of C1q and ACRP30

A structure-based sequence alignment between TNFs (alpha (a), beta (b), and CD40) and the globular C1q domains of ACRP30, C1q, and LP231 is indicated below. The alignment is based upon the alignments of Shapiro and Scherer, 1998 Curr. Bio. 8:335-338 and Karpusas, et al. 1995 Structure 3:1031-1039. Key conserved amino acid residues, which are proposed to be critical to the adoption of the superimposable three-dimensional higher-order structures of the proteins, are indicated with a diamond symbol (♦) for the residues that are identical in all proteins, and are indicated with a cloverleaf symbol ( [symbol] ) for those residues those that are conserved in five out of the six proteins. The ten beta-strand regions for ACRP30 and CD40L (A, A′, B, B′, C, D, E, F, G, and H) are indicated above the alignment (see Shapiro and Scherer, 1998 Curr. Bio. 8:335-338 for the three-dimensional topology).

Shapiro and Scherer (supra) superimposed the three-dimensional crystal structure of ACRP30 onto the three known structures of molecules from the TNF family (TNF alpha, TNF beta, and CD40 ligand (CD40L)). Shapiro and Scherer used the superpositions to generate structure-based sequence alignments which revealed key amino acid residues conserved between these proteins. LP231 has been added to the alignment to indicate it also possess such conserved residues. Each of the ten beta strands of ACRP30 (A, A′, B, B′, C, D, E, F, G, and H) can be simultaneously superposed with the ten beta strands of each TNF molecule; the relative positions and lengths of these beta strands are almost identical between ACRP30 and the TNFs. Four residues are conserved throughout both the C1q and TNF families: Tyr161, Gly159, Phe237, and Leu242 (ACRP30 numbering). These same key residues are conserved in LP231. Each of these residues is important in the packing of the protomer's hydrophobic core in both the C1q and TNF families. The structures of the hydrophobic cores of globular C1q domains and TNFs are similar; side chains in analogous positions often have similar orientations.

| | |
|---|---|
| | A A′ B′ B C |
| | ------→ ---→ ---→ ---→ ----------→ |
| ACRP30 | ATMYRSAFSVGLETRVTV-PNVPIRFTKIFYNQQN-KYDGSTGKFYCNIPGLYYFSYNITV------ |
| C1QA | GATQNVAFSALRTINSPLR-PNQVIRFEKVITNANE-NKPRNGKFTCKVPGLYYFTYNASS------ |
| TNF a | RTPSDKP-VAHVVANPQAEGQ-LQWLNRRANALLANGV-ELRD--NQLVVPSEGLYLIYSQVLFKGQGCP |
| TNF b | ---TLDP-AAHLIGDPSKQNS-LLWRANTDRAFLQDGF-SLSN--NSLLVPTSGIYFVYSQVVFSGKAYS |
| CD40L | -GDQNPQIAAHVISEASSKTTSVLQW-AEKGYYTMSNNLVTLENGKQLTVKRQGLYYIYAQVTF---CSN |
| LP231 | LSATFSGPKIAFYVGLKSPH-EGYEVLKFDDVVTNLGNHYDPTTGKFSCQVRGIYFFTYHILMRGGDGTSMWADLCK |
| | ♦ ♦ |
| | D E F G |
| | --------→ --------→ ---------→ -----→ |
| ACRP30 | TMKDVKVSLPNK-------DKAVLFTYDQYQ----------EKNVDQASGSVLLHLEVGDQVWLQVY |
| C1QA | -RGNLCVNLVRK-------GRDSMQKVVIFC----------DYAQNIFQVITGGVVLKLEQEEVVEL |
| TNF a | ----STHVLLTHTISRIAVSYQTKVNLLSAIKSPCQRETPEGAEAKPWYEPIYLGGVFQLEKGDRLSAEIN |
| TNF b | PKATSSPLYLAHEVQLFSSQYPFHVPLLSSQKHVY------PGLQEPWLHSMYHGAAFQLTQGDQLSTHTD |
| CD40L | REASSQAPFIASLCLKSPG-RFERILLRAANTHS-------SAKPCGQQSIHLGGVFELQPGASVFVNVT |
| LP231 | NGQVRGIYFFTYHILMRGGDGTSMWADLCKNGQVR--ASAIAQDADQNYDYASNSVVLHLDSGDEVYV--- |
| | [symbol] [symbol] [symbol] |
| | H |
| | ---------→ |
| ACRP30 | GDGDENGLYADNVNDSTFTGFLLYMDTN |
| C1QA | QATDKNSLLGIEGANSIFTGFLLFFNDDA |
| TNF a | RPDYLDFAESGQV---YFGIIAL |
| TNF b | TDGIPHLVLSPSTV--FFGAFAL |
| CD40L | VTDPSQVSHGTGFT--SFGLLKL |
| LP231 | KLDGGKAHGGNNNKYSTFSGFLLYPD |
| | ♦ ♦ |

Comparison of LP231 with C1q Signature Domains of Precerebellins

A BLOSUM62 amino acid substitution matrix was used to conduct a PILEUP sequence alignment (see, Henikoff and Henikoff 1992 Proc. Natl. Acad. Sci. USA 89: 10915-10919) of LP231 and the globular C1q signature domains (Koide, et al. 2000 J. Biol. Chem. 275 (36), 27957-27963) of precerebellin related proteins (Pang, et al 2000 Jour Neurosci 20(17):6333-39). C1q is a subunit of the C1 enzyme complex that activates the serum complement system. The globular C1q signature domain or aromatic zipper is a protein consensus motif about 130 amino acids in length that was defined in C terminus location of C1q (Smith, et al 1994 Biochem J 301:249-256).

Typically, the presence of such a domain indicates that a protein possessing it will undergo multimeric binding (either of a homo- or heteromeric nature). For example, within the atypical collagens, the globular C1q signature domain is responsible for the initial assembly of trimeric complexes that brings subunits into correct alignment, thereby permitting the single collagen domain in each subunit to associate in a triple helix (Brass et al., 1991 Biochem Soc Trans 19:365S). In collagen X, the multimer is a trimer consisting of three identical chains, however, in other instances, such as C1q, the multimer complex is composed of three distinct subunits. Therefore, individual globular C1q signature domain head groups not only align protomers but they also discriminate different molecular entities to ensure the correct subunit stoichiometry in a multimeric complex.

As shown below, the portion of LP231 from about Ser-151 to about Asp-287 (LP231ZIP for the aromatic zipper portion of LP231) exhibits sequence that conforms to a globular C1q signature-like domain suggesting that LP231 may also form multimeric complexes.

HC1QAROMZ=the aromatic zipper portion of human cerebellin; M2C1QAROMZ=the aromatic zipper portion of mouse cerebellin3; and MC1QAROMZIP=the aromatic zipper portion of mouse cerebellin precursor. Diamond (♦) and circle symbols (*) indicate respectively, identical and highly conserved amino acid residues found in the globular C1q signature domain (Smith, et al 1994 Biochem J 301:249-256).

| | |
|---|---|
| | 1 50 |
| HC1QAROMZ | ˜˜˜SGSAKVA FSAIRSTNHE PS.EMSNRTM .IIYFDQVLV NIGNNFDSER |
| M2C1QAROMZ | ˜˜˜˜˜SAKVA FSAIRSTNHE PS.EMSNRTM .IIYFDQVLV NIGNNFDSER |
| MC1QAROMZIP | ˜˜˜APPGRVA FAAVRSHHHE PAGETGNGTS GAIYFDQVLV NEGEGFDRTS |
| LP231ZIP | SATFSGPKIA FYVGLKSPHE ........GY EVLKFDDVVT NLGNHYDPTT |
| | 1 ♦ * ♦ ♦ * 50 |
| | 51 100 |
| HC1QAROMZ | STFIAPRKGI YSFNFHV.VK VYNRQTIQVS LMLNGWPVIS AFAGDQDVTR |
| M2C1QAROMZ | STFIAPRKGI YSFNFHV.VK VYNRQTIQVS LMLNGWPVIS AFAGDQDVTR |
| MC1QAROMZIP | GCFVAPVRGV YSPRFHV.VK VYNRQTVQVS LMLNTWPVIS AFANDPDVTR |
| LP231ZIP | GKPSCQVRGI YFFTYHILMR GGDGTSMWAD LCKNGQVRAS AIAQDADQNY |
| | 1 ♦ ♦ ♦ ♦ * ♦ 50 |
| | 101 145 |
| HC1QAROMZ | EAASNGVLIQ MEKGDRAYLK LERGNLMGG. .WKYSTFSGF LVFPL |
| M2C1QAROMZ | EAASNGVLIQ MEKGDRAYLK LERGNLMGG. .WKYSTFSGF LVFPL |
| MC1QAROMZIP | EAATSSVLLP LDPGDRVSLR LRRGNLLGG. .WKYSSFSGF LIFPL |
| LP231ZIP | DYASMSVVLH LDSGDEVYVK LDGGKAHGGN NNKYSTFSGF LLYPD |
| | 1 * * ♦ * ♦ ♦* ♦* 50 |

Particularly interesting portions or fragments of the full length LP231 polypeptide include, e.g., a discovered putative signal peptide-like sequence from Met-1 to Ala-15 (MALGLLIAVPLLLQA). An additionally interesting portion of LP231 is a C1q-like portion from about Ala-160 to about Asp-287 (AFYVGLKSPHEGYEVLKFDDVVTNLGNHYDPTTGKFSCQVRGIYFFTYHILMRGGDGTSMWADLCKNGQVR ASAIAQDADQNYDYASNSVVLHLDSGDEVYVKLDGGKAHGGNNNKYSTFSGFLLYPD). Other embodiments of the LP231 C1q-like portion encompassed herein include LP231 fragments that have at least 1, 2, 3, 4, 5, 6, 7, 8, 9, 10, 11, 12, 13, 14, 15, 16, or 17 contiguous amino acid residues subtracted from either and/or both (or any combination thereof) the amino- and/or carboxy-end of said LP231 C1q-like portion. C1q is a subunit of the C1 enzyme complex that activates the serum complement system. C1q is composed of nine disulfide-linked dimers of A, B, and C chains that share a common structure consisting of an N-terminal non-helical region, a (triple helical) collagenous region, and a C-terminal globular head which is also called the C1q globular domain or aromatic zipper domain. The C1q globular domain consists of about 136 amino acids that form ten beta strands interspersed by beta-turns and/or loops (Smith, et al. 1994 Biochem. J. 301:249-256). The C1q-like globular domain is found in the C-terminal ends of secreted (or membrane-bound) vertebrate proteins, which, typically, are short-chain collagens and/or collagen-like molecules (Smith, et al. 1994 Biochem. J. 301:249-256; Brass, et al. 1992 FEBS Lett. 303:126-128; Petry, et al. 1992 Eur. J. Biochem. 209:129-134). Proteins exhibiting C1q architecture include, for example: Complement C1q subcomponent chains A, B and C (Efficient activation of C1 takes place on interaction of the globular heads of C1q with the Fc regions of IgG or IgM antibody present in immune complexes.); Vertebrate short-chain collagen type VIII, which is the major component of the basement membrane of corneal endothelial cells (it is composed of a triple helical domain in between a short N-terminal and a larger C-terminal globule which contains the C1q domain); Vertebrate collagen type X; Bluegill inner-ear specific structural protein, which forms a microstructural matrix within the otolithic membrane; Chipmunk hibemation-associated plasma proteins HP-20, HP-25 and HP-27 (these proteins are involved in energy homeostasis and their expression is specifically extinguished during hibernation); Human precerebellins, which are located within postsynaptic structures of Purkinje cells, probably membrane-bound. (Cerebellin is involved in synaptic activity); Rat precerebellin-like glycoprotein (a probable membrane protein where the C1q domain is located at the C-terminal extracellular extremity); Human endothelial cell multimerin (ECM), which is a carrier protein for platelet factor V/VA; and Vertebrate 30 Kd adipocyte complement-related protein (ACRP30), also known as ApM1 or AdipoQ, which is made exclusively in adipocytes and whose expression dysregulated in various forms of obesity.

The C-terminal globular domain of the C1q subcomponents and of collagen type proteins such as collagen VIII and collagen X is important both for the correct folding and alignment of the triple helix and for protein-protein recognition events (Rosenbloom, et al. 1976 J. Biol. Chem. 251:2070-2076; Engel & Prockop 1991 Annu. Rev. Biophys. Chem. 20:137-152). For collagen type X it has been suggested that the C1q domain is important for initiation and maintenance of the correct assembly of the protein (Kwan, et al 1991 J. Cell Biol. 114:597-604). There are two well-conserved regions within the C1q domain. One is a collagenous-like region at the amino-wards portion (in LP231 this collagenous-like portion is from about Phe-73 to about Ala-149 (FIQGPKGDPGRPGKPGPRGPPGEPGPPGPRGPPGEKGDSGRPGLPGLQLTAGTASGVGVVGGGAGVGGDSE GEVTSA) while the other region is an aromatic zipper or globular portion located at carboxy-wards portion (in LP231 this aromatic zipper or globular-like portion is an from about Leu-150 to about Asp-272

(LSATFSGPKIAFYVGLKSPHEGYEVLKFDDVVTNLGNHYDPTTGKFSCQ
VRGIYFFTYHILMRGGDGTSMWADLCKNGQVRASAIAQDADQNYDYASNS
VVLHLDSGDEVYVKLDGGKAHGGNNNKYSTFSGFLLYPD).

Using the C1q aromatic zipper portion, a consensus recognition pattern was developed (F-x(5)-[ND]-x(4)-[FYWL]-x(6)-F-x(5)-G-x-Y-x-F-x-[FY]) to identify proteins exhibiting a C1q domain signature. Every protein sequence in the SWISS-PROT database that is recognized as having a C1q domain can be identified using this consensus signature. LP231 is also identified by this consensus pattern further supporting its characterization as exhibiting a C1q-like domain architecture. Recently, the crystal structure of the C1q signature domain of ACRP30/AdipoQ was solved (Shapiro and Scherer, 1998 Curr. Bio. 8:335-338). As described herein, ACRP30/adipoQ protein is synthesized by adipose tissue and subsequently released into plasma. The ACRP30 crystal structure was found to have a remarkable degree of similarity to the three-dimensional structure of TNFs such as tumor necrosis factor-alpha (TNF-alpha) despite the fact that TNF-alpha is not an atypical collagen (like ACRP30 or other proteins having C1q domain signatures). Furthermore, TNF-alpha has few amino acid residues that would be classified as characteristic of a C1q signature domain. However, a sequence comparison of ACRP30, C1q A, TNF-alpha, TNF-beta, and CD40L (informed by the superimposability of the three-dimensional structures of these proteins) reveals that four key amino acid residues are conserved throughout both the C1q and TNF families (Gly159, Tyr161, Phe237, and Leu242—using the ACRP30 numbering system). Each of the key conserved amino acid residues is important in contributing to the mature three-dimensional characteristics of these proteins (Shapiro and Scherer, 1998). Analysis of such data support a conclusion that the TNF and C1q domain group of proteins are members of a C1q/TNF molecular superfamily, which has arisen by divergence from a common precursor molecule.

Applicants have discovered that these four key amino acid residues are also conserved between TNF-alpha, TNF-beta, CD40L, ACRP30, and LP231 (using the LP231 numbering system of the full-length LP231, they are: Gly201, Tyr203, Phe279, and Leu284 (see Table 1 above). Therefore, it is likely that LP231 is also a member of the C1q/TNP superfamily and possesses a similar folding topology with ten beta-strand jelly-roll features. Consequently, it is also likely that secreted LP231 (or a portion thereof), or a LP231 complex interacts with a membrane receptor to activate an intracellular signal transduction cascade in a manner analogous to TNF-alpha.

An additionally interesting portion of LP231 (identified from the Pfam database of protein domains (Bateman, et al. 2000 Nucleic Acids Research 28:263-266)) is a collegenous-like portion from about Gly-76 to about Gly-146 (GPKGDPGRPGKPGPRGPPGEPGPPGPRGPPGEKGDSGRPGLPGLQLTAGTASGVGVVGGGAGV GGDSEGEV), which exhibits multiple copies of a Gly-Xaa-Yaa repeat (Mayne & Brewton 1993 Curr Opin Cell Biol 5:883-890). Typically, the first position of the repeat is glycine, the second and third positions can be any residue but are frequently proline and hydroxyproline. Characteristically, Gly-Xaa-Yaa repeats are predicted to form collagen-like triple helices through multimerization. In some proteins possessing such repeats, the multimerization (often in the form of trimers) is likely to result from the formation of stable collagen triple-helical and coiled-coil type structures. For example, in C1q complexes, similar collagen-like domains containing Gly-Xaa-Yaa repeats form triple-helical collagen-like structures, which are held together by both covalent and non-covalent bonds. The number of Gly-Xaa-Yaa repeats in LP231 (approximately 21) is similar to the number of such repeats in proteins exhibiting sequence similarity to LP231, such as, for example, 22 such repeats in ACRP30/adipoQ and 26-29 such repeats in the C1q chains. Members of the protein family characterized by such repeats belong to the collagen superfamily. Some members of the collagen superfamily, for example, such as the atypical collagens mentioned herein, are not involved in connective tissue structure even though they share the same triple helical structure. Other interesting segments of LP231 are discovered portions of LP231 from about Thr-37 to about Gly-46 (TAAPGGEPPG); from about Ala-47 to about Ala-58 (AKAQPPGPSTAA); from about Gly-76 to about Pro-89 (GPKGDPGRPGKPGP); from about Arg-90 to about Pro-101 (RGPPGEPGPPGP); from about Arg-102 to about Gly-115 (RGPPGEKGDSGRPG); from about Thr-122 to about Val-131 (TAGTASGVGV); from about Val-132 to about Ala-149 (VGGGAGVGGDSEGEVTSA); from about Pro-157 to about Lys-166 (PKIAFYVGLK); from about Tyr-172 to about Thr-182 (YEVLKFDDVVT); from about Gly-185 to about Gln-198 (GNHYDPTTGKFSCQ); from about Val-199 to about Met-211 (VRGIYFFTYHILM); from about Cys-224 to about Ile-234 (VRGIYFFTYHILM); from about Ala-235 to about Ser-246 (AQDADQNYDYAS); from about Asp-264 to about Tyr-276 (DGGKAHGGNNNKY); from about Ile-7 to about Ala-16 (IAVPLLLQAA); from about Pro-17 to about Gly-27 (PRGAAHYEMMG); from about Asp-34 to about Pro-45 (DPYTAAPGGEPP); from about Gly-46 to about Ser-55 (GAKAQPPGPS); from about Asp-64 to about Ile-74 (DLSANPPPPFI); from about Ile-74 to about Arg-83 (IQGPKGDPGR); from about Pro-84 to about Glu-95 (PGKPGPRGPPGE); from about Pro-96 to about Gly-106 (PGPPGPRGPPG); from about Glu-107 to about Leu-116 (EKGDSGRPGL); from about Pro-117 to about Ala-126 (PGLQLTAGTA); from about Ser-127 to about Val-138 (SGVGVVGGGAGV); from about Thr-147 to about Gly-156 (TSALSATFSG); from about Asn-183 to about Gln-198 (NLGNHYDPTTGKFSCQ); from about Val-199 to about Met-211 (VRGIYFFTYHILM); from about Gln-236 to about Ser-246 (QDADQNYDYAS); from about Lys-262 to about Thr-278 (KLDGGKAHGGNNNKYST); from about Ala-15 to about Met-31 (AAPRGAAHYEMMGTCRM); from about Ile-32 to about Glu-43 (ICDPYTAAPGGE); from about Pro-44 to about Glu-60 (PPGAKAQPPGPSTAALE); from about Val-61 to about Pro-72 (VMQDISANPPPP); from about Ile-74 to about Pro-89 (IQGPKGDPGRPGKPGP); from about Arg-90 to about Gly-100 (RGPPGEPGPPG); from about Pro-101 to about Pro-117 (PRGPPGEKGDSGRPGLP); from about Gln-120 to about Gly-130 (QLTAGTASGVG); from about Val-131 to about Ser-142 (VVGGGAGVGGDS); from about Glu-143 to about Lys-158 (EGEVTSALSATFSGPK); from about Leu-165 to about Val-174 (LKSPHEGYEV); from about Leu-175 to about Leu-184 (LKFDDVVTNL); from about Gly-185 to about Val-199 (GNHYDPTTGKFSCQV); from about Gly-201 to about Met-211 (GIYFFTYHILM); from about Arg-212 to about Ala-221 (RGGDGTSMWA); from about Asp-222 to about Ala-233 (DLCKNGQVRASA); from about Ile-234 to about Ser-248 (IAQDADQNYDYASNS); from about Leu-253 to about Leu-263 (LDSGDEVYVKL); and from about Asp-264 to about Phe-279 (DGGKAHGGNNNKYSTF); whose discoveries were based on an analysis of hydrophobicity, hydropathicity, and hydrophilicity plots. Additional interesting sections of LP231 are the discovered portions of LP231 from about Ala-20 to about Met-31 (AAHYEMMGTCRM); from about Ile-32 to about Gly-41 (ICDPYTAAPG); from about Gly-42 to about Pro-51 (GEPPGAKAQP); from about Met-1 to about Pro-72 (MQDLSANPPPP); from about Phe-73 to about Gly-82 (FIQGPKGDPG); from about Arg-83 to about Pro-92 (RPGKPGPRGP); from about Pro-93 to about Arg-102 (PGEPGPPGPR); from about Gly-103 to about Gly-112 (GPPGEKGDSG); from about Arg-113 to about Gly-124 (RPGLPGLQLTAG); from about Thr-125 to about Gly-134 (TASGVGVVGG); from about Gly-135 to about Val-146 (GAGVGGDSEGEV); from about Thr-147 to about Tyr-162 (TSALSATFSGPKIAFY); from about Asn-186 to about Arg-200 (NHYDPTGKFSCQVR); from about Gly-201 to about Arg-212 (GIYFFTYHILMR); from about Gly-213 to about Lys-225 (GGDGTSMWADLCK); from about Ala-233 to about Tyr-244 (GGDGTSMWADLCK); and from about Ala-245 to about Val-259 (ASNSVVLHLDSGDEV). These fragments were discovered based on analysis of antigenicity plots. Further, particularly interesting LP231 segments are LP secondary structures (e.g., such as a helix, a strand, or a coil). Particularly interesting LP231 coil structures are the following: from about Ala-15 to about Gly-19; from about Gly-27 to about Thr-28; from about Cys-33 to about Ser-55; from about Ser-66 to about Gly-118; from about Gly-124 to about Ser-127; from about Gly-140 to about Glu-143; from about Thr-153 to about Lys-158; from about Leu-165 to about Gly-171; from about Leu-184 to about Gly-193; from about Arg-200 to about Arg-200; from about Arg-212 to about Thr-217; from about Cys-224 to about Gln-228; from about Asp-239 to about Asn-247; from about Asp-254 to about Asp-257; from about Asp-264 to about Lys-275; from about Ser-280 to about Ser-280; and from about Pro-286 to about Asp-287. Particularly interesting helix structures are from about Val-61 to about Gln-63; and from about Ile-234 to about Gln-236. Particularly interesting strand structures are from about Arg-30 to about Ile-32; from about Val-129 to about Val-132; from about Ile-159 to about Val-163; from about Glu-173 to about Leu-175; from about Tyr-203 to about Met-211; from about Ser-248 to about Leu-253; from about Glu-258 to about Leu-263; and from about Phe-282 to about Leu-283. Further encompassed by the invention are contiguous amino acid residue combinations of any of the predicted secondary structures describcd above. For example, one strand-coil-coil-helix-coil-strand motif of LP231 combines the Tyr-203 to Met-211 strand; with the Arg-212 to Thr-217 coil; with the Cys-224 to Gln-228 coil; with the Ile-234 to Gln-236 helix; with the Asp-239 to Asn-247 coil; with the Ser-248 to Leu-253 strand to form an interesting fragment of contiguous amino acid residues from about Tyr-203 to about Leu-253. In vitro solution assays can be used to identify an LP231 substrate or inhibitor. Solid phase systems can also be used to identify a substrate or inhibitor of an LP231 polypeptide. For example, an LP231 polypeptide or LP231 fusion protein can be immobilized onto the surface of a receptor chip of a commercially available biosensor instrument (BIACORE, Biacore AB; Uppsala, Sweden). The use of this instrument is disclosed, for example, by Karlsson, Immunol. Methods 145:229 (1991), and Cunningham and Wells, J. Mol. Biol. 234:554 (1993). In brief, an LP231 polypeptide or fusion protein is covalently attached, using amine or sulfhydryl chemistry, to dextran fibers that are attached to gold film within a flow cell. A test sample is then passed through the cell. If an LP231 substrate or inhibitor is present in the sample, it will bind to the immobilized polypeptide or fusion protein, causing a change in the refractive index of the medium, which is detected as a change in surface plasmon resonance of the gold film. This system allows the determination on- and off-rates, from which binding affinity can be calculated, and assessment of the stoichiometry of binding, as well as the kinetic effects of an LP231 variant. This system can also be used to examine antibody-antigen interactions, and the interactions of other complement/anti-complement pairs. Given the sequence information and knowledge of the secondary structural features of, e.g., ACRP30 and TNFs, one can easily determine how such features map onto the LP231 sequence presented herein (see, e.g., Shapiro and Scherer, 1998 Curr. Bio. 8:335-338 and references cited therein, which is incorporated by reference herein). For example, higher order structural determination can be carried out (such as, for example, crystallization) using methods known in the art. Alternatively, computer programs can be used to determine higher order structures. Such techniques are also common in the art. Additionally, commercial services are available to rapidly produce three-dimensional configurations and higher order structures using proteins produced from known primary amino acid sequences thus avoiding undue experimentation when assessing higher order structures of a sequence of interest (see, e.g., Structural GenomiX, 10505 Roselle St., San Diego, Calif. 92121).

Further encompassed herein are LP231 variants, such as, e.g., fusion proteins, such as, for example, a fusion of, for example, an LP231 globular C1q-like domain portion to another protein (e.g., such as similar to the techniques of Kishore, et al. 1998 a, b Biochem. J. 333:27-32; Mol. Immunol. 35:375 in creating a fusion protein of the globular head regions of the C1q A, B, and C chains separately.). In a particular embodiment, Applicants claim a fusion comprising at least 18, 20, 25, 30, 35, 40, 45, 50, 55, 60, 65, 70, 75, 80, 85, 90, 95, 100, 105, 110, 115, 120, 125, or 128 consecutive amino acid residues in length of LP231 from the following C1q-like domain of LP231 from about Ala-160 to about Asp-287 (AFYVGLKSPHEGYEVLKFDDVVTNLGNHYDPTTGKFSCQVRGIYFFIYHILMRGGDGTSMWADL CKNGQVRASAIAQDADQNYDYASNSVVLHLDSGDEVYVKLDGGKAHGGNNNKYSTFSGFLLYPD). In another embodiment, Applicants claim a fusion comprising at least two portions each of which is at least 12, 13, 14, 15, 16, 17,18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 63 consecutive amino acid residues of LP231 from the following C1q-like domain in length of LP231 from about Ala-1 60 to about Asp-287 (AFYVGLKSPHEGYEVLKFDDVVTNLGNHYDPTTGKFSCQVRGIYFFTYHILMRGGDGTSMWADL CKNGQVRASAIAQDADQNYDYASNSVVLHLDSGDEVYVKLDGGKAHGGNNNKYSTFSGFLLYPD). In still another embodiment, Applicants claim a fusion comprising a plurality (three or more) of portions wherein any individual single portion being at least 12, 13,14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 63 consecutive amino acid residues in length of LP231 (or any combination thereof) from the following C1q-like domain of LP231 from about Ala-160 to about Asp-287 (AFYVGLKSPHEGYEVLKFDDVVTNLGNHYDPTTGKFSCQVRGIYFFTYHILMRGGDGTSMWADL CKNGQVRASAIAQDADQNYDYASNSVVLHLDSGDEVYVKLDGGKAHGGNNNKYSTFSGFLLYPD). In still another embodiment, said plurality is four, five, six, seven, or eight said portions of any combination of contiguous lengths described herein. Not being bound by theory, it is likely that such a fragment will be useful in a fusion since this portion of LP231 maps onto the globular domain portions of C1q that have been shown to activate complement component of the classical immune system pathway (Krem, et al. 1999 Jour. Biol. Chem. 274: 28063-28066). Accordingly, such a fusion protein as encompassed herein will be able to compete for binding with native activators of complement and therefore can be useful in modulating complement-related diseases, syndromes, and/or conditions that are due to activation of complement.

No undue experimentation is required in creating and or characterizing any LP231 or LP231 variant taught herein. One factor among others that can be considered in making changes in amino acid residues of a polypeptide is the hydropathic index of amino acid residues. The importance of the hydropathic amino acid index in conferring interactive biological function on a protein has been discussed by Kyte and Doolittle (1982) for example. It is accepted that the relative hydropathic character of amino acids contributes to the secondary structure of the resultant protein. This, in turn, affects the interaction of the protein with molecules such as enzymes, substrates, receptors, ligands, DNA, antibodies, antigens, etc. Based on its hydrophobicity and charge characteristics, each amino acid has been assigned a hydropathic index as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5). Like amino acids can also be substituted on the basis of hydrophilicity. U.S. Pat. No. 4,554,101 discloses that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0±1); serine (+0.3); asparagine/glutamine (+0.2); glycine (0); threonine (−0.4); proline (−0.5□1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4). Thus, one amino acid in a peptide, polypeptide, or protein can be substituted by another amino acid having a similar hydrophilicity score and still produce a resultant peptide, etc., having similar biological activity, i.e., still retaining correct biological function. In making such changes, amino acids having hydropathic indices within ±2 are preferably substituted for one another, those within ±1 are more preferred, and those within ±0.5 are most preferred. Moreover, one can easily determine the characteristics of particular amino acid residues to be used in a substitution and/or modification as described herein (e.g., such as in determining to substitute a large non-polar for a small non-polar residue, or a small polar vs. a large polar residue) using, for example, standard teachings in the art regarding amino acid residues (e.g., one could easily use a diagram (created by projecting Dayhoff's mutation odds matrix using multidimensional scaling) in which amino acid residues that have been shown to have similar properties in different proteins are represented as being physically closer to each other on the diagram, thus allowing the diagrams' physical distances to permit an informed and reasoned choice of functional amino acid residue substitutes) or, similarly, one of ordinary skill in the art could use a PAM250 scoring matrix to assist in choosing amino acid substitutions (see, e.g., W. A Pearson, 1990 in Methods in Enzymology, ed. R. Doolittle (Academic Press, San Diego) 183:63-98)).

LP231 Functions

Given the teachings supplied herein of: LP231 primary amino acid and domain architecture, the relationship of LP231 amino acid sequence and higher order structural features compared with known proteins and their higher order structural features (e.g., such as the recently described superimposability of the ten-strand jelly-roll folding topology of Acrp30 and TNFs (Shapiro & Scherer 1998 Curr Biol 8:335-338)), it is likely that an LP231, an LP231 variant, an LP231 agonist, an LP231 antagonist, an LP231 binding partner or an LP231 fragment as described herein plays a similar role in a variety of physiological processes. Some non-limiting examples of functions such a composition is likely to participate in are, for example, those such as: modulation of complement activation and/or modulation of various associated diseases, conditions, syndromes associated with complement activation such as, e.g.: human sepsis, post-operative myocardial dysfunction due to reperfusion injury, severe capillary leakage syndrome after transplantation (e.g., such as, bone marrow transplantation), angioneurotic edema, excessive activation of complement in severe inflammation in a clinical disorder associated with tissue destruction, septic shock (such as, e.g., activated by microorganisms), capillary leakage syndrome after transplantation (e.g., such as bone marrow transplantation), complement mediated inflammation in the CNS (e.g., such as after brain trauma), reperfusion injury (e.g., such as after lung transplantation or myocardial disorder due to reperfusion injury), modulation of toxicity caused by interleukin-2 immunotherapy, vascular leakage syndrome, ischemia/reperfusion injury, burn injury; inflammation (e.g., by maintaining balance within and/or between the inflammatory cascades such as, for example, inflammatory cascades of plasma factors); coagulation (e.g., such as during the contact phase of coagulation, however, LP231 or its variants may function as both a pro- and/or a anticoagulant depending on which part, time, or portion of a coagulation cascade LP231 is active in); regeneration (e.g., such as nerve regeneration); metabolism and disorders of metabolism (e.g., such as dislipidemia, atherosclerosis, diabetes, disorders of energy metabolism modulated by adipocytes, for example obesity and conditions related to obesity); lipid metabolism (e.g., such as lipogenesis, fatty acid uptake, and lipolysis); insulin resistance (e.g., such as induced in a variety of disease states, such as, e.g., cancer, sepsis, and trauma, or due to obesity); modulation of metabolism such as, e.g., including but not limited to weight modification, obesity, cachexia, bulimia, anorexia, insulin resistance; modulation of insulin action such as, e.g., free fatty acid levels, leptin secretion rates, glucose transporter number, and insulin receptor signaling capacity; modulation of obesity-related apoptosis (such as, e.g., in brown adipose tissue); cardiovascular disease; various immune responses (such as, e.g., during responses to parasite and/or bacterial infection); autoimmune diseases; blood coagulation and/or coagulative disorders; shock syndromes due to serious injury or septicemia; sepsis; vascularization (such as that, e.g., involved in diabetic conditions, regulation of blood pressure, modulation of tumor progression); mediation of apoptosis (such as, e.g., in neural cells, such as, e.g., in oligodendrocytes); extra-cellular matrix (ECM) activities (such as, e.g., modulation of cartilage or bone formation (or capsular remodeling)); tumorgenesis; cellular metastasis; cell proliferation; cytostatic; proliferative; energy homeostasis; vulnerary; immunomodulatory; antidiabetic; antiasthmatic; antirheumatic; antiarthritic; antiinflammatory; antithyroid; antiallergic; antibacterial; antiviral; dermatological; neuroprotective; cardiant; thrombolytic; coagulant; nootropic; vasotropic; antipsoriatic; and antiangiogenic.

LP231 & Inflammation

Systemic inflammatory states are frequently accompanied by activation of the coagulation system and activation of the coagulation system is an almost invariable consequence of septic shock. The simultaneous activation of the innate immune response and the coagulation system after injury is a phylogenetically ancient, adaptive response that can be traced back to the early stages of eukaryotic evolution. Most invertebrate species lack differentiated phagocytic cells and platelets. They possess a common cellular and humoral pathway of inflammation and clotting after a breach in their internal milieu by either trauma or infection. The close linkage between clotting and inflammation has been preserved throughout vertebrate evolution and is readily demonstrable in human physiologic responses to a variety of potentially injurious stimuli. The same pro-inflammatory stimuli that activate the human clotting cascade also activate phagocytic effector cells (such as, e.g., neutrophils, monocytes, and macrophages). Consequently, the role of LP231 in physiological functions will likely cross artificial boundaries designated solely as inflammation or immune responses and thus information suggesting a role for LP231 in inflammation is also indicative of a role for LP231 in an immune response and vice versa. Additionally, studies showing functions and reactions in TNFs or complement proteins related to LP231 (as evidenced by sequence identity and structural similarity) will also inform questions regarding similar functions and reactions with LP231. LP231's homology to proteins involved in the classical complement pathway (e.g., C1q B-chain) suggest that as described may also participate in immune system functions. Furthermore, due to the highly integrated linkage between systemic inflammation and coagulation that is maintained in all vertebrates (see, e.g., Opal S. M. 2000 Critical Care Med. (9 Suppl): S77-80), may also participate in inflammatory processes that modulate coagulation and vice versa. Accordingly, an LP231 variant, an LP231 agonist, an LP231 antagonist, an LP231 binding partner or an LP231 fragment may be involved in diseases, disorders, conditions associated with stimulation of both the coagulative and inflammatory systems, such as, for example, sepsis.

Consequently, LP231, an LP231 variant, an LP231 agonist, an LP231 antagonist, an LP231 binding partner or an LP231 fragment as described may also exhibit anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions (including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as; e.g., NF or IL-1. An LP231, an LP231 variant, an LP231 agonist, an LP231 antagonist, an LP231 binding partner or an LP231 fragment may also be useful to treat anaphylaxis and hypersensitivity to an antigenic substance or material.

Hemolyis Model

To test for the ability of LP231, an LP231 variant, an LP231 agonist, an LP231 antagonist, an LP231 binding partner or an LP231 fragment to mediate hemolysis, one can adapt the methods of Kishore, et al. 1998a Biochem j 333:27-32 or 1998b Mol Immunol 35:375 (incorporated herein for these teachings). In brief, a fusion protein comprising a C1q-like portion of LP231, such as, e.g., from about Ala-160 to about Asp-287, is tested for its ability to inhibit C1q-dependent hemolysis of IgG (EA_{IgG})- and IgM (EA_{IgM})-sensitized sheep erythrocytes. Sheep red blood cells (E) are sensitized with hemolysin (A), comprising purified antisheep blood cell immunoglobulin (IgG or IgM), to yield EA_{IgG} or EA_{IgM} sensitized cells. The C1q hemolytic assay requires C1q to be added back to C1q deficient serum to reconstitute the C1 complex. Typically, addition of C1q (1 ug/ml) back to C1q-deficient serum is sufficient to completely lyse EA cells (coated with IgG or IgM). This concentration is then used as the standard for degree of hemolysis in a series of studies to determine if pretreatment of EA_{IgG} or EA_{IgM} with an LP23 -fusion protein (comprising the LP231-C1q-like portion) will inhibit the C1q-dependent hemolysis. The method is adapted and carried out as described in Kishore, et al. 1998 Biochem J 333:27-32 but adjusted to test a LP231-C1q-like-fusion protein (e.g., such as one constructed by fusing the LP231 portion with maltose-binding protein (MBP)). Briefly, aliquots of sheep erythrocytes (EA cells), at about 10⁷/100 ml concentration, sensitized with IgG or IgM are preincubated for 1 h at 37° C. with various concentrations of a LP231 fusion protein (e.g., such as, 0.75, 1.25, 2.5, 5.0, and 10.0 ug) or a control (e.g., such as the non-LP231 portion of the fusion- construct, e.g., such as MBP alone). Pretreated cells are gently pelleted by centrifugation at 3000×g for 2 min, then washed and resuspended in 100 ul of DGVB⁺⁺ isotonic Veronal-buffered saline containing 0.1 mM CaCl2, 0.5 mM MgCl2, 0.1% (w/v) gelatin and 1.0% (w/v) glucose]. Each aliquot of EA is added to a mixture, composed of 1 ug of C1q in 10.0 ul, 2.5 ul of C1q-deficient serum and 87.5 ul of DGVB⁺⁺. After 1 h incubation at 37 C, the unlysed cells are pelleted and the amount of hemoglobin released is determined spectrophotometrically from the A₄₁₂. Total hemolysis is assessed as the amount of hemoglobin released upon cell lysis with water. The C1q-dependent hemolytic activity is expressed as a percentage of total hemolysis.

Results showing inhibition of hemolysis because of an LP231 fusion protein indicate that such fusion products are competing with whole C1q to bind IgG and/or IgM on blood cell surfaces. Positive assay results further support a suggestion that such recombinant fusion constructs comprising an LP231-C1q-like portion could be employed to modulate complement activation thus, for example, in one instance, modulating pathogenic effects of complement-related diseases, states, conditions, or syndromes such as, e.g., acute hemolytic anemia, autoimmune disease, or inflammatory tissue damage such as, for example, autoantibody dependent tissue damage, sepsis mediated tissue damage; ischemic reperfusion injury; transplantation-related damage; and organ specific damage via complement activation.

Similarly, other experimental models or techniques can be adapted to examine the effect an LP231, an LP231 variant, an LP231 agonist, an LP231 antagonist, an LP231 binding partner or an LP231 fragment has on complement-mediated diseases, states, conditions, or syndromes such as, for example, using models of: experimental allergic encephalomyelitis (Piddlesden, et al. 1994 J. Immunol. 152:5477-5484); dermal vascular reactions (Yeh, et al. 1991 J. Immunol. 146:250-6), collagen induced arthritis (Goodfellow, et al. 2000 Clin. Exp. Immunology. 119:210-6), traumatic brain injury (Kaczorowski, et al. 1995 J. Cereb. Blood Flow Metab. 15:860-864), myasthenia gravis (Piddlesden, et al. 1996 J. Neuroimmunol. 71:173-177), Guillian-Barré syndrome (Jung, et al. 1995 Neuroscience Letters 200:167-70), glomerulonephritis (Couser, et al. 1995 J. Am. Soc. Nephtol. 5:1888-1894), allergic reactions (Lima, et al. 1997 J. Leukocyte Biol. 61:286-262) and asthma Regal, et al., 1993 J. Pharmacol. Exp. Ther. 267:979-988).

Acute Inflammatory Response Model

To test an acute inflammation response for an LP231, an LP231 variant, an LP231 agonist, an LP231 antagonist, an LP231 binding partner or an LP231 fragment, one can adapt the method of Eberini, et al. 1999 Electrophoresis 20(4-5): 846-53 (incorporated herein for these teachings). In brief, rodents are injected with a phlogistic stimulus (e.g., turpentine), turpentine and daily doses of indomethacine, and indomethacine alone. In inflamed animals, peak changes for acute-phase reactants are evaluated between 48 and 72 h after the phlogistic stimulus by two-dimensional electrophoresis (2-DE) to check for, for example, plasma concentration of LP231 expression, among other expressed molecules. Presence of LP231 is indicative of it being an acute phase protein whose changes are modulated via anti-inflammatory reaction.

Acute Inflammation Response Model with LP231 Transgenics

Using a method based on Chen, et al., 1997 Life Sci 60(17): 1431-5 (which is incorporated herein for these teachings), the potential role of LP231 in inflammation is evaluated in transgenic mice by overexpressing the LP231 gene under the control for example, of mouse metallothionein metal-responsive promoter. Briefly, bacterial endotoxic lipopolysaccharide (LPS) is injected intraperitoneally into mice at a dose of 600 microg/25 g body weight. The death toll is recorded every 12 hours for 3 days. The survival rate of transgenic male mice is assessed versus that of control male mice 3 days post LPS injection. In comparison, the survival rate of transgenic female mice is assessed versus that of control female mice to assess LP231 response to hormonal differences. Recombinant LP231 levels in the circulation of these mice is assessed for increase after LPS treatment. The results are examined to determine if LP231 transgenic mice have a higher survival rate than their non-transgenic control littermates after endotoxin shock and whether there is a gender based resistance to lethality induced by endotoxin shock. These results will suggest if LP231 has a protective effect during acute phase inflammation.

Inflammation Model for Liver Disease

To determine if LP231 plays a role in hepatic disease (e.g., such as the result of inflammation response) one can adapt the method of Newsholme et al. 2000 Electrophoresis 21(11): 2122-8 (incorporated herein for these methods) and generate a drug-induced increase in heptocellular rough endoplasmic reticulum (RER) in Sprague-Dawley rats by giving a substituted pyrimidine derivative. Subsequently, the experimental subjects are checked for the presence of LP231 which is interpreted as being indicative of the presence of an acute phase protein whose changes follows an inflammatory reaction supporting the suggestion that LP231 plays a role in, for example, acute phase liver inflammation.

Inflammation and Neurological Disease

Cytokines such as interleukin-6 (IL6) have been detected in the cortices of Alzheimer disease (AD) patients, indicating a local activation of components of the unspecific inflammatory system. IL-6 may precede neuritic changes, and the immunological mechanism may be involved both in the transformation from diffuse to neuritic plaques in AD and in the development of dementia. To determine if LP231 plays a role in neurological disease (e.g., such as the result of an inflammation response) one can adapt the method of Hull, et al. 1996 Eur Arch Psychiatry Clin Neurosci 246(3): 124-8 (incorporated herein for these teachings) to determine if LP231 plays a role in such processes. Furthermore, in the brain, the acute phase protein antichymotrypsin is produced in response to pro-inflammatory cytokines by the reactive astrocytes, in particular those surrounding the amyloid plaques of Alzheimer's disease brains. Accordingly, one can also adapt the method of Cardinaux et al., 2000 Glia 29(1): 91-7 to determine if similar pro-inflammatory molecules (e.g., such as, lipopolysaccharides (LPS), IL-1beta, and TNF alpha) induce the expression of LP231 in mouse primary neuronal support cells and whether the results of such data support a role for the induction of LP231 expression by pro-inflammatory cytokines in the brain (e.g., using mouse cortical astrocytes as a model system).

Hemostatic and Thrombolytic Activity

LP231, an LP231 variant, an LP231 agonist, an LP231 antagonist, an LP231 binding partner or an LP231 fragment as described herein may also exhibit hemostatic or thrombolytic activity. As a result, such a composition is expected to be useful in treatment of various coagulation disorders (including hereditary disorders, such as hemophilias) or to enhance coagulation and other hemostatic events in treating wounds resulting from trauma, surgery or other causes. Such a composition may also be useful for dissolving or inhibiting formation of thromboses and for treatment and prevention of conditions resulting therefrom (such as, for example, infarction of cardiac and central nervous system vessels (e.g., stroke)). The activity of LP231, an LP231 variant, an LP231 agonist, an LP231 antagonist, an LP231 binding partner or an LP231 fragment as described herein may, among other means, be measured by the following methods: Assay for hemostatic and thrombolytic activity include, without limitation, those described in: Linet et al., J. Clin. Pharmacol. 26:131-140, 1986; Burdick et al., Thrombosis Res. 45:413-419,1987; Humphrey et al., Fibrinolysis 5:71-79 (1991); Schaub, Prostaglandins 35:467-474, 1988. A potential function of LP231 in vascular biology (such as, e.g., testing mitogenic responses via, for example, an induced MAPK pathway) can be investigated by studying the role of LP231 in the proliferation and migration of cultured primary aortic vascular smooth muscle cells (VSMCs) in vitro and in neointima formation in rat artery after balloon angioplasty in vivo based on the methods of Miao et al., 2000 Circ Res 86(4): 418-24 which is incorporated herein by reference for the teachings assay with modification for LP231 specificity). An LP231, an LP231 variant, an LP231 agonist, an LP231 antagonist, an LP231 binding partner or an LP231 fragment may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in- vivo or ex-vivo (i.e., in conjunction with bone marrow transplantation or with peripheral progenitor cell transplantation (homologous or heterologous)) as normal cells or genetically manipulated for gene therapy.

Blood Pressure Model

To examine if LP231 has an effect on the vasculature and on blood pressure homeostasis, an intravenous bolus injection of LP231 is given to a subject (e.g., such as an anesthetized rodent) to look for a rapid, potent, and transient reduction elevation of mean arterial blood pressures. Infusions of purified LP25 in the dosage of about 0.07-1.42 nmol/kg into cannulated rodent jugular veins are carried out and the effect on the mmHg reading of blood pressure is determined in a dose-dependent manner. Significant variation from controls indicates a role for LP231 in blood pressure homeostasis.

Alternatively, to investigate the role of LP231 in blood pressure regulation, LP231 can be delivered to hypotensive transgenic mouse lines by intramuscular injection (see, e.g., the method of Ma, et al. 1995 J Biol Chem 270(1): 451-5, which is incorporated herein for these teachings). Expression of the LP231 is examined for expression in skeletal muscle by reverse transcription-polymerase chain reaction and Southern blot analysis at 10, 20, 30, and 40 days post-injection. Immunoreactive LP231 levels in the muscle and serum of these mice is quantified by an LP231-specific enzyme-linked immunosorbent assay and Western blot analysis. The levels of LP231 mRNA and immunoreactive protein are examined at 10, 20, and 30 days post-injection. During this period, LP231 delivery is examined to determine its effect on systemic blood pressure compared to that of normotensive control mice. Furthermore, to elucidate therapeutic potentials of LP231 in hypertension, a LP231 polynucleotide encoding an LP231 or variant thereof (e.g., in an adenoviral vector) is directly introduced into spontaneously hypertensive rats (SHR) through portal vein injection (see, e.g., the method of Ma, et al. 1995 J Biol Chem 270(1): 451-5, which is incorporated herein for these teachings). Still furthermore, the following method (adapted from Gerova, M 1999 Physiol Res 48(4): 249-57, which is incorporated herein for these assay teachings) can be used to determine whether LP231 exerts a protective effect in chronic-inhibition-of-nitric-oxide-synthase-induced hypertension. Chronic-inhibition-of-nitric-oxide-synthase-induced hypertension is created by giving N omega-nitro-L-arginine methyl ester (L-NAME, 40 mg/100 ml water or given in a dose of 50 mg/kg into the jugular vein) orally to Sprague-Dawley rats, while controls receive regular tap water. Blood pressure is measured in the right carotid artery by a Statham pressure transducer in acute experiments, and on the tail artery by the plethysmographic method weekly in chronic experiments. Subsequently, LP231 mRNA levels are measured and compared with known vascularization effecting proteins such as, e.g., proteins of the kallikrein-kinin system. The results are used to assess whether enhanced LP231 synthesis has a protective role against the cardiovascular effects induced by chronic inhibition of nitric oxide synthesis.

Diabetes & Muscle Wasting Model

To investigate the role of LP231 as a factor contributing to muscle wasting (such as, e.g., observed in diabetes and fasting), one can adopt the method of Kuehn et al., 1988 Biol Chem Hoppe Seyler 369 Suppl:299-305 (which is incorporated herein by reference for these assay teachings). Briefly, using such techniques, LP231 expression levels are examined in the skeletal muscles of fasting rodents. Lowered levels of LP231 suggest that LP231 contributes to diseases of muscle wasting. Accordingly, increasing the level of LP231 in such conditions may ameliorate such conditions. To determine the involvement of LP231 in the development of diabetic retinopathy, one can adopt the method of Hatcher, et al., 1997 Invest Ophthalmol Vis Sci 38(3):658-64 (which is incorporated herein for these assay teachings). Briefly, diabetes is induced by streptozotocin (STZ) (55 mg/kg body weight in 0.05 M citrate buffer, pH 4.5) in male Sprague-Dawley rats (150 to 175 g, 6 weeks old) as confirmed by hyperglycemia and reduced body weight. Retinas are dissected from animals at 1, 2, and 4 months of induced diabetes-like conditions. The functional activity of LP231 in retinal homogenates is determined by immunoreactive LP231 levels measured by enzyme-linked immunosorbent assay. Additionally, LP231 messenger RNA (mRNA) levels in the retina are measured by Northern blot analysis using an LP231 complementary DNA probe. The activity of total Na+, K(+)-ATPase is determined by a radioassay. Total protein concentration is determined by a protein assay.

Spinal Cord Regeneration Model

To evaluate the role LP231 in a spinal cord regeneration response (based on the methods of O'Hara, and Chernoff 1994 Tissue and Cell, 26: 599-611; Chernoff, et al. 1998 Wound Rep. Reg. 6: 435-444; and Chernoff, et al, 2000 Wound Rep. Reg. 8: 282-291, which are incorporated herein for these teachings) a tissue culture system using axolotl spinal cord ependymal cells is used to test the effects of an LP231, an LP231 variant, an LP231 agonist, an LP231 antagonist, an LP231 binding partner or an LP231 fragment on, for example, nerve and tissue regeneration. Additionally using other techniques to investigate similar issues (see, e.g., Itasaki, et al, 1999 Nature Cell Biology Dec;1(8):E203-207; Momose, et al., 1999 Develop. Growth Differ. 41:335-344; and Atkins, et al., 2000 Biotechniques 28: 94-96, 98, 100; which are incorporated herein for these teachings), one can conduct localized transfection studies of LP231 constructs in frog limb cultures and frog spinal cord. Although the above referenced methods were first developed for use in the chick, they can also be adapted for use, for example, in a frog limb system to examine the role of an LP231, an LP231 variant, an LP231 agonist, an LP231 antagonist, an LP231 binding partner or an LP231 fragment in, for example, cellular regeneration. Similar models can be adapted to examine the role of an LP231, an LP231 variant, an LP231 agonist, an LP231 antagonist, an LP231 binding partner or an LP231 fragment in organ regeneration (e.g., such as hepatic regeneration using available liver models and assay techniques).

LP231 & Lipids

To examine the role of LP231, an LP231 variant, an LP231 agonist, an LP231 antagonist, an LP231 binding partner or an LP231 fragment in the regulation of lipoproteins many common methods exist in the art (see, e.g., the various methods and techniques discussed in, for example, Krieger & Herz 1994 Annu Rev Biochem 63:601-37, which is incorporated herein for the methods and techniques described therein). A non-limiting example of such an examination are the methods employed in Sugiyama, et al. 2000 Biochemistry 39:15817-15825, which is also incorporated herein by reference for the assay techniques described therein. For example, to examine if LP231 is capable of binding apoE-containing lipoproteins one can use beta-VLDL (an apoE-rich lipoprotein that is a mixture of cholesteryl ester-rich chylomicron remnants and cholesteryl ester-rich lipoproteins, which is detected in the peripheral plasma of patients with Type III hyperlipidemia and in animals fed with a cholesterol-supplemented diet). Briefly, LDL receptor deficient cells (e.g., a murine IL-3-dependent pro-B cell line Ba/F3 is cultured in RPMI1640 medium (Sigma) containing 10% fetal calf serum (FCS, Sigma) and 1 ng/ml recombinant murine IL-3 (Miyajima, et al. 1987 Gene 58, 273-281). CHO-K1 and the LDL receptor-deficient are maintained in Dulbecco's modified Eagle's medium (DMEM, 4.5 mg/ml glucose, Sigma) containing 1% MEM nonessential amino acid solution (Gibco BRL) and 5% FCS (known as medium A)) are constructed (see, e.g., I(rieger, et al. 1983 PNAS USA 80:5607-5611) and subsequently transfected with LP231 constructs (using common techniques and the sequences provided herein). After selection with 1 mg/ml G418 (Sigma), survived colonies are cloned. Immunoprecipitation and Western blot analysis using standard techniques quantitate the expression of LP231. The highest expressing LP231 clone is used for subsequent experiments. In binding or cholesteryl esterification assays, cells are seeded at a concentration of approximately 5×10⁵ per dish into 60 mm culture dishes containing 3 ml of medium A. On the following day, cells are washed twice with phosphate-buffered saline and fed again with medium containing 5% lipoprotein-deficient serum (LPDS). Twenty-four hours later, when the cells became confluent, the cells are harvested. To conduct binding and cholesteryl esterification assays, rabbit beta VLDL (d<1.006 g/ml) is prepared from 1.0% cholesterol-fed rabbits. Male Japanese white rabbits (Saitama Experimental Animal Supply) weighing 2.5-3.0 kg are fed a 1.0% cholesterol diet for 3 weeks and then fasted for 15 h. A single 50 ml unit of blood is collected in 0.1% EDTA. Fractionation is carried out as described in Kovanen, et al. 1981 Proc. Natl. Acad. Sci. U.S.A. 78, 1396-1400 (which is incorporated herein by reference for these method techniques). Beta-VLDL is labeled with 125-I as described in Goldstein, et al. 1983 Methods Enzymol 98:241-260 (which is incorporated herein by reference for these method techniques), and binding of 125-I-labeled beta-VILDL at 4° C. is measured as described Goldstein et al. (supra). Protein concentrations are determined using a DC protein assay kit (Bio-Rad). The incorporation of [14 C]oleate-albumin into cellular cholesteryl [¹⁴ C]oleate by cell monolayers is measured as described in Goldstein et al. (supra), with the exception that beta-VLDL and recombinant human apoE3 (Cosmo Bio, Tokyo, Japan) are pre-incubated together for 1 h at 37° C. in 250 μL of culture medium.

To verify the binding and internalization of beta-VLDL, one measures the ability of beta-VLDL to stimulate the incorporation of [¹⁴ C]oleate into cholesteryl esters (Goldstein et al. supra). Intracellular cholesteryl esterification is catalyzed by acyl-coenzyme A:cholesterol O-acyltransferase (ACAI). Cellular cholesterol synthesis itself does not stimulate ACAT activity, but rather ACAT is activated by cholesterol liberated from LDL or beta-VLDL following receptor-mediated uptake (Goldstein et al. supra). This assay is known to be sensitive with higher specificity than the surface binding assay using an 125 I-labeled ligand (Kowal, et al., 1989 PNAS USA 86:5810-5814). Stimulation of cholesteryl [¹⁴ C]oleate formation in LP231 transfected and controls by apoE-enriched beta-VLDL is conducted as follows: after 24 h of growth in medium containing lipoprotein-deficient serum, cell monolayers are incubated with varying concentrations of beta-VLDL pre-incubated with 0, 10, and 40 μg/ml apoE. After 5 h, the cells are pulse-labeled for 2 h with [¹⁴ C]oleate, and the content of cholesteryl [¹⁴ C]oleate is determined. Each value is the average of duplicate incubations, which are corrected for radioactivity observed in incubations containing no lipoproteins (e.g., such as, 0.65, 0.68, and 0.70 nmol/h/(mg of protein) for controls with 0, 10.0, and 40.0 μg/ml apoE, respectively; 0.97, 0.96, and 0.99 nmol/h/(mg of protein) for LP231 constructs with 0, 10.0, and 40.0 μg/ml apoE, respectively.

Investigation of Weight, Leptin Levels, Food Intake, Urine Production, Oxygen Consumption, and Triglyceride and Free Fatty Acid Levels in LP231 Transgenic Mice

Obesity refers to a condition whereby a mammal has a Body Mass Index (BMI), which is calculated as weight ) per height² (meters), of at least about 25.9. Conventionally, those persons with normal weight have a BMI of from about 19.9 to less than about 25.9. The obesity described herein may be due to any cause, whether genetic and/or environmental. Examples of disorders that may result in obesity or be the cause of obesity include overeating, bulimia, polycystic ovarian disease, craniopharyngioma, the Prader-Willi Syndrome, Frohlich's syndrome, Type II diabetes, GH-deficient subjects, normal variant short stature, Turner's syndrome, and other pathological conditions showing reduced metabolic activity or a decrease in resting energy expenditure as a percentage of total fat-free mass, e.g., children with acute lymphoblastic leukemia.

Conditions related to obesity refer to conditions that are the result of or which are exasperated by obesity, such as, but not limited to dermatological disorders such as infections, varicose veins, Acanthosis nigricans, psoriasis and eczema, exercise intolerance, diabetes mellitus, insulin resistance, hypertension, hypercholesterolemia, cholelithiasis, osteoarthritis, orthopedic injury, thromboembolic disease, cancer, and coronary (or cardiovascular) heart disease, particular those cardiovascular conditions associated with high triglycerides and free fatty acids in an individual. Methods for determining effects of an LP231, an LP231 variant, an LP231 agonist, an LP231 antagonist, an LP231 binding partner or an LP231 fragment on metabolism can be determined based on teachings known in the art, those in U.S.S.No. 60/264239, and methods taught or incorporated by reference herein. Additionally, to investigate metabolism-modulating functions of an LP231, an LP231 variant, an LP231 agonist, an LP231 antagonist, an LP231 binding partner or an LP231 fragment, transgenic mice can be generated that express human LP231 using techniques common in the art (see, e.g., the following texts: Burki, K. 1986. Experimental embryology of the mouse. In: Monographs in Developmental Biology, (ed.) H. W. Sauer. Vol. 19, Karger Publishers, Basel; Grosveld, R. and G. Kollias. 1992. Transgenic Animals. Academic Press, San Diego. ISBN 0-12-304530-4; Hogan, B., R. Beddington, F. Costantini and E. Lacy 1994. Manipulating the Mouse Embryo: A Laboratory Manual. Cold Spring Harbor Laboratory. Box 100, Cold Spring Harbor, N.Y. 11724 (1-800-843-4388); Pinkert, Calif. (ed). 1994. Transgenic Animal Technology: A Laboratory Handbook. Academic Press, San Diego. ISBN 0-12-557165-8; Monastersky, G. M. and J. M. Robl. (ed). 1995. Strategies in Transgenic Animal Science. American Society for Microbiology Press, Washington, D.C. ISBN 1-55581-096-9; Houdebine, L. M. (ed). 1997. Transgenic Animals: Generation and Use. Harwood Academic Publishers, Amsterdam. ISBN 90-5702-069-6, Genetic Modification of Animals; Tim Stewart; In Exploring Genetic Mechanisms pp565-598; 1997 Eds M Singer and P Berg; University Science Books; Sausalito, Calif).; or the following videos: RCA. Pedersen and J. Rossant. 1989. Transgenic Techniques in Mice: A Video Guide. Cold Spring Harbor Laboratory, Box 100, Cold Spring Harbor, N.Y. 11724 (1-800-8434388). (VHS, ISBN 0-87969-950-7; also PAL, BETA and SECAM or R. A. Pedersen and J. Rossant. 1993. Targeted Mutagenesis in Mice: A Video Guide. Cold Spring Harbor Laboratory, Box 100, Cold Spring Harbor, N.Y. 11724 (VHS, ISBN 0-87969-430-0; PAL, ISBN 0-87969-430-OP); and the following journal: Transgenic Research. http://www.wkap.nl/journalhome.htm/0962-8819, Kluwer Publishing, The Netherlands).

For instance, a cDNA encoding a human LP231 can be cloned into a plasmid containing the human apolipoprotein E (hApoE) gene promoter-5′ in operable linkage with the LP of interest using common art techniques. A splice acceptor and donor can also be included 5′ to the LP cDNA to increase the level of expression and a splice donor and acceptor with a poly A addition signal is included 3′ to the LP cDNA to increase the level of transcription and to provide a transcription termination site.

The DNA encompassing the promoter, the 5′ splice acceptor and donor, the LP cDNA and the 3′ splice acceptor and donor and the transcription termination site (the transgene) is released from a bacterial vector sequence using appropriate restriction enzymes and purified following size fractionation on agarose gels. The purified DNA is injected into one pronucleus of fertilized mouse eggs and transgenic mice are generated and identified as described in the literature above. The mice are approximately 6 weeks of age for measurements discussed below such as for water intake, food consumption, urine output and hematocrit. The leptin, triglycerides and free fatty acid measurements are taken on the same animals at 8 weeks of age. Data is collected to examine food intake and metabolic rate as evidenced by rate of oxygen consumption. Weight and percentage of body fat is examined in treated versus non-transgenic littermates. Decreased body weight is examined for being a consequence of decreased adiposity. Transgenic mice are assessed for normal linear growth such as by nose to rump length measurements. They are also assessed for normality with respect to body temperature, bone length and hematological values. Transgenic mice are also assessed for urine output. Increased urine output may be derived from an increased metabolism of food. Therefore, mice should be examined for the amount of water consumption and for signs of dehydration (as determined by a normal hematocrit) since absence of dehydration without increased water consumption may signal increased metabolism caused by the LP. A decrease in adiposity in treated mice without altering either muscle mass or long bone formation is indicative of an effective therapeutic for treating obesity and obesity related conditions.

Transgenic mice are also weighed at various times under different fasting and feeding conditions. More particularly, groups of female LP transgenic mice and their non-transgenic littermates are weighed at 6 weeks of age during ad libitum feeding, after 6 and 24 hour fasts and 24 hours after ending a 24 hour fast to test the transgenic mice under all conditions and to determine if LP transgenic mice weighed less than their wild type, non transgenic littermates. Sera of treated and controls can be assayed for various agents, such as, e.g., leptin. Evidence of decreased leptin levels in LP transgenic mice would be consistent with lower body weights being due to decreased adiposity. A group of 6-week-old transgenic mice are monitored for food intake, water intake, urine output and hematocrit Transgenic mice in which an LP is effective might be expected to consume more food and still have a decreased body weight, which could be explained by an increase in metabolic rate. Metabolic rate is determined by measuring oxygen consumption during both light cycles, following a 24-hour fast and 24 hours after ending a 24 hour fast. Obesity and elevated triglycerides and free fatty acids are risk factors for cardiovascular disease. To examine if an LP decreases one of the risk factors for cardiovascular disease (obesity), it can also be investigated if an LP of the invention also lowers other risk factors such as level of serum triglycerides and free fatty acids (FFA).

Investigation of Mice Treated with Recombinant LP231

Another method of testing an LP231, an LP231 variant, an LP231 agonist, an LP231 antagonist, an LP231 binding partner or an LP231 fragment for its effect on metabolism is to deliver recombinant or isolated LP encompassed herein (in dosages ranging from about 0.1 ug/mouse to 100 ug/mouse of daily injections) to test, for example, food intake, metabolic rate, activity level, body composition, etc. such as in Gloaguen, et al. 1997 Proc. Nat. Acad. Sci. USA 94:6456-6461 or Lambert, et al. 2001 Proc. Nat. Acad. Sci. USA 98:4652-4657 (both of which are incorporated herein by reference for their methods and assays regarding testing obesity compounds). Briefly, experiments are performed using groups of male 10- to 18-week-old C57BL/6] ob/ob and C57BL/KS db/db mice, and 19-week-old AKR/J mice rendered obese by feeding a high-fat diet (West, et al. 1992 Am. J. Physiol. 262, R1025-R1032, which is incorporated by reference herein) starting at 12 weeks of age. Typically, animals are housed in individual cages with ad libitum access to water and either standard or high-fat (AKR mice) rodent chow, under a 12-hr light-dark cycle (lights on at 0730, off at 1930). They are accustomed to daily (900 hr) intraperitoneal injections of vehicle (0.9% saliney 0.2 mg/ml endotoxin-free bovine serum albumin) for two days before the beginning of the treatment (day 0) with either vehicle or LP (dosages ranging from about 0.1 ug/mouse to 100 ug/mouse). Animals are weighed after injection and food intake is determined by recording the amount of chow remaining in food dishes. In pair-feeding experiments, vehicle-treated mice are either fed ad libitum or fed the amount of chow consumed by treated mice during the preceding 24-hr period, starting at day 1. Blood samples are taken from the retroorbital sinus 24 hr after the last injection (0900), or 7 hr after the last injection and the removal of food (1600). Serum glucose is determined by the glucose oxidase method and serum insulin by radioimmunoassay (Amersham), using rat insulin as standard. Locomotor activity is measured by scoring the number of times mice cross the middle of their home cages during 3 hr of the dark cycle (2100-2400). Grooming behavior is assessed by focal observations in home cages (five observations of 1 min each during 30 min of the light cycle), using a rating scale from 0 to 3 (0, no activity; 1, weak; 2, normal; and 3, hyperactive). Conditioned taste aversion experiments are performed using a two-bottle paradigm with 0.1% saccharin as a novel taste (Langhans, et al 1990 Physiol. Behav. 47, 805-813, which is incorporated by reference for such methods). Body Composition can be determined commercially (Covance Laboratory, Princeton, N.J.).

Generally, mice (and humans) on a high fat diet will gain weight and adiposity and will become either glucose intolerant or diabetic. To examine whether exposure to an LP of the invention will impact on adiposity and glucose tolerance, mice treated (as above) and controls are put onto a high fat diet essentially as described by Rebuffe-Scrive et al Metabolism Vol 42, No 11 1993 pp1405-1409 and Surwit et al Metabolism, Vol 44, No 5 1995 pp. 645-651 with the modification that the sodium content is normalized with respect to the normal chow (diets prepared by Research Diets Inc. Catalog no. D12330N). After ten weeks on the either normal mouse chow or on the high fat diet, the treated and control mice are subjected to a glucose tolerance test by injecting intraperitoneally 1.0 mg glucose per kg of body weight with the concentration of glucose present in the blood being measured at intervals following the injection using standard procedures with diabetic mice, for example, defined as those having 2 hour glucose levels greater than 200 mg/dl (see, e.g., the World Book of Diabetes in Practice. Vol 3; Ed Krall, L. P.; Elsevier))

Alternatively, the methods of Lambert, et al. 2001 Proc. Nat. Acad. Sci. USA 98:4652-4657 can be used. Briefly, male C57BL/6 mice (Taconic Farms), C57BL/6J-Lepob (ob/ob), and AKR/J are obtained at 7-8 wk of age and housed in 12h of light per day at 69-74° F. and 40-60% humidity. All experiments begin at 10 weeks of age. Mice are provided with Rodent Laboratory Chow 5001 (Purina, St. Louis, Mo.) ad libitum, except for pair-fed mice, which are restricted to the same amount of food as eaten by the treatment group or AKR/J mice placed on a high fat diet (45% of the calories as fat, Research Diets, New Brunswick, N.J.) ad libitum for 7 wk to produce a DIO (diet induced obesity). After 7 wk, DIO mice should weigh approximately 30% more than littermates eating standard chow. Water is provided ad libitum to all mice. Before the start of treatment, mice are transferred from group housing to single housing to facilitate food intake measurements. Body weight and food consumption are monitored daily. In some studies, carcass analysis is performed (Covance Laboratory, Princeton, N.J.) to determine body composition. In other studies, mice are killed by cervical dislocation, and wet weights of the epididymal fat pads (bilateral) and the tibialis anterior, extensor digitorum longus, and/or gastrocnemius muscles are obtained as measurements of visceral adiposity and lean muscle mass, respectively. Tissues are collected 18-20 h after the last injection. Terminal blood samples are collected and serum corticosterone levels are measured by using a commercially available RIA kit (Biotrak, Amersham). Activity is measured as “mobile time” in a 21×33 cm monitoring chamber (IlTC, Woodland Hills, Calif.; model AM1051). Mobile time is defined as the percentage of a 10-min test period during which more than two horizontally displaced photocell beams are interrupted per 5 seconds. Additionally, such method and techniques for investigating obesity and obesity related disorders can be determined from the literature in the field of metabolism such as, e.g., “Clinical Obesity” 1998. P. C. Kopelman and M. J. Stock eds. Blackwell (ISBN 0 632 04198 6) or the diet induced obesity model of Gloaguen, et al. (1997 Proc. Natl. Acad. Sci. USA 94, 6456-6461) that is particularly representative of human obesity, or the techniques to test obesity-like compounds such as the methods of Lambert, et al. 2001 Proc. Natl. Acad. Sci. USA 98, 4652-4657, which are all incorporated herein by reference for such methods and techniques related to obesity investigations.

LP231 and Glucose Uptake and Leptin Release from Adipocytes

To further investigate the mechanism by which for an LP231, an LP231 variant, an LP231 agonist, an LP231 antagonist, an LP231 binding partner or an LP231 fragment, alters metabolism, recombinant human LP is added to cultures of primary rat adipocytes and glucose uptake and leptin release by the cells are measured using standard methods in the art (see, e.g., WO 01/18210 Al, which is hereby incorporated by reference for methods used to assess obesity treatments). Data is examined to determine if an for an LP231, an LP231 variant, an LP231 agonist, an LP231 antagonist, an LP231 binding partner or an LP231 fragment, increases the release of leptin from and decreases the uptake of glucose into primary rat adipocytes. Additional assays or methods for assessing an activity of an for an LP231, an LP231 variant, an LP231 agonist, an LP231 antagonist, an LP231 binding partner or an LP231 fragment, of the invention may, among other means, be measured by the following methods: Suitable assays for thymocyte or splenocyte cytotoxicity include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. USA 78:2488-2492,1981; Herrmann et al., J. Immunol. 128:1968-1974,1982; Handa et al., J. Immunol. 135:1564-1572,1985; Takai et al., J. Immunol. 137:3494-3500,1986; Takai et al., J. Immunol. 140:508-512, 1988; Herrmann et al., Proc. Natl. Acad. Sci. USA 78:2488-2492, 1981; Herrmann et al., J. Immunol. 128:1968-1974, 1982; Handa et al., J. Immunol. 135:1564-1572,1985; Takai et al., J. Immunol. 137:3494-3500,1986; Bowmanet al.,. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028-3033, 1990; and Assays for B cell function: In vitro antibody production, Mond, J. J. and Brunswick, M. In Current Protocols in Immunology. J. E. Coligan eds. Vol 1 pp. 3.8.1-3.8.16, John Wiley and Sons, Toronto. 1994.

Mixed lymphocyte reaction (MLR) assays (which will identify, among others, proteins that generate predominantly Th1 and CTL responses) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992.

Dendritic cell-dependent assays (which will identify, among others, proteins expressed by dendritic cells that activate naive T-cells) include, without limitation, those described in: Guery et al., J. Immunol. 134:536-544, 1995; Inaba et al., Journal of Experimental Medicine 173:549-559, 1991; Macatonia et al., Journal of Immunology 154:5071-5079, 1995; Porgador et al., Journal of Experimental Medicine 182:255-260, 1995; Nair et al., Journal of Virology 67:4062-4069,1993; Huang et al., Science 264:961- 965, 1994; Macatonia et al., Journal of Experimental Medicine 169:1255-1264, 1989; Bhardwaj et al., Journal of Clinical Investigation 94:797-807,1994; and Inaba et al., Journal of Experimental Medicine 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, Journal of Immunology 145:4037-4045, 1990; Zamai et al., Cytometry 14:891-897, 1993; Gorczyca et al., International journal of Oncology 1:639-648, 1992.

Assays for proteins that influence early steps of T-cell commitment and development include, without limitation, those described in: Antica et al., Blood 84:111-117,1994; Fine et al., Cellular Immunology 155:111-122, 1994; Galy et al., Blood 85:27,70-2778, 1995; Toki et al., Proc. Nat. Acad Sci. USA 88:7548-7551, 1991.

Assays for embryonic stem cell differentiation (which will identify, among others, proteins that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, M. G. In Culture of Hematopoietic Cells. R. I. Freshney, et al. eds. Vol pp. 265-268, Wiley-Liss, Inc., New York, N.Y. 1994; Hirayama et al., Proc. Natl. Acad. Sci. USA 89:5907-5911, 1992; Primitive hematopoietic colony forming cells with high proliferative potential, McNiece, I. K. and Briddell, R. A. In Culture of Hematopoietic Cells. R. I. Freshney, et al. eds. Vol pp. 23-39, Wiley-Liss, Inc., New York, N.Y. 1994; Neben et al., Experimental Hematology 22:353-359, 1994; Cobblestone area forming cell assay, Ploemacher, R. E. In Culture of Hematopoietic Cells. R. I. Freshney, et al. eds. Vol pp. 1-21, Wiley-Liss, Inc., New York, N.Y. 1994; Long term bone marrow cultures in the presence of stromal cells, Spooncer, E., Dexter, M. and Allen, T. In Culture of Hematopoietic Cells. R. I. Freshney, et al. eds. Vol pp. 163-179, Wiley-Liss, Inc., New York, N.Y. 1994; Long term culture initiating cell assay, Sutherland, H. J. In Culture of Hematopoietic Cells. R. I. Freshney, et al. eds. Vol pp. 139-162, Wiley-Liss, Inc., New York, N.Y. 1994.

Features of LP NO: 2 (LP285)

Endogenous proteolytic enzymes provide a variety of useful functions, including the degradation of invading organisms, antigen-antibody complexes, and certain tissue proteins that are no longer necessary. The serine proteases comprise a large family of enzymes that use an activated serine residue in the substrate-binding site to catalytically hydrolyze peptide bonds. Typically, this serine residue can be identified by the irreversible reaction of its side chain hydroxyl group with diisopropylfluorophosphate. Serine proteases participate in carefully controlled processes, such as blood coagulation, fibrinolysis, complement activation, fertilization, and hormone production. These proteases are utilized in a variety of diagnostic and therapeutic contexts, and as industrial enzymes. Normally, serine proteases catalyze limited proteolysis, in that only one or two specific peptide bonds of the protein substrate are cleaved. Under denaturing conditions, serine proteases can hydrolyze multiple peptide bonds, resulting in the digestion of peptides, proteins, and even autolysis. Various diseases are thought to result from the lack of regulation of serine protease activity, including emphysema, arthritis, cancer metastasis, and thrombosis. The discovery of a new serine protease fulfills a need in the art by providing a new composition useful in diagnosis, therapy, or industry.

LP285 is a novel polypeptide (SEQ ID NO: 2) that exhibits sequence similarity and/or identity (at the amino acid level) to various vertebrate serine proteinases (see Table 2 below). LP285 exhibits a domain architecture that suggests that it is as a new primate (e.g., human) serine protease. Specifically, LP285 possesses, in its amino acid structure, characteristics of members of the trypsin family of serine proteinases including, e.g., exhibiting trypsin-like domains. Such evidence indicates that LP285 has serine protease activity and as such, it is involved in regulated turnover of extracellular matrix or extracellular matrix-like molecules. In one embodiment LP285 may be expressed as an inactive form which is subsequently activated by proteolytic cleavage. LP285 is expressed embryonically indicating a possible role in functions, such as, for example: morphogenesis, organogenesis, cell migration, etc. Other LP285 functions are described herein.

Proteolytic enzymes that exploit serine in their catalytic activity are ubiquitous, being found in viruses, bacteria and eukaryotes (Rawlings & Barrett, 1994 Families of serine peptidases. Meth. Enzymol. 244 19-61). They embrace a wide range of peptidase activity, including exopeptidase, endopeptidase, oligopeptidase, and omega-peptidase activity. Over 20 families (denoted S1-S27) of serine protease have been identified, which are grouped into 6 clans (SA, SB, SC, SE, SF and SG) on the basis of structural similarity and other functional evidence (Rawlings & Barrett, 1994 Families of serine peptidases. Meth. Enzymol. 244 19-61). Trypsin-like protein domains are recognized in all proteins in families having the S1, S2A, S2B, S2C, and S5 classification of peptidases (see, e.g., Rawlings & Barrett, 1994 Meth Enzymol 244:19-61; and Sprang, et al., 1987 Science 237:905-909). Possession of trypsin family, active-site-like domains is typically characteristic for proteins having serine protease activity. The catalytic activity of serine proteases of the trypsin family is provided by a charge relay system involving an aspartic acid residue hydrogen-bonded to a histidine, which itself is hydrogen-bonded to a serine. The architecture of the protease domain and of amino acid sequences in the vicinity of the active site serine and histidine residues are well conserved in this family of proteases (see, e.g., Brenner 1988 Nature 334:528-530, Doolittle & Feng 1987 Cold Spring Harbor Symp. Quant. Biol. 52: 869-874; Krem, et al. 1999 Jour. Biol. Chem. 274: 28063-28066; and Table 2 below) and possession of such a domain can be used as criteria to identify new members of the family and to predict function of a putative protease.

A partial list of proteases known to belong to the trypsin family of serine proteases include: Acrosin; Blood coagulation factors VII, IX, X, XI and XII, thrombin, plasminogen, and protein C; Cathepsin G; Chymotrypsins; Complement components C1r, C1s, C2, and complement factors B, D and I; Complement-activating component of RA-reactive factor; Cytotoxic cell proteases (ranzymes A to H); Duodenase I; Elastases 1, 2, 3A, 3B (protease E), and leukocyte (medullasin); Enterokinase (EC 3.4.21.9) (enteropeptidase); Hepatocyte growth factor activator; Hepsin; Glandular (tissue) kallikreins (including EGF-binding protein types A, B, and C, NGF-gamma chain, gamma-renin, prostate specific antigen (PSA) and tonin); Plasma kallikrein; Mast cell proteases (MCP) 1 (chymase) to 8; Myeloblastin (proteinase 3) (Wegener's autoantigen); Plasminogen activators (urokinase-type, and tissue-type); Trypsins I, II, III, and IV; Tryptases; Snake venom proteases such as ancrod, batroxobin, cerastobin, flavoxobin, and protein C activator; Collagenase from common cattle grub and collagenolytic protease from Atlantic sand fiddler crab; Apolipoprotein(a); Blood fluke cercarial protease; Drosophila trypsin like proteases: alpha, easter, and snake-locus; Drosophila protease stubble (gene sb); and major mite fecal allergen Der p III. All of these proteins belong to the S1 family classification of peptidases (see, e.g., Rawlings & Barrett 1994 Meth. Enzymol. 244:19-61; and http://www.expasy.ch/cgi-bin/lists?peptidas.txt).

One consensus pattern used to detect serine proteases is the following amino acid residue sequence pattern: [LIVM]-[ST]-A-[STAG]-H-C; where H (indicated in bold typeface and underlined) is the active histidine site residue. Sequences known to belong to the S1 family class of peptidases which have been detected using this consensus pattern include all known serine proteases except for complement components C1r and C1s, pig plasminogen, bovine protein C, rodent urokinase, ancrod, gyroxin, and two insect trypsins. LP285 is identified by such a serine-protease-identifying-consensus-pattern because it exhibits an amino acid sequence fragment from Ile-92 to Cys-97 (ITAAHC; where His-96 is the LP285 active site residue) that matches the [LIVM-[ST]-A-[STAG]-H-C consensus motif.

Another consensus pattern used for detecting serine proteases is [DNSTAGC]-[GSTAPIMVQH]-x(2)-G-[DE]-S-G-[GS]-[SAPHV]-[LVMFYWH]-[LIVMFYSTANQH], where S (indicated in bold typeface and underlined) is the active serine site residue. Sequences known to belong to the class of proteins detected by the pattern include all presently known serine proteases except for 18 different proteases that have lost a characteristic conserved glycine residue (see Table 2 below). LP285 is also identified by this serine-protease-identifying-consensus-pattern because it exhibits the sequence Asp-238 to Met-249 (DACQGDSGGSLM; where Ser-244 is the LP285 active site residue; see also Table 2 below) which matches the [DNSTAGC]-[GSTAPIMVQH]-x(2)-G-[DE]-S-G-[GS]-[SAPHV]-[LIVFYWH]-[LIVMFYSTANQH] consensus motif.

Typically, if a protein possesses both of the serine and the histidine active site signature motifs indicated above, then the probability of that protein being a member of the trypsin family of serine proteases approaches 100% (see, PROSITE documentation No. PDOC00124; Hofmann, et al. 1999 Nucleic Acids Res. 27:215-219; and Bucher & Bairoch 1994 “A generalized profile syntax for biomolecular sequences motifs and its function in automatic sequence interpretation” in ISMB-94; Proceedings 2nd International Conference on Intelligent Systems for Molecular Biology. Altman, et al., eds., pp53-61, AAAI Press, Menlo Park). Furthermore, another consensus sequence [GDSGG], which surrounds around the catalytic serine residue (indicated in bold typeface and underlined) is also considered to be diagnostic for identifying a protein as a serine protease (see, Krem, et al. 1999 Jour Bio. Chem. 274:28063-28066). LP285 also exhibits such a GDSGG consensus sequence (see, Gly-242 to Gly-246 in Table 2 below), further suppporting the characterization of LP285 as a serine protease. The chymotrypsin, subtilisin, and carboxypeptidase C clans of serine protease enzymes have in common a catalytic triad formed with three amino acid residues—serine, aspartic acid, and histidine; where the serine residue functions as a nucleophile, the aspartatic acid residue functions as an electrophile, and the histidine residue functions as a base (see, e.g., Rawlings & Barrett, 1994 Families of serine peptidases. Meth. Enzymol. 244 19-61). The geometric orientations of these catalytic residues are similar between families, despite different protein folds (Rawlings & Barrett, 1994 “Families of serine peptidases.” Meth. Enzymol. 244 19-61). The linear arrangements of the catalytic residues is used to define clan relationships among serine proteases. For example the catalytic triad in the chymotrypsin clan (SA) is ordered H-D-S, but in the subtilisin clan (SB) it is ordered D-H-S and in the carboxypeptidase clan is ordered S-D-H (SC) (Rawlings & Barrett 1993 “Evolutionary families of petidases.” Biochem. J. 290 205-218). In LP285, the catalytic triad is ordered H-D-S (see Table 2 below) further evidencing the enzymatic functionality of LP285 as a serine protease and further suggesting that LP285 is a chymotrypsin-like serine protease. Consequently, based on all available evidence, LP285 is a novel trypsin-family, serine-protease.

It has been discovered that LP285 nucleic acid sequence (SEQ ID NO: 3) is expressed in the following number of LIFESEQ GOLD™ database tissue and cDNA libraries: Embryonic Structures 1/23, and the Urogenital System 1/66.

Based on the expression pattern of LP285, its homology to proteins with known functions, and literature suggesting the role of such proteins in human conditions, diseases, syndromes, etc., it is likely that compositions comprising LP285 polypeptides (or fragments thereof), polynucleotides (or fragments thereof), and/or LP285 antibodies (or LP285 binding compositions), and related reagents are also useful for the diagnosis, prognosis, treatment, amelioration, and/or intervention of a disease, condition, or state including, but not limited to, e.g., cell proliferative, autoimmune/inflammatory, immunological disorders, blood coagulative disorders, coagulation disorders, cell proliferative disorders, cancer, cellular adhesion disorders, disorders of fibrinolysis, tissue disorders, joint disorder, disorders of complement activation, cardiovascular disorders, neurological disorders, and developmental disorders.

Table 2: Primate, e.g., human, LP285 polynucleotide sequence (SEQ ID NO: 3) and corresponding polypeptide (SEQ ID NO: 4). The ORF for LP285 is 1-921 bp (with the start (ATG) and stop codons (TAG) identified in bold typeface and underlined. In case the numbering is misidentified herein, one skilled in the art could easily determine the open-reading frame without undue experimentation given the teachings herein.

LP285 DNA sequence (921 bp) (ORF=1-921):

ATGAGTCTCAAAATGCTTATAAGCAGGAACAAGCTCATTTTACTACTAGGAATAGTCTTTTTTGAACGAGG
TAAATCTGCAACTCTTTCGCTCCCCAAACCTCCCAGTTGTGGGCAGAGTCTGGTTAAGGTACAGCCTTGGA
ATTATTTTAACATTTTCAGTCGCATTCTTGGAGGAAGCCAAGTGGAGAAGGGTTCCTATCCCTGGCAGGTA
TCTCTGAAACAAAGCCAGAAGCATATTTGTGGAGGAAGCATCGTCTCACCACAGTGGGTGATCACGGCGGC
TCACTGCATTGCAAACAGAAACATTGTGTCTACTTTGAATGTTACTGCTGGAGAGTATGACTTAAGCCAGA
CAGACCCAGGAGAGCAAACTCTCACTATTGAAACTGTCATCATACATCCACATTTCTCCACCAAGAAACCA
ATGGACTATGATATTGCCCTTTTGAAGATGGCTGGAGCCTTCCAATTTGGCCACTTTGTGGGGCCCATATG
TCTTCCAGAGCTGCGGGAGCAATTTGAGGCTGGTTTTATTTGTACAACTGCAGGCTGGGGCCGCTTAACTG
AAGGTGGCGTCCTCTCACAAGTCTTGCAGGAAGTGAATCTGCCTATTTTGACCTGGGAAGAGTGTGTGGCA
GCTCTGTTAACACTAAAGAGGCCCATCAGTGGGAAGACCTTTCTTTGCACAGGTTTTCCTGATGGAGGGAG
AGACGCATGTCAGGGAGATTCAGGAGGTTCACTCATGTGCCGGAATAAGAAAGGGGCCTGGACTCTGGCTG
GTGTGACTTCCTGGGGTTTGGGCTGTGGTCGAGGCTGGAGAAACAATGTGAGGAAAAGTGATCAAGGATCC
CCTGGGATCTTCACAGACATTAGTAAAGTGCTTTCCTGGATCCACGAACACATCCAAACTGGTAACTAA

LP285 Full-Lenath Secmence (306 aa):

>LP285 (SEQ ID NO: 4) The underlined portion indicates a predicted signal sequence (Met-1 to Ser-26). A predicted SP cleavage site is between Ser-26 and Ala-27 indicated as follows: 1 MSLKMLISRNKLILLLGIVFFERGKS\hat{ }AT 28. An LP encompassed herein includes fiul-length forms encoded by an ORF disclosed herein, as well as any mature forms therefrom. Such a mature LP could be formed, for example, by the removal of a signal peptide and/or by aminopeptidase modification. For example, a putative proteolytic activation recognition site (ILGG) for LP285 is present at the beginning of the LP285 protease domain thus suggesting that LP285 is synthesized as an inactive precursor zymogen and subsequently activated by proteolytic cleavage on the amino side of the conserved ILGG sequence in LP285 (this conserved sequence (ILGG) is similar to the conserved sequence of other serine proteases such as, for example, the amphibian Xesp1 and Xesp2 (IVGG), except that in the amphibian sequences the second amino residue position is Valine (V) rather than Leucine (L), however, an L for V substitution is a conservative change of one non-polar hydrophobic amino acid residue for another and thus it is likely that the consensus sequence acts as a conserved activation site in LP285). All forms of LP285 such as, both precursor and activated forms are encompassed herein. Further, as used herein, a “mature” LP encompasses, e.g., post-translational modifications other than proteolytic cleavages (such as, e.g., by way of a non-limiting example, glycosylations, gamma-carboxylations, beta-hydroxylations, myristylations, phosphorylations, prenylations, acylations, and sulfations). Such variants are also encompassed by an LP of the present invention. Further, an LP of the invention encompasses all fragments, analogs, homologs, and derivatives of an LP described herein, thus the invention encompasses both LP precursors and any modified versions (such as, e.g., by post-translational modification) of an LP encoded by an LP nucleic acid sequence described herein..

MSLKMLISRNKLILLLGIVFFERGKSATLSLPKAPSCGQSLVKVQPWNYFNIFSRILGGSQVEKGSYPWQV
SLKQRQKHICGGSIVSPQWVITAAHCIANRNIVSTLNVTAGEYDLSQTDPGEQTLTIETVIIHPHFSTKKP
MDYDIALLKMAGAFQFGHFVGPICLPELREQFEAGFICTTAGWGRLTEGGVLSQVLQEVNLPILTWEECVA
ALLTLKRPISGKTFLCTGFPDGGRDACQGDSGGSLMCRNKKGAWTLAGVTSWCLGCGRGWRNNVRKSDQGS
PGIFTDISKVLSWIHEHIQTGN*

An LP285 Mature Seouence (280aa):

A predicted mature LP285 sequence is as follows:

ATLSLPKAPSCGQSLVKVQPWNYFNIFSRILGGSQVEKGSYPWQVSLKQRQKHICGGSIVSPQWVITAAHCI
ANRNIVSTLNVTAGEYDLSQTDPGEQTLTIETVIIHPHFSTKKPMDYDIALLKMAGAFQFGHFVGPICLPEL
REQFEAGFICTTAGWGRLTEGGVLSQVLQEVNLPILTWEECVAALLTLKRPISGKTFLCTGFPDGGRDACQG
DSGGSLMCRNKKGAWTLAGVTSWGLGCGRGWRNNVRKSDQGSPGIFTDISKVLSWIHEHIQTGN*

Additional LP285 Mature Sequences:

A putative proteolytic activation site (ILGG, which is indicated by underling below) is located in the LP285 mature sequence suggesting that LP285 can be synthesized as a mature but inactive precursor that can be subsequently activated by proteolytic cleavage on the amino side of the conserved ILGG sequence. Thus, in additional embodiments of the invention, other forms of LP285 are also encompassed herein depending on the site of proteolytic cleavage activation amino wards to the ILGG recognition site. Accordingly, any 1, 2, 3, 4, 5, 6, 7, 8, 9, 10, 11, 12, 13, 14, 15, 16, 17, 18, 19, 20, 21, 22, 23, 24, 25, 26, 27, 28, or 29 contiguous amino acids from the following LP285 sequence: Ala-27 to Arg-55 (ATLSLPKAPSCGQSLVKVQPWNYFNIFSR) can be contiguous with the following LP285 sequence: Ile-56 to Asn-306

ILGGSQVEKGSYPWQVSLKQRQKHICGGSIVSPQWVITAAHCIANRNIVSTLNVTAGEYDLSQTDPGEQTLT
IETVIIHPHFSTKKPMDYDIALLKMAGAFQFGHFVGPICLPELREQFEAGFICTTAGWGRLTEGGVLSQVLQ
EVNLPILTWEECVAALLTLKRPISGKTFLCTGFPDGGRDACQGDSGGSLMCRNKKGAWTLAGVTSWGLGCGR
GWRNNVRKSDQGSPGIFTDISKVLSWIHEHIQTGN);

to generate LP285 active forms such as, e.g.,

SRILGGSQVEKGSYPWQVSLKQRQKHICGGSIVSPQWVITAAHCIANRNIVSTLNVTAGEYDLSQTDPGEQT
LTIETVIIHPHFSTKKPMDYDIALLKMAGAFQFGHFVGPICLPELREQFEAGFICTTAGWGRLTEGGVLSQV
LQEVNLPILTWEECVAALLTLKRPISGKTFLCTGFPDGGRDACQGDSGGSLMCRNKKGAWTLAGVTSWGLGC
GRGWRNNVRKSDQGSPGIFTDISKVLSWIHEHIQTGN (Ser-54 to Asn-306);
NYFNIFSRILGGSQVEKGSYPWQVSLKQRQKHICGGSIVSPQWVITAAHCIANRNIVSTLNVTAGEYDLSQT
DPGEQTLTIETVIIHPHFSTKKPMDYDIALLKMACAFQFGHFVGPICLPELREQFEAGFICTTAGWORLTEG
GVLSQVLQEVNLPILTWEECVAALLTLKRPISGKTFLCTGFPDGGRDACQGDSGGSLMCRNKKGAWTLAGVT
SWGLGCGRGWRNNVRKSDQGSPGIFTDISKVLSWIHEHIQTGN (Asn-48 to Asn-306);
GQSLVKVQPWNYFNIFSRILGGSQVEKGSYPWQVSLKQRQKHICGGSIVSPQWVITAAHCIANRNIVSTLN
VTAGEYDLSQTDPGEQTLTIETVIIHPHFSTKKPMDYDIALLKMAGAFQFGHFVGPICLPELREQFEAGFI
CTTAGWGRLTEGGVLSQVLQEVNLPILTWEECVALLTLKRPISGKTFLCTGFPDGGRDACQGDSGGSLMC
RNKKGAWTLAGVTSWGLGCGRGWRNNVRKSDQGSPGIFTDISKVLSWIHEHIQTGN (Gly-38 to
Asn-306);

etc. All similar such forms are encompassed herein.

Comparison of LP285 with Human Serine Protease Domains

A BLOSUM62 amino acid substitution matrix was used to conduct a PILEUP sequence alignment (see, Henikoff and Henikoff 1992 Proc. Natl. Acad. Sci. USA 89: 1091510919).

The amino acid sequences of the serine proteinases compared to LP285 correspond to the mature forms of the protease domains of alpha-tryptase (Vanderslice, et al. 1990 Proc. Natl. Acad. Sci. U.S.A. 87, 3811-3815) or the catalytic chains of acrosin (Adham, et al. 1990 Hum. Genet. 84, 125-128), plasma kallikrein (Chung, et al. 1986 Biochemistry 25:2410-2417), coagulation factor XI (Fujikawa, et al. 1986 Biochemistry 25:2417-2424), serine protease hepsin (Leytus, et al. 1988 Biochemistry 27:1067-1074), plasminogen (Forsgren, et al. 1987 FEBS Lett. 213:254-260), Xesp-1, and Xesp-2 (Yamada, et al., 2000 Gene 252:209-216).

Xesp-1=Xenopus embryonic serine trotease (Xesp-1), is a secreted trypsin-like serine protease, which is a protein that functions in the extracellular matrix during embryonic development. Xesp-1 protease activities may be localized in embryos, since Xesp-1 is likely to be translated as a proenzyme and activated by enzymes that may be localized. Alternatively, inhibitors of Xesp-1 may be present in restricted regions thus conferring localized activities (Yamada, et al., 2000 Gene 252:209-216).

Xesp-2=Xenopus embryonic serine Yrotease (Xesp-2), is a type II membrane trypsin-like serine protease with a multidomain structure containing low density lipoprotein receptor domains (LDLR) and one scavenger receptor cysteine-rich domain (SRCR). Xesp-2 functions during embryonic development. Overexpression of Xesp-2 causes defective gastrulation. (Yamada, et al., 2000 Gene 252:209-216).

Highly conserved residues are indicated in a consensus line located below the aligned sequences.

A catalytic triad of histidine (H), aspartic acid (D), and serine (S) amino acid residues, which have been shown to be essential for enzymatic activity in serine proteases (see, e.g., Yu, et al. 1995 J. Biol. Chem. 270 (22): 13483-89), are indicated by a heart symbol (♥) placed underneath the column of consensus amino acid residues for each His, Asp, and Ser, residue of the catalytic triad in these serine proteinases (see, Hartley, B. S. 1970 Phil. Trans. R. Soc. B 257:77-86).

In LP285, the His-96 (ITAAHCIANR), Asp-146 (PMDYDIALLK), and Ser-244 (QGDSGGSLM) form this catalytic His-Asp-Ser triad. The consensus sequence (GDSGG) around the catalytic serine site (here, for LP285 it is S244) is considered diagnostic for identifying a protein as a serine protease (Krem, et al. 1999 Jour Bio. Chem. 274:28063-28066). All the sequences below exhibit such a consensus sequence (indicated below by inverted triangle symbols (∇) below the residues flanking the active serine site of the catalytic triad). Additionally, the presence of the aspartic acid residue (D) at position Asp-146 (PMDYDIALLK) of the catalytic triad, is another indication that LP285 has trypsin-like activity (also, see below, the indicated trypsin-like domain and serine protease, trypsin family-like active sites of LP285, which further suggest that LP285 possesses trypsin-like activity).

The conserved cysteine at LP285 residue Cys-166 (VGPICLPEL) (marked by a cloverleaf symbol ( [symbol] )) is conserved in all of the serine proteinases in this alignment. In plasma kallikrein, coagulation factor XI, and acrosin this cysteine residue has been discovered to be involved in the formation of an interchain disulfide bond with the noncatalytic chain (see, e.g., McMullen, et al. 1991a Biochemistry 30, 2050-2056; McMullen, et al. 1991b Biochemistry 30, 2056-2060; and Topfer-Petersen, et al. 1990 FEBS Lett. 275, 139-142). Therefore, it is likely that this cysteine residue will play a similar role in LP285 or by facilitating LP285 multimer formation.

Six residues before the LP285 active serine site (Ser-244; QGDSGGS) a conserved aspartic acid residue D238 (GGRDACQG) is present in LP285 at position Asp-238 (indicated in the alignment by a cross inside a circle symbol (\mathring{X})). Similarly located Asp residues in other. serine proteinases (e.g., such as trypsin) have been shown to be located at the bottom of the S1 substrate-binding pocket (when the protein folds into its mature state). Such Asp residues have also been shown to be involved in an interaction with particular locations on cognate substrates (for example, such as, an arginine (R) or lysine (K) residue) (see, e.g., Ruhlmann et al., 1973 J. Mol. Biol. 77, 417-436; and Yu, et al. 1995 J. Biol. Chem. 270 (22): 13483-89).

Two glycine residues (Gly-266 (VWSWCKGCA), and Gly-286 (GSPGIF); indicated by spade symbols ( [symbol] ) below) are conserved in LP285 and other serine proteinases. The counterparts of these two Gly residues in serine proteases having a chymotrypsin fold have been shown to be present at the entrance of the S1 substrate-binding pocket and to permit entry of large amino acid side chains to the base of the pocket. The LP285 glycine residues are likely to perform similarly

There are six conserved cysteine residues (C) (marked by diamond symbols (♦)), which are located in the protease domain of LP285 at residues: Cys-81 (KHICGGS)., Cys-97 (AAHCIAN), Cys-211 (TWEECV), Cys-229 (TFLCTGF), Cys-240 (RDACQGD), and Cys-269 (GLGCGRG). Such cysteine residues may form intermolecular disulfide bonds in the mature LP285 protein. In serine proteases with a chymotrypsin-like fold, the S1 site specificity comprises: the amino acid residues of the catalytic His-Asp-Ser triad, a substrate binding pocket whose walls are formed by three beta strands connected by two surface loops and cysteine-forming disulfide bond (C240-C269 in LP285) up of two beta-barrels, and distal structural elements (Perona & Craik 1997 Jour. Biol. Chem. 272:29987-29990).

Based on comparisons with other serine proteases having a chymotrypsin-like fold, mapping the LP285 amino acid sequence onto higher order structures found in such serine proteases (such as, for example, the higher order structure of trypsin) suggests that the structure of the S1 site for LP285 sequence comprises: the catalytic residues His-96 (ITAAHCIANR), Asp-146 (PMDYDIALLK), and Ser-244 (QGDSGGSLM); the distal surface Loops 1-3 formed from about Gly-235 to about Ser-247 (GGRDACQGDSGGS) to define Loopl; from about Val-262 to about Ile-287 (VTSWGLGCGRGWRNNVRKSDQGSPGI) to define Loop2; and from about Val-215 to about Ile-230 (LLTLKRPISGKTFLCT) to define Loop3; the disulfide bond formed between the C240-C269 LP285 cysteines helps form the walls of the catalytic pocket; and other distal elements.

A conserved proteolytic consensus sequence I[TVLK]GG is indicated by a triangle symbol (Δ) below the arginines (R), which are the first amino acid residues located N-terminally from this proteolytic recognition site.

| | |
|---|---|
| | 1 50 |
| Prostasin | RITGGSSAVA GQWPWQVSIT YE...G.V.. HVCGGSLVSE QWVLSAAHCF |
| Xesp-1 | RIVGCTDTRQ GAWPWQVSLE .....G.S.. HICGGSIISD QWILTATHCI |
| Coagulation | RIVCGTASVR GEWPWQVTLH TTSPTQ.R.. HLCGGSIIGN QWILTAAHCF |
| Kallikrein | RIVGGTNSSW GEWPWQVSLQ VKLTAQ.R.. HLCGGSLIGH QWVLTAAHCF |
| Xesp-2 | RIVGGTFANL CNWPWQVNLQ YITGV..... .LCGGSIISP KWIVTAAHCV |
| Hepsin | RIVGGRDTSL GRWPWQVSLR YD...G.A.. HLCGGSLLSG DWVLTAAECF |
| Acrosin | RIVGGKAAQH GAWPWMVSLQ IFTYNS.HRY HTCCGSLLNS RWVLTAAECF |
| LP285 | RILGGSQVEK GSYPWQVSLK .....Q.RQK HICGGSIVSP QWVITAAHCI |
| T-Plasminoge | RIKGGLFADI ASHPWQAAIF AKHRRSPGER FLCCGILISS CWILSAAHCF |
| Consensus | RIVGGT-A-L G-WPWQVSLQ YKT----R-- HLCGGSLIS- QWVLTAAHCF |
| | Δ ♦ ♥♦ |
| | 51 100 |
| Prostasin | PSE.HHKEAY EVKLGAHQLD SYSEDAKVST LK.DII.... .PHPSYLQEG |
| Xesp-1 | EHP.DLPSGC GVRLGAYQL. .YVKNPHEMT VKVDIIY... .INSEFNGPG |
| Coagulation | YG.VESPKIL RVYSGILNQS EIKEDTSFFG VQEIIIHDQY ......KMAE |
| Kallikrein | DG.LPLQDVW RIYSGILNLS DITKDTPFSQ IKEIIIHQNY ......KVSE |
| Xesp-2 | YGSYSSASGW RVFAGTLTKP SYYNASAYF. VERIIVHPGY ......KSYT |
| Hepsin | PERNRVLSRW RVFAGAVAQA S..PHGLQLG VQAVVYHGGY LPFRDPNSEE |
| Acrosin | VGKNNVHD.W RLVFGAKEIT YGNNKPVKAP LQERYV.EKI IIHEKYNSAT |
| LP285 | ANR.NIVSTL NVTAGEYDLS QTDPGEQTLT IETVIIHPHF STKKPM.... |
| T-Plasminoge | QERFP.PHHL TVILGRTYRV VPGEEEQKFE VEKYIVH... ...KEFDDDT |
| Consensus | -GRN--PSGW RV--CAL-LS SY-ED-Q-FT V-EIIIHPGY --HKE--S-- |
| | 101 150 |
| Prostasin | SQGDIALLQL SRP....ITF SRYIRPICLP AANASFPNGL .HCTVTGWGH |
| Xesp-1 | TSGDIALLKL SSP....IKF TEYILPICLP ASPVTFSSGT .ECWITGWGQ |
| Coagulation | SGYDIALLKL ETT....VNY TDSQRPICLP SKGDRNVIYT .DCWVTGWGY |
| Kallikrein | GNHDIALIKL QAP....LNY TEFQKPICLP SKGDTSTIYT .NCWVTGWGF |
| Xesp-2 | YDNDIALMKL RDE....ITF GYTTQPVCLP NSGMFWEAGT .TTWISGWGS |
| Hepsin | NSNDIALVHL SSP....LPL TEYIQPVCLP AAGQALVDGK .ICTVTGWGN |
| Acrosin | EGNDIALVEI TPP....ISC GRFIGPGCLP HFKAGLPRGS QSCWVAGWGY |
| LP285 | .DYDIALLKM AGA....FQF GHFVGPICLP ELREQFEAG. FICTTAGWGR |
| T-Plasminoge | YDNDIALLQL KSDSSRCAQE SSVVRTVCLP PADLQLPDWT .ECELSGYGK |
| Consensus | -DNDIALLKL SSP----I-F TE-IRPICLP AAG---P-GT --CWVTGWGY |
| | ♥ [symbol] |
| | 151 200 |
| Prostasin | VAPSVSLLTP KPLQQLEVPL ISRETCNCLY NIDAKPEEPH .FVQEDMVCA |
| Xesp-1 | TGSEVPLQYP ATLQKVMVPI INRDSCEKMY HINSVISETE ILIQSDQICA |
| Coagulation | RKLRDKIQN. .TLQKAKIPL VTNEECQKRY .RGHK..... ..ITHKMICA |
| Kallikrein | SKEKGEIQN. .ILQKVNIPL VTNEECQKRY .QDYK..... ..ITQRMVCA |
| Xesp-2 | TYEGGSVST. .YLQYAAIPL IDSNVCNQSY VYNGQ..... ..ITSSMICA |
| Hepsin | TQYYGQQAG. .VLQEARVPI ISNDVCNGAD FYGNQ..... ..IKPKMFCA |
| Acrosin | IEEKAP.RPS SILMEARVDL IDLDLCNSTQ WYNGR..... ..VQPTNVCA |
| LP285 | LTEGGVL..S QVLQEVNLPI LTWEECVAAL LTLKRPISGK TFL.....CT |
| T-Plasminoge | HEALSPFYSE R.LKEAHVRL YPSSRCTSQH LLN.RTVTDN MLCAGDTRSG |
| Consensus | T-E-GPLQ-- --LQEA-VPL ITNEECNK-Y LYNG-P-E-- --I--DM-CA |
| | 151 ♦ ♦ |
| | 201 250 |
| Prostasin | GYVEGGKDAC QGDSGGPLSC PVE....GLW YLTGIVSWGD ACGARNR... |
| Xesp-1 | GYQAGQKDGC QGDSQGPLVC KIQ....GFW YQAGIVSWGE RCAAKNR... |
| Coagulation | GYREGGKDAC KGDSGGPLSC K....HNEVW HLVGITSWGE GCAQRER... |
| Kallikrein | GYKEGGKDAC KGDBGGPLVC K....HNGMW RLVGITSWQE GCARREQ... |
| Xesp-2 | GYLSGGVDTC QGDSGGPLVN K....RNGTW WLVGDTSWGD GCARANK... |
| Hepsin | GYPEGGIDAC QGDSGGPFVC EDSISRTPRW RLCGIVSWGT GCALAQK... |
| Acrosin | GYPVGKIDTC QGDSGGPLMC KD..SKESAY VVVGITSWGV GCARAKR... |
| LP285 | GFPDGGRDAC QGDSGGSLMC R...NKKGAW TLAGVTSWGL GCGRGWRNNV |
| T-Plasminoge | GPQANLHDAC QGDSGGPLVC ....LNDGRM TLVGIISWGL GCGQKD.... |
| Consensus | GYPEGGKDAC QGDSGGPLVC KD--S-NG-W -LVGITSWGE GCAR-NR-- |
| | 201 [symbol] ♦ ∇∇♥∇∇ [symbol] ♦ 250 |
| | 251 300 |
| Prostasin | .......PGV YTLASSYASW IQSK.VTELQ PRVVPQTQES QPDSNLCGSH |
| Xesp-1 | .......PGV YTFVPAYETW ISERSVISFK ....PFTSSS SPSSS..... |
| Coagulation | .......PGV YTNVVEYVDW ILEKTQAV˜˜˜ ˜˜˜˜˜˜˜˜˜˜ ˜˜˜˜˜˜˜˜˜˜ |
| Kallikrein | .......PGV YTKVAEYMDW ILETTQSSDG KAQMQSPA˜˜ ˜˜˜˜˜˜˜˜˜˜ |
| Xesp-2 | .......PGV YGNVTTFLEW IYSQMRTYR˜ ˜˜˜˜˜˜˜˜˜˜ ˜˜˜˜˜˜˜˜˜˜ |
| Hepsin | .......PGV YTKVSDFREW IFQAIKTHSE ASGMVTQL˜˜ ˜˜˜˜˜˜˜˜˜˜ |
| Acrosin | .......PGI YTATWPYLNW IASKIGSN.A LRMIQSATPP PPTTRPPPIR |
| LP285 | RKSDQGSPGI FTDISKVLSW IHEHIQTGN* ˜˜˜˜˜˜˜˜˜˜ ˜˜˜˜˜˜˜˜˜˜ |
| T-Plasminoge | ......VPGV YTKVTNYLDW IRDNMRP˜˜˜ ˜˜˜˜˜˜˜˜˜˜ ˜˜˜˜˜˜˜˜˜˜ |
| Consensus | -------PGV YTKVSEYLDW ILEKIQTS-- -R-M-ST--S -P-S------ |
| | 251 [symbol] 300 |

Particularly interesting portions or fragments of the full length LP285 polypeptide include, e.g., a discovered putative signal peptide-like sequence from Met-1 to Ala-20 (MGSGRVPGLCLLVLLVHARA). Additionally interesting portions of LP285 are: a trypsin-like domain from Ile-56 to Ile-298: (ILGGSQVEKGSYPWQVSLKQRQKHICGGSIVSPQWVITAAHCIANRNIVSTLNVTAGEYDLSQTDPGEQTL TIETVIIHPHFSTKKPMDYDIALLKMAGAFQFGHFVGPICLPELREQFEAGFICTTAGWGRLTEGGVLSQVL QEVNLPILTWEECVAALLTLKRPISGKTFLCTGFPDGGRDACQGDSGGSLMCRNKKGAWTLAGVTSWGLGCG RGWRNNVRKSDQGSPGIFTDISKVLSWI); serine protease, trypsin family-like active sites: Cys-81 to Cys-97 (CGGSIVSPQWVITAAHC), Ile-92 to Cys-97 (ITAAHC), Asp-238 to Met-249 (DACQGDSGGSLM); Asp-238 to Gly-261 (DACQGDSGGSLMCRNKKGAWTLAG), and Pro-285 to Ile-298 (PGIFTDISKVLSWI); and a chymotrypsin serine protease family (S1)-like signature Pro-142 to Phe-156 (PMDYDIALLKMAGAF) that was identified based on the PRINTS database consensus sequence signature of the chymotrypsin 3-element fingerprint, which provides a signature for the chymotrypsin (S1) family of serine proteases (see., e.g., Attwood, et al. 1994 PRINTS—A database of protein motif fingerprints. Nucleic Acids Research, in press; and Attwood & Beck 1994 Protein Engineering, 7 (7), 841-848).

Trypsin-like protein domains are recognized in all proteins in families having the S1, S2A, S2B, S2C, and S5 classification of peptidases (see, e.g., Rawlings & Barrett, 1994 Meth Enzymol 244:19-61; and Sprang, et al., 1987 Science 237:905-909). Generally, demonstration of serine protease, trypsin family, active site domains in a protein is characteristic for the protein possessing serine protease functionality. It is well established, that the catalytic activity of serine proteases of the trypsin family is provided by a charge relay system involving an aspartic acid residue hydrogen-bonded to a histidine, which itself is hydrogen-bonded to a serine residue. It has also been shown that amino acid sequences in the vicinity of the active site serine and histidine residues are also well conserved in this family of proteases (see, e.g., Brenner 1988 Nature 334:528-530; and see the alignments in Table 2 above). Chymotrypsin, subtilisin, and carboxypeptidase C clans have a catalytic triad of serine, aspartate, and histidine in common: serine acts as a nucleophile, aspartate as an electrophile, and histidine as a base (Rawlings & Barrett, 1994 “Families of serine peptidases.” Meth. Enzymol. 244 19-61). The geometric orientations of the catalytic residues are similar between families, despite different protein folds (Rawlings & Barrett, 1994 Families of serine peptidases. Meth. Enzymol. 244 19-61). The linear arrangements of the catalytic residues commonly reflect clan relationships. For example the catalytic triad in the chymotrypsin clan (SA) is ordered HDS (the HDS triad is found in LP285), but is ordered DHS in the subtilisin clan (SB) and SDH in the carboxypeptidase clan (SC) (Rawlings & Barrett 1993 Evolutionary families of peptidases Biochem. J. 290 205-218). The trypsin family is almost totally confined to animals. The enzymes are inherently secreted, being synthesized with a signal peptide that targets them to the secretory pathway. Animal enzymes are either secreted directly, packaged into vesicles for regulated secretion, or are retained in leukocyte granules. Members of the chymotrypsin family may occasionally function intracellularly (for example, the intracellular digestion of bacteria in neutrophils), but most function extracellularly. The essential catalytic unit of the chymotrypsin family is around 220 amino acids in length (here, for LP285, one estimate of the protease domain is approximately 250 amino acids in length), although the protein may be extended at the N-terminus with unrelated sequences, often containing modules. Proteolytic activation of the protein takes place extracellularly, or sometimes in storage organelles, creating a new N-terminal residue—this is often isoleucine, but may be leucine, valine, or methionine (Bode & Huber 1978 Febs Lett. 90 265-269). Salivary plasminogen activator from vampire bat contains serine as its new N-terminal residue (Rawlings & Barrett 1993 Evolutionary families of peptidases Biochem. J. 290 205-218). The N-terminus forms a salt-bridge with an aspartic acid, leading to the formation of the functional active site (Rawlings & Barrett, 1994 Families of serine peptidases. Meth. Enzymol. 244 19-61). The cleaved propeptide can be as small as two amino acids, but many are much larger peptides that may contain modules. The cleaved peptide, not uncommonly, remains disulphide-bonded to the active enzyme (Rawlings & Barrett, 1994 “Families of serine peptidases.” Meth. Enzymol. 244 19-61).

Analysis of the primary amino acid structure of LP285 is shown above in Table 2. Such an analysis demonstrates that LP285 possesses a characteristic HDS catalytic triad of histidine (H), aspartic acid (D), and serine (S) residues, which have been shown to be essential for enzymatic activity in other serine proteinases (see, e.g., Yu, et al. 1995 J. Biol. Chem. 270 (22): 13483-89). In LP285, His-96 (ITAAHCIANR), Asp-146 (PMDYDIALLK), and Ser-244 (QGDSGGSLM) form this catalytic His-Asp-Ser triad.

Analysis of this alignment also demonstrates that LP285 contains a conserved aspartic acid residue (D) at amino acid residue position Asp-146 (indicated here by bold and underlining; PMDYDIALLK). Similar placement of an Asp residue in other serine proteinases is interpreted as indicating trypsin-like activity (LP285 also possesses, as indicated above, both a trypsin-like domain and serine protease trypsin-family-like active sites, which further suggest that it possesses trypsin-like activity). A conserved Cys residue in LP285 (Cys-166 (indicated here by bold and underlining; VGPICLPEL)) is also conserved in all of the serine proteinases of the alignment. This cysteine residue has been shown to be involved in the formation of an interchain disulfide bond with the noncatalytic chain in plasma kallikrein, coagulation factor XI, and acrosin (see, e.g., McMullen, et al. 1991 a Biochemistry 30, 2050-2056; McMullen, et al. 1991b Biochemistry 30, 2056-2060; and Topfer-Petersen, et al. 1990 FEBS Lett. 275, 139-142). Therefore, it is likely that Cys-166 of LP285 plays a similar role (e.g., either by participating in binding with another molecule or by permitting LP285 hetero- or homodimer formation). Additionally, at LP285 position Asp-238 -six residues before the active Ser site (Ser-244)—is an important conserved aspartic acid residue (GGRDACQG); indicated here by bold and underlining). Similarly placed aspartic acid residues in other serine proteinases (such as, e.g., trypsin) have been shown to be located at the bottom of the substrate-binding pocket of trypsin and to interact, for example, with an Arg or Lys residue on a corresponding substrate (see, e.g., Ruhlmann et al., 1973 J. Mol. Biol. 77, 417-436; and Yu, et al. 1995 J. Biol. Chem. 270 (22): 13483-89). Moreover, two LP285 Glycine residues (Gly-252 (VTSWGLGCG) and Gly-262 (SPGIFTDI)) are also conserved in other serine proteinases. The counterparts of these two Gly residues in trypsin and prostatin have been shown to be present at the entrance of the substrate-binding pocket and to permit entry of large amino acid side chains. Consequently, upon analyzing the data presented herein as a whole, LP285's primary structure reinforces the view that it possesses the enzymatic-like functionality of a serine protease. Given its sequence homology to serine proteinases, its possession of a trypsin-like domain, its possession of serine protease, trypsin-family-like active sites, and the conservation of primary features with other serine proteinases, it is likely that LP285 possesses similar catalytic properties. Based on the teachings supplied herein, one skilled in the art would be able to easily determine enzymatic like activity for LP285 using common assay techniques that measure serine protease activity. For example, LP285 enzyme activity can be assessed by a standard in vitro serine protease assay (see, for example, Stief and Heimburger, U.S. Pat. No. 5,057,414 (1991), which is incorporated by reference herein for such methods). For instance, in a non-limiting example, LP285 could easily be tested for trypsin-like activities, using synthetic substrates (see, e.g., Yu et al. 1994J. Biol. Chem. 269, 18843-18848 and the teachings supplied therein, which are hereby incorporated by reference for these methods). Those of skill in the art are aware of a variety of substrates suitable for in vitro assays, such as Suc-Ala-Ala-Pro-Phe-pNA, fluorescein mono-p-guanidinobenzoate hydrochloride, benzyloxycarbonyl-L-Arginyl-S-benzylester, Nalpha-Benzoyl-L-arginine ethyl ester hydrochloride, and the like. For example to test LP285 for arginine amidolytic activities one could use the substrate D-Pro-Phe-Arg-MCA and D-Phe-Phe-Arg-MCA. To test for lysine amidolytic activity one would use, for example, a substrate such as succinyl-Ala-Phe-Lys-MCA and t-butyloxycarbonyl-Val-Leu-Lys-MCA. To test for enzymatic activity on chymotrypsin substrates one would use, for example, a substrate such as succinyl-Ala-Ala-Pro-Phe-MCA, Ala-Ala-Phe-AMC, or Suc-Leu-Leu-Val-Tyr-AMC. Trypsin-like activity could be assayed, for example with Boc-Leu-Ser-Thr-Arg-AMC. Other methods for testing are known in the art and would be easily available. For example, such as those described in the journal BioTechniques (September, 1994), entitled “A New Protease Activity Assay Using Fluorescence Polarization.”).

In addition, protease assay kits available from commercial sources, such as Calbiochem® (San Diego, Calif.) or the Beacon® Protease Activity Detection Kit from the PanVera Corporation, Madison, Wis. For general references, see Barrett (Ed.), Methods in Enzymology, Proteolytic Enzymes: Serine and Cysteine Peptidase (Academic Press Inc. 1994), and Barrett et al., ads.), Handbook of Proteoyltic Enzymes (Academic Press Inc. 1998). Testing a protein for trypsin activity is routine in the art and would not require undue experimentation given the teachings supplied herein (e.g., as to the LP285 sequence) and given teachings in the art for methods of determining whether a suspected protein has protease activity.

Given the sequence information and knowledge of the secondary structural features of serine proteases, one can easily determine how such features map onto the LP285 sequence presented herein (see, e.g., Perona & Craik 1997 J. Biol. Chem. 272: 29987-29990, which is incorporated by reference herein). Using such information, one of skill in the art of protein engineering would be able to design amino acid modifications of LP285 to affect LP285 function, such as, for example, by modifying the catalytic triad of HDS residues, by adjusting the placement of cysteines, by modifying the size of the S1 binding pocket, by modifying residues on loops 1-3, or by modifying the residues of the substrate binding pocket. For example, to examine LP285 or LP285 variants, and their relationship to potential substrate or binding partners (e.g., such as, a cognate serpin), higher order structural determination can be carried out (such as, for example, crystallization) using methods known in the art. Alternatively, computer programs can be used to determine higher order structures. Such techniques are also common in the art. Additionally, commercial services are available to rapidly produce three-dimensional configurations and higher order structures using proteins produced from known primary amino acid sequences thus avoiding undue experimentation when assessing higher order structures of a sequence of interest (see, e.g., Structural GenomiX, 10505 Roselle St., San Diego, Calif. 92121).

Protein-protein interactions of LP285 with binding partners (such as, e.g., LP285's cognate serpin binding partner (such as, e.g., a specific serpin) or, e.g., a serpin receptor that binds an LP285 serpin/serine protease complex (such as, e.g., the serpin receptor 1: a hepatic receptor that mediates the clearance of serpin-protease complexes such as, e.g., ATIII, alpha 1-protease inhibitor, heparin cofactor II, and alpha 1-antichymotrypsin protease complexes) can be easily determined using a commercially available methods (e.g., see, the BIACORE™ system from Biacore AB, Rapsgatan 7, SE-754 50 Uppsala, Sweden). Additional methods are known in the art and described herein. Other interesting segments of LP285 are discovered portions of LP285 from about Lys-11 to about Leu-29 (KLILLLGIVFFERGKSATL); from about Ser-20 to about Ser-40 (SLPKAPSCGQS); from about Leu-41 to about Gly-58 (LVKVQPWNYFNIFSRILG); from about Leu-11 to about Gly-83 (LKQRQKHICGG); from about Gly-112 to about Gln-124 (GEYDLSQTDPGEQ); from about Thr-125 to about Thr-139 (TLTIETVIIHPHFST); from about Lys-140 to about Leu-150 (KKPMDYDIALL); from about Lys-151 to about His-160 (KMAGAFQFGH); from about Phe-161 to about Ile-179 (FVGPICLPELREQFEAGFI); from about Cys-180 to about Gly-191 (CTTAGWGRLTEG); from about Gly-192 to about Phe-204 (GVLSQVLQEVNLP); from about Ile-205 to about Ala-214 (ILTWEECVAA); from about Leu-215 to about Thr-230 (LLTLKRPISGKTFLCT); from about Gly-231 to about Gln-241 (GFPDGGRDACQ); from about Gly-242 to about Thr-258 (GDSGGSLMCRNKKGAWT); from about Gly-266 to about Asn-275 (GLGCGRGWRN); from about Asn-276 to about Gly-286 (NVRKSDQGSPG); from about Ile-287 to about Ser-296 (

IFTDISKVLS); from about Lys-11 to about Arg-23 (KLILLLGIVFFER); from about Lys-25 to about Ala-34 (KSATISLPKA); from about Gly-38 to about Tyr-49 (GQSLVKVQPWNY); from about Gly-59 to about Gln-70 (GSQVEKGSYPWQ); from about Val-71 to about Gly-82 (VSLKQRQKHICG); from about Gly-83 to about Ile-92 (GSIVSPQWVI); from about Ala-94 to about Thr-110 (AAHCIANRNIVSTLNVT); from about Ala-111 to about Leu-126 (AAHCIANRNIVSTLNVT); from about Pro-135 to about Asp-146 (PHFSTKKPMDYD); from about Ile-147 to about Gln-157 (IALLKMAGAFQ); from about Phe-158 to about Pro-168 (FGHFVGPICLP); from about Ala-176 to about Trp-185 (AGFICTTAGW); from about Gly-191 to about Val-201 (GGVLSQVLQEV); from about Asn-202 to about Cys-211 (NLPILTWEEC); from about Val-212 to about Gly-224 (VAALLTLKRPISG); from about Gly-231 to about Asp-243 (GFPDGGRDACQGD); from about Ser-244 to about Thr-258 (SGGSLMCRNKKGAWT); from about Gly-268 to about Val-277 (GCGRGWRNNV); from about Arg-278 to about Ile-287 (RKSDQGSPGI); from about from about Lys-11 to about Glu-22 (KLILLLGIVFFE); from about Arg-23 to about Val-44 (RGKSATLSLPKAPSCGQSLVKV); from about Pro-46 to about Arg-55 (PWNYFNIFSR); from about Leu-57 to about Gln-71 (LGGSQVEKGSYPWQV); from about Ser-72 to about Pro-88 (SLKQRQKHICGGSIVSP); from about Ala-94 to about Ile-103 (AAHCIANRNI); from about Val-104 to about Ser-117 (VSTLNVTAGEYDLS); from about Gln-118 to about Glu-129 (QTDPGEQTLTIE); from about Pro-135 to about Ile-147 (PHFSTKKPMDYDI); from about Ala-148 to about Gln-157 (ALLKMAGAFQ); from about Phe-158 to about Leu-167 (FGHFVGPICL); from about Pro-168 to about Phe-178 (PELREQFEAGF); from about Ile-179 to about Leu-188 (ICTTAGWGRL); from about Thr-189 to about Leu-198 (TEGGVLSQVL); from about Gln-199 to about Glu-209 (QEVNLPILTWE); from about Glu-210 to about Lys-219 (ECVAALLTLK); from about Arg-220 to about Phe-232 (RPISGKTFLCTGF); from about Pro-233 to about Gly-242 (PDGGRDACQG); from about Asp-243 to about Thr-258 (DSGGSLMCRNKKGAWT); from about Leu-259 to about Trp-273 (LAGVTSWGLGCGRGW); and from about Arg-274 to about Ile-291 (RNNVRKSDQGSPGIFTDI) whose discoveries were based on an analysis of hydrophobicity, hydropathicity, and hydrophilicity plots. Additional interesting sections of LP285 are the discovered portions of LP285 from about Ile-7 to about Leu-16 (ISRNKLILLL); from about Gly-17 to about Ser-26 (GIVFFERGKS); from about Ser-40 to about Phe-50 (SLVKVQPWNYF); from about Asn-51 to about Tyr-67 (NIFSRILGGSQVEKGSY); from about His-79 to about Gln-89 (HICGGSIVSPQ); from about Trp-90 to about Asn-100 (WVITAAHCIAN); from about Arg-101 to about Asp-115 (RNIVSTLNVTAGEYD); from about Leu-116 to about His-134 (LSQTDPGEQTLTIETVIIH); from about Asp-146 to about Phe-158 (DIALLKMAGAFQF); from about Gly-159 to about Glu-169 (GHFVGPICLPE); from about Leu-170 to about Ile-179 (LREQFEAGFI); from about Cys-180 to about Gly-192 (CTTAGWGRLTEGG); from about Val-192 to about Cys-211 (VLSQVLQEVNLPILTWEEC); from about Pro-221 to about Thr-230 (VLSQVLQEVNLPILTWEEC); from about Gly-231 to about Gly-242 (GFPDGGRDACQG); from about Asp-243 to about Gly-255 (DSGGSLMCRNKKG) from about Ala-260 to about Gly-272 (AGVTSWGLGCGRG); from about Trp-273 to about Ser-284 (WRNNVRKSDQGS); from about Pro-285 to about Val-294 (PGIFTDISKV). These fragments were discovered based on analysis of antigenicity plots. Further, particularly interesting LP285 segments are LP secondary structures (e.g., such as a helix, a strand, or a coil). Particularly interesting LP285 coil structures are the following: from about Glu-22 to about Ser-26; from about Leu-31 to about Gln-39; from about Gln-45 to about Asn-48; from about Leu-57 to about Pro-68; from about Cys-81 to about Pro-88; from about Ala-95 to about Asn-102; from about Ser-117 to about Gln-124; from about Pro-135 to about Asp-144; from about Gly-159 to about Cys-166; from about Thr-182 to about Gly-191; from about Asn-202 to about Pro-204; from about Arg-220 to about Lys-225; from about Gly-231 to about Arg-237; from about Gly-242 to about Gly-246; from about Asn-252 to about Gly-255; from about Gly-268 to about Asn-275; from about Ser-280 to about Gly-286; and from about Thr-304 to about Asn-306. Particularly interesting helix structures are from about Lys-4 to about Ile-7; from about Ser-195 to about Glu-200; and from about Trp-208 to about Thr-217. Particularly interesting strand structures are from about Leu-41 to about Lys-43; from about Trp-69 to about Ser-72; from about Trp-90 to about Thr-93; from about Val-104 to about Asn-108; Glu-129 to about Ile-133; from about Phe-227 to about Cys-229; from about Ser-247 to about Cys-250; and from about Trp-257 to about Ala-260. Further encompassed by the invention are contiguous amino acid residue combinations of any of the predicted secondary structures described above. For example one coil-helix-coil-strand-coil motif of LP285 combines the coil of Asn-202 to Pro-204; the helix of Trp-208 to Thr-217; the coil of Arg-220 to Lys-225; the strand of Phe-227 to Cys-229; and the coil of Gly-231 to Arg-237 to form an interesting fragment of contiguous amino acid residues from about Asn-202 to about Arg-237. Other combinations of contiguous amino acids are contemplated as can be easily determined from the teachings herein.

LP285 Functions

Given the analysis taught herein of: LP285 primary amino acid and domain architecture, the relationship of LP285 amino acid sequence and higher order structural features compared with known serine proteases having chymotrypsin folds and their higher order structural features (including the known functions of these serine proteases and their higher order structures as described herein), it is likely that an LP285, an LP285 variant, an LP285 agonist, an LP285 antagonist, an LP285 binding partner or an LP285 fragment as described herein plays a similar role to a known serine proteases in a variety of physiological processes. Some non-limiting examples of functions such a composition is likely to participate in are, for example, those such as: modulation of the finely tuned set of checks and counterchecks in various proteolytic cascades (e.g., the kinin cascade or the blood coagulation cascade) involving fluids (e.g., such as in plasma) or solids (e.g., such as in tissues or in the extracellular matrix); inflammation (e.g., by maintaining balance within and/or between the inflammatory cascades such as, for example, inflammatory cascades of plasma factors); coagulation (e.g., such as during the contact phase of coagulation, however, LP285 or its variants may function as both a pro- and/or a anticoagulant depending on which part, time, or portion, of a coagulation cascade LP285 is active); complement activation; regeneration; various immune responses (such as, e.g., during complement activation, or responses to parasite and/or bacterial infection); blood coagulation and/or coagulative disorders; shock syndromes due to serious injury or septicemia (e.g., such as in conditions where massive consumption of plasma protease inhibitors result in uncontrolled proteolysis with subsequent activation of coagulation, fibrinolysis, the complement and kinin cascades ensuing in often fatal conditions of disseminated intravascular coagulation); sepsis; vascularization (such as that, e.g., involved in diabetic conditions, regulation of blood pressure, modulation of tumor progression); extra-cellular matrix (ECM) activities (such as, e.g., modulation of cartilage or bone formation (or capsular remodeling)); tumorgenesis; cellular metastasis; cell proliferation (e.g., such as in growing tissues where control of cell proliferation depends on the resorption of elements of the surrounding extracellular matrix. In some such cases, the regulation of tissue growth is achieved, e.g., via the regulation of a thin shell of proteolytic activity around a single cell (e.g., developing, proliferating, and/or migrating) balanced by cell surface secretion of specific cell surface protease inhibitor. Imbalance of these factors can lead to dysfunction at the cellular level (e.g., resulting in metastasis and tumorgenesis)); cytostatic; proliferative; vulnerary; immunomodulatory; antidiabetic; antiasthmatic; antirheumatic; antiarthritic; antiinflammatory; antithyroid; antiallergic; antibacterial; antiviral; dermatological; neuroprotective; cardiant; thrombolytic; coagulant; nootropic; vasotropic; antipsoriatic; antiangiogenic; and protein conformational disease (such as, e.g., errors of LP285 polymerization) that can result in suboptimal levels of LP285 and/or both disease and degeneration of the cells in which conformationally deficient LP285 is located.

LP285 & Inflammation

Systemic inflammatory states are frequently accompanied by activation of the coagulation system and activation of the coagulation system is an almost invariable consequence of septic shock. The simultaneous activation of the innate immune response and the coagulation system after injury is a phylogenetically ancient, adaptive response that can be traced back to the early stages of eukaryotic evolution. Most invertebrate species lack differentiated phagocytic cells and platelets. They possess a common cellular and humoral pathway of inflammation and clotting after a breach in their internal milieu by either trauma or infection. The close linkage between clotting and inflammation has been preserved throughout vertebrate evolution and is readily demonstrable in human physiologic responses to a variety of potentially injurious stimuli. The same pro-inflammatory stimuli that activate the human clotting cascade also activate phagocytic effector cells (such as, e.g., neutrophils, monocytes, and macrophages). Consequently, the role of LP285 in physiological functions will likely cross artificial boundaries designated solely as inflammation or immune responses and thus information suggesting a role for LP285 in inflammation is also indicative of a role for LP285 in an immune response and vice versa. Additionally, studies showing functions and reactions in serine proteases related to LP285 (as evidenced by sequence identity) will also inform questions regarding similar functions and reactions with LP285. For example, recently, it has been shown that serine proteases are intimately involved in the modulation of the activities of cytokines and their receptors. Particularly at sites of inflammation, high amounts of the active serine proteases elastase, Cathepsin G, and proteinase 3 are released from infiltrating polymorphonuclear cells in close temporal correlation to elevated levels of inflammatory cytokines, strongly indicating that these proteases are involved in the control of cytokine bioactivity and availability. For instance, a serine protease CD26/dipeptidyl-peptidase IV (CD26/DPP IV) plays an important role in immune function (Sozzani, et al. 2000 Pharm Acta Helv 74(2-3): 305-312). CD26/DPP IV functions by removing NH2-terminal dipeptides from several chemokines and thus, profoundly affects their biological activity. Chemokines are a superfamily of proteins that play a central role in immune and inflammatory reactions and in viral infections. Chemokine receptors can function as entry/fusion co-receptors for human immunodeficiency virus (HIV)-1 infection, and regulation of receptor expression by cytokines may be relevant for viral infection. Consequently, post-translational processing of chemokines can profoundly affect their interaction with receptors. For instance, Kaposi's sarcoma (KS)-associated herpes virus 8 encodes for three chemokine-like proteins that show homology with the MIP cluster of CC chemokines. These viral chcmokines possess a partial agonist activity for certain chemokine receptors and may function as receptor antagonists. This biological activity could represent a strategy developed by the KS-associated herpes virus 8 to subvert immunity impairing the generation of an effective anti-viral immune response. In a similar manner, LP285 may function to modulate immune activity by postranslation modification of known and useful chemokine proteins. Furthermore, CD26/DPP IV has been shown to play a role in T-cell proliferation and chemotaxis and of fibroblast activation in liver disease (e.g., human cirrhosis)(McCaughan, et al. 2000 Immunol Rev 174:172-191). Consequently, LP285 may play a similar role by activating immune cells in such conditions.

Moreover, growing evidence suggests that, through its interactions with cytokines and degradative enzymes, the extracellular matrix (ECM) microenvironment has a specialized role in providing intrinsic signals for coordinating actions of cells of the immune system (e.g., such as, leukocytes). Recent advances also reveal that enzymatic modifications (such as through serine proteases) to ECM moieties and cytokines induce distinctive cellular responses, and are likely to be part of a mechanism that regulates the perpetuation or arrest of inflammation. LP285 may be important in such a role by its ability to enzymatically modify the ECM microenvironment during the inflammatory response. Furthermore, since it has been shown that serine proteases facilitate several steps in cancer progression, it is useful to identify serine proteases that are most suitable for drug targeting by using indicators of actual enzyme activity in a biological sample and not simply characterizing levels of messenger RNA or an immunoassay of the suspect protein. Accordingly, an automated microtiter plate assay can be used to allow detection of a suspected protease (such as, e.g., LP285) in tissue samples of patients with a proliferative disease condition (for example, see, e.g., the proteomic screen for proteases in colorectal carcinomas developed by McKerrow, et al., 2000 Mol Med. (5): 450-460, which is incorporated by reference herein for these teachings). Such an analysis can identify proteases whose activities may be essential for tumor progression and are not completely balanced by endogenous inhibitors. Such proteases are logical targets for efforts to produce low molecular weight protease inhibitors as a potential chemotherapy. Employing such an assay on LP285 to test its serine protease-like activity in a biological sample would allow a determination of its role in diseases of cell proliferation, such as, e.g., colon cancer.

LP285's homology to proteins involved in blood coagulation (e.g., plasma kallikrein, coagulation factor IX, and plasminogen), which have been shown to be involved in effecting other members of the coagulation cascade (such as, e.g., kininogen, and factor X) suggest that LP285 may also be participate in the blood coagulation system. Furthermore, additional evidence suggests that LP285 may also participate in inflammatory processes due to the highly integrated linkage between systemic inflammation and coagulation that is maintained in all vertebrates (see, e.g., Opal S. M. 2000 Critical Care Med. (9 Suppl): S77-80). Accordingly, LP285 may be involved in diseases, disorders, conditions associated with stimulation of both the coagulative and inflammatory systems, such as, for example, sepsis.

Consequently, LP285, an LP285 variant, an LP285 agonist, an LP285 antagonist, an LP285 binding partner or an LP285 fragment as described may also exhibit anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. An LP285, an LP285 variant, an LP285 agonist, an LP285 antagonist, an LP285 binding partner or an LP285 fragment may also be useful to treat anaphylaxis and hypersensitivity to an antigenic substance or material.

Acute Inflammatory Response Model

To test an acute inflammation response for an LP285, an LP285 variant, an LP285 agonist, an LP285 antagonist, an LP285 binding partner or an LP285 fragment, one can adapt the method of Eberini, et al. 1999 Electrophoresis 20(4-5): 846-53 (incorporated herein for these teachings). In brief, rodents are injected with a phlogistic stimulus (e.g., turpentine), turpentine and daily doses of indomethacine, and indomethacine alone. In inflamed animals, peak changes for acute-phase reactants are evaluated between 48 and 72 h after the phlogistic stimulus by two-dimensional electrophoresis (2-DE) to check for, for example, plasma concentration of LP285 expression, among other expressed molecules. Presence of LP285 is indicative of it being an acute phase protein whose changes are modulated via anti-inflammatory reaction.

Acute Inflammation Response Model with LP285 Transgenics

Using a method based on Chen, et al., 1997 Life Sci 60(17): 1431-5 (which is incorporated herein for these teachings), the potential role of LP285 in inflammation is evaluated in transgenic mice by overexpressing the LP285 gene under the control for example, of mouse metallothionein metal-responsive promoter. Briefly, bacterial endotoxic lipopolysaccharide (LPS) is injected intraperitoneally into mice at a dose of 600 microg/25 g body weight. The death toll is recorded every 12 hours for 3 days. The survival rate of transgenic male mice is assessed versus that of control male mice 3 days post LPS injection. In comparison, the survival rate of transgenic female mice is assessed versus that of control female mice to assess LP285 response to hormonal differences. Recombinant LP285 levels in the circulation of these mice is assessed for increase after LPS treatment. The results are examined to determine if LP285 transgenic mice have a higher survival rate than their non-transgenic control littermates after endotoxin shock and whether there is a gender based resistance to lethality induced by endotoxin shock. These results win suggest if LP285 has a protective effect during acute phase inflammation.

Inflammation Model for Pancreatitis

To determine if LP285 plays a role in pancreatic disease (e.g., such as pancreatitis) and is useful as a diagnostic indicator in such conditions, peritoneal exudates in acute pancreatitis subjects are obtained and examined for the presence of LP285 or LP285 complexes. Peritoneal lavages effectively clear released serpin-like complexes in such conditions (e.g., tissue kallikrein in pancreatic conditions is found complexed to kallistatin both in plasma and in peritoneal fluid). The degree of complexing of serpins in such instances is the result of the interaction between enzyme and inhibitors and the turnover of the complexes formed. Levels of LP285 or LP285 complexes in patients with pancreatic necrosis can be used to suggest if LP285 may act as an early marker in pancreatic disease (e.g., as a marker of severity in acute pancreatitis).

Inflammation Model for Liver Disease

To determine if LP285 plays a role in hepatic disease (e.g., such as the result of inflammation response) one can adapt the method of Newsholme et al. 2000 Electrophoresis 21(11): 2122-8 (incorporated herein for these methods) and generate a drug-induced increase in heptocellular rough endoplasmic reticulum (RER) in Sprague-Dawley rats by giving a substituted pyrimidine derivative. Subsequently, the experimental subjects are checked for the presence of LP285 which is interpreted as being indicative of the presence of an acute phase protein whose changes follows an inflammatory reaction supporting the suggestion that LP285 plays a role in, for example, acute phase liver inflammation.

Inflammation and Neurological Disease

Cytokines such as interleukin-6 (IL-6) have been detected in the cortices of Alzheimer disease (AD) patients, indicating a local activation of components of the unspecific inflammatory system. IL-6 may precede neuritic changes, and the immunological mechanism may be involved both in the transformation from diffuse to neuritic plaques in AD and in the development of dementia. To determine if LP285 plays a role in neurological disease (e.g., such as the result of an inflammation response) one can adapt the method of Hull, et al. 1996 Eur Arch Psychiatry Clin Neurosci 246(3): 124-8 (incorporated herein for these teachings) to determine if LP285 plays a role in such processes.

Furthermore, in the brain, the acute phase protein antichymotrypsin is produced in response to pro-inflammatory cytokines by the reactive astrocytes, in particular those surrounding the amyloid plaques of Alzheimer's disease brains. Accordingly, one can also adapt the method of Cardinaux et al., 2000 Glia 29(1): 91-7 to determine if similar pro-inflammatory molecules (e.g., such as, lipopolysaccharides (LPS), IL-1beta, and TNF alpha) induce the expression of LP285 in mouse primary astrocytes and whether the results of such data support a role for the induction of LP285 expression by pro-inflammatory cytokines in the brain (e.g., using mouse cortical astrocytes as a model system).

Hemostatic and Thrombolytic Activity

LP285, an LP285 variant, an LP285 agonist, an LP285 antagonist, an LP285 binding partner or an LP285 fragment as described herein may also exhibit hemostatic or thrombolytic activity. As a result, such a composition is expected to be useful in treatment of various coagulation disorders (including hereditary disorders, such as hemophilias) or to enhance coagulation and other hemostatic events in treating wounds resulting from trauma, surgery or other causes. Such a composition may also be useful for dissolving or inhibiting formation of thromboses and for treatment and prevention of conditions resulting therefrom (such as, for example, infarction of cardiac and central nervous system vessels (e.g., stroke). The activity of LP285, an LP285 variant, an LP285 agonist, an LP285 antagonist, an LP285 binding partner or an LP285 fragment as described herein may, among other means, be measured by the following methods: Assay for hemostatic and thrombolytic activity include, without limitation, those described in: Linet et al., J. Clin. Pharmacol. 26:131-140, 1986; Burdick et al., Thrombosis Res. 45:413-419, 1987; Humphrey et al., Fibrinolysis 5:71-79 (1991); Schaub, Prostaglandins 35:467-474,1988. A potential function of LP285 in vascular biology (such as, e.g., testing mitogenic responses via, for example, an induced MAPK pathway) can be investigated by studying the role of LP285 in the proliferation and migration of cultured primary aortic vascular smooth muscle cells (VSMCs) in vitro and in neointima formation in rat artery after balloon angioplasty in vivo based on the methods of Miao et al., 2000 Circ Res 86(4): 418-24 which is incorporated herein by reference for the teachings assay with modification for LP285 specificity).

Blood Pressure Model

To examine if LP285 has an effect on the vasculature and on blood pressure homeostasis, an intravenous bolus injection of LP285 is given to a subject (e.g., such as an anesthetized rodent) to look for a rapid, potent, and transient reduction elevation of mean arterial blood pressures. Infusions of purified LP25 in the dosage of about 0.07-1.42 nmol/kg into cannulated rodent jugular veins are carried out and the effect on the mmHg reading of blood pressure is determined in a dose-dependent manner. Significant variation from controls indicates a role for LP285 in blood pressure homeostasis.

Alternatively, to investigate the role of LP285 in blood pressure regulation, LP285 can be delivered to hypotensive transgenic mouse lines by intramuscular injection (see, e.g., the method of Ma, et al. 1995 J Biol Chem 270(1): 451-5, which is incorporated herein for these teachings). Expression of the LP285 is examined for expression in skeletal muscle by reverse transcription-polymerase chain reaction and Southern blot analysis at 10, 20, 30, and 40 days post-injection. Immunoreactive LP285 levels in the muscle and serum of these mice is quantified by an LP285-specific enzyme-linked immunosorbent assay and Western blot analysis. The levels of LP285 mRNA and immunoreactive protein are examined at 10, 20, and 30 days post-injection. During this period, LP285 delivery is examined to determine its effect on systemic blood pressure compared to that of normotensive control mice.

Furthermore, to elucidate therapeutic potentials of LP285 in hypertension, a LP285 polynucleotide encoding an LP285 or variant thereof (e.g., in an adenoviral vector) is directly introduced into spontaneously hypertensive rats (SHR) through portal vein injection (see, e.g., the method of Ma, et al. 1995 J Biol Chem 270(1): 451-5, which is incorporated herein for these teachings). Still furthermore, the following method (adapted from Gerova, M 1999 Physiol Res 48(4): 249-57, which is incorporated herein for these assay teachings) can be used to determine whether LP285 exerts a protective effect in chronic-inhibition-of-nitric-oxide-synthase-induced hypertension. Chronic-inhibition-of-nitric-oxide-synthase-induced hypertension is created by giving N omega-nitro-L-arginine methyl ester (L-NAME, 40 mg/100 ml water or given in a dose of 50 mg/kg into the jugular vein) orally to Sprague-Dawley rats, while controls receive regular tap water. Blood pressure is measured in the right carotid artery by a Statham pressure transducer in acute experiments, and on the tail artery by the plethysmographic method weekly in chronic experiments. Subsequently, LP285 mRNA levels are measured and compared with known vascularization effecting proteins such as, e.g., proteins of the kallikrein-kinin system. The results are used to assess whether enhanced LP285 synthesis has a protective role against the cardiovascular effects induced by chronic inhibition of nitric oxide synthesis.

Diabetes & Muscle Wasting Model

To investigate the role of LP285 as a factor contributing to muscle wasting (such as, e.g., observed in diabetes and fasting), one can adopt the method of Kuehn et al., 1988 Biol Chem Hoppe Seyler 369 Suppl:299-305 (which is incorporated herein by reference for these assay teachings). Briefly, using such techniques, LP285 expression levels are examined in the skeletal muscles of fasting rodents. Lowered levels of LP285 suggest that LP285 contributes to diseases of muscle wasting. Accordingly, increasing the level of LP285 in such conditions may ameliorate such conditions. To determine the involvement of LP285 in the development of diabetic retinopathy, one can adopt the method of Hatcher, et al., 1997 Invest Ophthalmol Vis Sci 38(3):658-64 (which is incorporated herein for these assay teachings). Briefly, diabetes is induced by streptozotocin (STZ) (55 mg/kg body weight in 0.05 M citrate buffer, pH 4.5) in male Sprague-Dawley rats (150 to 175 g, 6 weeks old) as confirmed by hyperglycemia and reduced body weight. Retinas are dissected from animals at 1, 2, and 4 months of induced diabetes-like conditions. The functional activity of LP285 in retinal homogenates is determined by immunoreactive LP285 levels measured by enzyme-linked immunosorbent assay. Additionally, LP285 messenger RNA (mRNA) levels in the retina are measured by Northern blot analysis using an LP285 complementary DNA probe. The activity of total Na+, K(+)-ATPase is determined by a radioassay. Total protein concentration is determined by a protein assay.

LP285 & Extracellular Matrix

Extracellular matrix (ECM) degradation and turnover are important processes in tissue remodeling during development, wound healing, regeneration, metastasis, tumor necrosis, bone and cartilage degenerative disease (e.g., arthritic conditions), and inflammation. Particular molecules known to be involved in ECM turnover and regulation (such as, e.g., in tumor invasion and metastasis) are serine proteases and serpins. As described herein, LP285 may also play a role in effecting the role of the ECM in, for example, tissue remodeling during development or repair, cell proliferation conditions, metastatic disease, wound healing, tumorgenesis, tumor necrosis, and inflammation. Moreover, growing evidence suggests that, through its interactions with cytokines and degradative enzymes, the extracellular matrix (ECM) microenvironment has a specialized role in providing intrinsic signals for coordinating actions of cells of the immune system (e.g., leukocytes). Recent advances also reveal that enzymatic modifications (such as through serine proteases) to ECM moieties and cytokines induce distinctive cellular responses, and are likely to be part of a mechanism that regulates the perpetuation or arrest of inflammation. LP285 may be important in such a role by its ability to enzymatically modify the ECM microenvironment during the inflammatory response.

Furthermore, serpins such as alpha 1 -antitrypsin, alpha 1-antichymotrypsin, plasminogen activator inhibitor (PAI)-1 & 2, have been found to be located around loose hip prostheses suggesting that chymotrypsin-like serine enzymes in tissue interfaces directly weaken periprosthetic tissue thus, LP285 and/or antagonists to LP285 may have a role here also. The pseudocapsular tissues may induce cellular host response and proteolytic activation thus contributing to loosening of prosthetic devices via release of serine proteases into synovial fluid. A remedial pseudosynovial fluid with a high content of appropriate serpins would affect low proteolytic potential, and thus, could be produced to prevent the unfavorable elevation of proteolytic enzymes in loco as a local host response to implants. Accordingly, an antagonist to LP285 could play a role in ameliorating such conditions by localized inhibition of serine protease activity either through direct targeting or in a psuedosynovial fluid mixture that is appropriately placed.

Tissue Growth Activity

A protein of the present invention also may have utility in compositions used for bone, cartilage, tendon, ligament and/or nerve tissue growth or regeneration, as well as for wound healing and tissue repair and replacement, and in the treatment of burns, incisions and ulcers. A protein of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery. A protein of this invention may also be used in the treatment of petiodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendinitis, carpal tunnel syndrome and other tendon or ligament defects. The compositions may also include an appropriate matrix and/or sequestering agent as a carrier as is well known in the art.

The protein of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions, which may be treated in accordance with the present invention, include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like. It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity. A protein of the present invention may also be useful for gut protection or regeneration and treatment of lung or liver fibrosis, reperfusion injury in various tissues, and conditions resulting from systemic cytokine damage. A protein of the present invention may also be useful for promoting or inhibiting differentiation of tissues described above from precursor tissues or cells; or for inhibiting the growth of tissues described above.

Tissue Damage Model

To evaluate a role for LP285 in response to tissue damage, direct muscle injury can be induced in rodents (based on the method of Festoff, et al. 1994 J Cell Physiol 159(1):11-18, which is incorporated herein for these assay teachings). Applicants hypothesize that the magnitude and temporal sequence of serine protease serpin-like activation, and the activation of cognate proteases such as LP285 implicate the role of proper serpin:protease balance in tissue injury and repair. Participation of complex receptors, such as the alpha 2-macroglobulin receptor/low density lipoprotein receptor-related protein (LRP), various growth factors, cytokines, and other molecules, in regulating this balance have been also implicated in playing a role in tissue regeneration and repair. Consequently, it is likely that an LP285, an LP285 variant, an LP285 agonist, an LP285 antagonist, an LP285 binding partner or an LP285 fragment may play a similar role.

Additionally, assays for tissue generation activity include, without limitation, those described in: International Patent Publication No. W095/16035 (bone, cartilage, tendon); International Patent Publication No. W095/05846 (nerve, neuronal); International Patent Publication No. W091 /07491 (skin, endothelium). Additionally, assays for wound healing activity include, without limitation, those described in: Winter, Epidermal Wound Healing, pp. 71-112 (Maibach, H I and Rovee, D T, eds.), Year Book Medical Publishers, Inc., Chicago, as modified by Eaglstein and Mertz, J. Invest. Dermatol 71:382-84 (1978).

Spinal Cord Regeneration Model

To evaluate the role LP285 in a spinal cord regeneration response (based on the methods of O'Hara, and Chemoff 1994 Tissue and Cell, 26: 599-611; Chemoff, et al. 1998 Wound Rep. Reg. 6: 435-444; and Chemoff, et al, 2000 Wound Rep. Reg. 8: 282-291, which are incorporated herein for these teachings) a tissue culture system using axolotl spinal cord ependymal cells is used to test the effects of an LP285, an LP285 variant, an LP285 agonist, an LP285 antagonist, an LP285 binding partner or an LP285 fragment on, for example, nerve and tissue regeneration. Additionally using other techniques to investigate similar issues (see, e.g., Itasaki, et al, 1999 Nature Cell Biology December;1(8):E203-207; Momose, et al., 1999 Develop. Growth Differ. 41:335-344; and Atkins, et al., 2000 Biotechniques 28: 94-96, 98, 100; which are incorporated herein for these teachings), one can conduct localized transfection studies of LP285 constructs in frog limb cultures and frog spinal cord. Although the above referenced methods were first developed for use in the chick, they can also be adapted for use, for example, in a frog limb system to examine the role of an LP285, an LP285 variant, an LP285 agonist, an LP285 antagonist, an LP285 binding partner or an LP285 fragment in, for example, cellular regeneration. Similar models can bc adapted to examine the role of an LP285, an LP285 variant, an LP285 agonist, an LP285 antagonist, an LP285 binding partner or an LP285 fragment in organ regeneration (e.g., such as hepatic regeneration using available liver models and assay techniques). Furthermore, since it has been shown that serine proteases facilitate several steps in cancer progression, it is useful to identify serine proteases and their cognate serpins that are most suitable for drug targeting by using indicators of actual enzyme activity in a biological sample not simply levels of messenger RNA or an immunoassay of the suspect protein. Accordingly, an automated microtiter plate assay can be used to allow detection of a suspected serine protease (such as, e.g., LP285) in tissue samples of patients with a proliferative disease conditions (for example one can adapt the proteomic screen for proteases in colorectal carcinomas developed by McKerrow, et al., 2000 Mol Med. (5): 450-460, which is incorporated by reference herein for these teachings, to be used to detect protease inhibitors. In fact, using this method on can likely find potential LP285 binding partner proteases.). Such an analysis can identify protease inhibitors whose activities may be important during tumorgenesis or tumor progression. Additional assays or methods for assessing an activity of an LP of the invention may, among other means, be measured by other methods described herein.

Features of LP NO: 3 (LP272)

LP272 is a novel secreted polypeptide (SEQ ID NO: 6). It has been discovered that LP272 nucleic acid sequence (SEQ ID NO: 5) is expressed in the following number of LIFESEQ GOLD™ database tissue and cDNA libraries: embryonic Structures 1/23; Nervous System 4/221; Respiratory System 2/95; (the numerator represents the number of libraries positively expressing LP 272 sequence and the denominator represents the total number of libraries examined).

LP272 nucleic acid sequence has been localized to the 1q21 region of human chromosome number 1. Moreover, the following diseases, conditions, syndromes, disorders, or pathological states have also been mapped to this region of the human genome: Vohwinkel syndrome with ichthyosis (Camisa, et al. 1988 “Autosomal dominant keratoderma, ichthyosiform dermatosis and elevated serum beta-glucuronidase.” Dermatologica 177:341-347, Carnisa & Rossana 1984 “Variant of keratoderma hereditaria mutilans (Vohwinkels syndrome): treatment with oraly administered isotretinoin.” Arch. Derm. 120:1323-1328, Korge, et al. 1997 “Loricrin mutation in Vohwinkels keratoderma is unique to the variant mith ichthyosis” J. Invest. Derm. 109:604-610, and Maestrini, et al. 1996 “A molecular defect in loricrin, the major component of the cornified cell envelope, underlies Vohwinkel'syndrome.” Nature Genet. 13:70-77); progressive symmetric erythrokeratoderma, (Ishida-Yamamoto, et al. 1997 “The molecularpathology of progressive symmetric erythrokeratoderma: a frameshift mutation in the loricrin gene and perturbations in the cornified cell envelope.” Am. J. Hum. Genet. 61:581-589); Medullary cystic kidney disease (Christodoulou, et al 1998 “Chromosome 1 localization of a gene for autosomal dominant medullary cystic kidney disease (ADMCKD)” Hum. Molec. Genet 7:905-911); hemolytic anemia due to PK deficiency (Rockah, et al 1998 “Linkage disequilibrium of common Gaucher disease mutations with a polymorphic site in the pyruvate kinase (KLR) gene.” Am. J. Med. Genet. 78:233-236); papillary renal cell carcinoma (Schmidt, et al. 1997 “Germline and somatic mutations in the tyrosine kinase domain of the MET proto-oncogene in papillary renal carcinoma” Nature Genet. 16:68-73); thyroid carcinoma with papillary renal neoplasia (Malchoff, et al. 2000 “Papillay thyroid carcinoma associated with papillary renal neoplasia: genetic linkage anaylsis of a distrinct heritable tumor syndrome” J. Clin. Endocr. Metab. 85:1758-1764); nephropathic-hypertension (Cohn, et al. 2000 “A locus for an autosomal dominant form of progressive renal failure and hypertension at chromosome 1q21” Am. J. Hum. Genet. 67:647-651); familial nonchromaffin paragangliomas (Niemann, et al. 2001 “Assignment of PGL3 to chromosome 1 (q21-q23) in a family with autosomal dominant non-chromaffin paraganglioma” Am. J. Med. Genet 98:32-36); elliptocytosis, pyropoikilocytosis, and recessive spherocytosis (Gallagher, et al. 1998 “Hematologically important mutations: spectrin and ankyrin variants in hereditary spherocytosis” Blood Cells Molec. Dis. 24:539-543, and Rouleau, et al. 1990 “A genetic map of chromosome 1: comparison of different data sets and linkage programs.” Genomics 7: 313-318); susceptibility to Vivax malaria (McAlpine, et al. 1989 “Mapping the genes for erythrocytic alpha-spectin 1 (SPTA1) and coagulation factor V (F5)” Cytogenet. Cell Genet. 51:1042); congenital insensitivity to pain with anhidrosis (Shatzky, et al. “Congenital insensitivity to pain with anbidrosis (CIPA) in Israeli-Bedouins: genetic heterogeneity, novel mutations in the TRKA/NGF receptor gene, clinical findings, and results of nerve conduction studies” 2000 Am. J. Med. Genet. 92:353-360); and familial medullary thyroid carcinoma (Gimm, et al. 1999 “Mutation analysis reveals novel sequence variants in NTRK1 in sporadic human medullary thyroid carcinoma” J. Clin. Endocr. Metab. 84:2784-2787). Accordingly, an isolated and/or recombinant molecule comprising LP272 nucleic acid sequence meets the statutory utility requirement of 35 U.S.C. §101 since such a molecule can be used, for example, to hybridize near a nucleic acid sequence associated with one or more of the above stated diseases, conditions, syndromes, disorders, or pathological states and thus serve as a marker for such a disease locus.

Table 3: Primate, e.g., human, LP272 polynucleotide sequence (SEQ ID NO: 5) and corresponding polypeptide (SEQ ID NO: 6). The ORF for LP272 is 1-930 bp (with the start (ATG) and stop codons (TAG) identified in bold typeface and underlined. In case the numbering is misidentified herein, one skilled in the art could easily determine the open reading frame without undue experimentation given the teachings herein.

LP272 DNA sequence (930 bp) (ORF=1-930):

LP272 (start (atg) and stop (tga) codons are indicated in bold typeface and underlined).

ATGGAATGCATGGGGCTCCTGCGCCCCCTCTTCCTCCTTAGCGGCTGCTGCCAGGCCCTGGAGATCTCACT
GGACCAGGAACATATTCCCTTTGGACCCGTGGTGTATCAGACGCAAGCCACNCGTCGCATCCTCATGTTGA
ACACAGGCGATGTGGGTGCAAGGTTTAAATGGGACATCAAAAAATTTGAGCCTCATTTCTCCATTAGCCCA
GAAGAAGGCTATATTACCTCAGGCATGGAGGTTTCTTTTGAAGTGACCTACCATCCCACCGAGGTGGGAAA
GGAGAGCCTTTGTAAAAACATTCTCTGCTACATCCAGGGAGGCAGTCCTCTGAGTCTAACCCTGTCTGGAG
TCTGCGTGGGACCACCTGCGGTAAAAGAGGTAGTGAATTTCACGTGCCAGGTGCGCTCCAAGCACACGCAG
ACCATCCTGCTGTCAAACCGCACCAACCAGACCTGGAATCTGCACCCCATCTTTGAGGGCGAGCACTGGGA
GGGGCCTGAGTTCATCACCCTGGAGGCCCACCAGCAAAACAAGCCCTATGAGATCACCTACAGGCCCCGCA
CCATGAACTTGGAGAACCGCAAGCACCAGGGCACCCTCTTCTTCCCCCTCCCAGATGGGACCGGCTGGCTG
TATGCTCTGCATGGGACTTCTGAGCTCCCCAAAGCTGTAGCCAATATCTATCGTGAAGTGCCATGTAAGAC
CCCCTACACTGAGCTTCTGCCAATCACCAACTGGCTGAACAAGCCCCAGAGATTCCGGGTCATCGTGGAAA
TACTGAAACCAGAGAAGCCGGACCTAAGCATCACTATGAAGGGCCTTGATTACATTGATGTACTGTCTGGC
TCTAAGAAAGACTACAAGCTGAACTTCTTTTCCCACAAGGAGGGAACGTACGCYGCAAANNGATCTTGCGG
AAGCTGA

LP272 Full-Length Sequence (309 aa):

LP272 (SEQ ID NO: 5). The underlined portion indicates a predicted signal sequence (Met-1 to Ala-40). A predicted SP cleavage site is between Ala-40 and Thr-41 indicated as follows: 1 MECMGLLRPLFLLSGCCQALEISLDQEHIPFGPVVYQTQA. An LP encompassed herein includes full-length forms encoded by an ORF disclosed herein, as well as any mature forms therefrom. Such a mature LP could be formed, for example, by the removal of a signal peptide and/or by aminopeptidase modification. All forms of LP272 such as, both precursor and activated forms are encompassed herein. Further, as used herein, a “mature” LP encompasses, e.g., post-translational modifications other than proteolytic cleavages (such as, e.g., by way of a non-limiting example, glycosylations, gamma-carboxylations, beta-hydroxylations, myristylations, phosphorylations, prenylations, acylations, and sulfations). Such variants are also encompassed by an LP of the present invention. Further, an LP of the invention encompasses all fragments, analogs, homologs, and derivatives of an LP described herein, thus the invention encompasses both LP precursors and any modified versions (such as, e.g., by post-translational modification) of an LP encoded by an LP nucleic acid sequence described herein.

MECMGLLRPLFLLSGCCQALEISLDQEHIPFGPVVYQTQATRRILMLNTGDVGARFKWDIKKFEPHFSISPE
EGYITSGMEVSFEVTYHPTEVGKESLCKNILCYIQGGSPLSLTLSGVCVGPPAVKEVVNFTCQVRSKHTQTI
LLSNRTNQTWNLHPIFEGEHWEGPEFITLEAHQQNKPYEITYRPRTMNLENRKHQGTLFFPLPDGTGWLYAL
HGTSELPKAVANIYREVPCKTPYTELLPITNWLNKPQRFRVIVEILKPEKPDLSITMKGLDYIDVLSGSKKD
YKLNFFSHKEGTYAAXXSCGS*

An LP272 Mature Sequence (269aa):

A predicted mature LP272 sequence is as follows:

TRRILNLNTGDVGARFKWDIKKFEPHFSISPEEGYITSGMEVSFEVTYHPTEVGKESLCKNILCYIQGGSPL
SLTLSGVCVGPPAVKEVVNFTCQVRSKHTQTILLSNRTNQTWNLHPIFEGEHWEGPEFITLEAHQQNKPYEI
TYRPRTMNLENRKHQGTLFFPLPDGTGWLYALHGTSELPKAVANIYREVPCKTPYTELLPITNWLNKPQRFR
VIVEILKPEKPDLSITMKGLDYIDVLSGSKKDYKLNFFSHKEGTYAAXXSCGS*

Particularly interesting portions or fragments of the full length LP272 polypeptide include, e.g., a discovered putative signal peptide-like sequence from Met-1 to Ala-40 (MECMGILRPLFLLSGCCQALEISLDQEHIPFGPVVYQTQA).

Other interesting segments of LP272 are discovered portions of LP272 from about Phe-11 to about Glu-21 (FLLSGCCQALE); from about Ile-22 to about Thr-38 (ISLDQEHIPFGPVVYQT); from about Met-46 to about Arg-55 (MXNTGDVGAR); from about Phe-56 to about His-66 (FKWDIKKFEPH); from about Phe-67 to about Ser-78 (FSISPEEGYITS); from about Gly-79 to about Pro-90 (GMEVSFEVTYHP); from about Ser-97 to about Gly-109 (SLCKNILCYIQGG); from about Ser-110 to about Val-119 (SPLSLTLSGV); from about Cys-120 to about Val-130 (CVGPPAVKEVV); from about Phe-132 to about Thr-143 (FTCQVRSKHTQT); from about Trp-154 to about Glu-163 (WNLHPIFEGE); from about His-1 64 to about Leu-173 (HWEGPEFITL); from about Thr-190 to about Gln-199 (TMNLENRKHQ); from about Gly-200 to about Thr-210 (GTLFFPLPDGT); from about Gly-211 to about Lys-224 (GWLYALHGTSELPK); from about Ala-225 to about Leu-242 (AVANIYREVPCKTPYTEL); from about Leu-243 to about Pro-252 (LPITNWLNKP); from about Gln-253 to about Asp-268 (QRFRVIVEILKPEKPD); from about Leu-269 to about Gly-284 (LSITMKGLDYIDVLSG); from about Ser-285 to about His-296 (SKKDYKLNFFSH); from about Phe-11 to about Ser-23 (FLLSGCCQALEIS); from about Val-35 to about Gly-50 (VYQTQATRLMXNTG); Asp-51 to about His-66 (DVGARFKWDIKFEPH); from about Phe-67 to about Ser-78 (FSISPEEGYITS); from about Glu-85 to about Leu-98 (EVTYHPTEVGKESL); from about Cys-99 to about Gly-109 (CKNILCYIQGG)I; from about Ser-110 to about Val-119 (SPLSLTLSGV); from about Cys-120 to about Phe-132 (CVGPPAVKEVVNF); from about Thr-133 to about Leu-145 (TCQVRSKHTQTIL); from about Leu-146 to about Ile-159 (LSNRTNQTWNLHPI); from about Phe-160 to about Ile-171 (FEGEHWEGPEFI); from about Thr-172 to about Glu-183 (FEGEHWEGPEFI); from about Ile-184 to about Phe-203 (TTYRPRTMNLENRKHQGTLF); from about Leu-216 to about Ala-225 (LHGTSELPKA); from about Asn-228 to about Glu-241 (NIYREVPCKTPYTE); from about Pro-244 to about Arg-256 (PITNWLNKPQRFR); from about Lys-263 to about Thr-272 (KPEKPDLSIT); from about Val-281 to about Gly-299 (VLSGSKKDYKLNFFSHKEG); from about Gln-18 to about Phe-31 (QALEISLDQEHIPF); from about Gly-32 to about Leu-45 (GPVVYQTQATRRIL); from about Met-46 to about Ile-60 (MXNTGDVGARFKWDI); from about Lys-61 to about Gly-74 (KKFEPHFSISPEEG); from about Tyr-75 to about Phe-84 (YITSGMEVSF); from about Glu-85 to about Leu-103 (EVTYHPTEVGKESLCKNIL); from about Ile-106 to about Val-121 (IQGGSPLSLTLSGVCV); from about Gly-122 to about Ser-138 (GPPAVKEVVNFTCQVRS); from about Lys-139 to about Pro-158 (KHTQTILLSNRTNQTWNLHP); from about Ile-159 about Ile-171 (IFEGEHWEGPEFI); from about Thr-172 to about Thr-185 (TLEAHQQNKPYEIT); from about Tyr-186 to about Leu-202 (YRPRTMNLENRKHQGTL); from about Phe-203 to about Leu-213 (FFPLPDGTGWL); from about Ala-215 to about Asn-228 (ALHGTSELPKAVAN); from about Ile-229 to about Glu-241 (IYREVPCKTPYTE); from about Lys-263 to about Leu-276 (KPEKPDLSITMKGL); and from about Asp-277 to about Gly-299 (DYIDVLSGSKKDYKLNFFSHKEG); whose discoveries were based on an analysis of hydrophobicity, hydropathicity, and hydrophilicity plots.

Additional interesting sections of LP272 are the discovered portions of LP272 from about Leu-7 to about Glu-21 (LRPLFLLSGCCQALE); from about Ile-22 to about Thr-38 (ISLDQEHIPFGPVVYQT); from about Gln-39 to about Phe-56 (QATRRILMXNTGDVGARF); from about Phe-63 to about Tyr-75 (FEPHFSISPEEGY); from about Ile-76 to about Thr-91 (ITSGMEVSFEVTYHPT); from about Glu-96 to about Pro-111 (ESLCKNILCYIQGGSP); from about Leu-112 to about Gly-122 (LSLTLSGVCVG); from about Pro-1 23 to about Thr-133 (PPAVKEVVNFT); from about Thr-153 to about His-164 (TWNLHPIFEGEH); from about Trp-165 to about Ala-175 (WEGPEFTILEA); from about Arg-231 to about Leu-242 (REVPCKTPYTEL); from about Leu-243 to about Val-259 (LPITNWLNKPQRFRVIV); from about Glu-260 to about Leu-269 (EILKPEKPDL); from about Ser-270 to about Ile-279 (SITMKGLDYI); from about Leu-282 to about Leu-291 (LSGSKKDYKL); and from about Asn-292 to about Ala-302 (NFFSHKEGTYA). These fragments were discovered based on analysis of antigenicity plots.

Further, particularly interesting LP272 segments are LP secondary structures (e.g., such as a helix, a strand, or a coil). Particularly interesting LP272 coil structures are the following from about Met-1 to about Met-4; from about Asp-25 to about Pro-33; from about Asn-48 to about Val-52; from about Phe-63 to about His-66; from about Ser-70 to about Gly-74; from about His-89 to about Lys-95; from about Gln-107 to about Pro-111; from about Val-121 to about Pro-1 24; from about Ser-138 to about Thr-141; from about Asn-148 to about Thr-153; from about His-157 to about Glu-169; from about His-176 to about Pro-181; from about Glu-194 to about Gly-200; from about Pro-205 to about Thr-210; from about His-217 to about Glu-221; from about Glu-232 to about Tyr-239; from about Asn-247 to about Pro-252; from about Lys-263 to about Asp-268; from about Lys-274 to about Leu-276; from about Ser-283 to about Asp-288; from about Ser-295 to about Thr-300; and from about Ser-306 to about Ser-309. A particularly interesting helix structure is Lys-224 to Tyr-230. Particularly interesting strand structures are from about Ile-44 to about Met-46; from about Glu-81 to about Thr-87; from about Asn-131 to about Phe-132; from about Gln-142 to about Ser-147; from about Glu-183 to about Thr-185; from about Trp-212 to about Ala-215; Ser-270 to about Thr-272; and from about Tyr-278 to Val-281.

Further encompassed by the invention are contiguous amino acid residue combinations of any of the predicted secondary structures described above. For example one coil-strand-coil-helix motif of LP272 combines the coil of Pro-205 to Thr-210, the strand of Trp-212 to Ala-215, the coil of His-217 to Glu-221, and the helix of Lys-224 to Tyr-230 to form an interesting fragment of contiguous amino acid residues from about Pro-205 to about Tyr-230. Other combinations of contiguous amino acids are contemplated as can be easily determined.

It is likely that an LP272, an LP272 variant, an LP272 agonist, an LP272 antagonist, an LP272 binding partner or an LP272 fragment as described herein plays a role in a variety of physiological processes such as: cytostatic; hepatotropic; vulnerary; antipsoriatic; antiparkinsonian; nootropic; neuroprotective; anticonvulsant; osteopathic; antiarthritic; immunosuppressant; cardiant; immunostimulant; thrombolytic; coagulant; vasotropic; antidiabetic; hypotensive; dermatological; immunosuppressive; antiinflammatory; antiviral; antibacterial; antifungal; antirheumatic; antithyroid; antianaemic; gene therapy; cancer; proliferative disorder; hypertension; neurodegenerative disorder; osteoarthritis; graft vs host disease; cardiovascular disease; diabetes mellitus; hypothyroidism; SCID; AIDS; cholesterol ester storage; systemic lupus erythematosus; infection; severe combined immunodeficiency; malaria; autoimmune disorder; asthma; allergy; aplastic anaemia; nocturnal haemoglobinuria; burn; wound; bone damage; cartilage damage; antiinflammatory disease; coagulation; thrombosis. Additional assays or methods for assessing an activity of an LP of the invention may, among other means, be measured by other methods described herein.

Features of LP NO: 4 (LP357)

LP357 is a novel secreted polypeptide encoded by CDNA, when fully sequenced, exhibits an Ig-varible domain sequence and homology with the human polymeric Ig receptor (pIgR) secretory component (Krajci, P., et al., Hum. Genet. 87:642-648,1991). Additionally, LP357 is a splice variant of GPCR-7 (WO00/20590) and zsig57 (WO99/66040). The LP357 nucleotide sequence is believed to encode the entire coding sequence of the predicted protein. LP357 may be a new transcytosis receptor, immunomodulator, or the like, and is a novel member of the immunoglobulin superfamily of proteins.

The nucleotide sequence of a representative LP357-encoding DNA is described in SEQ ID NO:7, and its deduced 311 amino acid sequence is described in SEQ ID NO:8. In its entirety, LP357 polypeptide represents a full-length polypeptide segment (residue 1 (Met) to residue 311 (Ser) of SEQ ID NO:8). LP357 contains a signal sequence, single Ig-variable domain, a transmembrane domain, and a cytoplasmic sequence. These domains and structural features of LP357 are further described below.

Table 4: Primate, e.g., human, LP357 polynucleotide sequence (SEQ ID NO: 7) and corresponding polypeptide (SEQ ID NO: 8). The ORF for LP357 is 1-936 bp (with the start (ATG) and stop codons (TAA) identified in bold typeface and underlined. In case the numbering is misidentified herein, one skilled in the art could easily determine the open reading frame without undue experimentation given the teachings herein. Analysis of the DNA encoding LP357 polynucleotide (SEQ ID NO:7) revealed an open reading frame encoding 311 amino acids (SEQ'ID NO:8) comprising a predicted signal peptide of 15 amino acid residues (residue 1 (Met) to residue 15 (Gly) of SEQ ID NO:8), and a mature polypeptide of 296 amino acids (residue 16 (Gln) to residue 311 (Ser) of SEQ ID NO:8). LP357 contains the following 4 regions of conserved amino acids:

1) The first region, referred to hereinafter as the “Ig-variable domains” corresponds to amino acid residues 16 (Gln) to amino acid 125 (Pro) of SEQ ID NO:8.

2) The second region, referred to hereinafter as “acidic cleavage sites(s),” corresponds to amino acid 126 (Glu) to amino acid 130 (Glu) or SEQ ID NO:8, with potential cleavage at residue 126 (Glu); ans the di-acid Asp-Glu at residues 157 (Asp) and 158 (Glu) of SEQ ID NO:7, with potential cleavage at residue 157 (Asp). These acidic cleavage sites suggest that the portion of LP357 containing the Ig-variable domain is secreted.

3) The third region, referred to hereinafter as the “transmembrane domain” corresponds to amino acid residues 163 (Leu) to amino acid residue 190 (Gly) of SEQ ID NO:8.

4) The fourth region, referred to hereinafter as the “cytoplasmic C-terminal sequence” corresponds to amino acid residues 191 (Asn) to amino acid 311 (Ser) of SEQ ID NO:8).

LP357 DNA sequence (936 bp) (ORF=1-936)

LP357 (start (atg) and stop (taa) codons are indicated in bold typeface and underlined).

ATGGGCCTCACCCTGCTCTTGCTGCTGCTCCTGGGACTAGAAGGTCAGGGCATAGTTGGCAGCCTCCCTGAG
GTGCTGCAGGCACCCGTGGGAAGCTCCATTCTCGTGCAGTGCCACTACAGGCTCCAGGATGTCAAAGCTCAG
AAGGTGTGGTGCCGGTTCTTGCCGGAGGGGTGCCAGCCCCTGGTGTCCTCAGCTGTGGATCGCAGAGCTCCA
GCGGGCAGGCGTACGTTTCTCACAGACCTGGGTGGGCGCCTGCTGCAGGTGGAAATGGTTACCCTGCAGGAA
GAGGATGCTGGCGAGTATGGCTGCATGGTGGATGGGGCCAGGGGGCCCCAGATTTTGCACAGAGTCTCTCTG
AACATACTGCCCCCAGAGGAAGAAGAAGAGACCCATAAGATTGGCAGTCTGGCTGAGAACGCATTCTCAGAC
CCTGCAGGCAGTGCCAACCCTTTGGAACCCAGCCAGGATGAGAAGAGCATCCCCTTGATCTGGGGTGCTGTG
CTCCTGGTAGGTCTGCTGGTGGCAGCGGTGGTGCTGTTTGCTCTGATGGCCAAGAGGAAACAAGGCAACAGG
CTTGGTGTCTGTGGCCGATTCCTGAGCAGCAGAGTTTCAGGCATGAATCCCTCCTCAGTGGTCCACCACGTC
AGTGACTCTGGACCGGCTGCTGAATTGCCTTTGGATGTACCACACATTAGGCTTGACTCACCACCTTCATTT
GACAATACCACCTACACCAGCCTACCTCTTGATTCCCCATCAGGAAAACCTTCACTCCCAGCTCCATCCTCA
TTGCCCCCTCTACCTCCTAAGGTCCTGGTCTGCTCCAAGCCTGTGACATATGCCACAGTAATCTTCCCGGGA
GGGAACAAGGGTGGAGGGACCTCGTGTGGGCCAGCCCAGAATCCACCTAACAATCAGACTCCATCCACCTAA

LP357 Full-Length Sequence (311 aa):

LP357 (SEQ ID NO: 8). The underlined portion indicates a predicted signal sequence (Met-1 to Gly-15). A predicted SP cleavage site is between Gly-15 and Gln-16 indicated as follows: 1 MGLTLLLLLLLGLEG\hat{ }QG 17. An LP encompassed herein includes full-length forms encoded by an ORF disclosed herein, as well as any mature forms therefrom. Such a mature LP could be formed, for example, by the removal of a signal peptide and/or by aminopeptidase modification. All forms of LP357 such as, both precursor and activated forms, are encompassed herein. Further, as used herein, a “mature” LP encompasses, e.g., post-translational modifications other than proteolytic cleavages (such as, e.g., by way of a non-limiting example, glycosylations, gamma-carboxylations, beta-hydroxylations, myristylations, phosphorylations, prenylations, acylations, and sulfations). Such variants are also encompassed by an LP of the present invention. Further, an LP of the invention encompasses all fragments, analogs, homologs, and derivatives of an LP described herein, thus the invention encompasses both LP precursors and any modified versions (such as, e.g., by post-translational modification) of an LP encoded by an LP nucleic acid sequence described herein.

MGLTLLLLLLLGLEGQGIVGSLPEVLQAPVGSSILVQCHYRLQDVKAQKVWCRFLPEGCQPLVSSAVDRRAP
AGRRTFLTDLGGGLLQVEMVTLQEEDAGEYGCMVDGARGPQILHRVSLNILPPEEEEETHKIGSLAENAFSD
PAGSANPLEPSQDEKSIPLIWGAVLLVGLLVAAVVLFAVMAKRKQGNRLGVCGRFLSSRVSGMNPSSVVHHV
SDSCPAAELPLDVPHIRLDSPPSFDNTTYTSLPLDSPSGKPSLPAPSSLPPLPPKVLVCSKPVTYATVIFPG
GNKGGGTSCGPAQNPPNNQTPSS*

An LP357 Mature Sequence (296aa):

A predicted mature LP357 sequence is as follows below. Mature LP357 has a Ig-variable domain, Glu-16 to Pro-125 (indicated below by single underlining) which has 100% homology with aa residues 1-110 of polymeric immunoglobulin receptor; a transmembrane domain, Leu-163 to Gly-190 (indicated in bold letters); and, a cytoplasmic C-terminal sequence, Asn-191 to Ser-311 (indicated by italic letters).

QGIVGSLPEVLQAPVGSSILVQCHYRLQDVKAQKVWCRFLPEGCQPLVSSAVDRRAPAGRRTFLTDLGGGLL
QVEMVTLQEEDAGEYGCMVDGARGPQILHRVSLNILPPEEEEETHKIGSLAENAFSDPAGSANPLEPSQDEK
SIPLIWGAVLLVGLLVAAVVLFAVMAKRKQGNRLGVCGRFLSSRVSGMNPSSVVHHVSDSGPAAELPLDVPH
IRLDSPPSFDNTTYTSLPLDSPSGKPSLPAPSSLPPLPPKVLVCSKPVTYATVIFPGGNKGGGTSCGPAQNP
PNNQTPSS*

In addition, within the Ig-variable domain, LP357 contains conserved cysteines located at rcsidues 38, 52, 59, and 104. Disulfide bonds are predicted between cysteine residues 52 and 59 and between residues 38 and 104. These cysteines likely maintain a structurally important fold in the Ig-variable domain, and are conserved throughout the protein family. The presence of conserved motifs generally correlates with or defines important structural regions in proteins. The regions between such motifs may be more variable, but are often functionally significant because they can relate to or define important structures and activities such as binding domains, biological and enzymatic activity, signal transduction, tissue localization domains and the like. As described above, the novel LP357 polypeptide encoded by the polynucleotide described herein contains an Ig-variable domain. The structural topology of Ig- variable domains are conserved in the immunoglobulin superfamily of proteins. This domain may be involved in binding another immunoglobulin superfamily protein family member, and confer an essential function in transcytosis in tissues where it is expressed, such as the small intestine; similarly, the Ig-variable domain can also associate or bind with polypeptides or peptides involved in antigen presentation, or confer an immunomodulator activity in PBLs or bone marrow. Additionally, LP357 polypeptide could be involved in binding other immune effector proteins destined for translocation, for instance in bone marrow or small intestine. The highly conserved amino acids in the Ig-variable domain, transmembrane domain, or other regions of LP357 can be used as a tool to identify new family members. For instance, reverse transcription-polymerase chain reaction (RT-PCR) can be used to amplify sequences encoding the conserved regions from RNA obtained from a variety of tissue sources or cell lines. In particular, highly degenerate primers designed from the LP357 sequences are useful for this purpose. Designing and using such degenerate primers is readily performed by one of skill in the art. It has been discovered that LP357 nucleic acid sequence (SEQ ID NO:7) is expressed in the following number of LIFESEQGOLD™ database tissue and cDNA libraries: Connective Tissue 1/54; Digestive Tissue 1/155; Hernic and Immune System 3/179; Liver 1/37; Musculoskeletal System 1/50; and the Nervous System 1/231. Additionally, Northern Blot analysis utilizing a dsDNA, 541 bp probe, detected a strong signal in spleen and minor signals in placenta, kidney, liver and skeletal muscle. RT-PCR analysis was positive for bone marrow, spleen, thymus, and lymph node.

LP357 nucleic acid sequence has been localized to the 6p21 region of human chromosome number 6. Genetic aberration may be involved in the following diseases, conditions, syndromes, disorders, or pathological states which are also mapped to this region of the human genome: psoriasis, a chronic inflammatory dermatosis that affects approximately 2% of the population (Nair, R. P.;, et al., Am. J. Hum. Genet. 66: 1833-1844, 2000); polycystic kidney and hepatic disease (Zerres, K.; et al., Nature Genet. 7: 429-432, 1994); retinal cone dystrophy (Payne, A. M.; et al., Am. J. Hum. Genet. 61 (suppl.): A290 only, 1997); dyslexia (Smith, S. D.; et al., Reading Writing 3: 285-298, 1991); and diabetes mellitus (Todd, J. A., Immun. Today 11: 122-129, 1990). Accordingly, an isolated and/or recombinant molecule comprising LP357 nucleic acid sequence meets the statutory utility requirement of 35 U.S.C. §101 since such a molecule can be used, for example, to hybridize near a nucleic acid sequence associated with one or more of the above stated diseases, conditions, syndromes, disorders, or pathological states and thus serve as a marker for such a disease gene.

Other interesting segments of LP357 are discovered portions of LP357 from about Ile-18 to about Gln-27 (IVGSLPEVLQ); from about Val-30 to about Leu-42 (VGSSILVQCHYRL); from about Ser-65 to about Arg-76 (SAVDRRAPAGRR); from about Thr-77 to about Leu-94 (TFLTDLGGGLLQVEMVTL); from about Gln-95 to about Gln-113 (QEEDAGEYGCMVDGARGPQ); from about Ile-114 to about Pro-1 25 (ILHRVSLNILPP); from about Glu-126 to about Ser-136 (EEEEETHKIGS); from about Ala-138 to about Ser-148 (AENAFSDPAGS); from about Ala-1 49 to about Lys-159 (ANPLEPSQDEK); from about Ser-160 to about Leu-169 (SIPLIWGAVL); from about Leu-170 to about Met-184 (LVGLLVAAVVLFAVM); from about Ala-1 85 to about Gly-194 (AKRKQGNRLG); from about Val-213 to about Ala-223 (VHHVSDSGPAA); from about Glu-224 to about Pro-238 (ELPLDVPHIRLDSPP); from about Leu-259 to about Pro-270 (LPAPSSLPPLPP); from about Lys-271 to about Pro-287 (KVLVCSKPVTYATVIFP); from about Gly-288 to about Asn-305 (GGNKGGGTSCGPAQNPPN); from about Leu-7 to about Gln-16 (LLLLLGLEGQ); from about Gly-17 to about Ala-28 (GIVGSLPEVLQA); from about Pro-29 to about Cys-38 (PVGSSILVQC); from about His-39 to about Val-50 (HYRLQDVKAQKV); from about Val-67 to about Phe-78 (VDRRAPAGRRTF); from about Leu-79 to about Val-92 (LTDLGGGLLQVEMV); from about Pro-124 to about Gly-135 (PPEEEEETHKIG); from about Glu-139 to about Ala-149 (ENAFSDPAGSA); Asn-150 to about Ser-1 60 (NPLEPSQDEKS); from about Ile-161 to about Gly-172 (IPLIWGAVLLVG); from about Leu-173 to about Met-184 (LLVAAVVLFAVM); from about His-231 to about Ser-247 (HIRLDSPPSFDNTTYTS); from about Leu-250 to about Pro-260 (LDSPSGKPSLP); from about Leu-268 to about Lys-277 (LPPKVLVCSK); from about Pro-278 to about Pro-287 (PVTYATVIFP); from about Gly-288 to about Ala-305 (GGNKGGGTSCGPAQNPPN); from about His-39 to about Lys-11 (HYRLQDVKAQK); from about Pro-56 to about Val-67 (PEGCQPLVSSAV); from about Asp-68 to about Phe-78 (DRRAPAGRRTF); from abut Val-92 to about Gly-103 (VTLQEEDAGEYG); from about Cys-104 to abut Gln-113 (CMVDGARGPQ); from about Ile-114 to about Leu-123 (ILHRVSLNIL); from about Pro-124 to about Ser-136 (PPEEEEETHKIGS); from about Leu-137 to about Ser-148 (LAENAFSDPAGS); from about Ala-149 to about Ser-160 (ANPLEPSQDEKS); from about Leu-163 to about Gly-172 (LIWGAVLLVG); from about Leu-173 to about Met-184 (LLVAAVVLFAVM); from about Ala-185 to about Arg-198 (AKRKQGNRLGVCGR); from about Ser-201 to about Val-213 (SSRVSGMNPSSVV); from about His-214 to about Leu-225 (HHVSDSGPAAEL); from about Arg-233 to about Ser-247 (RLDSPPSFDNTTYTS); from about Leu-248 to about Pro-266 (LPLDSPSGKPSLPAPSSLP); and from about Pro-287 to about Asn-305 (PGGNKGGGTSCGPAQNPPN) whose discoveries were based on an analysis of hydrophobicity, hydropathicity, and hydrophilicity plots.

Additional interesting sections of LP357 are the discovered portions of LP357 from about Leu-11 to about Gly-20 (LGLEGQGIVG); from about Ser-21 to about Ile-34 (SLPEVLQAPVGSSI); from about Val-36 to about Lys-49 (VQCHYRLQDVKAQK); from about Trp-51 to about Asp-68 (WCRFLPEGCQPLVSSAVD); from about Gly-74 to about Gly-84 (GRRTFLTDLGG); from about Gly-85 to about Gln-95 (GLLQVEMVTLQ); from about Glu-96 to about Met-105 (EEDAGEYGCM); from about Val-106 to about Ile-122 (VDGARGPQILHRVSLNI); from about Leu-123 to about Ala-141 (LPPEEEEETHKIGSLAENA); from about Ser-143 to about Leu-152 (SDPAGSANPL); from about Glu-153 to about Leu-163 (EPSQDEKSIPL); from about Ile-164 to about Val-175 (IWGAVLLVGLLV); from about Ala-176 to about Ala-185 (AAVVLFAVMA); from about Arg-187 to about Leu-200 (RKQGNRLGVCGRFL); from about Ser-201 to about Val-216 (SSRVSGMNPSSVVHHV); from about Ser-217 to about Val-229 (SDSGPAAELPLDV); from about Leu-234 to about Leu-250 (LDSPPSFDNTTYTSLPL); from about Ser-263 to about Val-274 (SSLPPLPPKVLV); from about Cys-275 to about Gly-289 (CSKPVTYATVIFPGG); and from about Asn-290 to about Gln-301 (NKGGGTSCGPAQ). These fragments were discovered based on analysis of antigenicity plots. Further, particularly interesting LP357 segments are LP secondary structures (e.g., such as a helix, a strand, or a coil). Particularly interesting LP357 coil structures are the following: from about Met-1 to about Met-1; from about Leu-13 to about Glu-24; from about Ala-28 to about Ser-32; from about Leu-55 to about Pro-61; from about Arg-70 to about Arg-75; from about Asp-81 to about Gly-85; from about Glu-96 to about Thr-102; from about Asp-107 to about Pro-112; from about Ile-122 to about Glu-126; from about Asn-140 to about Ile-161; from about Gly-190 to about Arg-192; from about Ser-205 to about Ser-210; from about Ser-217 to about Pro-230; from about Asp-235 to about Thr-243; from about Pro-249 to about Lys-271; from about Ser-276 to about Val-279; from about Pro-287 to about Gly-294; and from about Gly-298 to about Ser-301. Particularly interesting helix structures are from about Thr-4 to about Leu-6; from about Val-67 to about Asp-68; from about Glu-129 to about Thr-131; and from about Val-183 to about Lys-186. Particularly interesting strand structures are from about Ile-34 to about Val-36; from about Thr-77 to about Leu-79; from about Leu-87 to about Val-89; from about Met-91 to about Thr-93; from about Gly-103 to about Val-106; from about Leu-163 to about Ile-164; from about Val-212 to about Val-216; from about His-231 to about Arg-233; from about Val-272 to about Val-274; and from about Tyr-281 to about Phe-286. Further encompassed by the invention are contiguous amino acid residue combinations of any of the predicted secondary structures described above. For example, one coil-helix-coil-strand-coil motif of LP357 combines the Leu-55 to about Pro-61 coil; with the Val-67 to about Asp-68 helix; with the Arg-70 to about Arg-75 coil; with the Thr-77 to about Leu-79 strand; with the Asp-81 to about Gly-85 coil to form an interesting fragment of contiguous amino acid residues from about Leu-55 to about Gly-85. Other combinations of contiguous amino acids are contemplated as can be easily determined.

LP357 Functions

The polypeptides, nucleic acid and/or antibodies of the present invention can be used in treatment of disorders associated with the immune system, gastrointestinal system, heart, inflammation, lymph system, bone marrow, blood and bones. The molecules of the present invention may used to modulate or to treat or prevent development of pathological conditions in such diverse tissue as small intestine and bone marrow. In particular, certain syndromes or diseases can be amenable to such diagnosis, treatment or prevention.

In addition, polypeptides of the present invention can be used for their ability to. modify inflammation. Methods to assess pro-inflammatory or anti-inflammatory qualities of LP357 are known in the art. For example, suppression of cAMP production is an indication of anti-inflammatory effects of the pIgR secretory component (SC) (Nihei, Y., et al., Arch. Dermatol. Res., 287:546-552, 1995). Free SC component of the poly-IgR suppressed cAMP and inhibited ICAM and HLA-Dr induced by IFN-y in keratinocytes. Moreover, free SC has been reported to inhibit PIA2 and is believed to act via the arachadonic acid anti-inflammatory cascade. LP357 likewise can exhibit similar anti-inflammatory effects, and may exert these effects in tissues in which it is expressed. For example, LP357 is expressed in the small intestine, and can be useful in treatment of inflammatory bowel disease, diverticulitis, inflammation during and after intestinal surgery, and the like. In addition, LP357 expressed in PBLs and bone marrow, can have other anti-inflammatory actions in heart, pelvic inflammatory disease (PID), psoriasis, arthritis, and other inflammatory diseases.

As such, LP357 polypeptide, or its antagonists, have potential uses in inflammatory diseases such as asthma and arthritis. For example, if LP357 is pro-inflammatory, antagonists would be valuable in asthma therapy or other anti-inflammatory therapies where migration of lymphocytes is damaging. Alternatively, LP357 can have an inhibitory or competitive effect on inflammatory agents and may serve directly as an asthma therapeutic or anti-infiammatory. In addition, LP357 can serve other important roles in lung function, for instance, bronchodilation, tissue elasticity, recruitment of lymphocytes in lung infection and damage. Assays to assess the activity of LP357 in lung cells are similar to the assays discussed in Laberge, S. et al., Am. J. Respir. Cell Mol. Biol. 17:193-202, 1997; Rumsaeng, V. et al., J. Immunol., 159:2904-2910, 1997; and Schluesener, H. J. et al.,J. Neurosci. Res. 44:606-611, 1996.

Methods to determine pro-inflammatory and anti-inflammatory qualities of LP357 or its antagonists are known in the art. Moreover, other molecular, biological, immunological, and biochemical techniques known in the art and disclosed herein can be used to determine LP357 activity and to isolate agonists and antagonists.

Moreover, based on high expression in PBLs, LP357 may exhibit antiviral functions by inhibiting viral replication by specific signaling via it's receptor(s) on a host cell (e.g. T-cell). LP357 may exhibit immune cell proliferative activity, as disclosed herein, and may stimulate the immune system to fight viral infections. Moreover, LP357 may bind CD4 or another lymphocyte receptor and exhibit antiviral effects, for example, against human immunodeficiency virus (HIV) or human T-cell lymphotropic virus (HTLV). In addition, LP357 physically interacts with different isoforms of fibrinogen from human plasma. Thus, LP357 may be useful in the regulation of fibrinogen-dependent processes.

Alternatively, LP357 polypeptide may compete for a viral receptor or co-receptor to block viral infection. LP357 may be given parentally to prevent viral infection or to reduce ongoing viral replication and re-infection (Gayowski, T. et al., Transplantation 64:422-426, 1997). Thus, LP357 may be used as an antiviral therapeutic, for example, for viral leukemias (HTLV), AIDS (HIV), or gastrointestinal viral infections caused by, for example, rotavirus, calicivirus (e.g., Norwalk Agent) and certain strains of pathogenic adenovirus.

The molecules of the present invention can be useful for proliferation of cardiac tissue cells, such as cardiac myocytes or myoblasts; skeletal myocytes or myoblasts and smooth muscle cells; chrondrocytes; endothelial cells; adipocytes and osteoblasts in vitro. For example, molecules of the present invention are useful as components of defined cell culture media, and can be used alone or in combination with other cytokines and hormones to replace serum that is commonly used in cell culture. Molecules of the present invention are particularly useful in specifically promoting the growth and/or development of myocytes in culture, and may also prove useful in the study of cardiac myocyte hyperplasia and regeneration.

The polypeptides, nucleic acids and/or antibodies of the present invention can be used in treatment of disorders associated with myocardial infarction, congestive heart failure, hypertrophic cardiomyopathy and dilated cardiomyopathy. Molecules of the present invention may also be useful for limiting infarct size following a heart attack, aiding in recovery after heart transplantation, promoting angiogenesis and wound healing following angioplasty or endarterectomy, to develop coronary collateral circulation, for revascularization in the eye, for complications related to poor circulation such as diabetic foot ulcers, for stroke following coronary reperfusion using pharmacologic methods, and other indications where angiogenesis is of benefit. Molecules of the present invention may be useful for improving cardiac function, either by inducing cardiac myocyte neogenesis and/or hyperplasia, by inducing coronary collateral development, or by inducing remodeling of necrotic myocardial area. Other therapeutic uses for the present invention include induction of skeletal muscle neogenesis and/or hyperplasia, kidney regeneration and/or for treatment of systemic and pulmonary hypertension.

The LP357 polypeptide is expressed in the small intestine. Thus, LP357 polypeptide pharmaceutical compositions of the present invention can also be useful in prevention or treatment of digestive disorders in the GI tract, such as disorders associated with pathological secretory cell expansion or differentiation. Assays and animal models are known in the art for monitoring such expansion or differentiation and for evaluating LP357 polypeptide, fragments fusion protein, antibody, agonist or antagonist in the prevention or treatment thereof.

Moreover, trefoil factors in the intestine are known to be involved in mucosal stabilization in the gut and repair processes associated with acute injury, particularly epithelial restitution (Poulsom, R., Bail. Clin. Gastro., 10; 113-134,1996; Sands, B. E., and Podolsky, D. K., Annu. Rev. Physiol., 58; 253-273, 1996. Also, trefoil proteins appear to have a role in healing wounds caused by intestinal inflammatory diseases, and resisting microbial invasion via mucosal secretion involvement (Palut, A. G., New Eng. J. Med., 336; 5-6-507,1997; Playford, R. J., J. Royal Coll. Phys. London, 31; 37- 41, 1997). Epidermal growth factor (EGF) receptor ligands may play a role in enhancing trefoil activity in the gut, however, repair of mucosal injury is not dependent in the main endogenous EGF receptor ligand in the gut, TNF-α, suggesting a role of other undiscovered ligands (Cook, G. A., et al., Am. Physiol. Soc., G1540-G1549, 1997). For example, the LP357 polypeptides can serve as such ligand, regulatory protein or other factor in the trefoil pathway, and hence play an important therapeutic role in diseases and injury associated with the gut and mucosal epithelium.

Also, LP357 polypeptide is expressed in the bone marrow and PBLs and can exert its effects in the vital organs of the body. Activity of LP357 expressed in PBLs and bone marrow may be independent of gastrointestinal function. Thus, LP357 polypeptide pharmaceutical compositions of the present invention can be useful in prevention or treatment of pancreatic disorders associated with pathological regulation of the expansion of neuroendocrine and exocrine cells in the pancreas, such as IDDM, pancreatic cancer, pathological regulation of blood glucose levels, insulin resistance or digestive function.

The LP357 polypeptide of the present invention may act in the neuroendocrine/exocrine cell fate decision pathway and is therefore capable of regulating the expansion of neuroendocrine and exocrine cells in the pancreas. One such regulatory use is that of islet cell regeneration. Also, it has been hypothesized that the autoimmunity that triggers IDDM starts in utero, and LP357 polypeptide is a developmental gene involved in cell partitioning. Assays and animal models are known in the art for monitoring the exocrine/neuroendocrine cell lineage decision, for observing pancreatic cell balance and for evaluating LP357 polypeptide, fragment, fusion protein, antibody, agonist or antagonist in the prevention or treatment of the conditions set forth above.

The present invention also provides reagents which will find use in diagnostic applications. For example, the LP357 gene, a probe comprising LP357 DNA or RNA or a subsequence thereof can be used to determine if the LP357 gene is present on chromosome 6 or if a mutation has occurred. LP357 is located at the 6p21 region of chromosome 6. Detectable chromosomal aberrations at the LP357 gene locus include, but are not limited to, aneuploidy, gene copy number changes, insertions, deletions, restriction site changes and rearrangements. Such aberrations can be detected using polynucleotides of the present invention by employing molecular genetic techniques, such as restriction fragment length polymorphism (RFLP) analysis, short tandem repeat (STR) analysis employing PCR techniques, and other genetic linkage analysis techniques known in the art (Sambrook et al., ibid.; Ausubel et. al., ibid.; Marian, Chest 108:255-65, 1995).

The precise knowledge of a gene's position can be useful for a number of purposes, including. 1) determining if a sequence is part of an existing contig and obtaining additional surrounding genetic sequences in various forms, such as YACs, BACs or cDNA clones; 2) providing a possible candidate gene for an inheritable disease which shows linkage to the same chromosomal region; and 3) cross-referencing model organisms, such as mouse, which can aid in determining what function a particular gene might have.

The LP357 gene is located within the major histocompatability (MHC) locus, which encodes proteins involved with antigen presentation to T-cells. Proteins and polypeptides are processed and then complexed with MHC molecules followed by transport to the cell surface for presentation to T-cells. A number of accessory molecules are encoded in the MHC locus that is essential for antigen processing and presentation. For example, TAP transporters and tapasin function to transport and assemble peptides plus MHC respectively (Herberg, J. A., et al., Eur. J. Immunol., 28:459-467, 1998). In a similar manner, LP357 polypeptide may be involved in antigen presentation, as a chaparone, transporter, trafficking element, or other processing and presentation function.

Antigen presentation can be measured in standard assays known in the art: for example, antigen presentation for cytotoxic T-cells, such as the chromium release assay (Hosken, N. A., and Bevan, M. J., J. Exp. Med. 175:719-729, 1992); and proliferation and IL-2 production by T-cells in response to antigen presenting cells (Rudensky, A. Y., et al., Nature 353:660-662, 1991; Roosnek, E., and Lanzavecchia, J. EXP. Med. 173:487-489, 1991).

In addition, LP357 polypeptides, agonists or antagonists thereof can be therapeutically useful for anti-microbial applications. To verify the presence of this capability in LP357 polypeptides, agonists or antagonists of the present invention, such LP357 polypeptides, agonists or antagonists are evaluated with respect to their anti-microbial properties according to procedures known in the art. See, for example, Barsum et al., Eur. Respir. J. 8: 709-14, 1995; Sandovsky-Losica et al., J. Med. Vet. Mycol (England) 28: 279-87, 1990; Mehentee et al., J. Gen. Microbiol (England) 135: 2181-88, 1989; Segal and Savage, Journal of Medical and Veterinary Mycology 24: 477-479, 1986, and the like. If desired, LP357 polypeptide performance in this regard can be compared to proteins known to be functional in this regard, such as proline-rich proteins, lysozyme, histatins, lactoperoxidase or the like. Moreover, LP357 may bind and protect immune molecules (e.g., IgA) from proteolytic or other microbial attack (Brandtzaeg, P. and Krajci, P., “Secretory Component (pIgR)” In: Encyclopedia of Immunology, Ivan M. Roitt and Peter J. Delves (eds.), pp.1360-1364, Academic Press, London, 1992). In addition, LP357 polypeptides or agonists or antagonists thereof can be evaluated in combination with one or more anti-microbial agents to identify synergistic effects.

TABLE 5

| | | | | | | | 5′ NT of | | | | | |
|---|---|---|---|---|---|---|---|---|---|---|---|---|
| | | | | | 3′ NT | | First | AA | First | | | |
| | | NT SEQ | Total | 5′ NT of | of | | AA of | SEQ | AA of | | First AA | |
| LP | CDNA | ID | NT | Clone | Clone | | signal | ID | Sig | Last AA | Secreted | Last AA |
| NO. | Clone ID | NO: X | (bp) | SEQ. | SEQ. | ORF | Pep. | NO Y: | Pep | of Sig Pep | Portion | of ORF |
| 1 | LP231 | 1 | 864 | 1 | 864 | 1-864 | 1 | 2 | Met-1 | Ala-15 | Ala-16 | Asp-287 |
| 2 | LP285 | 3 | 921 | 1 | 921 | 1-921 | 1 | 4 | Met-1 | Ser-26 | Ala-27 | Asn-306 |
| 3 | LP272 | 5 | 930 | 1 | 930 | 1-930 | 1 | 6 | Met-1 | Ala-40 | Thr-41 | Ser-309 |
| 4 | LP357 | 7 | 936 | 1 | 936 | 1-936 | 1 | 8 | Met-1 | Gly-15 | Gln-16 | Ser-311 |

Table 5 summarizes information corresponding to each “LP No.” of the invention as described herein. The column labeled, “Total NT Seq.” refers to the total number of nucleotides in a polynucleotide sequence identified by an “LP No.” The nucleotide position of SEQ ID NO: X of the putative start codon (methionine) is identified as “5′ NT of Start Codon.” Similarly, the nucleotide position of SEQ ID NO: X of a predicted signal sequence of an LP protein or polypeptide is identified as “5′ NT of First AA of Signal Pep.” The corresponding translated amino acid sequence of a particular NT SEQ ID NO:X, typically beginning with the methionine, is identified as “AA SEQ ID NO: Y,” although other reading frames can also be easily translated using techniques known in molecular biology. A polypeptide produced using an alternative open reading frame/s is also specifically encompassed by the present invention. The first and last amino acid position of a SEQ ID NO: Y of the predicted signal peptide is identified as “First AA of Signal Pep” and “Last AA of Signal Pep.” The predicted first amino acid position of SEQ ID NO: Y of the secreted portion is identified as “Predicted First AA of Secreted Portion.” Finally, the amino acid position of SEQ ID NO: Y of the last amino acid in the open reading frame is identified as “Last AA of ORF.”

An LP polypeptide or fragment thereof, identified from SEQ ID NO: Y may be used, e.g., as an immunogen to generate an antibody that specifically and/or selectively binds a protein comprising an LP polypeptide sequence (or fragment thereof) of the invention and/or to a mature LP polypeptide or secreted LP protein, e.g., encoded by a polynucleotide sequence described herein. An LP polypeptide of the invention can be prepared in any manner suitable to those known in the art. Such a polypeptide includes, e.g., naturally occurring polypeptides that are isolated, recombinantly produced polypeptides, synthetically produced polypeptides, or polypeptides produced by any combination of these methods. Means for preparing such polypeptides are well understood in the art. An LP polypeptide (or fragment thereof) may be in the form of, a mature polypeptide, a secreted protein (including the mature form), or it may be a fragment thereof, or it may be a part of a larger polypeptide or protein, such as, e.g., a fusion protein.

It is often advantageous to include with an LP polypeptide (or fragment thereof), e.g., additional amino acid sequence that contains, e.g., secretory or leader sequences, pro-sequences, sequences that aid in purification, such as, e.g., multiple histidine residues, or an additional sequence for stability during recombinant production. Such variants are also encompassed herein. An LP polypeptide (or fragment thereof) is preferably provided in an isolated or recombinant form, or it may be preferably substantially purified. A recombinantly produced version of an LP polypeptide of the invention, including a secreted polypeptide, can be substantially purified using techniques described herein or otherwise known in the art, such as, e.g., the single-step purification method (Smith and Johnson (1988) Gene 67(1):31 -40). An LP polypeptide (or fragment thereof) can also be purified from natural, synthetic or recombinant sources using techniques described herein or otherwise known in the art, such as, e.g., using an antibody of the invention raised against a secreted protein. The present invention provides an isolated or recombinant LP polynucleotide comprising, or alternatively consisting of, a nucleic acid molecule having a mature polynucleotide sequence of SEQ ID NO: X wherein said polynucleotide sequence or said cDNA encodes at least 12 contiguous amino acids of a mature polypeptide of SEQ ID NO: Y.

II. Definitions

LP Polynucleotide

As used herein, the term “LP polynucleotide” refers to a molecule comprising a nucleic acid sequence contained in a Table herein or in a sequence of SEQ ID NO:X. For example, the polynucleotide can contain the nucleotide sequence of the full length cDNA sequence, including the 5′ and 3′ untranslated sequences, the coding region, with or without the signal sequence, the secreted protein coding region, as well as fragments, epitopes, domains, and variants of the nucleic acid sequence. An “LP polynucleotide” also encompasses, e.g., those polynucleotides that stably hybridize, under stringent hybridization conditions to an LP sequence of a table herein, or to a sequence contained in SEQ ID NO:X. In specific embodiments, an LP polynucleotide sequence is at least 15, at least 30, at least 50, at least 100, at least 125, at least 500, or at least 1000 contiguous nucleotides but are less than or equal to 300 kb, 200 kb, 100 kb, 50 kb, 15 kb, 10 kb, 7.5 kb, 5 kb, 2.5 kb, 2.0 kb, or 1 kb, in length.

An LP polynucleotide sequence can be composed of any polyribonucleotide or polydeoxribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single-and double-stranded regions. In addition, the polynucleotide can be composed of triple-stranded regions comprising RNA or DNA or both RNA and DNA. A polynucleotide may also contain one or more modified bases or DNA or RNA backbones modified for stability or for other reasons. “Modified” bases can include, e.g., for example, tritylated bases and unusual bases such as inosine. A variety of modifications can be made to DNA and RNA; thus, the term “polynucleotide” embraces chemically, enzymatically, or metabolically modified forms. “Altered” nucleic acid sequences encoding LP include those sequences with deletions, insertions, or substitutions of different nucleotides, resulting in a polypeptide the same as LP or a polypeptide with at least one functional characteristic of LP. Included within this definition are polymorphisms which may or may not be readily detectable using a particular oligonuclcotide probe of the polynucleotide encoding LP, and improper or unexpected hybridization to allelic variants, with a locus other than the normal chromosomal locus for the polynucleotide sequence encoding LP.

“Substantial similarity” in a nucleic acid sequence comparison context means either that the segments, or their complementary strands, when compared, are identical when optimally aligned, with appropriate nucleotide insertions or deletions, in at least about 50% of the nucleotides, generally at least 56%, more generally at least 59%, ordinarily at least 62%, more ordinarily at least 65%, often at least 68%, more often at least 71%, typically at least 74%, more typically at least 77%, usually at least 80%, more usually at least about 85%, preferably at least about 90%, more preferably at least about 95 to 98% or more, and in particular embodiments, as high at about 99% or more of the nucleotides. Alternatively, substantial similarity exists when the segments will hybridize under selective hybridization conditions, to a strand, or its complement, typically using a sequence derived from SEQ ID X. Typically, selective hybridization will occur when there is at least about 55% similarity over a stretch of at least about 30 nucleotides, preferably at least about 65% over a stretch of at least about 25 nucleotides, more preferably at least about 75%, and most preferably at least about 90% over about 20 nucleotides. See Kanehisa (1984) Nuc. Acids Res. 12:203-213. The length of similarity comparison, as described, may be over longer stretches, and in certain embodiments will be over a stretch of at least about 17 nucleotides, usually at least about 20 nucleotides, more usually at least about 24 nucleotides, typically at least about 28 nucleotides, more typically at least about 40 nucleotides, preferably at least about 50 nucleotides, and more preferably at least about 75 to 100 or more nucleotides, e.g., 150, 200, etc.

For sequence comparison, typically one sequence acts as a reference sequence, to which test sequences are compared. When using a sequence comparison algorithm, test and reference sequences are input into a computer, subsequent coordinates are designated, if necessary, and sequence algorithm program parameters are designated. The sequence comparison algorithm then calculates the percent sequence identity for the test sequence(s) relative to the reference sequence, based on the designated program parameters. Optical alignment of sequences for comparison can be conducted, e.g., by the local homology algorithm of Smith and Waterman (1981) Adv. Appl. Math. 2:482, by the homology alignment algorithm of Needlman and Wunsch (1970) J. Mol. Biol. 48:443, by the search for similarity method of Pearson and Lipman (1988) Proc. Nat'l Acad. Sci. USA 85:2444, by computerized implementations of these algorithms (GAP, BESTFlT, FASTA, and TFASTA in the Wisconsin Genetics Software Package, Genetics Computer Group, 575 Science Dr., Madison, Wis.), or by visual inspection (see generally Ausubel et al., supra). One example of a useful algorithm is PILEUP. Another example of algorithm that is suitable for determining percent sequence identity and sequence similarity is the BLAST algorithm, which is described Altschul, et al. (1990) J. Mol. Biol. 215:403-410. A further indication that two nucleic acid sequences of polypeptides are substantially identical is that the polypeptide encoded by the first nucleic acid is immunologically cross reactive with the polypeptide encoded by the second nucleic acid. Another indication that two nucleic acid sequences are substantially identical is that the two molecules hybridize to each other under stringent conditions.

“Homologous” polynucleotide sequences, when compared, exhibit significant similarity (e.g., sequence identity at the nucleotide level). Generally, standards for determining homology between nucleic acid molecules (or polynucleotide sequences) use art known techniques which examine, e.g., the extent of structural similarity or sequence identity between polynucleotide sequences; and/or that determine a phylogenetic relationship (e.g., whether compared sequences are orthologs or paralogs); and/or that are based on the ability of sequences to form a hybridization complex. Hybridization conditions are described in detail herein.

“Hybridization” refers to the process by which a polynucleotide strand anneals with a complementary strand through base pairing under defined hybridization conditions. Specific hybridization is an indication that two nucleic acid sequences share a high degree of similarity and/or identity. Specific hybridization complexes form under permissive annealing conditions and remain hybridized after “washing.” Washing is particularly important in determining the stringency of the hybridization process, typically, with more stringent conditions allowing less non-specific binding (e.g., binding between polynucleotide sequences that demonstrate less sequence identity or similarity). Permissive conditions for annealing of nucleic acid sequences are routinely determinable by one of ordinary skill in the art and may be consistent among hybridization experiments, whereas wash conditions may be varied among experiments to achieve a desired stringency, and therefore, a particular hybridization specificity.

“Stringent conditions,” when referring to homology or substantial similarity and/or identity in the hybridization context, will be stringent combined conditions of salt, temperature, organic solvents, and other parameters, typically those controlled in hybridization reactions. Stringent temperature conditions will usually include temperatures in excess of about 30° C., more usually in excess of about 37° C., typically in excess of about 40° C., characteristically in excess of about 42° C., routinely in excess of about 45° C., usually in excess of about 47° C., preferably in excess of about 50° C., more typically in excess of about 55° C., characteristically in excess of about 60° C., preferably in excess of about 65° C., and more preferably in excess of about 70° C. In this context, the term “about” includes, e.g., a particularly recited temperature (e.g., 50° C.), and/or a temperature that is greater or lesser than that of the stated temperature by, e.g., one, two, three, four, or five degrees Celsius (e.g., 49° C. or 51° C.). Stringent salt conditions will ordinarily be less than about 500 mM, usually less than about 450 mM, even more usually less than about 400 mM, more usually less than about 350 mM, even more usually less than about 300 mM, typically less than about 250 mM, even more typically less than about 200 mM, preferably less than about 100 mM, and more preferably less than about 80 mM, even down to less than about 20 mM. In this context, the term “about” includes, e.g., a particularly recited molarity (e.g., 400 mM), and/or a molarity that is greater or lesser than that of the stated molarity by, e.g., three, five, seven, nine, eleven or fifteen millimolar (e.g., 389 mM or 415 mM). It is to be remembered that the combination of parameters is more important than the measure of any single parameter (see, e.g., Wetmur and Davidson (1968) J. Mol. Biol. 31:349-370).

A nucleic acid probe that binds to a target nucleic acid under stringent conditions to form a stable hybridization complex is said to be specific for said target nucleic acid. Preferably, hybridization under stringent conditions should give a signal of at least 2-fold over background, more preferably a signal of at least 3 to 5-fold over background or more. Typically, a hybridization probe is more than 11 nucleotides in length and is sufficiently identical (or complementary) to the sequence of the target nucleic acid (over the region determined by the sequence of the probe) to bind the target under stringent hybridization conditions to form a detectable stable hybridization complex. The term “hybridization complex” refers to a complex formed between two nucleic acid molecules by virtue of the formation of hydrogen bonds between complementary bases. A hybridization complex may be formed in solution (e.g., Cot or Rot analysis) or formed between one nucleic acid sequence present in solution and another nucleic acid sequence immobilized on a solid support (such as, e.g., without limitation, paper, plastic, a membrane, a filter, a chip, a pin, glass, or any other appropriate substrate to which cells or their nucleic acids can be complexed with either covalently or non-covalently).

An equation for calculating Tₘ and conditions for nucleic acid hybridization are well known (see, e.g., Sambrook, et al. (1990) Molecular Cloning: A Laboratory Manual (cur. ed.) Vol. 1-3, Cold Spring Harbor Laboratory, Cold Spring Harbor Press, NY, which is incorporated herein by reference and hereinafter referred to as “Sambrook, et al.”). A non-limiting example of a high stringency condition of the invention comprises including a wash condition of 68° C. in the presence of about 0.2×SSC and about 0.1% SDS, for 1 hour. Alternatively, temperatures of about 67° C., 63° C., 61° C., 59° C., 57° C., 53° C., 51° C., 49° C., 47° C., 43° C., or 41° C. may be used. SSC concentration may be varied from about 0.1 to 2.0×SSC, with SDS being present at about 0.1%. Typically, blocking reagents are used to block non-specific hybridization. Such blocking reagents include, for instance, sheared, and denatured salmon sperm DNA at about 100-200 ug/ml. Organic solvent, such as, e.g., formamide at a concentration of about 35-50% v/v, may also be used under particular circumstances, such as for a RNA:DNA hybridization. Useful variations on these wash conditions will be readily apparent to those of ordinary skill in the art. Hybridization, particularly under high stringency conditions, may be suggestive of evolutionary similarity between the nucleotides. Such similarity is indicative of a similar functional and/or biological role for the nucleotide sequence and its correspondingly encoded polypeptide sequence.

Another non-limiting example of a stringent hybridization condition comprises, e.g., an overnight incubation at 42° C. in a solution comprising 50% formamide, 5×SSC (750 mM NaCl, 75 mM trisodium citrate), 50 mM sodium phosphate (pH 7.6), 5×Denhardt's solution, 10% dextran sulfate, and 20 pg/ml denatured, sheared salmon sperm DNA, followed by washing the filters in 0.1×SSC at about 65° C. Also contemplated are nucleic acid molecules that hybridize to an LP polynucleotide sequence at lower stringency hybridization conditions. Changes in the stringency of hybridization and signal detection can be accomplished through the manipulation of formamide concentration (lower percentages of formamide result in lowered stringency); salt conditions, or temperature. For example, an alternate stringency condition can comprise, e.g., an overnight incubation at 37° C. in a solution comprising 6×SSPE (20×SSPE=3M NaCl; 0.2M NaH,PO, 0.02M EDTA, pH 7.4), 0.5% SDS, 30% formamide, 100ml salmon sperm blocking DNA; followed by washes at 50° C. with 1×SSPE, 0.1% SDS. In addition, to achieve another alternate stringency condition, washes are performed following stringent hybridization at higher salt concentrations (e.g. 5×SSC). Note that variations in the above conditions may be accomplished through the inclusion and/or substitution of alternate blocking reagents used to suppress background in hybridization experiments. Typical blocking reagents include, e.g., Denhardt's reagent, BLOTTO, heparin, denatured salmon sperm DNA, and commercially available proprietary formulations. The inclusion of specific blocking reagents may require modification of a hybridization conditions described herein. A polynucleotide that hybridizes only to polyA+ sequences (such as any 3′ terminal polyA+ tract of a CDNA of the invention), or to a complementary stretch of T (or U) residues, is not included, e.g., in the definition of an “LP polynucleotide” since such a polynucleotide would hybridize to any nucleic acid molecule containing a poly (A) stretch or the complement thereof (i.e., practically any double-stranded CDNA clone generated using oligo dT as a primer).

Still another non-limiting example of a stringent hybridization condition is one that employs, e.g.: low ionic strength and high temperature for washing (e.g., 15 mM sodium chloride/1.5 mM sodium citrate/0.1% sodium dodecyl sulfate at 50° C.); a denaturing agent (during hybridization) such as formamide (e.g., 50% (v/v) formamide with 0.1% bovine serum albumin/0.1% ficoll/0.1% polyvinylpyrrolidone/50 mM sodium phosphate buffer at pH 6.5 with 750 mM sodium chloride/75 mM sodium citrate at 42° C.); or 50% formamide, 5×SSC (750 μM sodium chloride, 75 mM sodium citrate), 50 mM sodium phosphate (pH 6.8), 0.1% sodium pyrophosphate, 5×Denhardt's solution, sonicated salmon sperm DNA (50 μg/mL), 0.1% SDS, and 10% dextran sulfate at 42° C. with washes at 42° C. in 0.2×SSC (30 mM sodium chloride/3 mM sodium citrate) and 50% formamide at 55° C., followed by a high-stringency wash consisting of 0.1×SSC containing EDTA at 55° C.

An “isolated” nucleic acid is a nucleic acid molecule or a polynucleotide sequence (e.g., an RNA, DNA, cDNA, genomic DNA, or a mixed polymer) which is substantially separated from other biologic components that naturally accompany a native sequence (e.g., proteins and flanking genomic sequences from the originating species). In a preferable embodiment, the isolated LP sequence is free of association with components that can interfere with diagnostic or therapeutic uses for the sequence including, e.g., enzymes, hormones, and other proteinaceous or non-proteinaceous agents. The term embraces a polynucleotide sequence removed from its naturally occurring environment. For example, an isolated polynucleotide sequence could comprise part of a vector or a composition of matter, or could be contained within a cell, and still be “isolated” because the vector, composition of matter, or cell is not the original environment of the polynucleotide sequence. Moreover, the term encompasses recombinant or cloned DNA isolates, chemically synthesized analogs, or analogs biologically synthesized using heterologous systems. Furthermore, the term includes both double-stranded and single-stranded embodiments. If single-stranded, the polynucleotide sequence may be either the “sense” or the “antisense” strand. A substantially pure molecule includes isolated forms of the molecule.

An isolated nucleic acid molecule will usually contain homogeneous nucleic acid molecules, but, in some embodiments, it will contain nucleic acid molecules having minor sequence heterogeneity. Typically, this heterogeneity is found at the polymer ends or portions of the LP sequence that are not critical to a desired biological function or activity.

The term “isolated” does not refer to genomic or cDNA libraries, whole cell total or mRNA preparations, genomic DNA preparations (including those separated by electrophoresis and transferred onto blots), sheared whole cell genomic DNA preparations, or other compositions where the art demonstrates no distinguishing features of a LP polynucleotide sequence of the present invention.

A “recombinant” nucleic acid or polynucleotide sequence is defined either by its method of production or its structure. In reference to its method of production, e.g., a product made by a process, the process is use of any genetic engineering technique, e.g., products made by transforming cells with any non-naturally occurring vector are encompassed, as are nucleic acids comprising sequence derived using any synthetic oligonucleotide process. A similar concept is intended for a recombinant LP polypeptide. Specifically included are synthetic nucleic acid molecules which, due to the redundancy of the genetic code, encode polypeptides similar to fragments of these antigens, and fusions of sequences from various different species variants.

LP Protein

As used herein, an “LP protein” shall encompass, when used in a protein context, a protein or polypeptide having an amino acid sequence shown in SEQ ID NO: Y or a significant fragment of such a protein or polypeptide, preferably a natural embodiment. The term “protein” or “polypeptide” is meant any chain of contiguous amino acid residues, regardless of length or postranslation modification (e.g., glycosylation, or phosphorylation).

Further, an LP protein or an LP polypeptide encompass polypeptide sequences that are pre- or pro-proteins. Moreover, the present invention encompasses a mature LP protein, including a polypeptide or protein that is capable of being directed to the endoplasmic reticulum (ER), a secretory vesicle, a cellular compartment, or an extracellular space typically, e.g., as a result of a signal sequence, however, a protein released into an extracellular space without necessarily having a signal sequence is also encompassed. Generally, the polypeptide undergoes processing, e.g., cleavage of a signal sequence, modification, folding, etc., resulting in a mature form (see, e.g., Alberts, et al. (1994) Molecular Biology of The Cell, Garland Publishing, New York, N.Y., pp. 557-560, 582-592.).

The invention also embraces polypeptides that exhibit similar structure to an LP polypeptide (e.g., one that interacts with an LP protein specific binding composition). These binding compositions, e.g., antibodies, typically bind an LP protein with high affinity, e.g., at least about 100 nM; usually, better than about 30 nM; preferably, better than about 10 nM; and more preferably, at better than about 3 nM.

Modifications

An LP polypeptide can be composed of amino acids joined to each other by peptide bonds or modified peptide bonds, i.e., peptide isosteres, and may contain amino acids other than the 20 gene-encoded amino acids. The polypeptides may be modified by either natural processes, such as post-translational processing, or by chemical modification techniques that are well known in the art. Such modifications are well described in basic texts and in more detailed monographs, as well as in a voluminous research literature.

Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination (see, e.g., Creighton (1993) 2nd ed. Proteins-Structure and Molecular Properties, W. H. Freeman and Company, New York; Johnson (1983) ed. Posttranslational Covalent Modification of Proteins, Academic Press, New York, pp.1-12; Seifter et al. (1990) Meth Enzymol 182:626-646; Rattan et al. (1992) Ann NY Acad Sci 663:48XX).

The encoded protein may also be “altered,” and may contain deletions, insertions, or substitutions of amino acid residues that produce a silent change and result in a functionally equivalent LP. Deliberate amino acid substitutions may be made based on similarity in polarity, charge, solubility, hydrophobicity, hydrophilicity, and/or the amphipathic nature of the residues, as long as the biological or immunological activity of the LP is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

“Substantially pure” refers to LP nucleic acid or LP protein or polypeptide that are removed from their natural environment and are isolated and/or separated from other contaminating proteins, nucleic acids, and other biologicals. Purity may be assayed by standard methods, and will ordinarily be at least about 50% pure, more ordinarily at least about 60% pure, generally at least about 70% pure, more generally at least about 80% pure, often at least about 85% pure, more often at least about 90% pure, preferably at least about 95% pure, more preferably at least about 98% pure, and in most preferred embodiments, at least 99% pure. Similar concepts apply, e.g., to LP antibodies or nucleic acids of the invention. For example, it may be desirable to purify an LP polypeptide from recombinant cell proteins or polypeptides. Various art known methods of protein purification may be employed (see, e.g., Deutscher, (1990) Methods in Enzymology 182: 83-9 and Scopes, (1982) Protein Purification: Principles and Practice, Springer-Verlag, NY.)

“Solubility” of an LP protein or polypeptide is reflected by sedimentation measured in Svedberg units, which are a measure of the sedimentation velocity of a molecule under particular conditions (see, Freifelder (1982) Physical Biochemistry (2d ed.) W.H. Freeman & Co., San Francisco, Calif.; and Cantor and Schimmel (1980) Biophysical Chemistry parts 1-3, W.H. Freeman & Co., San Francisco, Calif.). A soluble particle or polypeptide will typically be less than about 30 S, more typically less than about 15 S, usually less than about 10 S, more usually less than about 6 S, and, in particular embodiments, preferably less than about 4 S, and more preferably less than about 3 S. Solubility of a polypeptide or fragment depends upon the environment and the polypeptide. Many parameters affect polypeptide solubility, including temperature, electrolyte environment, size and molecular characteristics of the polypeptide, and nature of the solvent. Typically, the temperature at which the polypeptide is used ranges from about 4° C. to about 65° C. Usually the temperature at use is greater than about 18° C. and more usually greater than about 22° C. For diagnostic purposes, the temperature will usually be about room temperature or warmer, but less than the denaturation temperature of components in the assay. For therapeutic purposes, the temperature will usually be body temperature, typically about 37° C. for humans, though under certain situations the temperature may be raised or lowered in situ or in vitro.

The size and structure of the polypeptide should generally be in a substantially stable state, and usually not in a denatured state. The polypeptide may be associated with other polypeptides in a quaternary structure, e.g., to confer solubility, or associated with lipids or detergents in a manner which approximates natural lipid bilayer interactions.

The solvent will usually be a biologically compatible buffer, of a type used for preservation of biological activities, and will usually approximate a physiological solvent. Usually the solvent will have a neutral pH, typically between about 5 and 10, and preferably about 7.5. On some occasions, a detergent will be added, typically a mild non-denaturing one, e.g., CHS (cholesteryl hemisuccinate) or CHAPS (3-[3-cholamidopropyl)-dimethylammonio]-1 -propane sulfonate), or a low enough concentration as to avoid significant disruption of structural or physiological properties of the protein.

Signal Sequence

The present invention encompasses “mature” forms of a polypeptide comprising a polypeptide sequence listed in a Table herein, or a polypeptide sequence of SEQ ID NO: Y. Methods for predicting whether a protein has a signal sequence, as well as the cleavage point for that sequence, are known in the art (see, e.g., McGeoch, 1985 Virus Res. 3:271-286 and Henrik Nielsen et al. (1997) Protein Engineering 10: 1-6). Employing such known art methods a signal sequence for an LP polypeptide was made. However, cleavage sites may vary and cannot be predicted with absolute certainty. Accordingly, the present invention provides secreted LP polypeptides having a sequence listed in a Table herein, or a polypeptide sequence of SEQ ID NO: Y, in which a particular N-terminus variant polypeptide sequence can begin within five, four, three, two, or one amino acid residues (e.g., +5, +4, +3, +2, +1, or −5, −4, −3, −2, −1) from a particular cleavage point designated as such herein. Similarly, it is also recognized that in some cases, cleavage of a signal sequence of a secreted protein is not uniform, resulting in more than one secreted species for a given protein (e.g., a cleavage variant). Such cleavage variant LP polypeptides, and the polynucleotides encoding them, are also encompassed by the present invention.

Moreover, the signal sequence identified by the above analysis may not necessarily predict a naturally occurring signal sequence. For example, a naturally occurring signal sequence may be further upstream from a predicted signal sequence. However, it is likely that a predicted signal sequence will be capable of directing the secreted protein to the ER. Nevertheless, the present invention encompasses a mature LP polypeptide or protein produced by expression of a polynucleotide sequence listed in a Table herein or an LP polynucleotide sequence of SEQ ID NO: X. These LP polypeptides (and fragments thereof), and the polynucleotides encoding them, are also encompassed by the present invention.

LP Variants

The present invention encompasses variants of an LP polynucleotide sequence disclosed in a table herein or SEQ ID NO: X and/or the complementary strand thereto. The present invention also encompasses variants of a polypeptide sequence disclosed in a table herein or SEQ ID NO: Y. The term “variant” refers to a polynucleotide or polypeptide differing from an LP polynucleotide sequence or an LP polypeptide of the present invention, but retaining essential properties thereof Generally, variants are closely similar overall in structural and/or sequence identity, and, in many regions, identical to an LP polynucleotide or polypeptide of the present invention. For example, the present invention encompasses nucleic acid molecules that comprise, or alternatively consist of, a polynucleotide sequence that is at least: 80%, 85%, 90%, 95%, 96%, 97%, 98%, or 99% identical to, e.g., a polynucleotide coding sequence of SEQ ID NO: X (or a strand complementary thereto); a nucleotide sequence encoding a polypeptide of SEQ ID NO: Y; and/or polynucleotide fragments of any of these nucleic acid molecules (e.g., a fragment as defined herein). Polynucleotides, that stably hybridize to a polynucleotide fragment (as defined herein) under stringent hybridization conditions or lower stringency conditions, are also encompassed by the invention, as are polypeptides (or fragments thereof encoded by these polynucleotides.

The present invention is also directed to polypeptides that comprise, or alternatively consist of, an amino acid sequence that is at least: 80%, 85%, 90%, 95%, 96%, 97%, 98%, 99% identical to, e.g., a polypeptide sequence of SEQ ID NO: Y (or fragments thereof); a polypeptide sequence encoded by a cDNA contained in a deposited clone, and/or a polypeptide fragment of any of these polypeptides (e.g., those fragments as defined herein). A polynucleotide sequence having at least some “percentage identity,” (e.g., 95%) to another polynucleotide sequence, means that the sequence being compared (e.g., the test sequence) may vary from another sequence (e.g. the referent sequence) by a certain number of nucleotide differences (e.g., a test sequence with 95% sequence identity to a reference sequence can have up to five point mutations per each 100 contiguous nucleotides of the referent sequence). In other words, for a test sequence to exhibit at least 95% identity to a referent sequence, up to 5% of the nucleotides in the referent may differ, e.g., be deleted or substituted with another nucleotide, or a number of nucleotides (up to 5% of the total number of nucleotides in the reference sequence) may be inserted into the reference sequence. The test sequence may be: an entire polynucleotide sequence, e.g., as shown in a Table herein, the ORF (open reading frame), or any fragment, segment, or portion thereof (as described herein). As a practical matter, determining if a particular nucleic acid molecule or polynucleotide sequence exhibits at least about: 80%, 85%, 90%, 95%, 96%, 97%, 98% or 99% identity to an LP polynucleotide sequence can be accomplished using any art known method.

Variants encompassed by the present invention may contain alterations in the coding regions, non-coding regions, or both. Moreover, variants in which 1-2,1-5, or 5-10 amino acids are substituted, deleted, or added in any combination are also preferred. Of course, in order of ever-increasing preference, it is highly preferable for a peptide or polypeptide to have an amino acid sequence that comprises an amino acid sequence of the present invention, which contains at least: one, but not more than: 10, 9, 8, 7, 6, 5, 4, 3, 2, or 1 amino acid substitutions. In specific embodiments, the number of additions, substitutions, and/or deletions in an polypeptide sequence of the present invention or fragments thereof (e.g., a mature form and/or other fragments described herein), is at least: 1, 2, 3, 4, 5, 6, 7, 8, 9, 10, 11, 12, 13, 14,15,16, 17,18, 19, 20, 21, 22, 23, 24, 25, 10-50, or 50-150; wherein conservative amino acid substitutions are more preferable than non-conservative substitutions.

LP Polynucleotide and LP Polypeptide Fragments

The present invention is also directed to fragments of an LP polynucleotide. An LP polynucleotide “fragment” encompasses a short polynucleotide of a nucleic acid molecule, or a portion of a polynucleotide sequence of SEQ ID NO: X or a complementary strand thereto, or a portion of a polynucleotide sequence encoding a polypeptide of SEQ ID NO: Y (or fragment thereof). Polynucleotide fragments of the invention encompass a polynucleotide sequence that is preferably at least about 15 nucleotides, more preferably at least about: 20, 21, 22, 24, 26, or 29 nucleotides, favorably at least about: 30, 32, 34, 36, 38, or 39 nucleotides, and even more preferably, at least about: 40, 42, 44, 46, 48, or 49 nucleotides, desirably at least about: 50, 52, 54, 56, 58, or 59 nucleotides, particularly at least about 75 nucleotides, or at least about 150 nucleotides in length.

A polynucleotide fragment “at least 20 nucleotides in length,” e.g., is intended to include, e.g., 20 or more contiguous bases from a nucleotide sequence shown in SEQ ID NO: X or in a Table herein. In this context “at least about” includes, e.g., a specifically recited value (e.g., 20 nt), and a value that is larger or smaller by one or more nucleotides (e.g., 5, 4, 3, 2, or 1), at either terminus or at both termini. A polynucleotide fragment has use that includes without limit; e.g., diagnostic probes and primers as discussed herein. Larger fragments (e.g., 50,150, 500, 600, or 2000 nucleotides) are also useful and preferred.

Representative examples of various lengths of polynucleotide fragments encompassed by the invention, include, e.g., fragments comprising, or alternatively consisting of, a polynucleotide sequence of SEQ ID NO:X from about nucleotide number 1-50, 51-100, 101-1 50, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 101851-1900, 1901-1950,1951-2000, or 2001 to the end of SEQ ID NO:X, or a strand complementary thereto. In this context, the term “about” includes, e.g., a particularly recited polynucleotide fragment range herein, and/or ranges that have lengths that are larger or smaller by several nucleotides (e.g., 5, 4, 3, 2, or 1 nt), at either terminus or at both termini. Preferably, these fragments encode a polypeptide possessing biological activity as defined herein, e.g., immunogenicity, or antigenicity. More preferably, a polynucleotide fragment can be used as a probe or primer as discussed herein. Furthermore, the present invention also encompasses a polynucleotide that stably hybridizes to a polynucleotide fragment described herein under either stringent or lowered stringency hybridization conditions. Additionally incorporated are polypeptides encoded by a polynucleotide fragment or a hybridized polynucleotide stably bound to a polynucleotide fragment of the invention. Additionally encompassed by the invention is a polynucleotide encoding a polypeptide, which is specifically or selectively bound by an antibody directed to/or generated against a mature polypeptide of the invention (or fragment thereof), e.g., a mature polypeptide of SEQ ID NO: Y.

In the present invention, a “polypeptide fragment or segment” encompasses an amino acid sequence that is a portion of SEQ ID NO: Y. Protein and/or polypeptide fragments or segments may be “free-standing,” or they may comprise part of a larger polypeptide or protein, of which the fragment or segment forms a portion or region, e.g., a single continuous region of SEQ ID NO: Y connected in a fusion protein. Representative examples of lengths of polypeptide fragments or segments encompassed by the invention, include, e.g., fragments comprising, or alternatively consisting of, from about amino acid residue number 1-20, 21-40, 41-60, 61-80, 81-100, 102-120, 121-140, 141-160, 161-170, 171-180, 181-190, 191-200, 201-210, etc., to the end of the mature coding region of a polypeptide of the invention (or fragment thereof).

Preferably, a polypeptide segment of the invention can have a length of contiguous amino acids of a polypeptide of the invention (or fragment thereof) that is at least about: 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70,72, 74, 76, 78, 80, 82, 84, 86, 88, 90, 92, 94, 96, 98, 100, 110, 120, 130, 140, or 150 contiguous amino acids in length. In this context “about” includes, e.g., the specifically recited ranges or values described herein, and it also encompasses values that differ from these recited values by several amino acid residues (e.g., plus or minus 5, plus or minus 4, plus or minus 3, plus or minus 2, or; plus or minus 1 amino acid residues), at either or both ends of the fragment. Further, a polynucleotide encoding such a polypeptide fragment is also encompassed by the invention.

Moreover, a polypeptide comprising more than one of the above polypeptide fragments is encompassed by the invention; including a polypeptide comprising at least: one, two, three, four, five, six, seven, eight, nine, ten, or more fragments, wherein the fragments (or combinations thereof may be of any length described herein (e.g., a fragment of 12 contiguous amino acids and another fragment of 30 contiguous amino acids, etc.). The invention also encompasses proteins or polypeptides comprising a plurality of distinct, e.g., non-overlapping, segments of specified lengths. Typically, the plurality will be at least two, more usually at least three, and preferably four, five, six, seven, eight, nine, ten, or even more. While length minima are stipulated, longer lengths (of various sizes) may be appropriate (e.g., one of length seven, and two of lengths of twelve). Features of one of the different polynucleotide sequences should not be taken to limit those of another of the polynucleotide sequences. Preferred polypeptide fragments include, e.g., the secreted protein as well as the mature form. Further preferred polypeptide fragments include, e.g., the secreted protein or the mature form having a continuous series of deleted residues from the amino or the carboxy terminus, or both. For example, 1, 2, 3, 4, 5, 6, 7, 8, 9, 10, 11, 12, 13,14, 15, 16, 17, 18, 19, 20, 21, 22, 23, 24, 25, 26, 27, 28, 29, or 30 amino acids can be deleted from the amino terminus of either the secreted polypeptide or the mature form. Similarly, 1, 2, 3, 4, 5, 6, 7, 8, 9, 10, 11, 12, 13, 14,15, 16, 17, 18, 19, 20, 21, 22, 23, 24, 25, 26,27, 28, 29, or 30, can be deleted from the carboxy terminus of the secreted protein or mature form. Furthermore, any combination of the above amino and carboxy terminus deletions are preferred. Similarly, polynucleotides encoding these polypeptide fragments are also preferred.

Also preferred are polypeptide fragments or segments (and their corresponding polynucleotide fragments) that characterize structural or functional domains, such as, fragments, or combinations thereof, that comprise e.g., alpha-helix, and alpha-helix forming regions, beta-sheet, and beta-sheet-forming regions, turn, and turn-forming regions, coil, and coil-forming regions, hydrophilic regions, hydrophobic regions, alpha amphipathic regions, beta amphipathic regions, flexible regions, loop regions, hairpin domains, beta-alpa-beta motifs, helix bundles, alpha/beta barrels, up and down beta barrels, jelly roll or swiss roll motifs, transmembrane domains, surface-forming regions, substrate binding regions, transmembrane regions, linkers, immunogenic regions, epitopic regions, and high antigenic index regions. Polypeptide fragments of SEQ ID NO: Y falling within conserved domains are specifically encompassed by the present invention. Moreover, polynucleotides encoding these domains are also encompassed. Other preferred polypeptide segments are biologically active fragments. Biologically active fragments are those exhibiting activity similar, but not necessarily identical, to an activity of an LP polypeptide (or fragment thereof). The biological activity of the fragments may include, e.g., an improved desired activity, or a decreased undesirable activity. Polynucleotides encoding these polypeptide fragments are also encompassed by the invention.

Preferably, the polynucleotide fragments of the invention encode a polypeptide that demonstrates a functional activity. The phrase “functional activity” encompasses a polypeptide segment that can accomplish one or more known functional activities associated with a full-length (complete) polypeptide of invention protein. Such functional activities include, e.g., without limitation, biological activity, antigenicity [ability to bind (or compete with a polypeptide of the invention for binding) to an antibody to a polypeptide of the invention], immunogenicity (ability to generate antibody that binds to a polypeptide of the invention), ability to form multimers with a polypeptide of the invention, and the ability to bind to a receptor or ligand of a polypeptide of the invention.

The functional activity of a polypeptide of the invention (including fragments, variants, derivatives, and analogs thereof) can be assayed by various methods. For example, where one is assaying for the ability to bind or compete with a full-length polypeptide of the invention for binding to an antibody of a polypeptide of the invention, various immunoassays known in the art can be used, including, e.g., without limitation, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays, complement fixation assays, immunofluorescence assays, protein A assays, and immunoelectrophoresis assays, etc.)

In another embodiment, antibody binding is accomplished by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In another embodiment, where a ligand for a polypeptide of the invention is identified, or the ability of a polypeptide fragment, variant or derivative of the invention to multimerize is being evaluated, binding can be assayed, e.g., by using reducing and non-reducing gel chromatography, protein affinity chromatography, and affinity blotting (see generally, Phizicky, et al. (1995) Microbial. Rev. 59:94123). In another embodiment, physiological correlates of binding of a polypeptide of the invention to its substrates (signal transduction) can be assayed with common techniques. In addition, assays described herein (see, e.g., the “Examples” section of the application), or otherwise known in the art, can routinely be applied to measure the ability of a polypeptide of the invention (its fragments, variants derivatives and analogs thereof) to elicit a related biological activity (either in vitro or in vivo).

Epitopes and Antibodies

The present invention encompasses a polypeptide comprising, or alternatively consisting of, an epitope of SEQ ID NO: Y or a table herein; or encoded by a polynucleotide that stably hybridizes to form a hybridization complex, under stringent hybridization conditions (or lower stringency hybridization conditions) as defined herein, to a complement of a sequence of SEQ ID NO: X.

The present invention further encompasses a polynucleotide sequence encoding an epitope of a polypeptide sequence of the invention (such as, e.g., a sequence disclosed in SEQ ID NO: X or a Table herein), a polynucleotide sequence of the complementary strand of a polynucleotide sequence encoding an epitope of the invention, and a polynucleotide sequence that stably hybridizes to a complementary strand under stringent hybridization conditions or lower stringency hybridization conditions as defined herein.

The term “epitope,” as used herein, refers to a portion of a polypeptide having antigenic or immunogenic activity in an animal, preferably a mammal, and most preferably in a human. In a preferred embodiment, the present invention encompasses a polypeptide comprising an epitope, as well as the polynucleotide encoding this polypeptide.

An “immunogenic epitope,” as used herein, is defined as a portion of a protein or a linearized polypeptide (or fragment thereof) that elicits an antibody response in an animal, as determined by any art known method (e.g., by the methods for generating antibodies described herein or otherwise known, see, e.g., Geysen, et al. (1983) Proc. Natl. Acad. Sci. USA 308 1:3998-4002).

An “antigenic epitope,” as used herein, is defined as a portion of a protein or polypeptide to which a binding composition, e.g., an antibody or antibody binding fragment, selectively binds or is specifically immunoreactive with as determined by any known art method, e.g., by an immunoassay described herein. Selective binding excludes non-specific binding but does not necessarily exclude cross-reactivity with other antigens. An antigenic determinant may compete with the intact antigen (i.e., the immunogen used to elicit the immune response) for binding to an antibody. Antigenic epitopes need not necessarily be immunogenic.

The phrase “specifically binds to” or is “specifically immunoreactive with”, when referring to a protein or peptide, refers to a binding reaction which is determinative of the presence of a protein or fragment (e.g., an LP protein) in the presence of a heterogeneous population of proteins and/or other biological components. Typically, the interaction is dependent upon the presence of a particular structure, e.g., an antigenic determinant (or epitope) recognized by a binding composition. For example, if an antibody is specific for epitope “A,” the presence of a polypeptide comprising the epitope A, or the presence of free unlabeled A, in a reaction containing free labeled A and the antibody will reduce the amount of labeled A that binds to the antibody. Thus, under designated immunoassay conditions, the specified antibodies bind to a particular protein or polypeptide sequence and do not significantly bind other proteins or other polypeptide sequences that are present in the sample. Specific binding to an antibody under such conditions may require an antibody that is selected for its specificity and/or selectivity for a particular protein. For example, antibodies raised to the protein immunogen with an amino acid sequence depicted in SEQ ID NO: Y can be selected to obtain antibodies specifically immunoreactive with LP proteins or LP polypeptides and not with other proteins or polypeptides. These antibodies will also recognize proteins or polypeptide sequences that have an above average degree of similarity or identity to an LP protein or LP polypeptide sequence. Fragments that function as epitopes can be produced by any conventional means such as, e.g., (1985) Houghten, Proc. Natl. Acad. Sci. USA 82:5131-5135, further described in U.S. Pat. No.4,631,211.

In the present invention, an antigenic or immunogenic epitope preferably contains a polypeptide sequence of at least four, at least five, at least six, at least seven, more preferably at least eight, at least nine, at least 10, at least 11, at least 12, at least 13, at least 14, at least 15, at least 20, at least 25, at least 30, at least 40, at least 50, and, favorably, between about 15 to about 30 contiguous amino acids of a mature polypeptide of SEQ ID NO: Y or a Table herein. Preferred polypeptide fragments of contiguous amino acid residues of SEQ ID NO: Y comprising immunogenic or antigenic epitopes are at least 10, 15, 20, 25, 30, 35, 40, 45, 50, 55, 60, 65, 70, 75, 80, 85, 90, 95, or 100 contiguous amino acid residues in length.

Additional non-exclusive preferred antigenic epitopes include, e.g., the antigenic epitopes disclosed herein, as well as portions thereof. Antigenic epitopes are useful, e.g., to generate antibodies, including monoclonal antibodies that specifically bind the epitope. Preferred antigenic epitopes include, e.g., the antigenic epitopes disclosed herein, as well as any plurality thereof, e.g., at least: two, three, four, five or more of these antigenic epitopes in any combination or structural arrangement. Antigenic epitopes can be used as the target molecules in immunoassays (see, e.g., Wilson, et al. (1984) Cell 37:767-778; Sutcliffe, et al. (1983) Science 219:660-666). Similarly, immunogenic epitopes can be used, e.g., to induce antibodies according to any known art method (see, for instance, Sutcliffe, et al. supra, Wilson, et al. supra, Chow, et al. Proc. Natl. Acad. Sci. USA 82:910-25914; and Bittle, et al. (1985) J. Gen. Virol. 66:2347-2354.

Preferred immunogenic epitopes include, e.g., an immunogenic epitope disclosed herein, as well as a plurality or any combination thereof, e.g., of at least two, three, four, five or more of these immunogenic epitopes including, e.g., repeats of a particular epitope. A polypeptide comprising a plurality of epitopes may be used to elicit an antibody response with a carrier protein, such as, e.g., an albumin, to an animal system (such as, e.g., a rabbit or a mouse), or, if a polypeptide is of sufficient length (e.g., at least about 25 amino acids), the polypeptide may be presented without a carrier. However, immunogenic epitopes comprising as few as 8 to 10 amino acids have also been shown to be sufficient to generate antibodies and to be useful since they are capable of binding to, e.g., linear epitopes in a denatured polypeptide such as in Western blotting.

Polypeptides or proteins bearing an epitope of the present invention may be used to generate antibodies according to known methods including, e.g., without limitation, in vivo immunization, in vitro immunization, and phage display methods (see, e.g., Sutcliffe, et al. supra; Wilson, et al. supra, and Bittle, et al. (1985) J. Gen. Virol. 66:2347-2354.

“Binding Composition”

The term “binding composition” refers to molecules that bind with specificity and/or selectivity to an LP of the invention or fragment thereof (such as, e.g., in an antibody-antigen interaction). However, other compositions (e.g., antibodies, oligonucleotides, proteins (e.g., receptors), peptides, or small molecules) may also specifically and/or selectivity associate (bind) with the LP in contrast to other molecules. Typically, the association will be in a natural physiologically relevant protein-protein interaction (either covalent or non-covalent) and it may include members of a multi-protein complex (including carrier compounds or dimerization partners). The composition may be a polymer or chemical reagent. A functional analog may be a protein with structural modifications or may be a wholly unrelated molecule (such as, e.g., one that has a molecular shape that interacts with the appropriate binding determinants). The proteins may serve as agonists or antagonists of the binding partner, see, e.g., Goodman, et al. (eds.) (1990) Goodman & Gilman's: The Pharmacological Bases of Therapeutics (cur. ed.) Pergamon Press, Tarrytown, N.Y.

The LP may be used to screen for binding compositions that specifically and/or selectively bind an LP of the invention or fragment thereof (e.g., a binding composition can be a molecule, or part of one, that selectively and/or stoichiometrically binds, whether covalently or not, to one or more specific sites of an LP (or fragment thereof) such as, e.g., in an antigen-antibody interaction, a hormone-receptor interaction, a substrate-enzyme interaction, etc.). At least one and up to a plurality of test binding compositions can be screened for specific and/or selective binding with the LP.

In one embodiment, a binding composition thus identified is closely related to a natural ligand of an LP (such as, e.g., a ligand or fragment thereof, a natural substrate, a structural or functional mimetic, or a natural binding partner; see, e.g., Coligan, et al. (1991) Current Protocols in Immunology 1(2):Chapter 5.)

“Binding Agent:LP Complex”

The term “binding agent:LP complex,” as used herein, refers to a complex of a binding agent and a LP (or fragment thereof) which is formed by specific and/or selective binding of the binding agent to the respective LP (or fragment thereof). Specific and/or selective binding of the binding agent means that the binding agent has a specific and/or selective binding site that recognizes a site on the LP protein (or fragment thereof). For example, antibodies raised against a LP protein (or fragment thereof) that recognize an epitope on the LP protein (or fragment thereof) are capable of forming a binding agent:LP complex by specific and/or selective binding. Typically, the formation of a binding agent:LP complex allows the measurement of LP protein (or fragment thereof) in a mixture of other proteins and/or biologics.

“Antibody:LP Complex”

The phrase “antibody:LP complex” refers to an embodiment in which the binding agent, e.g., is an antibody. The antibody may be monoclonal, polyclonal, or a binding fragment of an antibody (including, without limit, e.g., Fv, Fab, or F(ab)2 fragments; diabodies; linear antibodies (Zapata, et al., (1995) Protein Engin. 8(10): 1057-62); single-chain antibody molecules; and multispecific antibodies formed from antibody fragments). Preferably, for cross-reactivity purposes, the antibody is a polyclonal antibody.

Antibodies

Antibodies can be raised to various LP proteins, including individual, polymorphic, allelic, strain, or species variants, and fragments thereof, both in their naturally occurring (full-length) forms and in their recombinant forms. Additionally, antibodies can be raised to LP proteins in either their active forms or in their inactive forms. Anti-idiotypic antibodies may also be used. Antibodies of the invention include, e.g., without limitation, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and an epitope-binding fragment of any of the above.

As used herein, the phrase “human antibodies” includes, e.g., without limitation, antibodies having an amino acid sequence of a human immunoglobulin including, e.g., without limitation, an antibody isolated from a human immunoglobulin library or from an animal transgenic for one or more human immunoglobulins and that do not express endogenous immunoglobulins, as described herein or, as taught, e.g., in U.S. Pat. No. 5,939,598. An antibody of the present invention may be monospecific, bispecific, trispecific or of greater multispecificity. Multispecific antibodies may be specific for different epitopes of an LP polypeptide (or fragment thereof or may be specific for both a polypeptide of the present invention as well as for a heterologous epitope, such as a heterologous polypeptide or solid support material (see, e.g., WO 2093/17715; WO 92/08802; WO 91/00360; WO 92/05793; Tutt, et al. (1991) J. Immunol. 147:60-69; U.S. Pat. Nos. 4,474,893; 4,714,681; 4,925,648; 5,573,920; or 5,601,819; or Kostelny, et al. (1992) J. Immunol. 148:1547-1553.

Further encompassed by the present invention is an antibody that selectively binds a polypeptide, which is encoded by a polynucleotide that stably hybridizes, under stringent hybridization conditions (as described herein), to an LP polynucleotide sequence. An antibody of the present invention may also be characterized or specified in terms of its binding affinity to a protein or polypeptide (fragment thereof), or epitope of the invention. A preferred binding affinity of a binding composition, e.g., an antibody or antibody binding fragment, includes, e.g., a binding affinity that demonstrates a dissociation constant or Kd of less than about: 5×10⁻²M, 10⁻²M, 5×10⁻³M, 10⁻³M, 5×10⁻⁴M, 10⁻⁴M, 5×10⁻⁵m, 10⁻⁵M, 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, or 10⁻¹⁵M.

The invention also encompasses antibodies that competitively inhibit binding of a binding composition to an epitope of the invention as determined by any known art method for determining competitive binding, e.g., the immunoassays described herein. In preferred embodiments, the antibody competitively inhibits binding to the epitope by at least 95%, at least 90%, at least 85%, at least 80%, at least 75%, at least 70%, at least 60%, or at least 50%.

Antibodies of the present invention may act as agonists or antagonists of an LP polypeptide (or fragment thereof). Likewise encompassed by the invention, are neutralizing antibodies that bind a ligand and prevent it binding to a receptor. Similarly encompassed are ligand-binding antibodies that inhibit receptor activation without inhibiting receptor binding. Alternatively, ligand-binding antibodies that activate a receptor are also included. Antibodies of the invention may act as receptor agonists, e.g., by potentiating or activating either all or a subset of the biological activities of the ligand-mediated receptor activation, e.g., by inducing dimerization of a receptor. The antibodies may be specified as agonists, antagonists, or inverse agonists for biological activities comprising the specific biological activities of a peptide of the invention disclosed herein. An antibody agonist can be made using known methods art (see, e.g., WO 96/40281; U.S. Pat. No. 5,811,097; Deng, et al., Blood 92(6):1981-1988 (1998); Chen, et al., Cancer Res. 58(16):3668-3678 (1998); Harrop, et al., J. Immunol. 161(4):1786-1794 (1998); Zhu, et al., Cancer Res. 58(15):3209-3214 (1998)

Antibodies of the present invention may be used, e.g., without limitation, to purify, detect, or target a polypeptide (or fragment thereof) of the present invention for, e.g., in vitro and/or in vivo diagnostic and therapeutic methods. For example, the antibodies have use in immunoassays for qualitatively and/or quantitatively measuring levels of a polypeptide (or fragment thereof) of the present invention in a biological sample (see, e.g., Harlow, et al., Antibodies: A Laboratory Manual, Cold Spring Harbor Laboratory Press, cur. ed.; incorporated by reference).

The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and known in the art. For an overview of the technology for producing human antibodies, see, e.g., Lonberg and Huszar, Int. Rev. Immunol. 13:65-93 (1995). In addition, commercial companies such as, e.g., Abgenix, Inc. (Freemont, Calif.) and Genpharm (San Jose, Calif.) can be hired to produce human antibodies.

Completely human antibodies that recognize a selected epitope can be generated by “guided selection” (see, e.g., Jespers, et al. (1988) BioTechnology 12:899-903). Further, antibodies of the invention can, in turn, be used to generate anti-idiotype antibodies that “mimic” a polypeptide (or fragment thereof) of the invention using known techniques (see, e.g., Greenspan & Bona, FASEB J. 7(5):437-444; (1989) and Nissinoff, J. (1991) Immunol. 147(8):2429-2438). The present invention encompasses antibodies recombinantly fused or chemically conjugated (including both covalent and non-covalent conjugations) to a polypeptide (or portion thereof, preferably comprising at least: 10, 20, 30, 40, 50, 60, 70, 80, 90, or 100 contiguous amino acids of a polypeptide of SED ID NO:X) of the present invention to generate fusion proteins. The fusion does not necessarily need to be direct, but may occur through linker sequences.

The antibodies may be specific for antigens other than a polypeptide of the invention (or portion thereof, preferably at least: 10, 20, 30, 40, 50, 60, 70, 80, 90, or 100 contiguous amino acids) of the present invention. For example, antibodies may be used to target an LP polypeptide (or fragment thereof) to particular cell types, either in vitro or in vivo, by fusing or conjugating a polypeptide (or fragment thereof) of the present invention to an antibody specific for a particular cell surface receptor. Antibodies fused or conjugated to a polypeptide of the invention may also be used in in vitro immunoassays and in purification methods using known art methods (see e.g., Harbor, et al., supra, and WO 9312 1232; EP 439,095; Naramura et al. (1994) Immunol. Lett. 39:9 1-99).

The present invention further includes compositions comprising a polypeptide of the invention (or fragment thereof) fused or conjugated to an antibody domain other than a variable region. For example, a polypeptide of the invention (or fragment thereof) may be fused or conjugated to an antibody Fc region, or portion thereof. The antibody portion that is fused to a polypeptide of the invention (or fragment thereof) may comprise a constant region, a hinge region, a CH1 domain, a CH2 domain, and/or a CH3 domain or any combination of whole domains or portions thereof. A polypeptide of the invention (or fragment thereof) may also be fused or conjugated to an antibody portion described herein to form multimers. For example, Fc portions fused to a polypeptide of the invention (or fragment thereof) can form dimers through disulfide bonding between the Fc portions. Higher multimeric forms can be made by fusing the polypeptides to portions of IgA and IgM. Methods for fusing or conjugating a polypeptide of the invention (or fragment thereof) to an antibody portion are known (see, e.g., U.S. Pat. Nos. 5,336,603; 5,622,929; 5,359,046; 5,349,053; 5,447,851; 5,112,946; EP 307,434; EP 367,166; WO 96/04388).

In many cases, the Fc part of a fusion protein is beneficial in therapy and diagnosis, and thus can result in, e.g., improved pharmacokinetic properties (see, e.g., EP A232,262). Alternatively, deleting the Fc part after the fusion protein has been expressed, detected, and purified, can be favored. Moreover, an antibody of the present invention (or fragment thereof) can be fused to marker sequences, such as a peptide to facilitate purification. Techniques for conjugating a therapeutic moiety to an antibody are known, see, e.g., Amon, et al., “Monoclonal Antibodies For Immunotargeting Of Drugs In Cancer Therapy”, in Monoclonal Antibodies And Cancer Therapy, Reisfeld, et al. (eds.), pp. 243-56 (Alan R. Liss, Inc. 1985); Hellstrom, et al., “Antibodies For Drug Delivery”, in Controlled Drug Delivery (2nd Ed.), Robinson, et al. (eds.), pp. 623-53 Marcel Dekker, Inc. 1987). Alternatively, an antibody can be conjugated to a second antibody to form an antibody heteroconjugate as described U.S. Pat. No. 4,676,980.

An antibody (or fragment thereof) of the invention may be utilized for immunophenotyping of cell lines and biological samples. The translation product of an LP polynucleotide sequence (or fragment thereof) may be useful as a cell specific marker, or more specifically, as a cellular marker (which is differentially expressed at various stages of differentiation and/or maturation of particular cell types). A particular protein can be measured by a variety of immunoassay methods see, e.g., Stites and Terr (eds.) (1991) Basic and Clinical Immunology (7th ed.); Price and Newman (eds.) (1991) Principles and Practice of Immunoassays Stockton Press, N.Y.; and Ngo (ed.) (1988) Non-isotopic Immunoassays Plenum Press, N.Y.; Stites and Terr (eds.) Basic and Clinical Immunology (7th ed.) supra; Maggio (ed.) Enzyme Immunoassay, supra; and Harlow and Lane Antibodies, A Laboratory Manual supra. The ability of the antibody of interest to immunoprecipitate a particular antigen can be assessed by, e.g., Western blot analysis. One of skill in the art would be knowledgeable as to the parameters are modifiable to increase binding of an antibody to an antigen and to decrease background (e.g., by pre-clearing the cell lysate with sepharose beads). Further discussion of immunoprecipitation protocols can be found in, e.g., Ausubel et al, eds., 1994, Current Protocols in Molecular Biology, Vol. 1, John Wiley & Sons, Inc., N.Y.

Therapeutic Uses

The present invention further encompasses antibody-based therapies that involve administering LP antibody to an animal, preferably a mammal, most preferably a primate (e.g., a human), to modulate, treat, inhibit, effect, or ameliorate one or more of the disclosed diseases, disorders, or conditions. An antibody of the invention can be used to modulate, treat, inhibit, ameliorate, or prevent diseases, disorders, or conditions associated with aberrant expression and/or activity of a polypeptide (or fragment thereof) of the invention, including, e.g., without limitation, any one or more of the diseases, disorders, syndromes or conditions described herein. The treatment, amelioration, and/or prevention of diseases, disorders, or conditions associated with aberrant expression and/or activity of a polypeptide of the invention includes, e.g., without limitation, ameliorating symptoms associated with those diseases, disorders or conditions. Antibodies of the invention may be provided in pharmaceutically acceptable compositions as known in the art or as described herein.

Making LP Proteins; Mimetics

DNAs which encode a LP protein or fragments thereof can be obtained by chemical synthesis, screening cDNA libraries, or by screening genomic libraries prepared from a wide variety of cell lines or tissue samples. Methods for doing so, or making expression vectors are either art known or are described herein.

These DNAs can be expressed in a wide variety of host cells for the synthesis of a full-length protein or fragments which can in turn, e.g., be used to generate polyclonal or monoclonal antibodies; for binding studies; for construction and expression of modified molecules; and for structure/function studies. Each LP protein or its fragments can be expressed in host cells that are transformed or transfected with appropriate expression vectors. By “transformed” is meant a cell into which (or into an ancestor of which) a DNA molecule has been introduced, by means of recombinant techniques, which encodes an LP polypeptide or fragment thereof.

Expression vectors are typically self-replicating DNA or RNA constructs containing the desired antigen gene or its fragments, usually operably linked to appropriate genetic control elements that are recognized in a suitable host cell. The specific type of control elements necessary to effect expression depends on the host cell used. Generally, genetic control elements include a prokaryotic promoter system or a eukaryotic promoter expression control system, and typically include a transcriptional promoter, an optional operator to control the onset of transcription, transcription enhancers to elevate the level of mRNA expression, a sequence that encodes a suitable ribosome binding site, and sequences that terminate transcription and translation. All of the associated elements both necessary and sufficient for the production of LP polypeptide are in operable linkage with the nucleic acid encoding the LP polypeptide (or fragment thereof). Usually, expression vectors also contain an origin of replication that allows the vector to replicate independently of the host cell.

An expression vector will preferably include, e.g., at least one selectable marker. Such markers include, e.g., without limit, dihydrofolate reductase, G418, or neomycin resistance for eukaryotic cell culture and tetracycline, kanamycin or ampicillin resistance genes for culturing in E. coli and other bacteria.

The vectors of this invention contain DNAs which encode an LP protein, or a fragment thereof, typically encoding, e.g., a biologically active polypeptide, or protein. The DNA can be under the control of a viral promoter and can encode a selection marker. This invention further contemplates use of expression vectors capable of expressing eukaryotic cDNA coding for a LP (or fragment) in a prokaryotic or eukaryotic host, where the vector is compatible with the host and where the eukaryotic cDNA coding for the protein is inserted into the vector such that growth of the host containing the vector expresses the cDNA in question. Usually, expression vectors are designed for stable replication in their host cells or for amplification to greatly increase the total number of copies of the desirable gene per cell. It is not always necessary to require that an expression vector replicate in a host cell, e.g., it is possible to effect transient expression of the protein or its fragments in various hosts using vectors that do not contain a replication origin that is recognized by the host cell. It is also possible to use vectors that cause integration of an LP protein gene or its fragments into the host DNA by recombination, or to integrate a promoter that controls expression of an endogenous gene.

Vectors, as used herein, encompass plasmids, viruses, bacteriophage, integratable DNA fragments, and other vehicles that enable the integration of DNA fragments into the genome of the host. Expression vectors are specialized vectors that contain genetic control elements that effect expression of operably linked genes. Plasmids are the most commonly used form of vector, but many other forms of vectors that perform an equivalent function are also suitable for use (see, e.g., Pouwels, et al. (1985 and Supplements) Cloning Vectors: A Laboratory Manual Elsevier, N.Y.; and Rodriquez, et al. (eds.) (1988) Vectors: A Survey of Molecular Cloning Vectors and Their Uses Buttersworth, Boston, Mass.).

Suitable host cells include prokaryotes, lower eukaryotes, and higher eukaryotes. Prokaryotes include both gram negative and gram positive organisms, e.g., E. coli and B. subtilis. Lower eukaryotes include yeasts, e.g., S. cerevisiae and Pichia, and species of the genus Dictyostelium. Higher eukaryotes include established tissue culture cell lines from animal cells, both of non-mammalian origin, e.g., insect cells, and birds, and of mammalian origin, e.g., human, primates, and rodents.

Prokaryotic host-vector systems include a variety of vectors for many different species. As used herein, E. coli and its vectors will be used generically to include equivalent vectors used in other prokaryotes. A representative vector for amplifying DNA is pBR322 or its derivatives. Vectors that can be used to express these proteins or protein fragments include, but are not limited to, such vectors as those containing the lac promoter (pUC-series); trp promoter (pBR322-trp); Ipp promoter (the pIN-series); lambda-pP or pR promoters (pOTS); or hybrid promoters such as ptac (pDR540). See Brosius, et al. (1988) “Expression Vectors Employing Lambda-, trp-, lac-, and Ipp-derived Promoters,” in Rodriguez and Denhardt (eds.) Vectors: A Survey of Molecular Cloning Vectors and Their Uses 10:205-236 Buttersworth, Boston, Mass. Other representative bacterial vectors include, e.g., without limit, pQE70, pQE60, and pQE-9, (available from QIAGEN, Inc.); pBluescript vectors, Phagescript vectors, pNH8A, pNH16a, pNH18A, pNH46A, (available from Stratagene Cloning Systems, Inc.); and ptrc99a, pKK223-3, pKK233-3, pDR540, pRIT5 (available from Pharmacia Biotech, Inc).

Higher eukaryotic tissue culture cells are typically the preferred host cells for expression of the functionally active LP protein. Non-limiting representative examples of suitable expression vectors include pCDNA1; pCD (Okayama, et al. (1985) Mol. Cell Biol. 5:1136-1142); pMC1neo Poly-A, (Thomas, et al. (1987) Cell 51:503-512); and a baculovirus vector such as pAC 373 or pAC 610. Additional eukaryotic vectors include, e.g., without limit, pWLNE0, pSV2CAT, pOG44, pXT1 and pSG (available from Stratagene); and pSVK3, pBPV, pMSG and pSVL (available from Pharmacia Biotech, Inc.).

A polypeptide (or fragment thereof) of the present invention, and preferably, a mature and/or secreted form, can also be recovered from natural sources, including, e.g., without limit, bodily fluids, tissues, and cells, (whether directly isolated or cultured); products of chemical synthetic procedures; and products produced by recombinant techniques from a prokaryotic or eukaryotic host including, e.g., bacterial, yeast, higher plant, insect, and mammalian cells).

It is likely that LP proteins need not be glycosylated to elicit biological responses. However, it will occasionally be desirable to express an LP protein or LP polypeptide in a system that provides a specific or defined glycosylation pattern. In this case, the usual pattern will be that provided naturally by the expression system. However, the pattern will be modifiable by exposing the polypeptide, e.g., in unglycosylated form, to appropriate glycosylating proteins introduced into a heterologous expression system. For example, the LP protein gene may be co-transformed with one or more genes encoding mammalian or other glycosylating enzymes. It is further understood that over glycosylation may be detrimental to LP protein biological activity, and that one of skill may perform routine testing to optimize the degree of glycosylation which confers optimal biological activity.

In addition, an LP polypeptide (or fragments thereof) may also include, e.g., an initial modified methionine residue (in some cases because of host-mediated processes). Typically, the N-terminal methionine encoded by the translation initiation codon removed with high efficiency from any protein after translation in all eukaryotic cells. While the N-terminal methionine on most proteins is also efficiently removed in most prokaryotes, for some proteins depending on the nature of the amino acid to which the N-terminal methionine is covalently linked, the removal process is inefficient. In one embodiment, the yeast Pichia pastors is used to express a polypeptide of the present invention(or fragment thereof in an eukaryotic system (see, e.g., Ellis, et al., Mol. Cell. Biol. 5:1111-21 (1985); Koutz, et al., Yeast 5: 167-77 (1989); Tschopp, et al., Nucl. Acids Res. 15:3859-76 (1987)). Thus, a heterologous coding sequence, such as, e.g., an LP polynucleotide sequence, (or fragment thereof under the transcriptional regulation of all or part of the AOX1 regulatory sequence is expressed at exceptionally high levels in Pichia yeast grown in the presence of methanol.

In one example, the plasmid vector pPIC9K is used to express polynucleotide sequence encoding a polypeptide of the invention, (or fragment thereof) as set forth herein, in a Pichea yeast system essentially as described in “Pichia Protocols: Methods in Molecular Biology,” D. R. Higgins and J. Cregg, eds. The Humana Press, Totowa, N.J., 1998. This expression vector allows expression and secretion of a protein of the invention by virtue of the strong AOX1 promoter linked to the Pichia pastoris alkaline phosphatase (PHO) secretory signal peptide located upstream of a multiple cloning site. Many other yeast vectors could be used in place of pPIC9K, such as, e.g., pYES2, pYD1, pTEFI/Zeo, pYES2/GS, pPICZ, pGAPZ, pGAPZalpha, pPIC9, pPIC3.5, PHIL-D2, PHIL-S1, pPIC3.5K, and, PA08, as a skilled in the artisan would appreciate, as long as the proposed expression construct provides appropriately located and operably linked signals for transcription, translation, secretion (if desired), and the like, (including an in-frame stop codon as required).

Furthermore, heterologously expressed proteins or polypeptides can also be expressed in plant cells. For plant cells viral expression vectors (e.g., cauliflower mosaic virus and tobacco mosaic virus) and plasmid expression vectors (e.g., T1 plasmid) are suitable. Such cells are available from a wide range of sources (e.g., the American Tissue Type Culture Collection, Rockland, Md.; also, see for example, Ausubel, et al. (cur. ed. and Supplements; expression vehicles may be chosen from those provided e.g., in Pouwels, et al. (Cur. ed.) Cloning Vectors. A Laboratory Manual).

A LP protein, or a fragment thereof, may be engineered to be phosphatidyl inositol (PI) linked to a cell membrane, but can be removed from membranes by treatment with a phosphatidyl inositol cleaving enzyme, e.g., phosphatidyl inositol phospholipase-C. This releases the antigen in a biologically active form, and allows purification by standard procedures of protein chemistry (see, e.g., Low (1989) Biochem. Biophys. Acta 988:427-454; Tse, et al. (1985) Science 230:1003-1008; and Brunner, et al. (1991) J. Cell Biol. 114:1275-1283).

Now that LP proteins have been characterized, fragments or derivatives thereof can be prepared by conventional processes for synthesizing peptides. These include processes such as are described in Stewart and Young (1984) Solid Phase Peptide Synthesis Pierce Chemical Co., Rockford, Ill.; Bodanszky and Bodanszky (1984) The Practice of Peptide Synthesis Springer-Verlag, New York, N.Y.; and Bodanszky (1984) The Principles of Peptide Synthesis Springer-Verlag, New York, N.Y. The prepared protein and fragments thereof can be isolated and purified from the reaction mixture by means of peptide separation, for example, by extraction, precipitation, electrophoresis and various forms of chromatography, and the like. An LP protein of this invention can be obtained in varying degrees of purity depending upon its desired use. Purification can be accomplished by use of known protein purification techniques or by the use of the antibodies or binding partners herein described (e.g., in immunoabsorbant affinity chromatography).

Recombinant Proteins

An LP polypeptide, or fragment thereof, can be used to generate a fusion protein. For example, when fused to a second polypeptide, an LP polypeptide, or fragment thereof, can be used as an antigenic tag or an immunogen.

Antibodies raised against an LP polypeptide (or fragment thereof) can be used to indirectly detect a second protein by binding thereto. In one embodiment, if an LP protein has amino acid sequence portion that targets a cellular location (e.g., based on trafficking signals), that portion of the polypeptide can be used by fusing it to another protein (or fragment) to target a protein. Examples of domains that can be fused to an LP polypeptide (or fragment thereof) include, e.g., not only heterologous signal sequences, but also other heterologous functional regions. A fusion does not necessarily need to be direct, but may occur, e.g., through linker sequences. Moreover, fusion proteins may also be engineered to improve characteristics of an LP polypeptide.

For instance, a region of additional amino acids, particularly charged amino acids, may be added to the N-terminus of the polypeptide to improve stability and persistence during purification from a host cell or during subsequent handling and storage. In addition, peptide moieties can be added to the polypeptide to facilitate purification. Such regions may be removed before final preparation of the polypeptide. Additions of peptide moieties to facilitate handling are familiar and routine art techniques. Moreover, an LP polypeptide (including any fragment thereof, and specifically an epitope) can be combined with parts of the constant domain of an immunoglobulin e.g., (IgA, IgE, IgG, IgM) portions thereof (CH1, CH2, CH3), and any combination thereof including both entire domains and portions thereof), resulting in a chimeric polypeptide. Such fusion proteins can facilitate purification and often are useful to increase the in vivo half-life of the protein (Fountoulakis, et al. (1995) J. Biochem.15 270:3958-3964). Enhanced delivery of an antigen across an epithelial barrier to the immune system has been demonstrated for antigens (e.g., insulin) conjugated to an FcRn binding partner such as IgG or Fc fragments (see, e.g., WO 96/22024 and WO 99/104813). IgG fusion proteins that have a disulfide-linked dimeric structure due to the IgG portion disulfide bonds have also been found more efficient in binding and neutralizing other molecules than monomeric polypeptides or fragments thereof alone (Fountoulakis, et al. (1995) J. Biochem. 270:3958-3964).

Additionally, a fusion protein can comprise various portions of the constant region of an immunoglobulin molecule together with a human protein (or part thereof) EP-A-O 464 533 (Canadian counterpart 2045869). In many cases, the Fc part in a fusion protein is beneficial in therapy and diagnosis, and thus, can result in, e.g., improved pharmacokinetic properties (EP-A 0232 262.). Alternatively, deleting the Fc part after the fusion protein has been expressed, detected, and purified, may be desired. For example, the Fc portion may hinder therapy and/or diagnosis if the fusion protein is used as an immunogen for immunizations. In drug discovery for example, human proteins, such as hIL-5, have been fused with Fc portions for the purpose of high-throughput screening assays to identify hIL-5 antagonists (Bennett, et al. (1995) I. Molecular Recognition 8:52-58; and Johanson, et al. (1995) J. Biol. Chem. 270:9459-9471).

Furthermore, new constructs may be made by combining similar functional domains from other proteins. For example, protein-binding or other segments may be “swapped” between different new fusion polypeptides or fragments (see, e.g., Cunningham, et al. (1989) Science 243:1330-1336; and O'Dowd, et al. (1988) J. Biol. Chem. 263:15985-15992).

Moreover, an LP polypeptide (or fragment thereof can be fused to a marker sequence, such as a peptide, to facilitate purification. In preferred embodiments, the marker amino acid sequence is a hexa-histidine peptide, such as, e.g., the tag provided in a pQE vector (QIAGEN, Inc., Chatsworth, Calif., 91311), which provides for convenient purification of the fusion protein (Gentz, et al. (1989) Proc. Natl. Acad. Sci. USA 86:821-824). Another useful peptide-purification tag is the “HA” tag, which corresponds to an epitope derived from an influenza hemagglutinin protein (Wilson, et al. (1984) Cell 37:767). Nucleic acid molecules containing LP polynucleotide sequences encoding an LP epitope can also be recombined with a gene of interest as an epitope tag (e.g., the “HA” or flag tag) to aid in detection and purification of the expressed polypeptide. For example, one system purifies non-denatured fusion proteins expressed in human cell lines (Janknecht, et al. (1991) Proc. Natl. Acad. Sci. USA 88:8972-897). In this system, a gene of interest is subcloned into a vaccinia recombination plasmid such that the open reading frame of the sequence of interest is translationally fused to an amino-terminal tag consisting of six histidine residues. The tag serves as a matrix-binding domain for the fusion protein. Extracts from cells infected with the recombinant vaccinia virus are loaded onto Ni2+ nitriloacetic acid-agarose column and histidine-tagged proteins can be selectively eluted with imidazole-containing buffers.

Additionally, LP fusion constructions may be generated through the techniques of gene-shuffling, motif-shuffling, exon shuffling, and/or codon shuffling (collectively referred to as “DNA shuffling”). DNA shuffling may be employed to modulate an activity of an LP polypeptide. Such methods can be used to generate LP polypeptides (or fragments thereof) with altered activity, as well as agonists and antagonists of an LP polypeptide (see, e.g., U.S. Pat. Nos. 5,605,793; 5,811,238; 5,830,721; 5,834,252; and 5,837,458, and Patten, et al. (1997) Cur. Opinion Biotechnol. 8:724-33 30; Harayama, (1998) Trends Biotechnol. 16(2):76-82; Hansson, et al. (1999) J. Mol. Biol. 287:265-76; and Lorenzo and Blasco, (1998) Biotechniques 24(2): 308-13; each of which is incorporated by reference for these DNA shuffling teachings).

VIII. Functional Variants

“Derivatives” of LP protein antigens include amino acid sequence mutants, glycosylation variants, and covalent or aggregate conjugates with other chemical moieties. Covalent derivatives can be prepared by linkage of functionalities to groups which are found in LP protein amino acid side chains or at the N- or C-termini, by any art known means. These derivatives can include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues, e.g., lysine or arginine. Acyl groups are selected from the group of alkyl-moieties including C3 to C18 normal alkyl, thereby forming alkanoyl aroyl species. Covalent attachment to carrier proteins may be important when immunogenic moieties are haptens.

Also provided by the invention is a chemically modified derivative of a polypeptide of the invention (or fragment thereof that may provide additional advantages such as increased solubility, increased stability increased circulating time, or decreased immunogenicity or antigenicity (see U.S. Pat. No. 4,179,337). A chemical moieties for derivatization may be selected from water soluble polymers such as, e.g., polyethyleneglycol, ethylene glycol, propylene glycol, copolymers, carboxymethylcellulose, dextran, polyvinyl alcohol, etc. A polypeptide of the invention, (or fragment thereof) may be modified at random or at predetermined positions within the molecule and may include, e.g., one, two, three, or more attached chemical moieties. The polymer may be of any molecular weight, and may be branched or unbranched. For polyethylene glycol, a preferred molecular weight is between about 1 kDa and about 100 kDa (the term “about” means that in polyethylene glycol preparations, some molecules will weigh more and some will weigh less, than the stated molecular weight).

Other sizes may be used, depending on the desired effect (e.g., the [period of sustained release, the effects, if any, on biological activity, ease in handling, the degree or lack of antigenicity, and other known effects of polyethylene glycol on a protein, polypeptide or an analog). Polyethylene glycol molecules (or other chemical moieties) should be attached with consideration of the effect on functional, immunogenic, and/or antigenic domains of a polypeptide (or fragment thereof). Attachment methods include; e.g., without limit, (coupling PEG to G-CSF); EP 0 401 384, pegylating GM-CSF using tresyl chloride (Malik, et al. (1992) Exp. Hematol. 20:1028-1035). For example, polyethylene glycol may be covalently bound through amino acid residues via a reactive group, such as, e.g., a free amino or carboxyl group. Reactive groups are those to which an activated polyethylene glycol molecule may be bound. Amino acid residues having a free amino group may include, e.g., lysine residues, and N-terminal amino acid residue. Amino acid residues having a free carboxyl group may include, e.g., aspartic acid residues, glutamic acid residues, and C-terminal amino acid residues. Sulfhydryl groups may also be used to attach to a polyethylene glycol molecule. For human, a preferred attachment is at an amino group, such as, e.g., an attachment at the N-terminus or a lysine group.

One may specifically desire a protein, or a polypeptide (or fragment thereof) that is chemically modified at the N-terminus. Using polyethylene glycol as an illustration of the present composition, one may select from a variety of polyethylene glycol molecules (by molecular weight, branching, etc.), the proportion of polyethylene glycol molecules to a protein (polypeptide) molecule in the reaction mix, the type of pegylation reaction to be performed, and the method of obtaining the selected N-terminally pegylated, e.g., polypeptide. The method of obtaining an N-terminally pegylated preparation (by, e.g., separating this moiety from other monopegylated moieties if necessary) may be by purification of the N-terminally pegylated material from a population of pegylated protein molecules. Selective protein chemical modification at the N-terminus may be accomplished by reductive alkylation, which exploits differential reactivity of different types of primary amino groups (lysine versus the N-terminal) available for derivatization in a particular protein. Under appropriate reaction conditions, substantially selective derivatization of a protein or polypeptide (or fragment thereof) at the N-terminus with a carbonyl-group-containing-polymer is achieved.

This invention also encompasses the use of derivatives of an LP protein other than variations in amino acid sequence or glycosylation. Such derivatives may involve covalent or aggregative association with chemical moieties. Generally, these derivatives fall into the three classes: (1) salts, (2) side chain and terminal residue covalent modifications, and (3) adsorption complexes (e.g., with cell membranes). Such covalent or aggregative derivatives are useful as immunogens, as reagents in immunoassays, or in purification methods such as for affinity purification of proteins or other binding proteins. For example, a LP protein antigen can be immobilized by covalent bonding to a solid support such as cyanogen bromide-activated SEPHAROSE, by methods which are well known in the art, or adsorbed onto polyolefin surfaces, with or without glutaraldehyde cross-linking, for use in an assay or purification of anti-LP protein antibodies or its respective binding partner. An LP protein can also be labeled for use in diagnostic assays with a detectable group (such as, e.g., radioiodinated by the chloramine T procedure; covalently bound to rare earth chelates; or conjugated to another fluorescent moiety). Purification of an LP protein may be effected by immobilized antibodies or a binding partner.

A polypeptide of the invention (or fragment thereof) may be as a monomer or a multimer (e.g., a dimer, a trimer, a tetramer, or a higher multimer). Accordingly, the present invention encompasses monomers and multimers of a polypeptide of the invention, (or fragment thereof) including, e.g., their preparation, and compositions (preferably, therapeutic compositions) containing them. In specific embodiments, the polypeptides and/or fragments of the invention are monomers, dimers, trimers, tetramers or higher multimers. In additional embodiments, a multimer of the invention is at least a dimer, at least a trimer, or at least a tetramer. Multimers encompassed by the invention may be homomers or heteromers. As used herein, the term “homomer,” refers to a multimer containing only a specific polypeptide (or fragment thereof) corresponding to an amino acid sequence of SEQ ID NO:Y or in a talbe herein (including fragments, variants, splice variants, and fusion proteins, corresponding to these polypeptides as described herein). A homomer may contain a polypeptide having identical or different amino acid sequences. In a specific embodiment, a homomer of the invention is a multimer containing only polypeptides (or fragments thereof) having identical amino acid sequences. In another specific embodiment, a homomer of the invention is a multimer containing polypeptides having different amino acid sequences.

In specific embodiments, a multimer of the invention is a homodimer (e.g., containing polypeptides having identical and/or different amino acid sequences) or a homotrimer (e.g., containing polypeptides having identical and/or different amino acid sequences). In additional embodiments, the homomeric multimer of the invention is at least a homodimer, at least a homotrimer, or at least a homotetramer. As used herein, the term “heteromeric,” refers to a multimer containing one or more heterologous polypeptides. In a specific embodiment, a multimer of the invention is a heterodimer, a heterotrimer, or a heterotetramer. In additional embodiments, the heteromeric multimer of the invention is at least a heterodimer, at least a heterotrimer, or at least a heterotetramer. Multimers of the invention may be the result of hydrophobic, hydrophilic, ionic and/or covalent associations and/or may be indirectly linked, by e.g., liposome formation. Thus, in one embodiment, a multimer of the invention, such as, e.g., homodimers or homotrimers, are formed when polypeptides of the invention (or fragments thereof) contact one another in solution.

In another embodiment, a heteromultimer of the invention, such as, e.g., a heterotrimer or a heterotetramer, is formed when, e.g., a polypeptide of the invention contacts an antibody (generated against a polypeptide; or fragment thereof of the invention (including antibodies to the heterologous polypeptide sequence in a fusion protein of the invention)) in solution. In other embodiments, a multimer of the invention is formed by covalent association with and/or between a polypeptide and a binding partner such as mentioned herein (or fragment thereof). Such covalent associations may involve one or more amino acid residues contained in a polypeptide sequence (e.g., as recited in a sequence listing herein, or contained in a polypeptide encoded by a deposited clone specified herein). In one instance, a covalent association is a cross-link, e.g., between cysteine residues. In another instance, the covalent associations are the consequence of chemical or recombinant manipulation. Alternatively, such covalent associations may involve one or more amino acid residues contained in a heterologous polypeptide sequence such as, e.g., a fusion protein of the invention. In one example, covalent associations form with a heterologous sequence contained in a fusion protein of the invention (see, e.g., U.S. Pat. No. 5,478,925). In a specific example, a covalent association is between a heterologous sequence contained in an Fc fusion protein of the invention (as described herein). In another specific example, a covalent association of a fusion protein of the invention is with a heterologous polypeptide sequence such as, e.g., oseteoprotegerin (see, e.g., WO 98149305, incorporated by reference for these teachings).

In another embodiment, two or more polypeptides of the invention (or fragment thereof) are joined through peptide linkers. Examples include, e.g., peptide linkers described in U.S. Pat. No. 5,073,627 (incorporated by reference for these teachings). A protein comprising multiple polypeptides of the invention that are separated by peptide linkers may be produced using conventional recombinant DNA technology.

Recombinant fusion proteins comprising a polypeptide of the invention (or fragment thereof) fused to a polypeptide sequence that dimerizes or trimerizes in solution can be expressed in a suitable host cell. The resulting soluble multimeric fusion protein can be recovered from a supernatant using any art known technique or method described herein. Trimeric polypeptides of the invention may offer an advantage of enhanced biological activity (as defined herein). Preferred leucine zipper moieties and isoleucine moieties are those that preferentially form trimers. An example is a leucine zipper derived from lung surfactant protein D (SPD), as described in Hoppe, et al. FEBS Letters 344: 19 1,15(1994) and in U.S. patent application Ser. No. 08/446,922, (each hereby incorporated by reference for these teachings). Other peptides derived from naturally occurring trimeric proteins may be employed when preparing a trimeric polypeptide of the invention.

In another example, polypeptides or proteins of the invention are associated by interactions with a Flag polypeptide sequence (e.g., contained in a fusion protein of the invention having a Flag sequence). In a further embodiment, a protein or a polypeptide of the invention is associated by an interaction with a heterologous polypeptide sequence (contained in a Flag fusion protein of the invention) and an anti-Flag antibody.

A multimer of the invention may be generated using chemical art known techniques. For example, polypeptides (or fragments thereof) desired to be contained in a multimer of the invention may be chemically cross-linked using a linker molecule e.g., linker molecules and linker molecule length optimization techniques are known in the art; see, e.g., U.S. Pat. No. 5,478,925, which is incorporated by reference for such teachings. Additionally, a multimer of the invention may be generated using techniques known in the art to form one or more inter-molecule cross-links between the cysteine residues (see, e.g., U.S. Pat. No. 5,478,925, incorporated by reference for these teachings). Further, a polypeptide of the invention modified by the addition of cysteine or biotin to the C or N-terminus of a polypeptide can be generated by art known methods (see, e.g., U.S. Pat. No. 5,478,925, incorporated by reference for these teachings).

Additionally, a multimer of the invention can be generated by art known methods (see, e.g., U.S. Pat. No. 5,478,925, incorporated by reference for these teachings). Alternatively, a multimer of the invention can be generated using other commonly known genetic engineering techniques. In one embodiment, a polypeptide contained in a multimer of the invention is produced recombinantly with fusion protein technology described herein or otherwise known in the art (see, e.g., U.S. Pat. No. 5,478,925, incorporated by reference for these teachings). In a specific embodiment, a polynucleotide encoding a homodimer of the invention can be generated by ligating a polynucleotide sequence encoding a polypeptide (or fragment thereof) of the invention to another sequence encoding a linker polypeptide and then subsequently, further to a synthetic polynucleotide encoding the translated product of the polypeptide in the reverse orientation from the original C-terminus to the N-terminus (lacking the leader sequence) (see, e.g., U.S. Pat. No. 5,478,925, incorporated by reference for these teachings).

In another embodiment, recombinant techniques described herein or otherwise known in the art can be applied to generate a recombinant polypeptide of the invention (or fragment thereof) that contains a transmembrane domain (or hyrophobic or signal peptide) and that can be incorporated by membrane reconstitution techniques into a liposome (see, e.g., U.S. Pat. No. 5,478,925, incorporated by reference for these teachings).

X. Uses

The present invention provides reagents that will find use in diagnostic and/or therapeutic applications as described herein, e.g., in the description of kits for diagnosis.

An LP polynucleotide sequence (or fragment thereof) can be used in numerous ways, e.g., such as a reagent. The following descriptions (using known art techniques) are non-limiting examples of ways to use an LP polynucleotide sequence (or fragment thereof). For example, an LP polynucleotide sequence (or fragment thereof) is useful for chromosome identification. There exists an ongoing need to identify new chromosome markers, since few chromosome-marking reagents, based on actual sequence data (repeat polymorphisms), are presently available. Each polynucleotide of the present invention can therefore, be used as a chromosome marker.

In another embodiment, the invention encompasses a kit, e.g., for analyzing a sample for the presence of a polynucleotide associated with a proliferative disease, syndrome, disorder, or condition. In a general embodiment, the kit includes, e.g., at least an LP polynucleotide sequence (or fragment thereof) probe containing a polynucleotide sequence that hybridizes with an LP polynucleotide sequence(or fragment thereof) and directions, e.g., such as for disposal. In another specific embodiment, a kit includes, e.g., two polynucleotide probes defining an internal region of an LP polynucleotide sequence, where each probe has one strand containing a 31 mer-end internal to a region the polynucleotide.

In a further embodiment, a probe may be useful as a primer for amplification using a polymerase chain reaction (PCR). Where a diagnosis of a disease, syndrome, disorder or condition has already been made according to conventional methods, the present invention is useful as a prognostic indicator, for a subject exhibiting an enhanced or diminished expression of an LP polynucleotide sequence (or fragment thereof) by comparison to a subject expressing the polynucleotide of the present invention (or fragment thereof) at a level nearer a standard level.

The phrase, “measuring level of a composition of the present invention” is intended to mean herein measuring or estimating (either qualitatively and/or quantitatively) a level of, e.g., a polypeptide (or fragment thereof), or a polynucleotide (or fragment thereof) including, e.g., mRNA, DNA, or cDNA, in a first sample (e.g., preferably a biological sample) either directly (e.g., by determining or estimating an absolute protein or mRNA level) or relatively (e.g., by comparing to a polypeptide or mRNA level in a second sample). In one embodiment, the level in the first sample is measured or estimated from an individual having, or suspected of having, a disease, syndrome, disorder or condition and comparing that level to a second level, wherein the second level is obtained from an individual not having and/or not being suspected of having a disease, syndrome, disorder or condition. Alternatively, the second level is determined by averaging levels from a population of individuals not having or suspected of having a disease, syndrome, disorder, or condition.

As is appreciated in the art, once a standard level is determined, it can be used repeatedly as a standard for comparison. A “biological sample” is intended to mean herein any sample comprising biological material obtained from, using, or employing, e.g., an organism, body fluid, exudate, lavage product, waste product, cell (or part thereof), cell line, organ, biopsy, tissue culture, or other source originating from, or associated with, a living cell, tissue, organ, or organism, which contains, e.g., a polypeptide (or fragment thereof), a protein (or fragment thereof), a mRNA (or fragment thereof), or polynucleotide sequence (or fragment thereof) of the present invention, including, e.g., without limitation, a sample such as from, e.g., hair, skin, blood, saliva, semen, vomit, synovial fluid, amniotic fluid, breast milk, lymph, pulmonary sputum, urine, fecal matter, a lavage product, etc.

As indicated, a biological sample can include, e.g., without limitation, body fluids (e.g., such as semen, lymph, sera, plasma, urine, synovial fluid and spinal fluid) that contain a polypeptide (or fragment thereof), mRNA (or fragment thereof), a protein (or fragment thereof), or polynucleotide (or fragment thereof) of the present invention, by product, or, waste product; and/or other tissue source found to express a polypeptide (or fragment thereof, mRNA (or fragment thereof, or nucleic acid (or fragment thereof), by product, or, waste product; of the present invention. Methods for obtaining biological samples, e.g., tissue biopsies, body fluids, cells, or waste products from mammals are known in the art. Where the biological sample is to include, e.g., mRNA, a tissue biopsy is a preferred source.

The present invention further encompasses an LP polynucleotide sequence (or fragment thereof that is chemically synthesized, or reproduced as a peptide nucleic acid (PNA) using art known methods. The use of a PNA is preferred if a polynucleotide (or a fragment thereof) is incorporated, e.g., onto a solid support, or genechip. For the purposes of the present invention, a peptide nucleic acid (PNA) is a polyamide type of polynucleotide analog in which, generally, e.g., the monomeric units for adenine, guanine, thymine and cytosine are available commercially (see, e.g., Perceptive Biosystems). Certain components of a polynucleotide, such as DNA, like phosphorus, phosphorus oxides, or deoxyribose derivatives, are not present in a PNA. Generally, PNAs bind specifically and tightly to complementary DNA strands and are not degraded by nucleases (Nielsen, et al. (1993) Nature 365: 666). In fact, a PNA binds more strongly to DNA than DNA binds to itself, probably, as there is no electrostatic repulsion between PNA/DNA; furthermore, the PNA polyamide backbone is more flexible than DNA. Because of this, PNA/DNA duplexes can bind under a wider range of stringency conditions than DNA/DNA duplexes thus, making it easier to perform multiplex hybridizations. Moreover, smaller probes can be used with PNA than with DNA due to the strong binding.

In addition, it is more likely that single base mismatches can be determined using a PNA/DNA hybridization since, e.g., a single mismatch in a PNA/DNA 15-mer lowers the melting point (Tₘ) by 8°-20° C., versus lowering the melting point 4°-16° C. for the DNA/DNA 15-mer duplex. In addition, the absence of charge groups in a PNA molecule means that hybridizations can be done at low ionic strengths and the absence of charge groups with the DNA reduces possible interference by salt.

An LP polypeptide (or fragment thereof), can be used in numerous ways. The following descriptions are non-limiting, exemplars that use art known techniques.

A polypeptide (or fragment thereof) can be used to assay a protein level, e.g., of a secreted protein, in a sample, e.g., such as a bodily fluid by using antibody-based techniques. For example, protein expression in a tissue can be studied by an immunohistological method (see, e.g., Jalkanen, et al. (1985) J. Cell Biol. 101:976-985; Jalkanen, et al. (1987) J. Cell Biol. 105:3087-303096). Another useful antibody-based method for detecting protein or polypeptide expression includes, e.g., an immunoassay like an enzyme linked immunosorbent assay or a radioimmunoassay (RIA). In addition to assaying, e.g., the level of a secreted protein in a sample, a protein can also be detected by in vivo imaging. Thus, the invention provides a means for detecting, marking, locating or diagnosing a disease, syndrome, syndrome, disorder, and/or condition comprising assaying the expression of a polynucleotide (or fragment thereof), or a polypeptide (or fragment thereof), of the present invention that is in a sample, e.g., cells or body fluid of an individual by comparing one level of expression with another level of expression, e.g., a standard level of expression to indicate, e.g., a disease, syndrome, disorder, and/or condition, (or predilection to the same), or to make a prognosis or prediction.

Furthermore, an LP polypeptide (or fragment thereof)can be used to treat, prevent, modulate, ameliorate, and/or diagnose a disease, syndrome, condition, and/or a disorder. For example, a subject can be administered a polypeptide (or fragment thereof) of the invention to replace absent or decreased levels of a polynucleotide or polypeptide (e.g., insulin); to supplement absent or decreased levels of a different polynucleotide or polypeptide (e.g., hemoglobin S for hemoglobin B; SOD to catalyze DNA repair proteins); to inhibit the activity of a polynucleotide or polypeptide (e.g., an oncogene or tumor suppressor); to activate a polynucleotide or polypeptide (e.g., by binding to a receptor), to reduce activity of a membrane bound receptor by competing with the receptor for free ligand (e.g., soluble TNF receptors can be used to reduce inflammation), or to bring about a desired response (e.g., blood vessel growth inhibition, enhancement of an immune response to proliferating cells or to an infectious agent).

Similarly, an antibody directed to a polypeptide (or fragment thereof) of the present invention can also be used to treat, prevent, modulate, ameliorate, and/or diagnose a condition, syndrome, state, disease or disorder. For example, administration of an antibody directed to an LP polypeptide (or fragment thereof) can bind and reduce the level of the targeted polypeptide. Similarly, administration of an antibody can activate an LP polypeptide (or fragment thereof), such as by binding to the polypeptide that is bound to a membrane (e.g., a receptor).

Diagnosis and Imaging Using an LP Antibody

Antibodies of the invention can be used to assay polypeptide levels in a sample, e.g., using classical immunohistological methods known to those of skill in the art (see e.g., Jalkanen, et al., J. Cell. Biol. 101:976-985 (1985); Jalkanen, et al., J. Cell . Biol. 105:3087-3096 (1987)). Other antibody-based methods typically useful for detecting polypeptide expression include, e.g., immunoassays, such as the enzyme linked immunosorbent assay (ELISA) and the radioimmunoassay (RIA).

Sequences encoding an LP polypeptide (or fragment thereof) are used for the diagnosis of disorders associated with LP (such as, e.g., LP misexpression, LP overexpression, LP underexpression, etc.). Examples of such disorders include, without limit, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, Hamartoma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a cardiovascular disorder such as congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, complications of cardiac transplantation, arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, Amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural edema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, post-therapeutic neuralgia, Tourette's disorder, progressive supranuclear palsy, corticobasal degeneration, and familial frontotemporal dementia; and a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephali, craniorachischisis, congenital glaucoma, cataract, and sensorineural hearing loss. Sequences encoding an LP polypeptide (or fragment thereof) are used in Southern or northern analysis; dot blot or other membrane-based technologies; PCR technologies; in dipstick, pin, and multiformat ELISA-like assays; and in microarrays utilizing fluids or tissues from a subject; to detect an altered LP polypeptide (or fragment thereof) expression. Such qualitative or quantitative methods are well known in the art.

Therapeutic Uses

This invention also provides reagents with significant therapeutic value. An LP protein or polypeptide (naturally occurring or recombinant), fragments thereof, and antibodies thereto, along with compounds identified as having binding affinity to an LP, are useful in the treatment of conditions associated with abnormal physiology or development, including abnormal proliferation, e.g., cancerous conditions, or degenerative conditions. Abnormal proliferation, regeneration, degeneration, and atrophy may be modulated by appropriate therapeutic treatment using a composition(s) provided herein. For example, a disease or disorder associated with abnormal expression or abnormal signaling by an LP protein is a target for an agonist or antagonist of the protein.

Other abnormal developmental conditions are known in cell types shown to possess LP mRNA by northern blot analysis (see, e.g., Berkow (ed.) The Merck Manual of Diagnosis and Therapy, Merck & Co., Rahway, N.J.; Thorn et al. Harrison's Principles of Internal Medicine, McGraw-Hill, N.Y.; and Rich (ed.) Clinical Immunology: Principles and Practice, Mosby, St. Louis (cur. ed.); and below). Developmental or functional abnormalities, (e.g., of the neuronal, immune, or hematopoetic system) cause significant medical abnormalities and conditions which may be susceptible to prevention or treatment using compositions provided herein.

Recombinant LP or LP antibodies can be purified and administered to a subject for treatment. These reagents can be combined for use with additional active or inert ingredients, e.g., in conventional pharmaceutically acceptable carriers or diluents, e.g., immunogenic adjuvants, along with physiologically innocuous stabilizers and excipients. These combinations can be sterile filtered and placed into dosage forms as by lyophilization in dosage vials or storage in stabilized aqueous preparations. This invention also contemplates use of antibodies or binding fragments thereof, including forms which are not complement binding. Another therapeutic approach included within the invention involves direct administration of reagents, formulations, or compositions by any conventional administration techniques (such as, e.g., without limit, local injection, inhalation, or systemic administration) to a subject The reagents, formulations, or compositions included within the bounds and metes of the invention may also be targeted to a cell by any of the methods described herein (e.g., polynucleotide delivery techniques). The actual dosage of reagent, formulation, or composition that modulates a disease, disorder, condition, syndrome, etc., depends on many factors, including the size and health of an organism, however one of one of ordinary skill in the art can use the following teachings describing methods and techniques for determining clinical dosages (see, e.g., Spilker (1984) Guide to Clinical Studies and Developing Protocols. Raven Press Books, Ltd., New York, pp. 7-13, 54-60; Spilker (1991) Guide to Clinical Trials. Raven Press, Ltd., New York, pp. 93-101; Craig and Stitzel (eds. 1986) Modern Pharmacology, 2d ed., Little, Brown and Co., Boston, pp. 127-33; Speight (ed. 1987) Avery's Drug Treatment: Principles and Practice of Clinical Pharmacology and Therapeutics, 3d ed., Williams and Wilkins, Baltimore, pp. 50-56; Tallarida, et al. (1988) Principles in General Pharmacology, Springer-Verlag, New York, pp. 18-20; and U.S. Pat. Nos. 4,657,760; 5,206,344; and 5,225,212.). Generally, in the range of about between 0.5 fg/ml and 500 μg/ml inclusive final concentration are administered per day to a human adult in any pharmaceutically acceptable carrier. Furthermore, animal experiments provide reliable guidance for the determination of effective does for human therapy. Interspecies scaling of effective doses can be performed following art known principles (e.g., see, Mordenti and Chappell (1989) “The Use of Interspecies Scaling in Toxicokinetics,” in Toxicokinetics and New Drug Development; Yacobi, et al. (eds.) Pergamon Press, NY).

Effective doses can also be extrapolated using dose-response curves derived from in vitro or animal-model test systems. For example, for antibodies a dosage is typically 0.1 mg/kg to 100 mg/kg of a recipients body weight. Preferably, a dosage is between 0.1 mg/kg and 20 mg/kg of a recipients body weight, more preferably 1 mg/kg to 10 mg/kg of a recipients body weight. Generally, homo-specific antibodies have a longer half-life than hetero-specific antibodies, (e.g., human antibodies last longer within a human host than antibodies from another species, e.g., such as a mouse, probably, due to the immune response of the host to the foreign composition). Thus, lower dosage of human antibodies and less frequent administration is often possible if the antibodies are administered to a human subject. Furthermore, the dosage and frequency of administration of antibodies of the invention may be reduced by enhancing uptake and tissue penetration (e.g., into the brain) by using modifications such as, e.g., lipidation.

The invention also provides a pharmaceutical pack or kit comprising one or more containers filled with one or more of the ingredients of the compositions of the invention and instructions such as, e.g., for disposal (typically, in a form prescribed by a governmental agency regulating the manufacture, use or sale of pharmaceuticals or biological products). The quantities of reagents necessary for effective treatment will depend upon many different factors, including means of administration, target site, physiological state of the patient, and other medicaments administered. Thus, treatment dosages should be titrated to optimize safety and efficacy. Typically, dosages used in vitro may provide useful guidance in the amounts useful for in situ administration of these reagents. Animal testing of effective doses for treatment of particular disorders will provide further predictive indication of human dosage. Various considerations are described, e.g., in Gilman, et al. (eds.) (1990) Goodman and Gilman's: The Pharmacological Bases of Therapeutics (8th ed.) Pergamon Press; and (1990) Remington's Pharmaceutical Sciences (17th ed.) Mack Publishing Co., Easton, Pa. Methods for administration are discussed therein and below, e.g., for oral, intravenous, intraperitoneal, or intramuscular administration, transderrnal diffusion, and others. Pharmaceutically acceptable carriers will include water, saline, buffers, and other compounds described, e.g., in the Merck Index, Merck & Co., Rahway, N.J. Dosage ranges would ordinarily be expected to be in amounts lower than 1 mM concentrations, typically less than about 10 μM concentrations, usually less than about 100 nM, preferably less than about 10 pM (picomolar), and most preferably less than about 1 fM (femtomolar), with an appropriate carrier. Slow release formulations, or a slow release apparatus will often be utilized for continuous administration.

LP protein, fragments thereof, and antibodies to it or its fragments, antagonists, and agonists, may be administered directly to the host to be treated or, depending on the size of the compounds, it may be desirable to conjugate them to carrier proteins such as ovalbumin or serum albumin prior to their administration. Therapeutic formulations may be administered in any conventional dosage formulation. While it is possible for the active ingredient to be administered alone, it is preferable to present it as a pharmaceutical formulation. Formulations typically comprise at least one active ingredient, as defined above, together with one or more acceptable carriers thereof. Each carrier should be both pharmaceutically and physiologically acceptable in the sense of being compatible with the other ingredients and not injurious to the patient. Formulations include those suitable for oral, rectal, nasal, or parenteral (including subcutaneous, intramuscular, intravenous and intradermal) administration. The formulations may conveniently be presented in unit dosage form and may be prepared by any methods well known in the art of pharmacy. See, e.g., Gilman, et al. (eds.) (1990) Goodman and Gilman's: The Pharmacological Bases of Therapeutics (8th ed.) Pergamon Press; and (1990) Remington's Pharmaceutical Sciences (17th ed.) Mack Publishing Co., Easton, Pa.; Avis, et al. (eds.) (1993) Pharmaceutical Dosage Forms: Parenteral Medications Dekker, N.Y.; Lieberman, et al. (eds.) (1990) Pharmaceutical Dosage Forms: Tablets Dekker, N.Y.; and Lieberman, et al. (eds.) (1990) Pharmaceutical Dosage Forms: Disperse Systems Dekker, N.Y. The treatment of this invention may be combined with or used in association with other therapeutic agents.

The present invention also provides a pharmaceutical composition. Such a composition comprises, e.g., a therapeutically effective amount of a composition of the invention in a pharmaceutically acceptable carrier. As used herein, the term “pharmaceutically acceptable carrier” means a carrier approved by a federal regulatory agency of the United States of America, or a regulatory/administrative agency of a state government of the United States or a carrier that is listed in the U.S. Pharmacopeia or other pharmacopeia; which is generally recognized by those in the art for use in an animal, e.g., a mammal, and, more particularly, in a primate, e.g., a human primate.

Various delivery systems are known and can be used to administer, e.g., a composition, formulation, antibody polypeptide (or fragment thereof), or polynucleotide (or fragment thereof) of the invention. For example, delivery can use liposomes, microparticles, microcapsules, recombinant cells, receptor-mediated endocytosis (see, e.g., Wu and Wu (1987) J. Biol. Chem. 262:4429-4432), inclusion of a nucleic acid molecule as part of a retroviral or other vector, etc. Methods of administration include, e.g., without limit, intradermal, intramuscular, intraperitoneal, intravenous, subcutaneous, intranasal, epidural, and oral routes.

An LP can be useful in ameliorating, treating, preventing, modulating, and/or diagnosing a disease, disorder, syndrome, or condition of the immune system, by, e.g., activating or inhibiting the proliferation, differentiation, or mobilization (chemotaxis or directed movement) of an immune cell. Typically, immune cells develop through a process called hematopoiesis, producing myeloid (platelets, red blood cells, neutrophils, and macrophages) and lymphoid (B and T lymphocytes) cells from pluripotent stem cells. The etiology of an immune disease, disorder, syndrome, or condition may be genetic and/or somatic, (e.g., such as some forms of cancer or some autoimmune conditions acquired by e.g., chemotherapy or toxins or an infectious agent, e.g., a virus or prion-like entity. Moreover, an LP can be used to mark or detect a particular immune system disease, syndrome, disorder, state, or condition.

An LP can be useful in ameliorating, treating, preventing, modulating, and/or diagnosing a disease, disorder, syndrome, and/or a condition of a hematopoietic cell. An LP could be used to increase or inhibit the differentiation or proliferation of a hematopoietic cell, including a pluripotent stem cell such an effect can be implemented to treat, prevent, modulate, or ameliorate a disease, disorder, syndrome, and/or a condition associated with a decrease in a specific type of hematopoietic cell. An example of such an immunologic deficiency, disease, disorder, syndrome, and/or condition includes, e.g., without limitation, a blood condition (e.g. agammaglobulinemia, digammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

Moreover, an LP can be used to modulate hemostatic or thrombolytic activity. For example, increasing hemostatic or thrombolytic activity can treat or prevent a blood coagulation condition such as e.g., afibrinogenemia, a factor deficiency, a blood platelet disease (e.g. thrombocytopenia), or a wound resulting from e.g., trauma, surgery, etc. Alternatively, a composition of the invention can be used to decrease hemostatic or thrombolytic activity or to inhibit or dissolve a clotting condition. Such compositions can be important in a treatment or prevention of a heart condition, e.g., an attack infarction, stroke, or mycardial scarring.

An LP may also be useful in ameliorating, treating, preventing, modulating and/or diagnosing an autoimmune disease, disorder, syndrome, and/or condition such as results, e.g., from the inappropriate recognition by a cell of the immune system of the self as a foreign material. Such an inappropriate recognition results in an immune response leading to detrimental effect destruction on the host, e.g., on a host cell, tissue, protein, or moiety, e.g., a carbohydrate side chain. Therefore, administration of an LP which inhibits a detrimental immune response, particularly, e.g., a proliferation, differentiation, or chemotaxis of a T-cell, may be effective in detecting,-diagnosing, ameliorating, or preventing such an autoimmune disease, disorder, syndrome, and/or condition. Examples of autoimmune conditions that can be affected by the present invention include, e.g., without limit Addison's Disease syndrome hemolytic anemia, anti-phospholipid syndrome, rheumatoid arthritis, dermatitis, allergic encephalomyelitis, glomerulonephritis, Goodpasture's Syndrome, Graves' Disease syndrome, Multiple Sclerosis, Myasthenia Gravis, Neuritis, Ophthalmia, Bullous Pemphigoid, Pemphigus, Polyendocrinopathies, Purpura, Reiter's Disease syndrome, Stiff-Man Syndrome, Autoimmune Thyroiditis, Systemic Lupus Erythematosus, Autoimmune Pulmonary Inflammation, Guillain-BarreSyndrome, insulin dependent diabetes mellitis, and autoimmune inflammatory eye disease.

Similarly, allergic reactions and conditions, such as asthma (e.g., allergic asthma) or other respiratory problems, may also be ameliorated, treated, modulated or prevented, and/or diagnosed by an LP polynucleotide or polypeptide (or fragment thereof, or an agonist or antagonist thereto. Moreover, such inventive compositions can be used to effect, e.g., anaphylaxis, hypersensitivity to an antigenic molecule, or blood group incompatibility. An LP may also be used to modulate, ameliorate, treat, prevent, and/or diagnose organ rejection or graft-versus-host disease (GVHD). Generally speaking, organ rejection occurs by a host's, immune-cell destruction of a transplanted tissue or cell. A similarly destructive immune response is involved in GVHD, however, in this case, transplanted foreign immune cells destroy host tissues and/or cells. Administration of a composition of the invention, which ameliorates or modulates such a deleterious immune response (e.g., a deleterious proliferation, differentiation, or chemotaxis of a T cell), can be effective in modulating, ameliorating, diagnosing, and/or preventing organ rejection or GVHD.

Similarly, an LP may also be used to detect, treat, modulate, ameliorate, prevent, and/or diagnose an inflammation, e.g., by inhibiting the proliferation and/or differentiation of a cell involved in an inflammatory response, or an inflammatory condition (either chronic or acute), including, e.g., without limitation, chronic prostatitis, granulomatous prostatitis and malacoplakia, an inflammation associated with an infection (such as, e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease syndrome, Crohn's disease syndrome, or a condition resulting from an over production of a cytokine(s) (e.g., TNF or IL-1.)

An LP can be used to modulate, ameliorate, treat; prevent, and/or diagnose a hyperproliferative disease, condition, disorder, or syndrome (such as, e.g., a neoplasm) via direct or indirect interactions. For example, such as by initiating the proliferation of cells that, in turn, modulate a hyperproliferative state; or by increasing an immune response (e.g., by increasing the antigenicity of a protein involved in a hyperproliferative condition); or by causing the proliferation, differentiation, or mobilization of a specific cell type (e.g., a T-cell). A desired effect using a composition of the invention may also be accomplished either by, e.g., enhancing an existing immune response, or by initiating a new immune response. Alternatively, the desired result may be effected either by, e.g., diminishing or blocking an existing immune response, or by preventing the initiation of a new immune response.

Examples of such hyperproliferative states, diseases, disorders, syndromes, and/or conditions include, e.g., without limitation, a neoplasm of the colon, abdomen, bone, breast, digestive system, liver, pancreas, peritoneum, endocrine system (e.g., an adrenal gland, a parathyroid gland, the pituitary, the testicles, the ovary, the thymus, or the thyroid), eye, head, neck, nervous system (central or peripheral), the lymphatic system, pelvis, skin, spleen, thorax, and urogenital system. Similarly, other hyperproliferative conditions, include, e.g., without limit hypergammaglobulinemia, lymphoproliferative conditions, paraproteinemias, purpura, sarcoidosis, Hamartoma, Sezary Syndrome, Waldenstron's Macroglobulinernia, Gaucher's Disease syndrome, histiocytosis, and other hyperproliferative states.

One preferred embodiment utilizes an LP to inhibit aberrant cellular division, through a polynucleotide delivery technique. Thus, the present invention provides a method for treating, preventing, modulating, ameliorating, preventing, inhibiting, and/or diagnosing cell proliferative diseases, disorders, syndromes, and/or conditions described herein by inserting into an abnormally proliferating cell a composition of the present invention, wherein said composition beneficially modulates an excessive condition of cell proliferation, e.g., by inhibiting transcription and/or translation. Another embodiment comprises administering one or more active copies of an LP polynucleotide sequence to an abnormally proliferating cell. For example in one embodiment, an LP polynucleotide sequence is operably linked in a construct comprising a recombinant expression vector that is effective in expressing a polypeptide (or fragment thereof) corresponding to the polynucleotide of interest. In another preferred embodiment, the construct encoding a polypeptide or fragment thereof, is inserted into a targeted cell utilizing a retrovirus or an adenoviral vector (see, e.g., Nabel, et al. (1999) Proc. Natl. Acad. Sci. USA 96: 324-326). In a still preferred embodiment, the viral vector is defective and only transforms or transfects a proliferating cell but does not transform or transfects a non-proliferating cell. Moreover, in a still further preferred embodiment, an LP polynucleotide sequence is inserted into a proliferating cell either alone, (or in combination with, or fused to, another polynucleotide sequence, which can subsequently be modulated via an external stimulus (e.g., a magnetic signal, a specific small molecule, a chemical moiety or a drug administration, etc.) that acts on an upstream promoter to induce expression of the LP polypeptide (or fragment thereof). As such, a desired effect of the present invention (e.g., selectively increasing, decreasing, or inhibiting expression of an LP polynucleotide sequence) may be accomplished based on using an external stimulus.

An LP sequence may be useful in repressing the expression of a gene or an antigenic composition, e.g., an oncogenic retrovirus. By “repressing the expression of a gene” is meant, e.g., the suppression of the transcription of a ‘gene’, the degradation of a ‘gene’ transcript (pre-message RNA), the inhibition of splicing of a ‘gene’, the destruction of mRNA, the prevention of a post-translational modification of a polypeptide, the destruction of a polypeptide, or the inhibition of a normal function of a protein.

Local administration to an abnormally proliferating cell may be achieved by any art known method or technique discussed herein including, e.g., without limit to transfection, electroporation, microinjection of cells, or in vehicles (such as a liposome, lipofectin, or a naked polynucleotide). Encompassed delivery systems include, without limit, retroviral vectors (Gilboa, J. Virology 44:845 (1982); Hocke, Nature 320:275 (1986); Wilson, et al., Proc. Natl. Acad. Sci. U.S.A. 85:3014); vaccinia virus systems (Chakrabarty, et al., Mol. Cell Biol. 5:3403 (1985); Yates, et al., Nature 3 13:8 12 (1985). Preferably a retroviral, or adenoviral delivery system (as known in the art or described herein) is used to specifically deliver a recombinant construct or to transfect a cell that is abnormally proliferating. An LP polynucleotide sequence may be delivered directly to the site of a cell proliferation, e.g., in an internal organ, body cavity, and the like by use of, e.g., an imaging device used to guide the recombinant construct. Alternatively, administration to an appropriate location may be carried out at a time of surgical intervention.

By “cell proliferative condition” is meant any human or animal disease, syndrome, disorder, condition, or state, affecting any cell, tissue, any site or any combination of organs, tissues, or body parts, which is characterized by a single or multiple local abnormal proliferation of cells, groups of cells, or tissues, whether benign or malignant. Any amount of LP may be administered as long as it has a desired effect on the treated cell, e.g., a biologically inhibiting effect on an abnormally proliferating cell. Moreover, it is possible to administer more than one LP polynucleotide or polypeptide (or fragment thereof), or an agonist or antagonist thereto, simultaneously to the same site.

By “biologically inhibiting” is meant a partial or total inhibition of mitotic activity and/or a decrease in the rate of mitotic activity or metastatic activity of a targeted cell. A biologically inhibitory dose can be determined by assessing the effects of an LP on abnormally proliferating cell division in a cell or tissue culture, tumor growth in an animal or any other art known method. In another embodiment, an LP can be useful to inhibit angiogenesis associated with abnormally proliferative cells or tissues, either alone, or as a protein fusion, or in combination with another LP polynucleotide or polypeptide (or fragment thereof), or an agonist or antagonist, thereto. In a preferred embodiment, a desired anti-angiogenic effect may be achieved indirectly, e.g., through the inhibition of hematopoietic, tumor-specific cells, such as, e.g., tumor-associated macrophages (see e.g., Joseph, et al. (1998) J Natl. Cancer Inst. 90(21): 1648-53). Alternatively, in a desired anti-angiogenic effect may be achieved directly, (e.g., see Witte, et al., (1998) Cancer Metastasis Rev. 17(2): 155-61).

An LP, including a protein fusion, may be useful in inhibiting an abnormally proliferative cell or tissue, via an induction of apoptosis. An LP may act either directly, or indirectly to induce apoptosis in a proliferative cell or tissue, e.g., by activating the death-domain FA receptor, such as, e.g., tumor necrosis factor (TNF) receptor-1, CD95 (F&APO-I), TNF-receptor-related apoptosis-mediated protein (TRAMP) and TNF-related apoptosis-inducing ligand (TRAIL) receptor-1 and -2 (see, e.g., Schulze-Osthoff, et al., Eur J Biochem 254 (3): 439-59 (1998), which is hereby incorporated by reference for teachings on apoptotic cell death). Moreover, in another preferred embodiment, an LP may induce apoptosis via other mechanisms, such as, e.g., through the activation of a pathway that subsequently activates apoptosis, or through stimulating the expression of a protein(s) that activates an apoptotic pathway, either alone or in combination with small molecule drugs or adjuvants, such as apoptonin, galectins, thioredoxins, anti-inflammatory proteins (see e.g., Mutat Res 400 (I-2):447-55 (1998), Med Hypotheses. 50(5): 423-33 (1998), Chem Biol Interact. Apr 24; 111-112:23-34 (1998), J Mol Med. 76(6): 402-12(1998), Int J Tissue React; 20 (1):3-15 (1998), which are all hereby incorporated by reference for these teachings).

An LP is useful in inhibiting cell metastasis either directly as a result of administering a polynucleotide or polypeptide (or fragment thereof), or an agonist or antagonist thereto, (as described elsewhere herein), or indirecdy, such as, e.g., by activating or increasing the expression of a protein known to inhibit metastasis, such as, e.g., an alpha integrin, (see, e.g., Cur. Top Microbial Immunol 1998; 23 1: 125-41, which is hereby incorporated by reference for these teachings). Such a desired effect can be achieved either alone using an LP or in combination with e.g., a small molecule drug or an adjuvant.

An LP, or a protein fusion thereto, is useful in enhancing the immunogenicity and/or antigenicity of a proliferating cell or tissue, either directly, (such as would occur if e.g., an LP polypeptide (or fragment thereof) ‘vaccinated’ the immune system to respond to a proliferative antigen or immunogen), or indirectly, (such as in activating, e.g., the expression a of protein known to enhance an immune response (e.g. a chemokine), to an antigen on an abnormally proliferating cell).

An LP may be used to, modulate, ameliorate, effect, treat, prevent, and/or diagnose a cardiovascular disease, disorder, syndrome, and/or condition. As described herein, including, e.g., without limitation, cardiovascular abnormalities, such as arterio-arterial fistula, arteriovenous fistula, cerebral arteriovenous malformations, congenital heart defects, pulmonary atresia, and Scimitar Syndrome peripheral artery disease, syndrome, such as limb ischemia. Additional cardiovascular disorders encompass, e.g., congenital heart defects which include, e.g., aortic coarctation, car triatriatum, coronary vessel anomalies, crisscross heart, dextrocardia, patent ductus arteriosus, Ebstein's anomaly, Eisenmenger complex, hypoplastic left heart syndrome, levocardia, tetralogy of fallot, transposition of great vessels, double outlet right ventricle, tricuspid atresia, persistent truncus arteriosus, and heart septal defects, such as e.g., aortopulmonary septal defect, endocardial cushion defects, Lutembacher's Syndrome, trilogy of Fallot, and ventricular heart septal defects. Further cardiovascular conditions include, e.g., heart disease syndrome, such as, e.g., arrhythmias, carcinoid heart disease syndrome, high cardiac output, low cardiac output, cardiac tamponade, endocarditis (including bacterial endocarditis), heart aneurysm, cardiac arrest, congestive heart failure, congestive cardiomyopathy, paroxysmal dyspnea, cardiac edema, heart hypertrophy, congestive cardiomyopathy, left ventricular hypertrophy, right ventricular hypertrophy, post-infarction heart rupture, ventricular septal rupture, heart valve disease, myocardial disease, myocardial ischemia, pericardial effusion, pericarditis (including constrictive and tuberculous pericarditis), pneumopericardium, post-pericardiotomy syndrome, pulmonary heart disease syndrome, rheumatic heart disease syndrome, ventricular dysfunction, hyperemia, cardiovascular pregnancy complications, Scimitar Syndrome, cardiovascular syphilis, and cardiovascular tuberculosis. Further cardiovascular disorders include, e.g., arrhythmias including, e.g., sinus arrhythmia, atrial fibrillation, atrial flutter, bradycardia, extra systole, Adams-Stokes Syndrome, bundle-branch block, sinoatrial block, long QT syndrome, parasystole, Lown-Ganong-Levine Syndrome, Mahaim-type pre-excitation syndrome, Wolff-Parkinson-White syndrome, sick sinus syndrome, and ventricular fibrillation tachycardias. Tachycardias encompassed with the cardiovascular condition described herein include, e.g., paroxysmal tachycardia, supraventricular tachycardia, accelerated idioventricular rhythm, atrioventricular nodal re-entry tachycardia, ectopic atrial tachycardia, ectopic junctional tachycardia, sinoatrial nodal re-entry tachycardia, sinus tachycardia, Torsades de Pointes Syndrome, and ventricular tachycardia. Additional cardiovascular disorders include, e.g., heart valve disease such as, e.g., aortic valve insufficiency, aortic valve stenosis, heart murmurs, aortic valve prolapse, mitral valve prolapse, tricuspid valve prolapse, mitral valve insufficiency, mitral valve stenosis, pulmonary atresia, pulmonary valve insufficiency, pulmonary valve stenosis, tricuspid atresia, tricuspid valve insufficiency, and tricuspid valve stenosis. Myocardial conditions associated with cardiovascular disease include, e.g., myocardial diseases such as, e.g., alcoholic cardiomyopathy, congestive cardiomyopathy, hypertrophic cardiomyopathy, aortic subvalvular stenosis, pulmonary subvalvular stenosis, restrictive cardiomyopathy, Chagas cardiomyopathy, endocardial fibroelastosis, endomyocardial fibrosis, Kearns Syndrome, myocardial reperfusion injury, and myocarditis.

Cardiovascular conditions include, e.g., myocardial ischemias such as, e.g., coronary disease syndrome, such as e.g., angina pectoris, coronary aneurysm, coronary arteriosclerosis, coronary thrombosis, coronary vasispasm, myocardial infarction, and myocardial stunning. Cardiovascular diseases also encompassed herein include, e.g., vascular diseases such as e.g., aneurysms, angiodysplasia, angiomatosis, bacillary angiomatosis, Hippel-Lindau Disease syndrome, Klippel-Trenaunay-Weber Syndrome, Sturge-Weber Syndrome, angioneurotic edema, aortic disease, Takayasu's Arteritis, aortitis, Leriche's Syndrome, arterial occlusive disease, arteritis, enarteritis, polyarteritis nodosa, cerebrovascular disease, diabetic angiopathies, diabetic retinopathy, embolism, thrombosis, erythromeialgia, hemorrhoids, hepatic veno-occlusive disease syndrome, hypertension, hypotension, ischemia, peripheral vascular diseases, phlebitis, pulmonary veno-occlusive disease syndrome, Raynaud's disease syndrome, CREST syndrome, retinal vein occlusion, Scimitar syndrome, superior vena cava syndrome, telangiectasia, ataxia telangiectasia, hereditary hemorrhagic telangiectasia, varicocele, varicose veins, varicose ulcer, vasculitis, and venous insufficiency. Cardiovascular conditions further include, e.g., aneurysms such as, e.g., dissecting aneurysms, false aneurysms, infected aneurysms, ruptured aneurysms, aortic aneurysms, cerebral aneurysms, coronary aneurysms, heart aneurysms, and iliac aneurysms. Arterial occlusive cardiovascular conditions include, e.g., arteriosclerosis, intermittent claudication, carotid stenosis, fibromuscular dysplasias, mesenteric vascular occlusion, Moyamoya disease syndrome, renal artery obstruction, retinal artery occlusion, and thromboangiitis obliterans.

Cerebrovascular cardiovascular conditions include, e.g., carotid artery disease, cerebral amyloid angiopathy, cerebral aneurysm, cerebral anoxia, cerebral arteriosclerosis, cerebral arteriovenous malformation, cerebral artery disease, cerebral embolism and thrombosis, carotid artery thrombosis, sinus thrombosis, Wallenberg's syndrome, cerebral hemorrhage, epidural hematoma, subdural hematoma, subarachnoid hemorrhage, cerebral infarction, cerebral ischemia (including transient cerebral ischemia), subclavian steal syndrome, periventricular leukomalacia, vascular headache, cluster headache, migraine, and vertebrobasilar insufficiency. Embolic cardiovascular conditions include, e.g., air embolisms, amniotic fluid embolisms, cholesterol embolisms, blue toe syndrome, fat embolisms, pulmonary embolisms, and thromboembolisms. Thrombotic cardiovascular conditions include, e.g., coronary thrombosis, hepatic vein thrombosis, retinal vein occlusion, carotid artery thrombosis, sinus thrombosis, Wallenberg's syndrome, and thrombophlebitis. Ischemic conditions include, e.g., cerebral ischemia, ischemic colitis, compartment syndromes, anterior compartment syndrome, myocardial ischemia, reperfusion injuries, and peripheral limb ischemia. Vasculitic conditions include, e.g., aortitis, arteritis, Behcet's Syndrome, Churg-Strauss Syndrome, mucocutaneous lymph node syndrome, thromboangiitis obliterans, hypersensitivity vasculitis, Schoenlein-Henoch purpura, allergic cutaneous vasculitis, and Wegener's granulomatosis. An LP can be beneficial in ameliorating critical limb ischemia and coronary disease. An LP may be administered using any art known method, described herein An LP may administered as part of a therapeutic composition or formulation, as described in detail herein. Methods of delivering an LP are also described in detail herein.

Anti-Hemopoietic Activity

The naturally occurring balance between endogenous stimulators and inhibitors of angiogenesis is one in which inhibitory influences typically predominate (see, e.g., Rastinejad, et al., Cell 56345-355 (1989)). When neovascularization occurs under normal physiological conditions, such as wound healing, organ regeneration, embryonic development, and female reproductive processes, angiogenesis is stringently regulated, and delimited spatially and temporally. In pathological angiogenesis such as, e.g., during solid tumor formation, these regulatory controls fail and unregulated angiogenesis can become pathologic by sustaining progression of many neoplastic and non-neoplastic diseases. A number of serious diseases are dominated by abnormal neovascularization (including, e.g., solid tumor growth and metastases, arthritis, some types of eye conditions, and psoriasis; see, e.g., reviews by Moses, et al., Biotech. 9630-634 (1991); Folkman, et al., N. Engl. J. Med., 333: 1757-1763 (1995); Auerbach, et al., J. Microvasc. Res. 29:401-411 (1985); Folkman, “Advances in Cancer Research”, eds. Klein and Weinhouse, Academic Press, New York, pp. 175-203 (1985); Patz, Am. J. Opthalmol. 94:7 15-743 (1982); and Folkman, et al., Science 221:7 19-725 (1983).

In a number of pathological conditions, angiogenesis contributes to a disease-state, e.g., for example, significant data have accumulated suggesting that solid tumor formation is dependent on angiogenesis (see, e.g., Folkman and Klagsbrun, Science 235:442-447 (1987)). In another embodiment of the invention, administration of an LP provides for the treatment, amelioration, modulation, diagnosis, and/or inhibition of a disease, disorder, syndrome, and/or condition associated with neovascularization. Malignant and metastatic conditions that can be effected in a desired fashion using an LP include, e.g., without limitation, a malignancy, solid tumor, and a cancer as described herein or as otherwise known in the art (for a review of such disorders, syndromes, etc. see, e.g., Fishman, et al., Medicine, 2d Ed., J. B. Lippincott Co., Philadelphia (1985)). Thus, the present invention provides a method of ameliorating, modulating, treating, preventing, and/or diagnosing an angiogenesis-related disease and/or disorder, comprising administering to a subject in need thereof a beneficially effective amount of an LP. For example, cancers that may be so affected using a composition of the invention includes, e.g., without limit a solid tumor, including e.g., prostate, lung, breast, ovarian, stomach, pancreas, larynx, esophagus, testes, liver, parotid, biliary tract, colon, rectum, cervix, uterus, endometrium, kidney, bladder, thyroid cancer; primary tumors and metastases; melanomas; glioblastoma; Kaposi's sarcoma; leiomyosarcoma; non-small cell lung cancer, colorectal cancer; advanced malignancies; and blood born tumors such as e.g., leukemia.

Moreover, an LP may be delivered topically, to treat or prevent cancers such as, e.g., skin cancer, head and neck tumors, breast tumors, and Kaposi's sarcoma. Within yet another aspect, an LP may be utilized to treat superficial forms of bladder cancer by, e.g., intravesical administration into the tumor, or near the tumor site; via injection or a catheter. Of course, the appropriate mode of administration will vary according to the cancer to be treated. Other modes of delivery are discussed herein. An LP may also be useful in modulating, ameliorating, treating, preventing, and/or diagnosing another disease, disorder, syndrome, and/or condition, besides a cell proliferative condition (e.g., a cancer) that is assisted by abnormal angiogenic activity. Such close group conditions include, e.g., without limitation, benign tumors, e.g., such as hemangiomas, acoustic neuromas, neurofibromas, trachomas, and pyogenic granulomas; atherosclerotic plaques; ocular angiogenic diseases, e.g., diabetic retinopathy, retinopathy of prematurity, macular degeneration, cornea graft rejection, neovascular glaucoma, retrolental fibroplasia, rubeosis, retinoblastoma, uvietis and Pterygia (abnormal blood vessel growth) of the eye; rheumatoid arthritis; psoriasis; delayed wound healing, endometriosis; vasculogenesis; granulations; hypertrophic scars (keloids); nonunion fractures; scleroderma; trachoma; vascular adhesions; myocardial angiogenesis; coronary collaterals; cerebral collaterals; arteriovenous malformations; ischemic limb angiogenesis; Osler-Webber Syndrome; plaque neovascularization; telangiectasia; hemophiliac joints; angiofibroma; fibromuscular dysplasia; wound granulation; Crohn's disease; and atherosclerosis.

For example, within another aspect of the present invention methods are provided for modulating, ameliorating, treating, preventing, and/or diagnosing hypertrophic scars and keloids, comprising administering an LP to a site of hypertrophic scar or keloid formation. Within one embodiment, the method involves a direct injection into a hypertrophic scar or keloid, to provide a beneficial effect, e.g., by preventing progression of such a lesion. This method is of particular value to a prophylactic treatment of a condition known to result in the development of a hypertrophic scar or a keloid (e.g., burns), and is preferably initiated after the proliferative phase of scar formation has had time to progress (approximately, e.g., 14 days after the initial injury), but before hypertrophic scar or keloid development. As noted above, the present invention also provides methods for ameliorating, treating, preventing, and/or diagnosing neovascular diseases of the eye, including e.g., corneal graft neovascularization, neovascular glaucoma, proliferative diabetic retinopathy, retrolental fibroplasia and macular degeneration. Moreover, ocular diseases, disorders, syndromes, and/or conditions associated with neovascularization that can be modulated ameliorated, treated, prevented, and/or diagnosed with an LP include, e.g., without limit; neovascular glaucoma, diabetic retinopathy, retinoblastoma, retrolental fibroplasia, uveitis, retinopathy of premature macular degeneration, corneal graft neovascularization, as well as other inflammatory eye diseases, ocular tumors, and diseases associated with choroidal or iris neovascularization (see, e.g., reviews by Waltman, et al., (1978) Am. J. Ophthal. 8.51704-710 and Gartner, et al., (1978) Sun. Ophthd. 22:291-312). Thus, within one aspect of the present invention methods are provided for treating or preventing neovascular diseases of the eye such as corneal neovascularization (including corneal graft neovascularization), comprising administering to a patient a therapeutically effective amount of an LP composition to the cornea, such that the formation of blood vessels is inhibited or delayed. Briefly, the cornea is a tissue that normally lacks blood vessels. In certain pathological conditions however, capillaries may extend into the cornea from the pericorneal vascular plexus of the limbus. When the cornea becomes vascularized, it also becomes clouded, resulting in a decline in the patient's visual acuity. Visual loss may become complete if the cornea completely opacifies. A wide variety of diseases, disorders, syndromes, and/or conditions can result in corneal neovascularization, including e.g., corneal infections (e.g., trachoma, herpes simplex keratitis, leishmaniasis and onchocerciasis), immunological processes (e.g., graft rejection and Stevens-Johnson's syndrome), alkali burns, trauma, inflammation (of any cause), toxic and nutritional deficiency states, and as a complication of using contact lenses.

Within particularly preferred embodiments, an LP composition may be prepared for topical administration in saline (combined with any of the preservatives and anti-microbial agents commonly used in ocular preparations), and administered in drop form to the eye. The solution or suspension may be prepared in its pure form and administered several times daily. Alternatively, anti-angiogenic compositions, prepared as described herein, may also be administered directly to the cornea. Within preferred embodiments, an anti-angiogenic composition is prepared with a muco-adhesive polymer, which binds to the cornea.

Within further embodiments, an anti-angiogenic factor or anti-angiogenic LP composition may be utilized as an adjunct to conventional steroid therapy. Topical therapy may also be useful prophylactically in corneal lesions that are known to have a high probability of inducing an angiogenic response (such as, e.g., a chemical burn). In these instances, the treatment (likely in combination with steroids) may be instituted immediately to help prevent subsequent complications. Within other embodiments, an LP composition may be injected directly into the corneal stroma using microscopic guidance by an ophthalmologist. The preferred site of injection may vary with the morphology of the individual lesion, but the goal of the administration is to place a composition of the invention at the advancing front of the vasculature (i.e., interspersed between the blood vessels and the normal cornea). In most instances, this would involve perilimbic corneal injection to “protect” the cornea from advancing blood vessels. This method may also be utilized shortly after a corneal insult to prophylactically prevent corneal neovascularization. In such a situation, the composition could be injected into the perilimbic cornea interspersed between the corneal lesion and its undesired potential limbic blood supply. Such methods may also be utilized in a similar fashion to prevent capillary invasion of transplanted corneas. In a sustained-release form, injections might only be required 2-3 times per year. A steroid could also be added to the injection solution to reduce inflammation resulting from the injection itself.

Within another aspect, methods are provided for treating or preventing neovascular glaucoma, comprising administering to a patient a therapeutically effective amount of an LP to the eye, such that the formation of blood vessels is inhibited. In one embodiment, the composition may be administered topically to the eye to treat or prevent early forms of neovascular glaucoma. Within other embodiments, the composition may be implanted by injection into the region of the anterior chamber angle. Within other embodiments, the composition may also be placed in any location such that the composition is continuously released into the aqueous humor. Within another aspect, methods are provided for treating or preventing proliferative diabetic retinopathy, comprising administering to a patient a therapeutically effective amount of an LP to the eyes, such that the formation of blood vessels is inhibited. Within a particularly preferred embodiment, proliferative diabetic retinopathy may be treated by injection into the aqueous or the vitreous humor, to increase the local concentration of a composition of the invention in the retina. Preferably, this treatment should be initiated before the acquisition of severe disease requiring photocoagulation. Within another aspect of the present invention, methods are provided for treating or preventing retrolental fibroplasia, comprising administering to a patient a beneficially effective amount of an LP to the eye, such that the formation of blood vessels is inhibited. The composition may be administered topically, via intravitreous injection and/or via intraocular implants. Additional, diseases, disorders, syndromes, and/or conditions that can be modulated, ameliorated, treated, prevented, and/or diagnosed with an LP include, e.g., without limitation, hemangioma, arthritis, psoriasis, angiofibroma, atherosclerotic plaques, delayed wound healing, granulations, hemophilic joints, hypertrophic scars, nonunion fractures, Osler-Weber syndrome, pyogenic granuloma, scleroderma, trachoma, and vascular adhesions.

Moreover, diseases, disorders, states, syndromes, and/or conditions that can be modulated, ameliorated, treated, prevented, and/or diagnosed with an LP include, e.g., without limitation, solid tumors, blood born tumors such as leukemias, tumor metastasis, Kaposi's sarcoma, benign tumors (e.g., hemangiomas), acoustic neuromas, neurofibromas, trachomas, and pyogenic granulomas, rheumatoid arthritis, psoriasis, ocular angiogenic diseases, e.g., diabetic retinopathy, retinopathy of prematurity, macular degeneration, corneal graft rejection, neovascular glaucoma, retrolental fibroplasia, rubeosis, retinoblastoma, and uvietis, delayed wound healing, endometriosis, vasculogenesis, granulations, hypertrophic scars (keloids), nonunion fractures, scleroderma, trachoma, vascular adhesions, myocardial angiogenesis, coronary collaterals, cerebral collaterals, arteriovenous malformations, ischemic limb angiogenesis, Osler-Webber Syndrome, plaque neovascularization, telangiectasia, hemophiliac joints, angiofibroma fibromuscular dysplasia, wound granulation, Crohn's disease, syndrome, atherosclerosis, birth-control inhibition of vascularization necessary for embryo implantation during the control of menstruation, and diseases that have angiogenesis as a pathologic consequence such as, e.g., cat scratch disease (Rochele minalia quintosa), ulcers (Helicobacter pylon), Bartonellosis and bacillary angiomatosis.

In another embodiment as a birth control method, an amount of an LP sufficient to block embryo implantation is administered before or after intercourse and fertilization have occurred, thus providing an effective method of birth control, possibly a “morning after” method. An LP may also be used in controlling menstruation or administered either as a peritoneal lavage fluid or for peritoneal implantation in the treatment of endometriosis.

An LP may be utilized in a wide-variety of surgical procedures. For example, within one aspect of the present invention a compositions (in the form of, e.g., a spray or film) may be utilized to coat or spray an area before removal of a tumor, to isolate normal surrounding tissues from malignant tissue, and/or to prevent the spread of disease to surrounding tissues. Within other aspects, an LP composition (e.g., in the form of a spray) may be delivered via endoscopic procedures to coat tumors, or inhibit angiogenesis in a desired locale. Within yet another aspect, surgical meshes that have been coated with an anti-angiogenic composition of the invention may be utilized in a procedure in which a surgical mesh might be utilized. For example, a surgical mesh laden with an anti-angiogenic composition may be utilized during cancer resection surgery (e.g., abdominal surgery subsequent to colon resection) to provide support to the structure, and to release an amount of the anti-angiogenic factor. Within further aspects of the present invention, methods are provided for treating tumor excision sites, comprising administering an LP to the resection margins of a tumor after excision, such that the local recurrence of cancer and the formation of new blood vessels at the site is inhibited.

Within one embodiment, an anti-angiogenic composition of the invention is administered directly to a tumor excision site (e.g., applied by swabbing, brushing or otherwise coating the resection margins of the tumor with the anti-angiogenic composition). Alternatively, an anti-angiogenic composition may be incorporated into a known surgical paste before administration. Within a particularly preferred embodiment, an anti-angiogenic composition of the invention is applied after hepatic resections for malignancy, and after neurosurgical operations. Within another aspect, administration can be to a resection margin of a wide variety of tumors, including e.g., breast, colon, brain, and hepatic tumors. For example, within one embodiment, anti-angiogenic compositions may be administered to the site of a neurological tumor after excision, such that the formation of new blood vessels at the site is inhibited.

Diseases at the Cellular Level

Diseases associated with increased cell survival or the inhibition of apoptosis that could be modulated, ameliorated, treated, prevented, and/or diagnosed by an LP include, e.g., cancers (such as, e.g., follicular lymphomas, carcinomas with p53 mutations, and hormone-dependent tumors, including, e.g., but without limit, colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer); autoimmune conditions (such as, e.g., multiple sclerosis, Sjogren's syndrome, Hashimoto's thyroiditis, biliary cirrhosis, Behcet's disease syndrome, Crohn's disease syndrome, polymyositis, systemic lupus erythematosus, immune-related glomerulonephritis, and rheumatoid arthritis); viral infections (such as, e.g., herpes viruses, pox viruses, and adenoviruses); inflammation; graft v. host disease syndrome, acute graft rejection, and chronic graft rejection.

In preferred embodiments, an LP is used to inhibit growth, progression, and/or metastases of cancers such as, in particular, those listed herein. Additional diseases, states, syndromes, or conditions associated with increased cell survival that could be modulated, ameliorated, treated, prevented, or diagnosed by an LP include, e.g., without limitation, progression, and/or metastases of malignancies and related disorders such as leukemia including acute leukemias (such as, e.g., acute lymphocytic leukemia, acute myelocytic leukemia, including myeloblastic, promyelocytic, myelomonocytic, monocytic, and erythroleukemia) and chronic leukemias (e.g., chronic myelocytic, chronic granulocytic, leukemia, and chronic lymphocytic leukemia)), polycythemia Vera, lymphomas (e.g., Hodgkin's disease, and non-Hodgkin's disease), multiple myeloma, Waldenstrom's macroglobulinemia, heavy chain disease, syndrome, and solid tumors including, e.g., without limitation, sarcomas and carcinomas (such as, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma).

Diseases associated with increased apoptosis that could be modulated, ameliorated, treated, prevented, and/or diagnosed by an LP include, e.g., AIDS, conditions (such as, e.g., Alzheimer's disease syndrome, Parkinson's disease syndrome, Amyotrophic lateral sclerosis, Retinitis pigmentosa, Cerebellar degeneration and brain tumor, or prion associated disease); autoimmune conditions (such as, e.g., multiple sclerosis, Sjogren's syndrome, Hashimoto's thyroiditis, biliary cirrhosis, Behcet's disease syndrome, Crohn's disease syndrome, polymyositis, systemic lupus erythematosus, immune-related glomerulonephritis, and rheumatoid arthritis); myelodysplastic syndromes (such as aplastic anemia), graft v. host disease syndrome; ischemic injury (such as that caused by myocardial infarction, stroke and reperfusion injury); liver injury (such as, e.g., hepatitis related liver injury, ischemia reperfusion injury, cholestosis (bile duct injury), and liver cancer); toxin-induced liver disease (such as, e.g., that caused by alcohol), septic shock, cachexia, and anorexia.

Wound Healing and Epithelial Cell Proliferation

In accordance with yet a further aspect of the invention, there is provided a process for using an LP to stimulate epithelial cell proliferation and basal keratinocytes for the purpose of, e.g., wound healing, to stimulate hair follicle production, and to heal a dermal wound. An LP composition may be clinically useful in stimulating wound healing including e.g., surgical wounds, excisional wounds, deep wounds involving damage of the dermis and epidermis, eye tissue wounds, dental tissue wounds, oral cavity wounds, diabetic ulcers, dermal ulcers, cubitus ulcers, arterial ulcers, venous stasis ulcers, burns resulting from exposure heat or chemicals, abnormal wound healing conditions associated with e.g., uremia, malnutrition, vitamin deficiency and wound healing complications associated with systemic treatment with steroids, radiation therapy, anti-neoplastic drugs, and anti-metabolites. An LP could be used to promote dermal reestablishment after dermal loss.

An LP could be used to increase the adherence of skin grafts to a wound bed and to stimulate re-epithelialization from the wound bed. The following is a non-exhaustive list of grafts that an LP could be used to increase adherence to: a wound bed, autografts, artificial skin, allografts, autodermic grafts, autoepidermic grafts, avascular grafts, Blair-Brown grafts, bone grafts, brephoplastic grafts, cutis grafts, delayed grafts, dermic grafts, epidermic grafts, fascia grafts, full thickness grafts, heterologous grafts, xenografts, homologous grafts, hyperplastic grafts, lamellar grafts, mesh grafts, mucosal grafts, Ollier-Thiersch grafts, omenpal grafts, patch grafts, pedicle grafts, penetrating grafts, split skin grafts, and thick split grafts. An LP can be used to promote skin strength and to improve the appearance of aged skin. It is believed that an LP will also produce changes in hepatocyte proliferation, and epithelial cell proliferation in, for example, the lung, breast, pancreas, stomach, small intestine, and large intestine. Epithelial cell proliferation can be effected in epithelial cells such as, e.g., sebocytes, hair follicles, hepatocytes, type II pneumocytes, mucin-producing goblet cells, and other epithelial cells or their progenitors which are contained within the skin, lung, liver, and gastrointestinal tract.

An LP may: promote proliferation of endothelial cells, keratinocytes, and basal keratinocytes; it could also be used to reduce the side effects of gut toxicity that result from radiation, chemotherapy treatments or viral infections, it may have a cytoprotective effect on the small intestine mucosa; it may also stimulate healing of mucositis (mouth ulcers) that result from chemotherapy and viral infections, it could further be used in full regeneration of skin in full and partial thickness skin defects, including burns, (i.e., re-population of hair follicles, sweat glands; and sebaceous glands), treatment of other skin defects such as psoriasis, it also could be used to treat epidermolysis bullosa, a defect in adherence of the epidermis to the underlying dermis which results in frequent, open and painful blisters by accelerating re-epithelialization of these lesions; it could also be used to treat gastric and doudenal ulcers and help heal by scar formation of the mucosal lining and regeneration of glandular mucosa and duodenal mucosal lining more rapidly. Inflammatory bowel diseases, such as Crohn's disease and ulcerative colitis, are diseases that result in destruction of the mucosal surface of the small or large intestine, respectively. Thus, an LP could be used to promote resurfacing of a mucosal surface to aid more rapid healing and to prevent progression of inflammatory bowel disease resulting in a desired effect, e.g., such as on the production of mucus throughout the gastrointestinal tract and the protection of intestinal mucosa from injurious substances that are ingested or following surgery. An LP could be used to treat a condition associated with the under expression of an LP polynucleotide sequence or an LP polypeptide of the present invention (or fragment thereof), or an agonist or antagonist thereto.

Moreover, an LP could be used to prevent and heal damage to the lungs due to various pathological states, such as, e.g., stimulating proliferation and differentiation to promote repair of alveoli and bronchiolar epithelium. For example, emphysema, inhalation injuries, that (e.g., from smoke inhalation) and burns, which cause necrosis of the bronchiolar epithelium and alveoli could be effectively ameliorated, treated, prevented, and/or diagnosed using a polynucleotide or polypeptide of the invention (or fragment thereof), or an agonist or antagonist thereto. Also, an LP could be used to stimulate the proliferation of and differentiation of type II pneumocytes, to help treat or prevent hyaline membrane diseases, such as e.g., infant respiratory distress syndrome and bronchopulmonary displasia, (in premature infants). An LP could stimulate the proliferation and/or differentiation of a hepatocyte and, thus, could be used to alleviate or treat a liver condition such as e.g., fulminant liver failure (caused, e.g., by cirrhosis), liver damage caused by viral hepatitis and toxic substances (e.g., acetaminophen, carbon tetrachloride, and other known hepatotoxins). In addition, an LP could be used treat or prevent the onset of diabetes mellitus. In patients with newly diagnosed Types I and II diabetes, where some islet cell function remains, an LP could be used to maintain the islet function so as to alleviate, modulate, ameliorate, delay, or prevent permanent manifestation of the disease. In addition, an LP could be used as an auxiliary in islet cell transplantation to improve or promote islet cell function.

Neurological Diseases

Nervous system diseases, disorders, syndromes, states, and/or conditions that can be modulated, ameliorated, treated, prevented, and/or diagnosed with an LP composition include, e.g., without limitation, nervous system injuries diseases, disorders, states, syndromes, and/or conditions that result in either a disconnection or misconnection of an axon or dendrite; a diminution or degeneration of a cell (or part of a cell) of the nervous system (such as, e.g., without limitation, neurons, astrocytes, microglia, macroglia, oligodendroglia, Schwann cells, and ependymal cells); demyelination or improper mylenation; neural cell dysfunction (such as, e.g., failure of neurotransmitter release or uptake); or interference with mylenization. Nervous system lesions that may be modulated, ameliorated, treated, prevented, and/or diagnosed in a subject using an LP composition of the invention, include, e.g., without limitation, the following lesions of either the central (including spinal cord and brain) or peripheral nervous system: (1) ischemic lesions, in which a lack of oxygen in a portion of the nervous system results in neuronal injury or death, including e.g., cerebral infarction (or ischemia), or spinal cord infarction (or ischemia); (2) traumatic lesions, including, e.g., lesions caused by physical injury or associated with surgery (e.g., lesions that sever a portion of the nervous system), or compression injuries; (3) malignant lesions, in which a portion of the nervous system is comprised by malignant tissue, which is either a nervous system associated malignancy or a malignancy derived from non-nervous-system tissue; (4) infectious lesions, in which a portion of the nervous system is comprised because of infection (e.g., by an abscess or associated with infection by human immunodeficiency virus, herpes zoster, or herpes simplex virus or with Lyme disease, syndrome, tuberculosis, syphilis); (5) degenerative lesions, in which a portion of the nervous system is comprised because of a degenerative process including, without limit, degeneration associated with Parkinson's disease syndrome, Alzheimer's disease syndrome, Huntington's chorea, or Amyotrophic lateral sclerosis (ALS); (6) lesions associated with a nutritional condition, in which a portion of the nervous system is comprised by a nutritional disorder (or a disorder of metabolism including, without limit, vitamin B 12 deficiency, folic acid deficiency, Wernicke disease, syndrome, tobacco-alcohol amblyopia, Marchiafava-Bignami disease (primary degeneration of the corpus callosum), and alcoholic cerebellar degeneration; (7) neurological lesions associated with systemic diseases including, e.g., without limitation, diabetes (diabetic neuropathy, Bell's palsy), systemic lupus erythematosus, carcinoma, or sarcoidosis; (8) lesions caused by toxic substances including e.g., alcohol, lead, or a neurotoxin; and (9) demyelinating lesions in which a portion of the nervous system is comprised by a demyelinating cause (including, e.g., without limitation, multiple sclerosis, human immunodeficiency virus-associated myelopathy, transverse myelopathy or various etiologies, progressive multifocal leukoencephalopathy, and central pontine myelinolysis).

In a preferred embodiment, an LP can be used to protect a neuronal cell from the damaging effects of cerebral hypoxia; cerebral ischemia, cerebral infarction; stroke; or a neural cell injury associated with a heart attack. An LP, which is useful for producing a desired effect in a nervous system condition, may be selected by testing for biological activity in promoting survival and/or differentiation of neural cell. For example, an LP that elicits any of the following effects may be useful according to the invention: (1) increased survival time of neurons in culture; (2) increased or decreased sprouting of a neural in culture or in vivo; (3) increased or decreased production of a neuron-associated molecule e.g., such as a neurotransmitter in culture or in vivo, e.g., choline acetyltransferase or acetylcholinesterase with respect to a motor neuron; or (4) decreasing a symptom of neuronal dysfunction in vivo or in a model system, e.g., such as a mouse model for Parkinsons Syndrome. Such an effect may be measured by any known art method.

In a preferred, non-limiting embodiment any art known method can be used to: measure increased neuronal survival (such as, e.g., described in Arakawa, et al. (1990) J. Neurosci. 10:3507-3515); detect increased or decreased sprouting (such as, e.g., described in Pestronk, et al. (1980) Exp. Neurol. 70:65-82; Brown, et al. (1981) Ann. Rev. Neurosci. 4:17-42); measure increased production of a neuron-associated molecule (e.g., by bioassay, enzymatic assay, antibody binding, Northern blot assay, etc., depending on the molecule to be measured); and measure motor neuron dysfunction (by, e.g., assessing the physical manifestation of motor neuron disorder, e.g., weakness, motor neuron conduction velocity, or functional disability in a model system). In specific embodiments, motor neuron diseases, disorders, syndromes, and/or conditions that may be modulated, ameliorated, treated, prevented, and/or diagnosed using an LP composition include, e.g., without limitation, infarction, infection, exposure to toxin, trauma, surgical damage, degenerative disease or malignancy (that may affect motor neurons as well as other components of the nervous system), as well as conditions that selectively affect neurons such as, e.g., without limitation, Amyotrophic lateral sclerosis progressive spinal muscular atrophy, progressive bulbar palsy, primary lateral sclerosis, infantile and juvenile muscular atrophy, progressive bulbar paralysis of childhood (Fazio-Londe syndrome), poliomyelitis post polio syndrome, and Hereditary Motorsensory Neuropathy (Charcot-Marie-Tooth Disease).

Infectious Disease

An LP composition can be used to modulate, ameliorate, treat, prevent, and/or diagnose an effect of an infectious agent in a subject or associated with a condition. For example, by increasing an immune response e.g., particularly increasing the proliferation and differentiation a of B and/or a T cell, infectious diseases may be modulated, ameliorated, treated, prevented, and/or diagnosed. The immune response may be increased either by enhancing an existing immune response, or by initiating a new immune response. Alternatively, an LP may also directly inhibit an infectious agent, without necessarily eliciting an immune response. Viruses are a type of an infectious agent that can cause diseases, disorders, syndromes, and/or conditions that may be modulated, ameliorated, treated, prevented, and/or diagnosed using an LP composition of the invention. Examples of such viruses, include, e.g., without limitation, the following DNA and RNA viruses and viral families: Arbovirus, Adenoviridae, Arenaviridae, Arterivirus, Birnaviridae, Bunyaviridae, Caliciviridae, Circoviridae, Coronaviridae, Dengue, EBV, HIV, Flaviviridae, Hepadnaviridae (Hepatitis), Herpesviridae (such as, e.g., Cytomegalovirus, Herpes Simplex, Herpes Zoster), Mononegavirus (e.g., Paramyxoviridae, Morbillivirus, Rhabdoviridae), Orthomyxoviridae (e.g., Influenza A, Influenza B, and parainfluenza), Papilomavirus, Papovaviridae, Parvoviridae, Picornaviridae, Poxviridae (such as, e.g., Smallpox or Vaccinia), Reoviridae (e.g., Rotavirus), Retroviridae (such as, e.g., HTLV-I, HTLV-II, Lentivirus), and Togaviridae (e.g., Rubivirus). Typically, viruses of these families can cause a variety of undesired conditions, including, but not limited to: e.g., arthritis, bronchiollitis, respiratory syncytial virus, encephalitis, eye infections (e.g., conjunctivitis, keratitis), chronic fatigue syndrome, hepatitis (e.g., of type A, B, C, E, Chronic Active, or Delta), Japanese Bencephalitis, Junin, Chikungunya, Rift Valley fever, yellow fever, meningitis, opportunistic infections (e.g., AIDS), pneumonia, Burkitt's Lymphoma, chickenpox, hemorrhagic fever, Measles, Mumps, Parainfluenza, Rabies, a common cold, Polio, leukemia, Rubella, sexually transmitted diseases, skin diseases (e.g., Kaposi's, warts), and viremia. An LP can be used to modulate, ameliorate, treat, prevent, and/or diagnose any of these symptoms or diseases.

In specific embodiments, an LP composition is used to modulate, ameliorate, treat, prevent, and/or diagnose e.g., meningitis, Dengue, EBV, and/or hepatitis (e.g., hepatitis B). In a further specific embodiment, an LP is administered to a subject that is non-responsive to one or more currently established commercially available, hepatitis vaccines. In a further specific embodiment an LP can be used to modulate, ameliorate, treat, prevent, and/or diagnose AIDS or an AIDS-related syndrome or condition. Similarly, bacterial or fungal agents that can cause a disease, disorder, condition, syndrome, or symptom and that can be ameliorated, treated, prevented, and/or diagnosed by an LP composition of the invention include, e.g., but without limitation, the following: Gram-Negative and Gram-positive bacteria and bacterial families and fungi such as: Actinomycetales (e.g., Corynebacterium, Mycobacterium, Norcardia), Cryptococcus neoformans, Aspergillosis, Baccillaceae (e.g., Anthrax, Clostridium), Bacteroidaceae, Blastomycosis, Bordetella, Borrelia (e.g., Borrelia burgdorferi), Brucellosis, Candidiasis, Campylobacter, Coccidioidomycosis, Cryptococcosis, Dermatocycoses, E. coli (e.g., EnterotoxigenicE. coli and Enterohemorrhagic E. coli), Enterobacteriaceae (Klebsiella, Salmonella (e.g., Salmonella typhi, and Salmonella paratyphi), Serratia, Yersinia), Erysipelothrix, Helicobacter, Legionellosis, Leptospirosis, Listeria, Mycoplasmatales, Mycobacterium leprae, Vibrio cholerae, Neisseriaceae (e.g., Acinetobacter, Gonorrhea, Menigococcal), Meisseria meningitidis, Pasteurelilacea Infections (e.g., Actinobacillus, Heamophilus (e.g., Heamophilus influenza type B), Pasteurella), Pseudomonas, Rickettsiaceae, Chlamydiaceae, Syphilis, Shigella spp., Staphylococcal, Meningiococcal, Pneumococcal and Streptococcal (e.g., Streptococcus pneumoniae and Group B Streptococcus).

These bacterial or fungal families can cause the following diseases, disorders, conditions, syndromes, or symptoms including, e.g., without limitation, bacteremia, endocarditis, eye infections (conjunctivitis, tuberculosis, uveitis), gingivitis, opportunistic infections (e.g., AIDS related infections), paronychia, prosthesis-related infections, Reiter's Disease syndrome, respiratory tract infections, such as Whooping Cough or Empyema, sepsis, Lyme Disease syndrome, Cat-Scratch Disease syndrome, Dysentery, Paratyphoid Fever, food poisoning, Typhoid, pneumonia, Gonorrhea, meningitis (e.g., meningitis types A and B), Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections and wound infections. An LP can be used to modulate, ameliorate, treat, prevent, and/or diagnose any of these diseases, disorders, conditions, syndromes, or symptoms.

In specific embodiments, an LP composition can be used to modulate, ameliorate, treat, prevent, and/or diagnose: tetanus, Diptheria, botulism, and/or meningitis type B. Moreover, parasitic agents causing diseases, disorders, conditions, syndromes, or symptoms that can be modulated, ameliorated, treated, prevented, and/or diagnosed by an LP include, e.g., without limitation, a parasitic agent from any of the following groupings: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, Trichomona, Sporozoans (e.g., Plasmodium virax, Plasmodium falciparium, Plasmodium malariae, and Plasmodium ovale). These parasites can cause a variety of diseases or symptoms, including, e.g., without limitation: Scabies, Trombiculiasis, eye infections, intestinal disease (e.g., dysentery, giardiasis), liver disease syndrome, lung disease syndrome, opportunistic infections (e.g., AIDS related conditions), malaria, complications of pregnancy, and toxoplasmosis. An LP composition of the invention can be used to modulate, ameliorate, treat, prevent, and/or diagnose any of these diseases, disorders, conditions, syndromes, or symptoms. In specific embodiments, an LP can be used to modulate, ameliorate, treat, prevent, and/or diagnose malaria.

Preferably, treatment or prevention using an LP is accomplished either by administering an effective amount of an LP composition to a subject, or by removing cells from a subject, delivering an LP then returning the resulting engineered cell to the patient (ex vivo therapy). Furthermore, an LP sequence can be used as an antigen in a vaccine to raise an immune response against an infectious disease.

Regeneration

An LP composition of the invention can be used e.g., to differentiate a cell, tissue; or biological structure, de-differentiate a cell, tissue; or biological structure; cause proliferation in cell or a zone (similar to a ZPA in a limb bud), have an effect on chemotaxis, remodel a tissue (e.g., basement membrane, extra cell matrix, connective tissue, muscle, epithelia), or initiate the regeneration of a tissue, organ, or biological structure (see, e.g., Science (1997) 276:59-87). Regeneration using an LP composition of the invention could be used to repair, replace, remodel, or protect tissue damaged by, e.g., congenital defects, trauma (such as, e.g., wounds, burns, incisions, or ulcers); age; disease (such as, e.g., osteoporosis, osteoarthritis, periodontal disease syndrome, or liver failure), surgery, (including, e.g., cosmetic plastic surgery); fibrosis; re-perfusion injury; or cytokine damage. Tissues that can be regenerated include, e.g., without limitation, organs (e.g., pancreas, liver, intestine, kidney, epithelia, endothelium), muscle (smooth, skeletal, or cardiac), vasculature (including vascular and lymphatics), nervous system tissue, cells, or structures; hematopoietic tissue; and skeletal (bone, cartilage, tendon, and ligament) tissue. Preferably, regeneration occurs with little or no scarring. Regeneration also may include, e.g., angiogenesis.

Moreover, an LP composition may increase the regeneration of an aggregation of special cell types, a tissue, or a matrix that typically is difficult to heal. For example, by increasing the rate at which a tendon/ligament heals after damage. Also encompassed is using an LP prophylactically to avoid damage (e.g., in an interstitial space of a joint or on the cartalagenous capsule of a bone). Specific diseases that could be modulated, ameliorated, treated, prevented, and/or diagnosed using an LP composition include, e.g., without limitation, tendinitis, carpal tunnel syndrome, and other tendon or ligament defects. Examples of non-healing wounds include, wounds that would benefit form regeneration treatment, e.g., without limit pressure ulcers, ulcers associated with vascular insufficiency, surgical wounds, and traumatic wounds.

Similarly, nerve and brain tissue also could be regenerated using an LP. Such nervous system conditions that could be modulated, ameliorated, treated, prevented, and/or diagnosed using an LP composition include, e.g., without limitation, central and peripheral nervous system diseases, neuropathies, or mechanical and traumatic conditions (e.g., spinal cord disorders or syndromes, head trauma, cerebrovascular disease syndrome, and stoke). Specifically, diseases associated with peripheral nerve injuries include, e.g., without limitation, peripheral neuropathy (e.g., resulting from chemotherapy or other medical therapies), localized neuropathies, and central nervous system diseases (e.g., Alzheimer's disease syndrome, Parkinson's disease syndrome, Huntington's disease syndrome, Amyotrophic lateral sclerosis, and Shy-Drager syndrome). All could be ameliorated, treated, prevented, and/or diagnosed using an LP.

An LP may have an effect on a chemotaxis activity. Briefly, chemotactic molecules can attract or mobilize (but may also repeal) cells (e.g., monocytes, fibroblasts, neutrophils, T-cells, mast cells, eosinophils, epithelial and/or endothelial cells) or cell processes (e.g., filopodia, psuedopodia, larnellapodia, dendrites, axons, etc.) to a particular site (e.g., such as inflammation, infection, site of hyperproliferation, the floor plate of the developing spinal cord, etc.). In some instances, such mobilized cells can then fight off and/or modulate a particular trauma, abnormality, condition, syndrome, or disease. An LP may have an effect on a chemotactic activity of a cell (such as, e.g., an attractive or repulsive effect).

A chemotactic molecule can be used to modulate, ameliorate, treat, prevent, and/or diagnose inflammation, infection, hyperproliferative diseases, disorders, syndromes, and/or conditions, or an immune system disorder by increasing the number of cells targeted to a particular location in the body. For example, a chemotactic molecule can be used to attract an immune cell to an injured location in a subject. An LP that had an effect on a chemotactant could also attract a fibroblast, which can be used to modulate, ameliorate, and/or treat a wound. It is also contemplated that an LP may inhibit a chemotactic activity to modulate, ameliorate, treat, prevent, and/or diagnose a disease, disorder, syndrome, and/or a condition.

XI. Kits

This invention also contemplates use of LP proteins, fragments thereof, peptides, and their fusion products in a variety of diagnostic kits and methods for detecting the presence of LP protein or a binding partner. Typically, the kit will have a compartment containing either a defined LP protein peptide or gene segment or a reagent, which recognizes one or the other, e.g., binding partner fragments or antibodies.

A preferred kit for determining the concentration of, e.g., a LP protein in a sample would typically comprise a labeled compound, e.g., binding partner or antibody, having known binding affinity for the LP protein, a source of LP protein (naturally occurring or recombinant), and a means for separating the bound from free labeled compound, for example, a solid phase for immobilizing the LP protein. Compartments containing reagents, and instructions, will normally be provided. Another diagnostic aspect of this invention involves use of oligonucleotide or polynucleotide sequences taken from the sequence of a LP protein. These sequences are used as probes for detecting levels of the LP protein message in samples from natural sources, or patients suspected of having an abnormal condition, e.g., cancer or developmental problem. The preparation of both RNA and DNA nucleotide sequences, the labeling of the sequences, and the preferred size of the sequences has received ample description and discussion in the literature.

In specific embodiments, a kit may include, e.g., a recombinantly produced or chemically synthesized polypeptide antigen. The polypeptide antigen of the kit may also be attached to a solid support. In a more specific embodiment the detecting means of the above-described kit includes, e.g., a solid support to which said polypeptide antigen is attached. Such a kit may also include, e.g., a non-attached reporter-labeled anti-human antibody. In this embodiment, binding of the antibody to the polypeptide antigen is detected by binding of the reporter-labeled antibody.

### Other Preferred Embodiments

Other preferred embodiments of the claimed invention include an isolated or recombinant nucleic acid molecule comprising a polynucleotide sequence that is at least 95% identical to a polynucleotide sequence of at least about: 10, 12, 14, 16,18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 120, 130, 140, or 150 contiguous nucleotides of a sequence of SEQ ID NO:X wherein X is any integer as defined in a Table herein. Other preferred embodiments of the claimed invention include an isolated or recombinant nucleic acid molecule comprising a polynucleotide sequence that is at least 95% identical to a polynucleotide sequence of at least about: 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90,92, 94, 96,98, 100, 110, 120, 130, 140, or 150 contiguous nucleotides of a mature coding portion of SEQ ID NO:X wherein X is any integer as defined in a Table herein. Also preferred is a nucleic acid molecule wherein said sequence of contiguous nucleotides is include, e.g. in the nucleotide sequence of SEQ ID NO:X in the range of positions beginning with the nucleotide at about the position of the 5′ nucleotide of the Clone Sequence and ending with the nucleotide at about the position of the 3′ nucleotide of the Clone Sequence as defined for SEQ ID NO:X in a Table herein. Also preferred is a nucleic acid molecule wherein said sequence of contiguous nucleotides is included, e.g., in the nucleotide sequence of SEQ ID NO:X in the range of positions beginning with the nucleotide at about the position of the 5′ nucleotide of the Start Codon and ending with the nucleotide at about the position of the 3′ nucleotide of the Clone Sequence as defined for SEQ ID NO:X in a Table herein. Similarly preferred is a nucleic acid molecule comprising polynucleotide sequence of SEQ ID NO:X in the range of positions beginning with the nucleotide at about the position of the 5′ nucleotide of a correspondingly encoded First Amino Acid of a Signal Peptide and ending with the nucleotide at about the position of the 3′ nucleotide of a Clone Sequence as defined for SEQ ID NO:X in a Table herein. Also preferred is an isolated or recombinant nucleic acid molecule comprising a polynucleotide sequence that is at least 95% identical to a polynucleotide sequence of at least about: 10, 12, 14, 16, 18, 20, 22, 24, 26, 28, 30, 32, 34, 36, 38, 40, 42, 4.4, 46, 48, 50, 52, 54, 56, 58, 60, 62, 64, 66, 68, 70, 72, 74, 76, 78, 80, 82, 84, 86, 88, 90, 92, 94, 96, 98, 100, 110, 120, 130, 140, or 150 contiguous nucleotides in at least one polynucleotide sequence fragment of SEQ ID NO:X. More preferably said polynucleotide sequence that is at least 95% identical to one, exhibits 95% sequence identity to at least: 2, 3, 4, 5, 6, 7, 8, 9, 10, or more polynucleotide fragments 22, 24, 26, 28, 30, 32, 34, 36, 38, 40, 42, 44, 46, 48, 50, 52, 54, 56, 58, 60, 62, 64, 66, 68, 70, 72, 74, 76,78, 80, 82, 84, 86, 88, 90,92,94, 96,98, 100, 110, 120, 130, 140, or 150 contiguous nucleotides in length of the mature coding portion of SEQ ID NO:X, wherein any one such fragment is at least 21 contiguous nucleotides in length. Further preferred is an isolated or recombinant nucleic acid molecule comprising a polynucleotide sequence that is at least 95% identical to a polynucleotide sequence of at least about: 200, 250, 300, 350, 400, 450, or 500 contiguous nucleotides of the mature coding portion of SEQ ID NO:X. Also preferred is an isolated or recombinant nucleic acid molecule comprising a polynucleotide sequence that is at least 95% identical to a sequence of at least about: 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 120, 130, 140, or 150 contiguous nucleotides in at least one nucleotide sequence fragment of SEQ ID NO:X, wherein the length of at least one such fragment is about 200, 250, 300, 350, 400, 450, or 500 contiguous nucleotides of SEQ ID NO:X. Another preferred embodiment is an isolated or recombinant nucleic acid molecule comprising a polynucleotide sequence that is at least 95% identical to a sequence of SEQ ID NO:X beginning with the nucleotide at about the position of the 5′ Nucleotide of the First Amino Acid of the Signal Peptide and ending with the nucleotide at about the position of the 3′ Nucleotide of a Clone Sequence as defined for SEQ ID NO:X in a Table herein. A further preferred embodiment is an isolated or recombinant nucleic acid molecule comprising a polynucleotide sequence, which is at least 95% identical to the complete mature coding portion of SEQ ID NO:X or a species variant thereof. Also preferred is an isolated or recombinant nucleic acid molecule comprising polynucleotide sequence that hybridizes under stringent hybridization conditions to a mature coding portion of a polynucleotide of the invention (or fragment thereof), wherein the nucleic acid molecule that hybridizes does not hybridize under stringent hybridization conditions to a nucleic acid molecule having a nucleotide sequence consisting of only A residues or of only T residues. Thus, the invention provides an assay system or kit for carrying out a diagnostic method. The kit generally includes, e.g., a support with surface-bound recombinant antigens, and a reporter-labeled anti-human antibody for detecting surface-bound anti-antigen antibody.

The broad scope of this invention is best understood with reference to the following examples, which are not intended to limit the invention to specific embodiments.

### EXAMPLES

General Methods

Many of the standard methods described herein are described or referenced, e.g., in Maniatis, et al. (Cur. ed.) Molecular Cloning, A Laboratory Manual Cold Spring Harbor Laboratory, Cold Spring Harbor Press, N.Y.; Sambrook, et al.; Ausubel, et al., Biology Greene Publishing Associates, Brooklyn, N.Y.; or Ausubel, et al. (1987 and Supplements) Current Protocols in Molecular Biology Wiley/Greene, NY; Innis, et al. (eds.) (1990) PCR Protocols: A Guide to Methods and Applications Academic Press, NY. Methods for protein purification include such methods as ammonium sulfate precipitation, column chromatography, electrophoresis, centrifugation, crystallization, and others. See, e.g., Ausubel, et al. (1987 and periodic supplements); Deutscher (1990) “Guide to Protein Purification,” Methods in Enzymology vol. 182, and other volumes in this series; Coligan, et al. (1995 and supplements) Current Protocols in Protein Science John Wiley and Sons, New York, N.Y.; P. Matsudaira (ed.) (1993) A Practical Guide to Protein and Peptide Purification for Microsequencing, Academic Press, San Diego, Calif.; and manufacturer's literature on use of protein purification products, e.g., Pharmacia, Piscataway, N.J., or Bio-Rad, Richmond, Calif. Combination with recombinant techniques allow fusion to appropriate segments (epitope tags), e.g., to a FLAG sequence or an equivalent which can be fused, e.g., via a protease-removable sequence. See, e.g., Hochuli (1989) Chemische Industrie 12:69-70; Hochuli (1990) “Purification of Recombinant Proteins with Metal Chelate Absorbent” in Setlow (ed.) Genetic Engineering, Principle and Methods 12:87-98, Plenum Press, NY; and Crowe, et al. (1992) QIAexpress: The High Level Expression and Protein Purification System QUIAGEN, Inc., Chatsworth, Calif. Standard immunological techniques are described, e.g., in Hertzenberg, et al. (eds. 1996) Weir's Hanbook of Experimental Immunology vols. 1-4, Blackwell Science; Coligan (1991) Current Protocols in Immunology Wiley/Greene, NY; and Methods in Enzymology volumes. 70, 73, 74, 84, 92, 93, 108, 116, 121, 132,150, 162, and 163. Assays for neural cell biological activities are described, e.g., in Wouterlood (ed. 1995) Neuroscience Protocols modules 10, Elsevier; Methods in Neurosciences Academic Press; and Neuromethods Humana Press, Totowa, N.J. Methodology of developmental systems is described, e.g., in Meisami (ed.) Handbook of Human Growth and Developmental Biology CRC Press; and Chrispeels (ed.) Molecular Techniques and Approaches in Developmental Biology Interscience. FACS analyses are described in Melamed, et al. (1990) Flow Cytometry and Sorting Wiley-Liss, Inc., New York, N.Y.; Shapiro (1988) Practical Flow Cytometry Liss, New York, N.Y.; and Robinson, et al. (1993) Handbook of Flow Cytometry Methods Wiley-Liss, New York, N.Y.

### Example 1

### Isolation of LP Clones

Standard methods are used to isolate fill length genes from a cDNA library made from an appropriate source, e.g., human cells. The appropriate sequence is selected, and hybridization at high stringency conditions is performed to find a full length corresponding gene using standard techniques. The full length, or appropriate fragments, of human genes are used to isolate a corresponding monkey or other primate gene. Preferably, a full length coding sequence is used for hybridization. Similar source materials as indicated above are used to isolate natural genes, including genetic, polymorphic, allelic, or strain variants. Other species variants are also isolated using similar methods. With a positive clone, the coding sequence is inserted into an appropriate expression vector. This may be in a vector specifically selected for a prokaryote, yeast, insect, or higher vertebrate, e.g., mammalian expression system. Standard methods are applied to produce the gene product, preferably as a soluble secreted molecule, but will, in certain instances, also be made as an intracellular protein. Intracellular proteins typically require cell lysis to recover the protein, and insoluble inclusion bodies are a common starting material for further purification. With a clone encoding a vertebrate LP protein, recombinant production means are used, although natural forms may be purified from appropriate sources. The protein product is purified by standard methods of protein purification, in certain cases, e.g., coupled with immunoaffinity methods. Immunoaffinity methods are used either as a purification step, as described above, or as a detection assay to determine the separation properties of the protein.

Preferably, the protein is secreted into the medium, and the soluble product is purified from the medium in a soluble form. Alternatively, as described above, inclusion bodies from prokaryotic expression systems are a useful source of material. Typically, the insoluble protein is solubilized from the inclusion bodies and refolded using standard methods. Purification methods are developed as described herein. The product of the purification method described above is characterized to determine many structural features. Standard physical methods are applied, e.g., amino acid analysis and protein sequencing. The resulting protein is subjected to CD spectroscopy and other spectroscopic methods, e.g., NMR, ESR, mass spectroscopy, etc. The product is characterized to determine its molecular form and size, e.g., using gel chromatography and similar techniques. Understanding of the chromatographic properties will lead to more gentle or efficient purification methods. Prediction of glycosylation sites may be made, e.g., as reported in Hansen, et al. (1995) Biochem. J. 308:801-813. The purified protein is also be used to identify other binding partners of an LP of the invention as described, e.g., in Fields and Song (1989) Nature 340:245-246.

### Example 2

### Tissue Distribution of an LP Polynucleotide

Tissue distribution of mRNA expression of a polynucleotide of the present invention (or fragment thereof) is determined using protocols for Northern blot analysis, described (among others) by, e.g., Sarnbrook, et al. For example, a cDNA probe produced using common techniques is labeled with p³² using the Rediprime™ DNA labeling system (Amersham Life Science), according to manufacturer's instructions. After labeling, the probe is purified using CHROMA SPIN-100™ column (Clontech Laboratories, Inc.), according to manufacturer's protocol number PT1200-1. The purified, labeled probe is then used to examine various human tissues for mRNA expression. Multiple Tissue Northern (MTN) blots containing various human tissues (H) or human immune system tissues (IM) (Clontech) are examined with the labeled probe using Express Hyb™ hybridization solution (Clontech) according to manufacturer's protocol number PT1190-1. After hybridization and washing, blots are mounted and exposed to film (overnight at −70° C.), and the films are subsequently developed according to standard procedures.

### Example 3

### Chromosomal Mapping of an LP Polynucleotide

An oligonucleotide primer set is designed according to the sequence at the 5′ end of a SEQ ID NO:X identified sequence. This primer preferably spans about 100 nucleotides. This primer set is then used in a polymerase chain reaction under the following set of conditions: 30 seconds, 95° C.; 1 minute, 56° C.; 1 minute, 70° C. This cycle is repeated 32 times followed by one 5-minute cycle at 70° C. Human, mouse, and hamster DNA is used as template in addition to a somatic cell hybrid panel containing individual chromosomes or chromosome fragments (Bios, Inc). The reaction is analyzed on either 8% polyacrylamide gels or 3.5% agarose gels. Chromosome mapping is determined by the presence of an approximately 100 bp PCR fragment in a particular somatic cell hybrid.

### Example 4

### Production of a Secreted LP Protein for a High-Throughput Screening Assay

The following protocol produces a supernatant containing an LP polypeptide (or fragment thereof) to be tested. This supernatant can then be used in a variety of screening assays (such as, e.g., those taught herein). First, dilute Poly-D-Lysine (644 587 Boehringer-Mannheim) stock solution (1 mg/ml in PBS) 1:20 in PBS (w/o calcium or magnesium 17-5 16 F Biowhittaket) to obtain a working stock solution of 50 ug/ml. Add 200 ul of this solution to each well (24-well plates) and incubate (RT for 20 min). Distribute the solution over each well (a 12-channel pipetter may be used with tips on every other channel). Aspirate off the Poly-D-Lysine solution and rinse with 1 ml PBS (Phosphate Buffered Saline). The PBS should remain in the well until just before plating the cells and plates may be coated (up to two weeks in advance) with poly-lysine. Plate 2933: cells (do not carry cells past P+20) at 2×10⁵ cells/well in 0.5 ml DMEM (Dulbecco's Modified Eagle Medium) (with 4.5 G/L glucose and L-glutamine5 (12-604 F Biowhittaker))/10% heat inactivated FBS (14-503 F Biowhittaker)/1× Pinstripe (17-602E Biowhittaker). Let the cells grow overnight.

The next day, mix in a sterile solution basin: 300 ul Lipofectamine (18324-012 Gibco; BRL) and 5 ml Optimem I (31985070 Gibco; BRL) per 96-well plate. With a small volume multi-channel pipetter, aliquot approximately 2 ug of an expression vector containing an LP polynucleotide insert of the invention, produced by any art known methods or as taught herein, into an appropriately labeled 96-well round-bottom plate. With a multi-channel pipetter, add 50 μl of the Lipofectamine/Optimem I mixture to each well. Pipette up and down gently to mix. Incubate at RT for 15-45 minutes. After about 20 minutes, use a multi-channel pipetter to add 150 μl of Optimem I to each well. As a control, transfect one plate of vector DNA lacking an insert with each set of transfections.

Preferably, transfections should be performed by splitting the following tasks between two individuals to reduce the time, and to insure that the cells do not spend too much time in PBS. First, person A aspirates off the media from four 24-well plates of cells, and then person B rinses each well with 0.5-1 ml of PBS. Person A then aspirates off the PBS rinse, and person B (using a 12-channel pipetter with tips on every other channel) adds 200 μl of DNA/Lipofectamine/Optimem I complex first to the odd wells, then to the even wells (of each row on the 24-well plates). Incubate at 37° C. for 6 hours. While cells are incubating, prepare appropriate media, either 1% BSA in DMEM with 1× penstrep, or CHO-5 media (116.6 mg/L of CaCl₂ (anhyd); 0.00130 mg/L CuSO₄—5H₂O; 0.050 mg/L of Fe(NO₃)₃—9H₂O; 0.417 mg/L of FeSO₄—7H₂O; 311.80 mg/L of KCl; 28.64 mg/L of MgCl₂; 48.84 mg/L of MgSO₄; 6995.50 mg/L of NaCl; 2400.0 mg/L of NaHCO₃; 62.50 mg/L of NaH₂PO₄—H₂O; 71.02 mg/L of Na₂HPO₄; 0.4320 mg/L of ZnSO₄—7H₂O; 0.002 mg/L of Arachidonic Acid; 1.022 mg/L of Cholesterol; 0.070 mg/L of DL-alpha-Tocopherol-Acetate; 0.0520 mg/L of Linoleic Acid; 0.010 mg/L of Linolenic Acid; 0.010 mg/L of Myristic Acid; 0.010 mg/L of Oleic Acid; 0.010 mg/L of Palmitric Acid; 0.010 mg/L of Palmitic Acid; 100 mg/L of Pluronic F-68; 0.010 mg/L of Stearic Acid; 2.20 mg/L of Tween 80; 4551 mg/L of D-Glucose; 130.85 mg/ml of L-Alanine; 147.50 mg/ml of L-Arginine-HCL; 7.50 mg/ml of L-Asparagine-H₂O; 6.65 mg/ml of L-Aspartic Acid; 29.56 mg/ml of L-Cystine-2HCL-H₂O; 31.29 mg/ml of L-Cystine-2HCL; 7.35 mg/ml of L-Glutamic Acid; 365.0 mg/ml of L-Glutamine; 18.75 mg/ml of Glycine; 52.48 mg/ml of L-Histidine-HCL-H₂O; 106.97 mg/ml of L-Isoleucine; 111.45 mg/ml of L-Leucine; 163.75 mg/ml of L-Lysine HCL; 32.34 mg/ml of L-Methionine; 68.48 mg/ml of L-Phenylalanine; 40.0 mg/ml of L-Proline; 26.25 mg/ml of L-Serine; 101.05 mg/ml of L-Threonine; 19.22 mg/ml of L-Tryptophan; 91.79 mg/ml of L-Tryrosine-2Na-2H₂O; 99.65 mg/ml of L-Valine; 0.0035 mg/L of Biotin; 3.24 mg/L of D-Ca Pantothenate; 11.78 mg/L of Choline Chloride; 4.65 mg/L of Folic Acid; 15.60 mg/L of i-Inositol; 3.02 mg/L of Niacinamide; 3.0 mg/L of Pyridoxal HCL; 0.031 mg/L of Pyridoxine HCL; 0.319 mg/L of Riboflavin; 3.17 mg/L of Thiamine HCL; 0.365 mg/L of Thymidine; and 0.680 mg/L of Vitamin B₁₂; 25 mM of HEPES Buffer; 2.39 mg/L of Na Hypoxanthine; 0.105 mg/L of Lipoic Acid; 0.081 mg/L of Sodium Putrescine-2HCL; 55.0 mg/L of Sodium Pyruvate; 0.0067 mg/L of Sodium Selenite; 20 uM of Ethanolamine; 0.122 mg/L of Ferric Citrate; 41.70 mg/L of Methyl-B-Cyclodextrin complexed with Linoleic Acid; 33.33 mg/L of Methyl-B-Cyclodextrin complexed with Oleic Acid; and 10 mg/L of Methyl-B-Cyclodextrin complexed with Retinal) with 2 mm glutamine, and 1× penstrep (BSA (81-068-3 Bayer) 100 gm dissolved in 1L DMEM for a 10% BSA stock solution). Filter the media and collect 50 ul for endotoxin assay in 15 ml polystyrene conical.

The transfection reaction is terminated, preferably by splitting tasks (as above) at the end of the incubation period. Person A aspirates off the transfection media, while person B adds 1.5 ml appropriate media to each well. Incubate at 37° C. for 45 or 72 hours depending on the media used (1% BSA for 45 hours or CHO-5 for 72 hours). On day four, using a 300 ul multichannel pipetter, aliquot 600 μl in one 1 ml deep well plate and the remaining supernatant into a 2 ml deep well. The supernatants from each well can then be used in an assay taught herein. It is specifically understood that when activity is obtained in an assay described herein using a supernatant, the activity originates either from the polypeptide (or fragment thereof) directly (such as, e.g., from a secreted protein or fragment thereof) or by the polypeptide (or fragment thereof) inducing expression of another protein(s), which is/are then released into the supernatant. Thus, the invention provides a method of identifying a polypeptide (or fragment thereof) in a supernatant characterized by an activity in a particular assay taught herein.

### Example 5

### Construction of a GAS Reporter Construct

One signal transduction pathway involved in cellular differentiation and proliferation is a Jaks-STATS pathway. Activated proteins in a Jaks-STATS pathway have been shown to bind to gamma activation site “GAS” elements or interferon-sensitive responsive element (“ISRE”), which are located, e.g., in the promoter region of many genes. Typically, binding, e.g., by a protein, to such an element alters expression of an associated gene. GAS and ISRE elements are recognized by a class of transcription factors called Signal Transducers and Activators of Transcription, or “STATS.” The Stat1 and Stat3 members of the STATS family are present in many cell types, (as is Stat2) probably, because the response to IFN-alpha is widespread. Stat4, however, is more restricted to particular cell types though, it has been found in T helper class I cells after their treatment with IL-12. Stat 5 (originally designated mammary growth factor) has been found at higher concentrations in cells besides breast cells, e.g., myeloid cells. Stat 5 is activated in tissue culture cells by many cytokines.

After tyrosine phosphorylation (by kinases known as the Janus Kinase Family or “Jaks”), members of the STATS family typically translocate from the cytoplasm to the nucleus of the cell. Jaks represent a distinct family of soluble tyrosine kinases and include, e.g., Tyk2, Jak1, Jak2, and Jak3. These Jak kinases display significant sequence similarity to each other and, generally, are catalytically inactive in resting cells. However, Jaks are catalytically activated by a wide range of receptors (summarized in the Table below, adapted from Schidler and Darnell (1995) Ann. Rev. Biochem. 64:621-51). One cytokine receptor family, which is capable of activating a Jak, is divided into two groups (Class 1 and 2). Class 1 includes, e.g., receptors for IL-2, IL-3, IL-4, IL-6, IL-7, IL-9, IL-11, IL-12, IL-15, Epo, PRL, GH, G-CSF, GM-CSF, LIF, CNTF, and thrombopoietin; while Class 2 includes, e.g., IFN-a, IFN-g, and IL-10. The Class 1 receptors share a conserved cysteine motif (a set of four conserved cysteines and one tryptophan) and a WSXWS motif (a membrane proximal region encoding Trp-Ser-Xxx-Trp-Ser). Thus, after a ligand binds a receptor, Jaks are typically activated and, in turn, subsequently activate STATS, which translocate and bind to GAS transcriptional elements (located in the nucleus of the cell). This entire process of sequential activation is encompassed in a typical Jaks-STATS signal transduction pathway. Therefore, activation of a Jaks-STATS pathway (reflected by binding of a GAS or 1SRE element) is used to indicate that an LP polypeptide (or fragment thereof) is involved in the proliferation and/or differentiation of a cell. For instance, growth factors and cytokines are examples of proteins that are known to activate a Jaks-STATS pathway. Consequently, by using a GAS element linked to a reporter molecule, an activator of a Jaks-STATS pathway is identified.

| | | JAKS | | | | |
|---|---|---|---|---|---|---|
| Ligand | tyk2 | Jak1 | Jak2 | Jak3 | STATS | GAS(elements) or ISRE |
| IFN family | | | | | | |
| IFN-a/B | + | + | − | − | 1, 2, 3 | ISRE |
| IFN-G | | + | + | − | 1 | GAS (IRF1 > Lys6 > IFP) |
| IL-10 | + | ? | ? | − | 1, 3 | |
| gp130 family | | | | | | |
| IL-6 (Pleiotrophic) | + | + | + | ? | 1, 3 | GAS (IRF1 > Lys6 > IFP) |
| IL-11 (Pleiotrophic) | ? | + | ? | ? | 1, 3 | |
| OnM (Pleiotrophic) | ? | + | + | ? | 1, 3 | |
| LIF (Pleiotrophic) | ? | + | + | ? | 1, 3 | |
| CNTF (Pleiotrophic) | −/+ | + | + | ? | 1, 3 | |
| G-CSF (Pleiotrophic) | ? | + | ? | ? | 1, 3 | |
| IL-12 (Pleiotrophic) | + | − | + | + | 1, 3 | |
| g-C family | | | | | | |
| IL-2 (lymphocytes) | − | + | − | + | 1, 3, 5 | GAS |
| IL-4 (lymph/myeloid) | − | + | − | + | 6 | GAS (IRF1 = IFP >> Ly6) (IgH) |
| IL-7 (lymphocytes) | − | + | − | + | 5 | GAS |
| IL-9 (lymphocytes) | − | + | − | + | 5 | GAS |
| IL-13 (lymphocyte) | − | + | ? | ? | 6 | GAS |
| IL-15 | ? | + | ? | + | 5 | GAS |
| gp140 family | | | | | | |
| IL-3 (myeloid) | − | − | + | − | 5 | GAS (IRF1 > IFP >> Ly6) |
| IL-5 (myeloid) | − | − | + | − | 5 | GAS |
| GM-CSF (myeloid) | − | − | + | − | 5 | GAS |
| Growth hormone family | | | | | | |
| GH | ? | − | + | − | 5 | |
| PRL | ? | +/− | + | − | 1, 3, 5 | |
| EPO | ? | − | + | − | 5 | GAS (B − CAS > IRF1 = IFP >> Ly6) |
| Receptor Tyrosine | | | | | | |
| Kinases | | | | | | |
| EGF | ? | + | + | − | 1, 3 | GAS (IRF1) |
| PDGF | ? | + | + | − | 1, 3 | |
| CSF-1 | ? | + | + | − | 1, 3 | GAS (not IRF1) |

To construct a synthetic GAS containing promoter element, like that described in an assays taught herein, a PCR based strategy is employed to generate a GAS-SV40 promoter sequence. The 5′ primer contains four tandem copies of the GAS binding site found in the IRF1 promoter, which has previously been shown to bind STATS after induction by a range of cytokines (see, e.g., Rothman, et al. (1994) Immunity 1:457-468). Although, however, it is possible to use other GAS or ISRE elements. The 5′ primer also contains 18 bp of sequence complementary to the SV40 early promoter sequence and is flanked with an XhoI site. The sequence of the 5′ primer is:

| | |
|---|---|
| 5′:GCGCCTCGAGATTTCCCCGAAATCTAGATTTCCC | (SEQ ID NO:9) |
| CGAAATGATTTCCCCGAAATGATTTCCCCGAAATATC | |
| TGCCATCTCAATTAG:3′ | |

The downstream primer, which is complementary to the SV40 promoter and is flanked with a Hind III site, is:

5′:GCGGCAAGCTTTTTGCAAAGCCTAGGC:3′ (SEQ ID NO:10). PCR amplification is performed using the SV40 promoter template present in a B-gal:promoter plasmid (Clontech). The resulting PCR fragment is digested with XhoI/Hind III and subcloned into BLSK2-(Stratagene). Sequencing with forward and reverse primers confirms that the insert contains the following sequence:

| | |
|---|---|
| 5′:CTCGAGATTTCCCCGAAATCTAGATTTCCCCGA | (SEQ ID NO:11) |
| AATGATTCCCCGAAATGATTTCCCCGAAATATCTGC | |
| CATCTCAATTAGTCAGCAACCATAGTCCCGCCCCTA | |
| ACTCCGCCCATCCCGCCCCTAACTCCGCCCAGTTCC | |
| GCCCATTCTCCGCCCCATGGCTGACTAATTTTTTTT | |
| ATTTATGCAGAGGCCGAGGCCGCCTCGGCCTCTGAG | |
| CTATTCCAGAAGTAGTCAGGAGGCTTTTTTGGAGGC | |
| CTAGGCTTTTGCAAAAAGCTT:3′ | |

With this GAS promoter element linked to the SV40 promoter, a GAS:SEAP2 reporter construct is next engineered. Here, the reporter molecule is a secreted alkaline phosphatase (SEAP). Clearly, in this or in any of the other assays described herein, any applicable reporter molecule is used instead of SEAP without undue experimentation. For example, using art known methods, such as, e.g., without limitation, chloramphenicol acetyltransferase (CAT), luciferase, alkaline phosphatase, B-galactosidase, green fluorescent protein (GFP), or any protein (detectable by an antibody or detectable binding partner) could be substituted for SEAP. Once the above sequence is confirmed, the synthetic GAS-SV40 promoter element is subcloned into a pSEAP-Promoter vector (Clontech) using HindIII and XhoI. This, effectively, replaces the SV40 promoter with the amplified GAS:SV40 promoter element to create a GAS-SEAP vector. However, since the resulting GAS-SEAP vector does not contain a neomycin resistance gene it is not a preferred embodiment for use in mammalian expression systems. To generate stable mammalian cell lines that express a GAS-SEAP reporter, the GAS-SEAP cassette is removed (using Sal1 and Not1) from the GAS-SEAP vector and inserted into a backbone vector containing a neomycin resistance gene, such as, e.g., pGFP-1 (Clontech), using these restriction sites in the multiple cloning site, to create a GAS-SEAP/Nco vector. Once the GAS-SEAP/Neo vector is transfected into a mammalian cell, it can also be used as a reporter molecule for GAS binding as taught in an assay as described herein.

Similar constructs is made using the above description and replacing GAS with a different promoter sequence. For example, construction of reporter-molecules containing NFK-B and EGR promoter sequences are applicable. Additionally, however, many other promoters is substituted using a protocols described herein, e.g., SRE, IL-2, NFAT, or Osteocalcin promoters is substituted, alone or in combination with another (e.g., GAS/NF-KB/EGR, GAS/NF-KB, Il-2/NFAT, or NF-KB/GAS). Similarly, other cell lines is used to test reporter construct activity, such as, e.g., without limitation, HELA (epithelial), HUVEC (endothelial), Reh (B-cell), Saos-2 (osteoblast), HUVAC (aortic), or Cardiomyocyte cell lines. Alternatively, testing whether an LP polypeptide (or fragment thereof) is involved in a JAK/STATs signal transduction pathway can be performed (without undue experimentation) by adopting a method as described, e.g., in Ho, et al. (1995) Mol. Cell. Biol. 15:5043-5-53. Furthermore, it may be possible to test the JAK/STATs signal transduction pathway for blockage using an LP composition of the invention. Additionally, standard methods exist for testing whether an LP polypeptide (or fragment thereof) of the invention is involved in a STAT signaling pathway (e.g., such methods are described, e.g., in Starr, et al. (1997) Nature 387:917-921; Endo, et al. (1997) Nature 387:921-924; and Naka, et al. Nature 387:924-929 and can be employed here without undue experimentation).

### Example 6

### High-Throughput Screening Assay for T-cell Activity.

The following protocol is used to assess T-cell activity by identifying factors and/or determining whether a supernate (described herein) containing an LP polypeptide (or fragment thereof modulates the proliferation and/or differentiation of a T-cell. T-cell activity is assessed using a GAS/SEAP/Neo construct. Thus, a factor that increases SEAP activity indicates an ability to activate a Jaks-STATS signal transduction pathway. One type of T-cell used in this assay is, e.g., a Jurkat T-cell (ATCC Accession No. TIB-152), although other cells can also be used such as, e.g., without limitation, Molt-3 cells (ATCC Accession No. CRL-1552) or Molt-4 cells (ATCC Accession No. CRL-1582).

Jurkat T-cells are lymphoblastic CD4+ Th1 helper cells. To generate stable cell lines, approximately 2 million Jurkat cells are transfected with a GAS-SEAP/Neo vector using DMRIE-C (Life Technologies) in a transfection procedure as described below. Transfected cells are seeded to a density of approximately 20,000 cells per well and any resulting transfectant (resistant to 1 mg/ml genticin) is subsequently selected. Resistant colonies are then expanded and tested for their response to increasing concentrations of interferon gamma. The dose response of a selected clone is then established. Typically, the following method yields a number of cells sufficient for 75 wells (each containing approximately 200 ul of cells). The method can be modified easily (e.g., it can either be scaled up or performed in multiples to generate sufficient numbers of cells for multiple 96 well plates). Jurkat cells are maintained in RPM1+10% serum with 1% Pen-Strep. Combine 2.5 mls of OPTI-MEM (LifeTechnologies) with 10 ug of plasmid DNA in a T25 flask. Add 2.5 ml OPTI-MEM containing 50 μl of DMRIE-C and incubate (RT) for 15-45 min. During incubation, determine the cell concentration, spin down the required number of cells (˜10⁷ per transfection), and resuspend in OPTI-MEM to a final concentration of 10⁷ cells/ml. Then add 1 ml of 1×10⁷ cells in OPTI-MEM to a T25 flask and incubate at 37° C. for 6 hrs. After incubation, add 10 ml of RPMI+15% serum.

The Jurkat:GAS-SEAP stable reporter lines are maintained in RPMI+10% serum, 1 mg/ml Genticin, and 1% Pen-Strep. These cells are treated with supernatants containing an LP polypeptide (or fragment thereof) and/or an induced polypeptide of the invention (or fragment thereof) as produced by a protocol taught herein. On the day of treatment with the supernatant, the cells should be washed, and re-suspended in fresh RPM1+10% serum to a density of 500,000 cells per ml. The exact number of cells required depends on the number of supernatants being screened. For one 96 well plate, approximately 10 million cells are required (for 10 plates, 100 million cells). Transfer the cells to a triangular reservoir boat, to dispense the cells into a 96 well dish, using a 12 channel pipette to transfer 200 ul of cells into each well (therefore adding 100,000 cells per well). After all the plates have been seeded, 50 ul of the supernatants are transferred directly from the 96 well plate containing the supernatants into each well using a 12 channel pipette. In addition, a dose of exogenous interferon gamma (0.1 ng, 1.0 ng, 10.0 ng) is added to wells H9, H10, and H11 to serve as additional positive controls for the assay. The 96 well dishes containing Jurkat cells treated with supernatants are placed in an incubator for 48 hrs (note: this time is variable between 48-72 hrs). Then, 35 ul samples from each well are transferred to an opaque 96 well plate using a 12-channel pipette. The opaque plates should be covered (using cellophane), and stored at −20° C. until SEAP assays are performed as described herein or known in the art. Plates containing the remaining treated cells are placed at 4° C., and can serve as a source of material for repeated assays on a specific well if so desired. As a positive control, 100 Unit/ml interferon gamma is used to activate Jurkat T cells. Typically, a 30-fold induction or greater is observed in positive control wells. As will be apparent to those of ordinary skill in the art, the above protocol may be used in the generation of both transient, as well as, stably transfected cells.

### Example 7

### High-Throughout Screening Assay to Identify Myeloid Activity

The following protocol is used to assess myeloid activity by determining whether an LP polypeptide (or fragment thereof) mediates the proliferation, and/or differentiation of a myeloid cell. Myeloid cell activity is assessed using a GAS/SEAP/Neo construct as described herein. Thus, a factor that increases SEAP activity indicates the ability to activate a Jaks-STATS signal transduction pathway. A typical myeloid cell used in such an assay is U937 (a pre-monocyte cell line) although, other myeloid cells can be used, such as, e.g., without limitation, TF-1, HL60, or KG1.

To transiently transfect U937 cells with a GAS/SEAP/Neo construct a DEAE-Dextran method is used (Kharbanda, et al. (1994) Cell Growth & Differentiation, 5: 259-265). First, 2×10⁷ U937 cells are harvested and then washed with PBS. Typically, U937 cells are grown in RPMI 1640 medium containing 10% heat-inactivated fetal bovine serum (FBS) supplemented with 100 units/ml penicillin, and 100 mg/ml streptomycin. Next, suspend the cells in 1 ml of 20 mM Tris-HCl (pH 7.4) buffer containing 0.5 mg/ml DEAE-Dextran, 8 ug GAS-SEAP2 plasmid DNA, 140 mM NaCl, 5 mM KCl, 375 uM Na₂HPO₄—7H₂O, 1 mM MgCl₂, and 675 uM CaCl₂. Incubate at 37° C. for 45 min. Wash the cells with RPMI 1640 medium containing 10% FBS and then resuspend in 10 ml complete medium and incubate at 37° C. for 36 hr. The GAS-SEAP/U937 stable cells are obtained by growing the cells in 400 ug/ml G418. The G418-free medium is used for routine growth but periodically (every one to two months), the cells should be re-grown in 400 ug/ml G418 for several passages. These cells are tested by harvesting 1×10⁸ cells (approximately enough for ten 96-well plate assays) and then washing with PBS. Suspend the cells in 200 ml of the above described growth medium to a final density of 5×10⁵ cells/ml. Plate 200 ul cells/well in a 96-well plate (or 1×10⁵ cells/well). Add 50 ul of supernatant as described herein then, incubate at 37° C. for 48 to 72 hr. As a positive control, 100 Unit/ml interferon gamma is used to activate U937 cells. Typically, a 30-fold induction is observed in wells containing the positive controls. Assay a supernatant according to a SEAP protocol taught herein or art-known.

### Example 8

### High-Throughput Screening Assay to Identify Neuronal Activity.

When cells undergo differentiation and proliferation, genes are activated through many different signal transduction pathways. One such gene, EGR1 (early growth response gene 1), is induced in various tissues and cell types upon activation. The promoter of EGR1 is responsible for such induction. The activation of particular cells is assessed using the EGR1 promoter linked to a reporter molecule. Specifically, the following protocol is used to assess neuronal activity in a PC12 cell (rat phenochromocytoma cell). PC12 cells show proliferative and/or differentiative responses (e.g., EGR1 expression) upon activation by a number of stimulators, such as, e.g., TPA (tetradecanoyl phorbol acetate), NGF (nerve growth factor), and EGF (epidermal growth factor). Thus, PC12 cells (stably transfected with a construct comprising an EGR promoter operably linked to SEAP reporter) are used in an assay to determine activation of a neuronal cell by an LP polypeptide (or fragment thereof). A EGR/SEAP reporter construct is created as follows: the EGR-I promoter sequence (−633 to +1; Sakamoto, et al. (1991) Oncogene 6:867-871) is PCR amplified from human genomic DNA using the following primers:

| 5′ GCGCTCGAGGGATGACAGCGATAGAACCCCGG- | (SEQ ID NO:12) |
|---|---|
| 3′ | |
| 5′ GCGAAGCTTCGCGACTCCCCGGATCCGCCTC- | (SEQ ID NO:13) |
| 3′ | |

Using a GAS:SEAP/Neo vector (described herein), the EGR1 amplified product is inserted into this vector by linearizing the GAS:SEAP/Neo vector (XhoI/HindIII) and removing the GAS/SV40 stuffer. The EGRI amplified product is restricted using these same enzymes (XhoI/HindIII). Then, the EGR1 promoter is ligated to the vector. To prepare 96 well-plates for cell culture, add two mls of a coating solution (dilute (1:30) collagen type I (Upstate Biotech Inc. Cat#08-115) in filter sterilized 30% ethanol) per one 10 cm plate or 50 ml per well of the 96-well plate, and then air dry for 2 hr. Routinely grow PC12 cells on pre-coated 10 cm tissue culture dishes using RPMI-1640 medium (Bio Whittaker) containing 10% horse serum (JRH BIOSCIENCES, Cat. #12449-78P), 5% heat-inactivated fetal bovine serum (FBS) supplemented with 100 units/ml penicillin and 100 ug/ml streptomycin. Every three to four days, perform a one to four split of the cells. Cells are removed from a plate by scraping and re-suspending (typically, by pipetting up and down more than 15 times). To transfect an EGR/SEAP/Neo construct into PC12 cells use the Lipofectamine protocol taught herein. Produce stable EGR-SEAP/PC12 cells by growing transfected cells in 300 ug/ml G418. The G418-free medium is used for routine growth but periodically (every one to two months), the PC12 cells should be re-grown in 300 ug/ml G41830 for several passages.

To assay a PC12 cell for neuronal activity, a 10 cm plate (containing cells that are around 70 to 80% confluent) is screened by removing the old medium and washing the cells once with PBS. Then, starve the cells overnight in low serum medium (RPMI-1640 containing 1% horse serum, and 0.5% FBS with antibiotics). The next morning, remove the medium, and wash the cells with PBS. Scrape off the cells from the plate and suspend them thoroughly in 2 ml low serum medium. Count the cell number, and add more low serum medium to achieve a final cell density of approximately 5×10⁵ cells/ml. Add 200 ul of the cell suspension to each well of 96-well plate (equivalent to 1×10⁵ cells/well). Add 50 ul of supernatant and store at 37° C. for 48 to 72 hr. As a positive control, use a growth factor known to activate PC12 cells through EGR, such as, e.g., 50 ng/ul of Neuronal Growth Factor (NGF). Typically, a fifty-fold or greater induction of SEAP is achieved with a positive control. Assay the supernatant according to a SEAP method described herein.

### Example 9

### High-Throughput Screening Assay to Identify T-cell Activity

NF-KB (Nuclear Factor kappa B) is a transcription factor activated by a wide variety of agents including, e.g., inflammatory cytokines (such as, e.g., IL-1, TNF, CD30, CD40, lymphotoxin-alpha, and lymphotoxin-beta); LPS, thrombin; and by expression of certain viral gene products. As a transcription factor, NF-KB typically regulates: the expression of genes involved in immune cell activation; the control of apoptosis (NF-KB appears to shield cells from apoptosis); the development of B-cells or T-cells; anti-viral or antimicrobial responses; and multiple stress responses. Under non-stimulating conditions, NF-KB is retained in the cytoplasm with I-KB (Inhibitor KB). However, upon proper stimulation, I-KB is phosphorylated and degraded, leading to NF-KB translocating into the nucleus of the cell, thereby activating transcription of specific target genes, such as, e.g., IL-2, IL-6, GM-CSF, ICAM-I, and Class 1 MHC. Due to NF-KB's role in transcriptional activation and its ability to respond to a range of stimuli, reporter constructs utilizing the NF-KB promoter element are useful in screening a supernatant produced as described herein. Activators or inhibitors of NF-KB are useful in treating diseases, e.g., inhibitors of NF-KB is used to treat diseases, syndromes, conditions, etc., related to the acute or chronic activation of NF-KB, such as, e.g., rheumatoid arthritis. To construct a vector comprising a NF-KB promoter element, a PCR based strategy is employed. The upstream primer should contain four tandem copies of the NF-KB binding site (GGGGACTTTCCC; SEQ ID NO:14), 18 bp of sequence that is complementary to the 5′ end of the SV40 early promoter sequence, and that is flanked by the XhoI site:

| | |
|---|---|
| 5′:GCGGCCTCGAGGGGACTTTCCCGGGGACTTTCC | (SEQ ID NO:15) |
| GGGGATCCGGGACTTTCCATCCTGCCATCTCAATTA | |
| G:3′ | |

The downstream primer is complementary to the 3′ end of the SV40 promoter and is flanked by the Hind III site:

5′:GCGGCAAGCTTTTTGCAAAGCCTAGGC:3′ (SEQ ID NO:16).

A PCR amplification is performed using the SV40 promoter template present in a pB-gal promoter plasmid (Clontech). The resulting PCR fragment is digested with XhoI, and Hind III, then subcloned into BLSK2 (Stratagene). Sequencing with the T7, and T3 primers should confirm that the insert contains the following sequence:

| | |
|---|---|
| 5′:CTCGAGGGGACTTTCCCGGGGACTTTCCGGGGA | (SEQ ID NO:17) |
| CTTTCCGGGACTTTCCATCTGCCATCTCAATTAGTC | |
| AGCAACCATAGTCCCGCCCCTAACTCCGCCCATCCC | |
| GCCCCTAACTCCGCCCAGTTCCGCCCATTCTCCGCC | |
| CCATGGCTGACTAATTTTTTTTATTTATGCAGAGGC | |
| CGAGGCCGCCTCGGCCTCTGAGCTATTCCAGAAGTA | |
| GTGAGGAGCCTTTTTTGGAGGCCTAGGCTTTTGCAA | |
| AAAGCTT:3′ | |

Next, replace the SV40 minimal promoter element present in the pSEAP2-promoter plasmid (Clontech) with the NF-KB/SV40 fragment using XhoI, and HindIII (note, this vector does not contain a neomycin resistance gene, and therefore, is not preferred for use in a mammalian expression system). To generate a stable mammalian cell line, the NF-KB/SV40/SEAP construct is removed from the above NF-KB/SEAP vector using restriction enzymes SalI, and NotI, and then inserted into a vector having neomycin resistance. For example, the NF-KB/SV40/SEAP construct is inserted into pGFP-1 (Clontech), replacing the GFP gene, after restricting pGFP-1 with SalI, and NotI. After a NF-KB/SV40/SEAP/Neo vector is established, then stable Jurkat T-cells are created and maintained as described herein. Similarly, a method for assaying supernatants with these stable Jurkat T-cells is used as previously described herein. As a positive control, exogenous TNF alpha (at, e.g., concentration of 0.1 ng, 1.0 ng, and 10 ng) is added to a control well (e.g., wells H9, H10, and H11). Typically, a 5- to 10-fold activation is observed in the control.

### Example 10

### Assay for Reporter Activity (e.g., SEAP)

As a reporter molecule for the assays taught herein, SEAP activity is assessed using the Tropix Phospho-light Kit (Cat. BP-400) according to the following general procedure. The Tropix Phospho-light Kit supplies the dilution, assay, and reaction buffers described below. Prime a dispenser with the 2.5× dilution buffer and dispense 15 ul of 2.5× dilution buffer into Optiplates containing 35 ul of a supernatant. Seal the plates with a plastic sealer and incubate at 65° C. for 30 min. Separate the Optiplates to avoid uneven heating. Cool the samples, until they are maintained at RT for 15 minutes. Empty the dispenser and prime with the assay buffer. Add 50 ml assay buffer and incubate (5 min. at RT). Empty the dispenser and prime with the reaction buffer (see the table below). Add 50 ul reaction buffer and incubate (20 min. at RT). Since the intensity of the chemiluminescent signal is time dependent, and it takes about 10 minutes to read five plates on luminometer, treat five plates at each time and start the second set 10 minutes later. Read the relative light unit in the luminometer using the H12 location on the plate as blank, and print the results. An increase in chemiluminescence indicates reporter activity.

### Reaction Buffer Formlation

| | Rxn buffer | |
|---|---|---|
| # of plates | diluent (ml) | CSPD (ml) |
| 10 | 60 | 3 |
| 11 | 65 | 3.25 |
| 12 | 70 | 3.5 |
| 13 | 75 | 3.75 |
| 14 | 80 | 4 |
| 15 | 85 | 4.25 |
| 16 | 90 | 4.5 |
| 17 | 95 | 4.75 |
| 18 | 100 | 5 |
| 19 | 105 | 5.25 |
| 20 | 110 | 5.5 |
| 21 | 115 | 5.75 |
| 22 | 120 | 6 |
| 23 | 125 | 6.25 |
| 24 | 130 | 6.5 |
| 25 | 135 | 6.75 |
| 26 | 140 | 7 |
| 27 | 145 | 7.25 |
| 28 | 150 | 7.5 |
| 29 | 155 | 7.75 |
| 30 | 160 | 8 |
| 31 | 165 | 8.25 |
| 32 | 170 | 8.5 |
| 33 | 175 | 8.75 |
| 34 | 180 | 9 |
| 35 | 185 | 9.25 |
| 36 | 190 | 9.5 |
| 37 | 195 | 9.75 |
| 38 | 200 | 10 |
| 39 | 205 | 10.25 |
| 40 | 210 | 10.5 |
| 41 | 215 | 10.75 |
| 42 | 220 | 11 |
| 43 | 225 | 11.25 |
| 44 | 230 | 11.5 |
| 45 | 235 | 11.75 |
| 46 | 240 | 12 |
| 47 | 245 | 12.25 |
| 48 | 250 | 12.5 |
| 49 | 255 | 12.75 |
| 50 | 260 | 13 |

### Example 11

### High-Throughput Screening Assay Identifying Changes in Small Molecule Concentration and Membrane Permeability

Binding by a ligand to a receptor can affect: intracellular levels of small molecules (such as, e.g., without limitation, calcium, potassium, and sodium); pH, and a membrane potential of the cell. These alterations are measured in an assay to identify supernatants that bind to a receptor. The following protocol is a non-limiting exemplar for assaying the effects on calcium ions in a cell (such as, e.g., without limitation, Ca⁺⁺ sequestration, removal, uptake, release, etc.) however, this assay can easily be modified to detect other cellular changes (such as, e.g., potassium, sodium, pH, membrane potential) effected by binding of a ligand with a receptor.

The following assay uses Fluorometric Imaging Plate Reader (“FLIPR”) to measure changes in fluorescent molecules (Molecular Probes) that bind small molecules, such as, e.g., Ca⁺⁺. Clearly, as would be recognized by the skilled artisan, other fluorescent molecules that can detect a small composition (such as, e.g., a small molecule) can be employed instead of the calcium fluorescent molecule, fluo-4 (Molecular Probes, Inc.; No. F-14202), used here. For adherent cells, seed the cells at 10,000-20,000 cells/well in a Co-starblack 96-well plate with a clear bottom. Incubate the plate in a CO₂ incubator for 20 hours. The adherent cells are washed twice in a Biotek washer with 200 ul of HBSS (Hank's Balanced Salt Solution) leaving 100 ul of buffer after the final wash. A stock solution of 1 mg/ml fluo-4 is made in 10% pluronic acid DMSO. To load the cells with fluo-4, 50 ul of 12 ug/ml fluo-4 is added to each well. The plate is incubated at 37° C. in a CO₂ incubator for 60 min. Wash the plate four times in a Biotek washer with 200 ul of HBSS leaving 100 ul of buffer (as described above). For non-adherent cells, the cells are spun down from culture media. Cells are re-suspended in a 50-ml conical tube to 2-5×10⁶ cells/ml with HBSS. Then, 4 ul of 1 mg/ml fluo-4 solution in 10% pluronic acid DMSO is added to each ml of cell suspension. Subsequently, the tube is placed in a 37° C. water bath for 30-60 min. The cells are washed twice with HBSS, re-suspended to 1×10⁶ cells/ml, and dispensed into a microplate (100 ul/well). The plate is centrifuged at 1000 rpmXg (times gravity) for 5 min. The plate is then washed once in 200 ul Denley Cell Wash followed by an aspiration step to 100 ul final volume. For a non-cell based assay, each well contains a fluorescent molecule, such as, e.g., fluo-4. The supernatant is added to the well, and a change in fluorescence is detected. To measure the fluorescence of intracellular calcium, the FLIPR is set for the following parameters: (1) System gain is 300-800 mW; (2) Exposure time is 0.4 second; (3) Camera F/stop is F/2; (4) Excitation is 488 nm; (5) Emission is 530 nm; and (6) Sample addition is 50 ul. Observance of an increased emission at 530 nm indicates an extracellular signaling event, which has resulted in an increase in the concentration of intracellular Ca⁺⁺.

### Example 12

### High-Throughput Screening Assay to Identify Tyrosine Kinase Activity

The Protein Tyrosine Kinases (PTK) represent a diverse group of transmembrane and cytoplasmic kinases. Within the Receptor Protein Tyrosine Kinase (RPTK) group are receptors for a range of mitogenic and metabolic growth factors including, e.g., the PDGF, FGF, EGF, NGF, HGF, and Insulin receptor subfamilies. In addition, a large number of RPTKs have no known corresponding ligand. Ligands for RPTKs include, e.g., mainly secreted small proteins, but also can include membrane-bound proteins, and extracellular matrix proteins.

Activation of an RPTK by a ligand typically involves dimerization of a ligand-mediated receptor resulting in the transphosphorylation of a receptor subunit(s) and subsequent activation of a cytoplasmic tyrosine kinase. Typically, cytoplasmic tyrosine kinases include, e.g., receptor associated tyrosine kinases of the src-family (such as, e.g., src, yes, lck, lyn, and fyn); non-receptor linked tyrosine kinases, and cytosolic protein tyrosine kinases (such as, e.g., Jaks, which mediate, e.g., signal transduction triggered by the cytokine superfamily of receptors such as, e.g., the Interleukins, Interferons, GM-CSF, and Leptin). Because of the wide range of factors that stimulate tyrosine kinase activity, the identification of a novel human secreted protein capable of activating tyrosine kinase signal transduction pathways would be useful. Therefore, the following protocol is designed to identify a novel human secreted protein (or fragments thereof) that activates a tyrosine kinase signal transduction pathway. Seed target cells (e.g., primary keratinocytes) at a density of approximately 25,000 cells per well in a 96 well Loprodyne Silent Screen Plates purchased (Nalge Nunc, Naperville, Ill.). Sterilize the plates using two 30-minute rinses with 100% ethanol, then rinse with doubly deionized water, and dry overnight. Coat some plates for 2 hr with 100 ml of cell culture grade type I collagen (50 mg/ml), gelatin (2%), polylysine (50 mg/ml) (Sigma Chemicals, St. Louis, Mo.); 10% Matrigel (Becton Dickinson, Bedford, Mass.); or calf serum. Then rinse the plates (PBS) and store at 4° C. Seed 5,000 cells/well in growth medium on a plate and then (after 48 hrs) assay cell growth by estimating the resulting cell number using the Alamar Blue method (Alamar Biosciences, Inc., Sacramento, Calif.). Use Falcon plate covers (#3071 from Becton Dickinson, Bedford, Mass.) to cover the Loprodyne Silent Screen Plates. Falcon Microtest III cell culture plates can also be used in some proliferation experiments.

To prepare extracts, seed A431 cells onto nylon membranes of Loprodyne plates (20,000/200 ml/well) and culture overnight in complete medium. Quiesce the cells by incubation in serum-free basal medium for 24 hr. Treat the cells with EGF (60 ng/ml) or 50 ul of a supernatant described herein, for 5-20 minutes. After removing the medium, add 100 ml of extraction buffer to each well (20 mM HEPES pH 7.5, 0.15M NaCl, 1% Triton X-100, 0.1% SDS, 2 mM Na₃VO₄, 2 mM Na₄P₂O₇ and a cocktail of protease inhibitors (Boeheringer Mannheim, Cat No. 1836170; Indianapolis, Ind.) and shake the plate on a rotating shaker for 5 minutes at 4° C. Then place the plate in a vacuum transfer manifold and extract filter through the 0.45 mm membrane bottom of each well (using house vacuum). Collect the extracts of a 96-well catch/assay plate in the bottom of the vacuum manifold and immediately place on ice. To clarify an extract by centrifugation, remove the content of a well (after detergent solubilization for 5 min) and centrifuge (15 min at 16,000×G at 4° C.). Test the filtered extracts for levels of tyrosine kinase activity. Although many methods of detecting tyrosine kinase activity are known and can be used without undue experimentation, a non-limiting method is described here for exemplar purposes. Generally, the tyrosine kinase activity of a supernatant is evaluated by determining its ability to phosphorylate a tyrosine residue on a specific substrate (e.g., a biotinylated peptide). An example of a biotinylated peptide useful for this purpose includes, e.g., without limitation, PSK1 (corresponding to amino acid residue numbers 6-20 of the cell division kinase cdc2-p34) and PSK2 (corresponding to amino acid residue numbers 1-17 of gastrin). Both of these biotinylated peptides are substrates for a number of tyrosine kinases and are commercially available (Boehringer Mannheim, Indianapolis, Ind.).

The tyrosine kinase reaction is set up by adding the following components as follows: First, add 10μl of 5 uM biotinylated peptide, then 10 μl ATP/Mg⁺² (5 mM ATP/50 mM MgCl₂), then 10 μl of 5× Assay Buffer (40 mM imidazole hydrochloride, pH 7.3, 40 mM beta-glycerophosphate 1 mM EGTA, 100 mM MgCl₂, 5 mM MnCl₂, 0.5 mg/ml BSA), then 5 μl of Sodium Vanadate (1 mM), and then 5 μl of water. Mix the components gently and pre-incubate the reaction mix at 30° C. for 2 min. Initialize the reaction by adding 10 μl of the control enzyme or the filtered supernatant. Stop the tyrosine kinase assay reaction by adding 10 ul of 120 mm EDTA and place the reactions on ice. Determine tyrosine kinase activity by transferring 50 ul of the reaction mixture to a microtiter plate (MTP) module and incubating at 37° C. for 20 min. This allows the streptavadin coated 96 well plate to associate with the biotinylated peptide. Wash the MTP module four times with 300 ul of PBS per well. Next add 75 ul of anti-phosphotyrosine antibody conjugated to horseradish peroxidase (anti-P-Tyr-POD (0.5 μl/ml)) to each well and incubate for one hour at 37° C. Wash each well as described above. Next, add 100 μl of peroxidase substrate solution (Boehringer Mannheim, Indianapolis, Ind.) and incubate for a minimum of five minutes (up to 30 min) at RT. Measure the absorbance of the sample at 405 nm using an ELISA reader (the level of bound peroxidase activity reflects the level of tyrosine kinase activity and is quantitated using an ELISA reader).

LP-induced tyrosine phosphorylation is determined as follows using any appropriate cell line (such as, e.g., Saos, GH4C1, LNCAP, LLC-PK1, L6, GT1-7, SK-N-MC, U373MG, MCF-7, Ishikawa, PA1, HEP-G2, ECV304, GLUTag, BTC6, HuVEC, TF-1, Balb/C 3T3, HDF, M07E, T1165, THP-1, or Jurkat). On day 1, approximately 2.0×10⁴ cells per are plated onto poly-D-lysine-coated wells (96 well plates) containing 100 μL cell propagation media (DMEM:F12 at a 3:1 ratio, 20 mM Hepes at pH 7.5, 5% FBS, and 50 μg/ml Gentamicin) then incubated overnight. On day 2, the propagation media is replaced with 100 μL starvation medium (DMEM:F12 at a 3:1, 20 mM Hepes at pH 7.5, 0.5% FBS, and 50 μg/ml Gentamicin) and incubated overnight. On day 3, a 100× stock of pervanadate solution is prepared (100 μL of 100 mM sodium orthovanadate and 3.4 μL of H₂O₂). Cells are stimulated with varying concentrations of an LP of the invention (e.g., 0.1, 0.5, 1.0, 5, and 10 μL of an LP stock solution) and incubated (10 min. at RT). After stimulation, the medium is aspirated and 75 μL lysis buffer (50 mM Hepes at pH 7.5, 150 mM NaCl, 10% glycerol, 1% TRITON X-100, 1 mM EDTA, 1 mM pervanadate, and BM protease inhibitors) is added to each well (4° C. for 15 minutes). Subsequently, 25 μL of 4X loading buffer is added to the cell lysates and the resulting solution is mixed and then heated to 95° C.

Detection of tyrosine phosphorylation is accomplished by Western immunoblotting. Samples of the treated cells (20 μl) are separated using SDS-PAGE 8-16% AA ready gels (Bio-Rad). Separated proteins are subsequently electrotransferred (˜1 hr at 250 mA) in transfer buffer (25 mM Tris base at pH 8.3, 0.2 M glycine, 20% methanol) to a nitrocellulose membrane that is incubated (1 hr at RT) in a blocking buffer (20 mM Tris HCl at pH 7.5, 150 mM NaCl, 0.1% TWEEN-20; 1% BSA). To detect the presence of LP-induced phosphorylated proteins any appropriate commercially available anti-phosphotyrosine antibody is added to a membrane (such as, e.g., a monoclonal antibody that can detect, e.g., Erk-1, Erk-2 kinase, Raf, JNK, p38 MAP, Map kinase kinase (MEK), MEK kinase, Src, Muscle specific kinase (MUSK), IRAK, Tee, and Janus, etc.). The membrane is incubated overnight (4° C. with gentle rocking) in a first solution (primary antibody, TBST, and 1% BSA), followed by TBST washing (X3 for 5 min/wash at RT) and incubation (1 hr at RT with gentle rocking) with a second solution (secondary antibody, TBST, and 1% BSA). After the secondary incubation, another series of TBST washes is carried out (X4 for 10 min/wash at RT) and detection of the immuno-identified proteins is visualized by incubating the membranes (10-30 ml of SuperSignal Solution for approximately 1 min at RT). After excess developing solution is removed, the membrane is wrapped (plastic wrap) and exposed to X-ray film (20 sec., 1 min., and 2 min. or longer if needed). LP-induced tyrosine phosphorylation is determined by comparing the number and intensity of immunostained protein bands from treated cells (visual inspection) with the number and intensity of immunostained protein bands from negative control cells (buffer only without LP solution).

### Example 13

### High-Throughput Screening Assay To Identify Phosphorylation Activity

An alternative and/or complimentary tyrosine kinase assay, which can also be used detects activation (e.g., phosphorylation) of intracellular signal transduction intermediates. For example, as described herein, such an assay detects tyrosine phosphorylation of an Erk-1 and/or Erk-2 kinase. However, detecting phosphorylation of other molecules, such as, e.g., Raf, JNK, p38 MAP, Map kinase kinase (MEK), MEK kinase, Src, Muscle specific kinase (MUSK), IRAK, Tee, and Janus; as well as any other phosphoserine, phosphotyrosine, or phosphothreonine molecule, can be determined by substituting one of these molecules for an Erk-1 or Erk-2 molecule used as follows. Specifically, assay plates are made by coating the wells of a 96-well ELISA plate with 0.1 ml of protein G (1 ug/ml) for 2 hr at RT. Then, the plates are rinsed with PBS and blocked with 3% BSA/PBS for 1 hr at RT. The protein G plates are subsequently treated for one hour at RT (100 ng/well) using a commercial monoclonal antibody directed against Erk-1 and/or Erk-2 (Santa Cruz Biotechnology). After 3-5 rinses with PBS, the plates are stored at 4° C. until further use. To detect phosphorylation of another molecule (as stated above) modify this step of the method by substituting an appropriate monoclonal antibody, which can detect one of the above-described molecules (such as, e.g., Raf, JNK, p38 MAP, Map kinase kinase (MEK), MEK kinase, Src, Muscle specific kinase (MUSK), IRAK, Tee, Janus, etc.)). Seed A431 cells at 20,000 cells/well in a 96-well Loprodyne filterplate and culture in an appropriate growth medium overnight. Then starve the cells for 48 hr in basal medium (DMEM) and treat for 5-20 minutes with EGF (6.0 ng/well) or with 50 ul of a supernatant described herein. Then, solubilize the cells and filter the cell extract directly into the assay plate. After incubation with the filtered extract for 1 hr at RT, rinse the wells again. As a positive control, use a commercial preparation of MAP kinase (10 ng/well) in place of the extract. Treat the plates (1 hr at RT) with a commercial polyclonal antibody (rabbit; 1 ug/ml) that recognizes a phosphorylated epitope of an Erk-1 and/or an Erk-2 kinase. Biotinylate the antibody using any standard, art-known procedure. Quantitate the amount of bound polyclonal antibody by successive incubations with Europium-streptavidin and Europium fluorescence enhancing reagent in a Wallac DELFIA instrument (using time-resolved fluorescence). Observance of an increased fluorescent signal over background indicates that phosphorylation has occurred.

### Example 14

### Method of Detecting Abnormal Levels of an LP Polypeptide in a Sample

An LP polypeptide (or fragment thereof) can be detected in a sample (such as, e.g., a biological sample as described herein). Generally, if an increased or decreased level of the LP polypeptide (compared to a normal level) is detected, then this level of the polypeptide (or fragment thereof) is a useful marker such as, e.g., for a particular cellular phenotype. Methods to detect the level of a polypeptide (or fragment thereof) are numerous, and thus, it is to be understood that one skilled in the art can modify the following exemplar assay to fit a particular need without incurring undue experimentation.

For example, an antibody-sandwich ELISA is used to detect an LP polypeptide (or fragment thereof) in a sample. Wells of a microtiter plate are coated with specific antibodies, at a final concentration of 0.2 to 10 ug/ml. The antibodies (either monoclonal or polyclonal) are produced by any art known method (or as described herein). The wells are treated with an appropriate blocking reagent so that non-specific binding of the LP polypeptide (or fragment thereof) to the well is reduced and/or prevented. The coated wells are then incubated for greater than 2 hours at RT with the sample containing the LP polypeptide (or fragment thereof). Preferably, serial dilutions of the sample containing the suspected polypeptide (or fragment thereof) should be used to validate results. The plates are then washed three times with doubly deionized or distilled water to remove unbound polypeptide. Next, 50 ul of specific antibody-alkaline phosphatase conjugate (at a concentration of 25-400 ng) is added and incubated (2 hours at RT). The plates are again washed three times with doubly deionized or distilled water to remove unbound conjugate. Subsequently, 75 ul of 4-methylumbelliferyl phosphate (MUP) or p-nitrophenylphosphate NAPP) substrate solution is added to each well and incubated (approximately one hour at RT). The reaction is then measured by a microtiter plate reader. A standard curve is prepared, using serial dilutions of a control sample, and the polypeptide concentration is plotted on the X-axis (log scale) with fluorescence or absorbance plotted on the Y-axis (linear scale). The concentration of the polypeptide in the sample can then be interpolated using the standard curve.

### Example 15

### Detecting Stimulation or Inhibition of B cell Proliferation and Differentiation

Generation of functional humoral immune responses requires both soluble and cognate signaling between B-lineage cells and their microenvironment a signal may impart a positive stimulus that allows a B-lineage cell to continue its programmed development, or a negative stimulus that instructs the cell to arrest its current developmental pathway. To date, numerous stimulatory and inhibitory signals have been found that influence B cell responsiveness (including, e.g., signals from: IL-2, IL-4, IL-5, IL-6, IL-7, IL-10, IL-4, IL-13, IL-1 4, and IL-15). Interestingly, a signal by itself can be a weak effector but, in combination with various co-stimulatory proteins, the signal can induce, e.g., activation, proliferation, differentiation, homing, tolerance, and death among B cell populations. One of the best-studied examples of a B-cell co-stimulatory protein is the class of molecules represented by the TNF-superfamily. Within this family, it has been demonstrated that CD40, CD27, and CD30 along with their respective ligands (CD154, CD70, and CD 153) regulate a variety of immune responses. Assays which allow for the detection and/or observation of the proliferation and/or differentiation of a B-cell population and/or its precursors are useful in determining the effect of a composition of the invention on a B-cell population (e.g., in terms of proliferation and differentiation). Taught herein below are two assays designed to detect the effect of a composition of the invention on the differentiation, proliferation, and/or inhibition of a B-cell population or its precursor.

In vitro Assay: An LP polypeptide of the invention (or fragment thereof), is assessed for its ability to induce activation, proliferation, differentiation, inhibition, and/or death in a B-cell and its precursors. The activity of the LP polypeptide on purified human tonsillar B cells (measured qualitatively over the dose range from 0.1 to 10,000 ng/mL) is assessed using a standard B-lymphocyte co-stimulation assay in which purified, tonsillar B cells are cultured in the presence a priming agent (such as, e.g., either formalin-fixed Staphylococcus aureus Cowan I (SAC) or immobilized anti-human IgM antibody). A second signal (such as, e.g., IL-2, and IL-15) synergizes with SAC and IgM crosslinking to elicit B cell proliferation (measured by tritiated-thymidine incorporation). A novel synergizing agent can readily be identified using this assay. The assay involves isolating human tonsillar B cells by magnetic-bead-depletion (MACS) of CD3-positive cells. The resulting cell population is greater than 95% B cells as assessed by expression of CD45R(B220). Various dilutions of each sample are placed into individual wells of a 96-well plate to which are added 10⁵B-cells suspended in culture medium (RPM1 1640 containing 10% 5FBS, 5×10⁻⁵M 2ME, 100 U/ml penicillin, 10 ug/ml streptomycin, and 10⁻⁵ dilution of SAC) in a total volume of 150 μl. Proliferation or inhibition is quantitated by a 20 h pulse (1 uCi/well) with 3H-thymidine (6.7 Ci/mM) beginning 72 h post factor addition. The positive and negative controls are respectively, IL2 and medium.

In vivo Assay: BALB/C mice are injected (i.p.) twice daily either with buffer alone or with 10 mg/Kg of an LP polypeptide of the invention (or fragment thereof). Mice receive this treatment for four consecutive days, at which time they are sacrificed and various tissues and serum collected for analyses. Comparison of sections (hemotoxylin and eosin stained) from normals and spleens treated with an LP polypeptide (or fragment thereof) are assessed to identify an effect of the activity of the LP polypeptide (or fragment thereof) on spleen cells (such as, e.g., the diffusion of peri-arterial lymphatic sheaths, and/or significant increases in the nucleated cellularity of the red pulp regions, which may indicate activation of differentiation and proliferation of a B-cell population). Any immunohistochemical technique using any appropriate B cell marker (such as, e.g., anti-CD45R) is used to determine whether a physiological change to a splenic cell (such as, e.g., splenic disorganization) is due to an increased B-cell representation within a loosely defined B-cell zone that infiltrates an established T-cell region. Flow cytometric analyses of spleens from treated mice are used to indicate whether the tested LP polypeptide (or fragment) specifically increases the proportion of ThB+, CD45R dull B cells over control levels. Similarly, an indication of an increased representation of mature B-cells in vivo is the detection in a relative increase in serum titers of Ig. Furthermore, determining whether increased B-cell maturation has occurred can also be achieved by comparing serum IgM and IgA levels between LP polypeptide-treated mice and mice treated with buffer only.

### Example 16

### T-Cell Proliferation Assay

To assess the effect of an LP polypeptide (or fragment thereof) of the invention on T-cell proliferation (e.g., by measuring CD3-induced proliferation), an assay is performed on PBMCs to measure ³H-thymidine uptake. Ninety-six well plates are coated with 100 μl/well of monoclonal antibody to CD3 (such as, e.g., HIT3a, Pharmingen) or an isotype-matched control mAb (e.g., B33.1) overnight at 4° C. (1 μg/ml in 0.05M bicarbonate buffer, pH 9.5), then washed X3 (PBS). PBMC are isolated by F/H gradient centrifugation from human peripheral blood and added to quadruplicate wells (5×10⁴/well) of mAb coated plates in RPM1 containing 10% FCS and P/S in the presence of varying concentrations of an LP polypeptide (or fragment thereof) (total volume 200 ul). Relevant protein buffer (or medium only) is used as a control. After 48 hr culture at 37° C., plates are spun for 2 min. at 1000 rpm and 100 μl of supernatant is removed and stored at −20° C. for measurement of IL-2 (or other cytokines) if an effect on proliferation is observed. Wells are supplemented with 100 μl of medium containing 0.5 uCi of ³H-thymidine and cultured at 37° C. for 18-24 hr. Wells are harvested and the amount of incorporation of ³H-thymidine is used as a measure of proliferation. Anti-CD3 by itself is used as a positive control for proliferation. IL-2 (100 U/ml) is also used as a control that enhances proliferation. A control antibody that does not induce proliferation of T cells is used as a negative control for the effect of an LP polypeptide (or fragment thereof).

### Example 17

### Effect of an LP Polypeptide (or Fragment Thereof) on the Expression of MHC Class II, Co-stimulatory and Adhesion Molecules and Cell Differentiation of Monocytes and Monocyte-Derived Human Dendritic Cells

Dendritic cells are generated by the expansion of proliferating precursors found in the peripheral blood: adherent PBMC or elutriated monocytic fractions are cultured for 7-10 days with GM-CSF (50 ng/ml) and IL4 (20 ng/ml). These dendritic cells have the characteristic phenotype of immature cells (e.g., expression of CD1, CD80, CD86, CD40, and MHC class II antigens). Treatment with an activating factor (such as, e.g., TNF-alpha) causes a rapid change in surface phenotype (e.g., an increased expression of MHC class I and II, co-stimulatory and adhesion molecules, down regulation of FQRII, and/or an up regulation of CD83). Typically, these changes correlate with an increased antigen-presenting capacity and/or with a functional maturation of a dendritic cell. A FACS analysis of surface antigens is performed as follows: cells are treated 1-3 days with increasing concentrations of an LP polypeptide (or fragment thereof) or LPS as a positive control, washed with PBS containing 1% BSA and 0.02 mM NaN₃, and then incubated with 1:20 dilution of appropriate FITC- or PE-labeled monoclonal antibodies for 30 minutes at 4° C. After an additional wash, the labeled cells are analyzed by flow cytometry on a FACScan (Becton Dickinson).

Effect on the Production of Cytokines

Cytokines generated by dendritic cells, in particular IL-12, are important in the initiation of T-cell dependent immune responses. IL-12 strongly influences the development of Th-1 helper T-cell immune response, and induces cytotoxic T and NK cell function. An ELISA is used to measure IL-12 release in a dendritic cell that has been exposed to an LP polypeptide of the invention (or fragment thereof as follows: dendritic cells (10⁶/ml) are treated with increasing concentrations of an LP polypeptide (or fragment thereof) for 24 hours. LPS (100 ng/ml) is added to a cell culture as a positive control. Supernatants from the cell cultures are then collected and analyzed for IL-12 using a commercial ELISA kit (e.g., R & D Systems; Minneapolis, Minn.). The standard protocol provided with the kit is used to measure IL-12 expression.

Effect on the Expression of MHC Class II, Co-Stimulatory, and Adhesion Molecules.

Three major families of cell surface antigens is identified on monocytes: adhesion molecules, molecules involved in antigen presentation, and Fc receptor. Modulation of the expression of MHC class II antigens and other co-stimulatory molecules (such as, e.g., B7 and ICAM-1) may result in changes in the antigen presenting capacity of a monocyte and in an ability to induce T cell activation. Increased expression of Fc receptors may correlate with improved monocyte cytotoxic activity, cytokine release, and phagocytosis. A FACS analysis is used to examine surface antigens as follows: monocytes are treated for 1-5 days with increasing concentrations of an LP polypeptide (or fragment thereof) or LPS (as a positive control), washed with PBS containing 1% BSA and 0.02 mM sodium azide (NaN₃), and then incubated with a 1:20 dilution of appropriate FITC- or PE-labeled monoclonal antibodies for 30 minutes at 4° C. After an additional wash, the labeled cells are analyzed by flow cytometry on a FACS scanner (Becton Dickinson).

Monocyte Activation and/or Increased Survival

Assays for molecules that: activate (or, alternatively, inactivate) monocytes; and/or increase monocyte survival (or, alternatively, decrease monocyte survival) are known in the art and may routinely be applied to determine whether a composition of the invention (such as, e.g., a polypeptide or fragment thereof) functions as an inhibitor or activator of a monocyte. Polypeptides (fragments thereof), agonists, or antagonists of the invention is screened using any of the assays described below. For each of these assays, peripheral blood mononuclear cells (PBMC) are purified from single donor leukopacks (American Red Cross, Baltimore, Md.) by centrifugation through a Histopaque gradient (Sigma). Monocytes are isolated from PBMC by counterflow centrifugal elutriation.

Monocyte Survival Assay

Human, peripheral-blood monocytes progressively lose viability when cultured in the absence of serum or other stimuli. Their death typically results from internally regulated processes (such as, e.g., apoptosis). Addition to a culture of activating factors, such as, e.g., TNF-alpha dramatically improves PBMC survival and prevents DNA fragmentation. Propidium iodide (PI) staining is used to measure apoptosis as follows: monocytes are cultured for 48 hours in polypropylene tubes in serum-free medium (positive control), in the presence of 100 ng/ml TNF-alpha (negative control), and in the presence of varying concentrations of a composition of the invention (such as, e.g., an LP polypeptide or fragment thereof). Cells are suspended at a concentration of 2×10⁶/ml in PBS containing PI at a final concentration of 5 μg/ml, and then incubated at RT for 5 minutes before FACS scan analysis. PI uptake has been demonstrated to correlate with DNA fragmentation in this method.

Effect on Cytokine Release

An important function of monocytes/macrophages is their regulatory activity on other cellular populations of the immune system (e.g., through the release of cytokines after appropriate stimulation). An ELISA assay to measure cytokine release is performed as follows: human monocytes are incubated at a density of 5×10⁵ cells/ml with increasing additions of varying concentrations of an LP polypeptide (or fragment thereof of the invention (controls employ the same conditions without the LP polypeptide). For IL-12 production, the cells are primed overnight with IFN (100 U/ml) in presence of an LP polypeptide (or fragment thereof). LPS (10 ng/ml) is then added. Conditioned media are collected after 24 h and kept frozen until use. Measurement of TNF-alpha, IL-1, MCP-1, and IL-8 is then performed using any commercially available ELISA kit (e.g., R & D Systems; Minneapolis, Minn.) according to a standard protocol provided with the kit.

Oxidative Burst

Purified monocytes are plated in 96-w plate at approximately 1×10⁵ cells/well. Increasing concentrations of a polypeptide of the invention (or fragment thereof) are added to the wells in a total volume of 0.2 ml culture medium (RPM1 1640+10% FCS, glutamine and antibiotics). After 3 days incubation, the plates are centrifuged and the medium is removed from the wells. To the macrophage monolayers, 0.2 ml per well of phenol red solution (140 mM NaCl, 10 mM potassium phosphate buffer pH 7.0, 5.5 mM dextrose, 0.56 mM phenol red and 19 U/ml of HRPO) is added, together with a stimulant (200 nM PMA). The plates are incubated at 37° C. for 2 hours and the reaction is stopped by adding 20 μl (1N NaOH) per well. The absorbance is read at 610 nm. To calculate the amount of H₂O₂ produced by the macrophages, a standard curve of a H₂O₂ solution of known molarity is performed for each experiment.

### Example 18

### Biological Effects of an LP Polypeptide (or Fragment Thereof)

Astrocyte and Neuronal Cell Assays

An LP polypeptide of the invention (or fragment thereof) is tested for its capacity to promote survival, neurite outgrowth, and/or phenotypic differentiation of a cell of the nervous system (such as, e.g., a cortical neuronal cell) and/or for it capacity to induce the proliferation of a cell of the nervous system (such as, e.g., a glial fibrillary acidic protein immunopositive cell like, e.g., an astrocyte). The use of a cortical cell for this assay is based on the prevalent expression of FGF-1 and FGF-2 (basic FGF) in cortical structures and on reported enhancement of cortical neuronal survival after FGF-2 treatment. A thymidine incorporation assay, e.g., is used to assess the effect of the LP on the nervous system cell.

An in vitro effect of FGF-2 on cortical or hippocampal neurons shows increased neuronal survival and neurite outgrowth (see, e.g., Walicke, et al. (1986) Proc. Natl. Acad. Sci. USA 83:3012-3016). However, reports from experiments on PC-12 cells suggest that neuronal survival and neurite outgrowth are not necessarily synonymous and that a specific effect may depend not only on which FGF is tested but also on the particular receptor(s) that are expressed on a target cell. Using a primary cortical neuronal culture paradigm, the ability of an LP polypeptide (or fragment thereof) to induce neurite outgrowth and effect neuronal survival compared to FGF-2 is assessed using, e.g., a thymidine incorporation assay.

Fibroblast and Endothelial Cell Assays.

For proliferation assays, human lung fibroblasts (Clonetics; San Diego, Calif.) and/or dermal microvascular endothelial cells (Cell Applications; San Diego, Calif.) are cultured at 5,000 cells/well in a 96-well plate for one day in growth medium. The cells are then incubated for one day in 0.1% BSA basal medium. After replacing the medium with fresh 0.1% BSA medium, the cells are incubated (72 hr) with varying concentrations of an LP polypeptide of the invention (or fragment thereof). Then, Alamar Blue (Alamar Biosciences, Sacramento, Calif.) is added to each well to a final concentration of 10% and the cells are incubated for 4 hr. Cell viability is measured using a CytoFluorfluorescence reader. For a PGE assay, the human lung fibroblasts are cultured at 5,000 cells/well in a 96-well plate for one day. After a medium change to 0.1% BSA basal medium, the cells are incubated with FGF-2 or an LP polypeptide (or fragment thereof) with (or without) IL-1 alpha for 24 hours. Then supernatants are collected and assayed for PGE, by EIA (Cayman; Ann Arbor, Mich.). For an IL-6 assay, the human lung fibroblasts are cultured at 5,000 cells/well in a 96-well plate for 24 hrs. After a medium change to 0.1% BSA basal medium, the cells are incubated with FGF-2 or an LP polypeptide (or fragment thereof) with (or without) IL-1 alpha for 24 hours. The supernatants are collected and assayed for IL-6 by ELISA kit (Endogen; Cambridge, Mass.). Human lung fibroblasts are cultured with FGF-2 or an LP polypeptide (or fragment thereof) for 3 days in basal medium before the addition of Alamar Blue to assess any effect on growth of the fibroblasts. FGF-2 should show a stimulatory effect at about 10-2500 ng/ml, which can then be used to compare any stimulatory effect of an LP polypeptide (or fragment thereof).

Parkinson Models

The loss of motor function in Parkinson's syndrome is attributed to a deficiency of striatal dopamine due to the degeneration of nigrostriatal dopaminergic projection neurons. A Parkinsonian animal model involves systemic administration of 1-methyl-4 phenyl 1,2,3,6-tetrahydropyridine (MPTP). In the central nervous system, MPTP is taken-up by astrocytes and catabolized to 1-methyl-4-phenyl pyridine (MPP⁺), which is subsequently released. Released MPP⁺ is accumulated in dopaminergic neurons by the high-affinity re-uptake transporter for dopamine, MPP⁺ is then concentrated in mitochondria via an electrochemical gradient where it selectively inhibits nicotinamide adenine disphosphate: ubiquinone oxidoreductionase (complex I) thereby, interfering with electron transport and eventually generating oxygen radicals. In tissue culture, FGF-2 (basic FGF) has trophic activity towards nigral dopaminergic neurons (Ferrari, et al. (1989) Dev. Biol. 133(1):140-147), and administering a striatal gel foam implant containing FGF-2 protects nigral dopaminergic neurons from MPTP toxicity (Otto and Unsicker, (1990) J. Neuroscience 10(6):1912-1921). Based on these reported data for the effect of FGF-2, an LP polypeptide (or fragment thereof) of the invention is evaluated to determine whether it has a similar effect as FGF-2 (such as, e.g., by modulating dopaminergic neuronal survival (either in vitro or in vivo) from an effect of MPTP treatment). An in vitro dopaminergic neuronal cell culture is prepared by dissecting the midbrain floor plate from gestation day 14 Wistar rat embryos. The tissue is dissociated with trypsin and seeded at a density of 200,000 cells/cm² on polyorthinine-laminin coated glass coverslips. The cells are maintained in Dulbecco's Modified Eagle's medium and F12 medium containing hormonal supplement (N 1). After 8 days in vitro, cultures are fixed with paraformaldehyde and processed for immunohistochemical staining of tyrosine hydroxylase (a specific marker for dopaminergic neurons). Dissociated cell cultures are prepared from embryonic rats. The culture medium is changed every third day and the factors are added at that time. Typically, dopaminergic neurons isolated from gestation-day-14 animals are past a point when dopaminergic precursor cells are believed to be proliferating, therefore, an increase in the number of tyrosine hydroxylase immunopositive neurons is interpreted to suggest that a similar increase in the number of surviving dopaminergic neurons would occur if the treatment had occurred in vitro. Therefore, if an LP polypeptide (or fragment thereof) prolongs the survival of dopaminergic neurons in an assay as taught herein, it suggests that the polypeptide (or fragment) is used to ameliorate, modulate, treat, or effect a Parkinson's disease, syndrome, condition, or state.

### Example 19

### The Effect of an LP Polypeptide on Endothelial Cells

An LP polypeptide (or fragment thereof) is tested for its effect on an endothelial cell (such as, e.g., the effect on the growth of vascular endothelial cells) using the following assay: on day 1, human umbilical vein endothelial cells (HUVEC) are seeded at 2-5×10² cells/35 mm dish density in M199 medium containing 4% fetal bovine serum (FBS), 16 units/ml heparin, and 50 units/ml endothelial cell growth supplements (ECGS, Biotechnique, Inc.). On the following day, the medium is replaced with M199 containing 10% FBS, 8 units/ml heparin. An LP polypeptide (or fragment thereof), and positive controls (such as, e.g., VEGF, and basic FGF (bFGF)) are added to the cells at varying concentrations. On days 4, and 6, the medium is replaced. On day 8, cell number is determined with a Coulter Counter. An increase in the number of HUVEC cells indicates that the polypeptide (or fragment thereof) mediates proliferation of vascular endothelial cells.

### Example 20

### Stimulatory Effect of an LP Polypeptide on the Proliferation of Vascular Endothelial Cells

An LP polypeptide (or fragment thereof) is tested for its stimulatory effect on an endothelial cell (such as, e.g., a vascular endothelial cell) to evaluate a mitogenic effect. A calorimetric MTS (3-(4,5-dimethylthiazol-2-yl)-5-(3-carboxymethoxyphenyl)-2-(4-sulfophenyl)2H-tetrazolium) assay with the electron coupling reagent PMS (phenazine methosulfate) is performed (Cell Titer 96 AQ, Promega) based on Leak, et al. (1994) In vitro Cell. Dev. Biol. 30A:512-518 (incorporated herein for its assay teachings). Briefly, cells are seeded in a 96-well plate (5,000 cells/well) in 0.1 mL serum-supplemented medium and allowed to attach overnight. After serum-starvation for 12 hours (in 0.5% FBS conditions), bFGF, VEGF, or an LP polypeptide (or fragment thereof), in 0.5% FBS (either with or without Heparin (8 U/ml), is added to a well of the plate. After 48 hours, 20 mg of MTS/PMS mixture (1:0.05) is added per well and incubated (1 hour at 37° C.) before measuring the absorbance (490 nm in an ELISA plate reader). Background absorbance from control wells (some media, no cells) is subtracted, and seven wells are performed in parallel for each condition to test for the presence of mitogenic activity (Leak, et al. supra).

### Example 21

### Inhibition of PDGF-Induced Vascular Smooth Muscle Cell Proliferation

An LP polypeptide (or fragment thereof) is tested for its effect on vascular smooth muscle cell proliferation (e.g., by measuring BrdUrd incorporation) according to an assay of Hayashida, et al. (1996) J. Biol. Chem. 6:271(36): 21985-21992 (incorporated herein for its assay teachings).

Briefly, subconfluent, quiescent HAoSMC cells grown on 4-chamber slides are transfected with CRP or FITC-labeled AT2-3LP. Then, the cells are pulsed with 10% calf serum and 6 mg/ml BrdUrd. After 24 h, immunocytochemistry is performed using BrdUrd Staining Kit (Zymed Laboratories). In brief, after being exposed to denaturing solution, the cells are incubated with biotinylated mouse anti-BrdUrd antibody (4° C. for 2 h) and then incubated with streptavidin-peroxidase and diaminobenzidine. After counterstaining with hematoxylin, cells are mounted for microscopic examination, and BrdUrd-positive cells are counted. A BrdUrd index is calculated as a percentage of the number of BrdUrd-positive cells per number of total cells. Additionally, simultaneous detection of BrdUrd staining (nucleus) and FITC uptake (cytoplasm) is performed for an individual cell by the concomitant use of bright field illumination and dark field, UV fluorescent illumination (see, Hayashida, et al., supra, for details).

### Example 22

### Stimulation of Endothelial Migration by an LP

An LP polypeptide (or fragment thereof) is tested for its effect on lymphatic endothelial cell migration. Endothelial cell migration assays are performed using a 48 well micro-chemotaxis chamber (Neuroprobe Inc.; Falk, et al. (1980) J. Immunological Methods: 33:239-247). Polyvinylpyrrolidone-free polycarbonate filters with a pore size of 8 μm (Nucleopore Corp.; Cambridge, Mass.) are coated with 0.1% gelatin (at least 6 hours at RT) and dried under sterile air. Test substances are diluted to appropriate concentrations in M199 supplemented with 0.25% bovine serum albumin (BSA), and 10 ul of the final dilution is placed in the lower chamber of a modified Boyden apparatus. Subconfluent, early passage (2-6) HUVEC or BMEC cultures are washed and trypsinized for the minimum time required to achieve cell detachment. After placing the filter between lower and upper chamber, 2.5×10⁵ cells (suspended in 50 ul M199 containing 1% FBS) are seeded to the upper compartment. The apparatus is then incubated (5 hrs 37° C. in a humidified chamber (5% CO₂)) to allow cell migration. After the incubation period, the filter is removed and the upper side of the filter (containing non-migrated cells) is scraped to remove cells. Then the filters are fixed with methanol and stained with Giemsa solution (Diff-Quick, Baxter, McGraw Park, Ill.). Migration is assessed by counting the number of cells occupying three random high-power fields (40×) in each well (measurements in all groups are performed in quadruplicate).

### Example 23

### LP Stimulation of Nitric Oxide Production by Endothelial Cells

An LP polypeptide (or fragment thereof) is tested for its effect on nitric oxide production by an endothelial cell according to the following assay.

Nitric oxide released by the vascular endothelium is believed to be a mediator of vascular endothelium relaxation. Nitric oxide is measured in 96-well plates of confluent microvascular endothelial cells after 24 hours starvation and a subsequent 4 hr exposure to various levels of an LP polypeptide (or fragment thereof) or a positive control (such as, e.g., VEGF-1). The presence of nitric oxide in the medium is determined by use of the Griess reagent to measure total nitrite after reduction of nitric oxide-derived nitrate by nitrate reductase. The effect of an LP polypeptide (or fragment thereof) on nitric oxide release is examined on HUVEC cells. Briefly, NO release from a cultured HUVEC monolayer is measured with a NO-specific polarographic electrode connected to a NO meter (Iso-NO, World Precision Instruments Inc.) (1049). Calibration of the electrodes is performed with air-saturated distilled water (ISO) or acidified nitrite (Iso-NO) according to the procedure recommended by the manufacturer. The Iso-NO is prepared by the addition of KNO to a helium-gassed solution of 0.14 M KSO and 0.1 M KI in 0.1 M HSO. The standard calibration curve is obtained by adding graded concentrations of KNO₂ (e.g., 0, 5.0, 10.0, 25, 50, 100, 250, and 500 nmol/L) into the calibration solution containing KI and H₂SO₄. The specificity of the Iso-NO electrode to NO is previously determined by measurement of NO from authentic NO gas (1050). The culture medium is removed and HUVECs are washed twice with Dulbecco's phosphate buffered saline. The cells are then bathed in 5 ml of filtered Krebs-Henseleit solution in 6-well plates, and the cell plates are kept on a slide warmer (Lab Line Instruments Inc.) To maintain the temperature at 37° C., the NO sensor probe is inserted vertically into the wells, keeping the tip of the electrode 2 mm under the surface of the solution, before addition of the different conditions. S-nitroso acetyl penicillamin (SNAP) is used as a positive control. The amount of released NO is expressed as picomoles per 1×10⁶ endothelial cells. All values should be established from the means of four to six measurements in each group (number of cell culture wells). See, e.g., Leak, et al. (1995) Biochem. and Biophys. Res. Comm. 217:96-105 (incorporated by reference for teachings on NO assays).

### Example 24

### Effect of an LP Polypeptide on Cord Formation/Hematopoiesis

An LP polypeptide (or fragment thereof) is tested in the following assay for its effect on angiogenesis (such as, e.g., endothelial cell differentiation during cord formation such as, e.g., the ability of microvascular endothelial cells to form capillary-like hollow structures when cultured in vitro). Microvascular endothelial cells (CADMEC; Cell Applications, Inc.) purchased as proliferating cells (passage 2) are cultured in CADMEC growth medium (Cell Applications, Inc.) and used at passage 5. For an in vitro angiogenesis assay, the wells of a 4% cell culture plate are coated (200 ml/well) with attachment factor medium (Cell Applications, Inc.) for 30 min. at 37° C. CADMEC cells are seeded onto the coated wells at 7,500 cells/well and cultured overnight in the growth medium. The growth medium is then replaced with 300 mg chord formation medium (Cell Applications, Inc.) containing either a control buffer or an LP polypeptide (or fragment thereof) (ranging from 0.1 to 100 ng/ml). Commercial VEGF (50 ng/ml; R&D) is used as a positive control. Beta-esteradiol (1 ng/ml) is used as a negative control. An appropriate buffer (without the polypeptide) is also utilized as a control. Treated cells are then cultured for 48 hr. Any resulting capillary-like chords are quantitated (numbers and lengths) using a video image analyzer (e.g., Boeckeler VIA-170). All assays are done in triplicate.

### Example 25

### Effect of an LP Polypeptide on Angiogenesis in a Chick Chorioallantoic Membrane

An LP polypeptide (or fragment thereof) is tested in the following assay for its effect on angiogenesis (such as, e.g., the formation of blood vessels on a chick chorioallantoic membrane (CAM)). The chick chorioallantoic membrane (CAM) is a well-established system to examine angiogenesis. Blood vessel formation on CAM is easily visible and quantifiable.

Fertilized eggs of the White Leghorn chick (Gallus gallus) and the Japanese quail (Cotumix cotumix) are incubated (37.8° C. and 80% humidity). Differentiated CAM of 16-day-old chick and 13-day-old quail embryos is studied as follows: On day 4 of development, a window is made on the shell of a chick egg. The embryos are checked for normal development and the eggs sealed with cellotape. The eggs are further incubated until development day 13 (using standard development stages). Thermanox coverslips (Nunc, Naperville, Ill.) are cut into disks of about 5 mm in diameter. Sterile and salt-free growth factors and an LP polypeptide (or fragment thereof) (ranging from 0.1 to 100 ng/ml) are dissolved in distilled water and about 3.3 mg/5 ml of the mixture are pipetted on the disks. After air-drying, the inverted disks are applied on a CAM. After 3 days, the specimens are fixed in 3% glutaraldehyde and 2% formaldehyde and rinsed in 0.12 M sodium cacodylate buffer. They are then photographed with a stereo microscope [Wild M8] and embedded for semi- and ultra-thin sectioning using any art known method. Controls are performed with carrier disks alone. The extent of angiogenesis due to a growth factor only, an LP polypeptide only, or a combination of a growth factor and an LP is measured with respect to the degree of angiogenesis found on the untreated controls.

### Example 26

### An In Vivo Angiogenesis Assay Using a Matrigel Implant

An LP polypeptide (or fragment thereof) is tested in the following assay for its effect on angiogenesis (such as, e.g., its effect on the ability of an existing capillary network to form new vessels in a capsule of extracellular matrix material (Matrigel) which is implanted in a living rodent). Briefly, varying concentrations of an LP polypeptide (or fragment thereof) are mixed with liquid Matrigel (Becton Dickinson Labware; Kollaborative Biomedical Products) at 4° C. and then injected subcutaneously into a rodent (e.g., a mouse) where it subsequently solidifies into a plug. After 7 days, the plug is removed and examined for the presence of new blood vessels. More specifically, an LP polypeptide (or fragment thereof), preferably a secreted protein, (e.g., such as, 150 ng/ml) is mixed with Matrigel at 4° C. (the Matrigel material is liquid at 4° C.) and then drawn into a cold 3 ml syringe. A female C57BY6 mouse (approximately 8 weeks old) is then injected with approximately 0.5 ml of the mixture at two separate locations (preferably, around the midventral aspect of the abdomen). After 7 days, all injected mice are sacrificed, the Matrigel plugs are removed and cleaned (i.e., all clinging membranes and fibrous tissue is removed). The plugs are then fixed in neutral buffered formaldehyde (10%), embedded in paraffin, sectioned for histological examination, and stained (e.g., Masson's Trichrome). Cross sections from three different regions of each plug arc so processed while other elected sections are stained for the presence of vWF. A positive control for this assay is bovine basic FGF (150 ng/ml). Matrigel alone (without an LP polypeptide or FGF) is used as a control to determine basal levels of angiogenesis.

### Example 27

### Effect of LP on Ischemia in a Rabbit Lower Limb Model

An LP polypeptide (or fragment thereof) is tested in the following assay for its effect on ischemia using a rabbit hindlimb ischemia model (created by surgical removal of a femoral artery as described by Takeshita, et al. (1995) Am J. Patho 147:1649-16605 and Howell et al., (2000) Nonviral Delivery of the Developmentally Regulated Endothelial Locus-1 (del-1) Gene Increases Collateral Vessel Formation to the Same Extent as hVEGF165 in a Rabbit Hindlimb Ischemia Model, Program No.: 536, Third Annual Meeting of the American Society of Gene Therapy; each of which are incorporated by reference herein for the teachings of this assay).

### Example 28

### Effect of an LP Polypeptide on Vasodialation

An LP polypeptide (or fragment thereof) is tested in the following assay for its ability to affect blood pressure in spontaneously hypertensive rats (SHR), such as, e.g., by modulating dilation of the vascular endothelium. In one embodiment, a retrovirally-mediated recombinant construct comprising an LP polypeptide (or fragment thereof) at varying dosages (e.g., 0.5, 1, 10, 30, 100, 300, and 900 mg/kg) is delivered intracardiacally to determine the affect on the development of high blood pressure in a spontaneously hypertensive (SH) rat model of human essential hypertension to determine whether attenuation of high BP is associated with prevention of other pathophysiological changes induced by a hypertensive state. Intracardiac delivery of a polypeptide (or fragment thereof is administered to 13-14 week old spontaneously hypertensive rats (SHR) according to a method of Martens, et al. (1998) Proc Natl Acad. Sci U S A 95(5):266-9 (incorporated herein for the teachings of this method). Control SHR and Wister-Kyoto rats (WKY) receive a placebo for the same period. The duration and initiation of treatment, site of administration, among other factors, can influence the reversal of pathophysiological alterations associated with hypertension. At the end of treatment, the effect on arterial systolic blood pressure and the level of perivascular collagen concentration is compared to controls. In addition, the medial cross-sectional area of the aorta is compared to that of untreated SHR. Data on vasuclar lumen changes is expressed as the mean (±) of a SEM. Other measurements used to determine treatment outcome are: (1) coronary flow (using the Langendorff-perfused heart model at baseline) after maximum vasodilation in response to adenosine (10(−5) M), after endothelium-dependent vasodilation in response to bradykinin (10(−8) M), and after ecNOS inhibition by nitro-L-arginine methyl ester (L-NAME) (10(−4) M); (2) medial thickening of coronary microvessels and perivascular collagen on histological heart sections; and (3) ecNOS expression by immunohistochemical staining in appropriate vessels using 20-week-old spontaneously hypertensive (SHR) and Wistar-Kyoto control rats (WKY). These measurements are determined by computer-directed color analysis. Statistical analysis are performed with a paired t-test and statistical significance is defined as p<0.05 vs. the response to buffer alone.

### Example 29

### Effect of an LP Polypeptide in a Rat Ischemic Skin Flap Model

Current estimates indicate that over 2,000,000 U.S. citizens have chronic wounds each year, and the problem is increasing as the population ages. The cost of caring for chronic wounds reaches into the billions of dollars a year. Clearly, there is a need for better treatment to promote healing of chronic wounds. Ischemia is a major factor contributing to the failure of most chronic wounds to heal. Wound healing involves, e.g., soluble factors that control a series of processes including inflammation, cellular proliferation, and maturation (see, e.g., Robson, M. C. (1997) Wound Repair and Regeneration 5:12-17). Pro-inflammatory cytokines such as tumor necrosis factor (TNF) and Interleukin-1 (IL-1), proteases, protease inhibitors, and growth factors play important roles in normal wound healing. Excessive production of these proteins can impede wound healing (see, e.g., Mast, & Schultz (1996) Wound Repair and Regeneration 4:411-420). Ischemia of wound tissues occurs frequently in subjects having vascular disease (such as, e.g., venous hypertension, arterial insufficiency, or diabetes). Also, extended periods of pressure can cause ischemia in tissue pressure points in persons without nerve function who have lost nerve functions but are otherwise healthy (such as, e.g., quadriplegics or paraplegics). Thus, methods to restore reverse local tissue ischemia would promote healing of many chronic wounds. Delivery of an LP polypeptide (or fragment thereof) to wound cells (e.g., in a recombinant construct encoding the polypeptide or fragment) is used to test a polypeptide of the invention for its ability to treat ischemic, non-healing wounds. In one embodiment an LP polypeptide (or fragment thereof) is used in a rodent single pedicle dorsal skin flap method based on a technique of McFarlane, et al. (1965) Plastic and Reconstructive Surgery 35:177-182 to test angiogenesis.

### Example 30

### Effect of an LP Polypeptide in a Peripheral Arterial Disease Model

Angiogenic treatment using an LP polypeptide (or fragment thereof) is a novel therapeutic strategy to obtain restoration of blood flow around an ischemia (e.g., in a case of peripheral arterial disease). To test the ability of an LP polypeptide (or fragment thereof) to modulate such a peripheral arterial disease, the following experimental protocol is used: a) Using a rodent (as in the above described method) one side of the femoral artery is ligated to create ischemic damage to a muscle of the hindlimb (the other non-damaged hindlimb functions as the control); b) an LP polypeptide (or fragment thereof) is delivered to the animal either intravenously and/or intramuscularly (at the damaged limb) at least ×3 times per week for 2-3 weeks at a range of dosages (20 mg-500 mg); and c) the ischemic muscle tissue is collected after at 1, 2, and 3 weeks post-ligation for an analysis of expression of an LP polypeptide (or fragment thereof) and histology. Generally, (as above) parameters for evaluation include determining viability and vascularization of tissue surrounding the ischemia, while more specific evaluation parameters may include, e.g., measuring skin blood flow, skin temperature, and factor VIII immunohistochemistry, and/or endothelial alkaline phosphatase reaction. Polypeptide expression during the ischemia, is studied using any art known in situ hybridization technique. Biopsy is also performed on the other side of normal muscle of the contralateral hindlimb for analysis as a control.

### Example 31

### Effect of an LP Polypeptide in an Ischemic Myocardial Disease Mouse Model

An LP polypeptide (or fragment thereof) is evaluated as a treatment capable of stimulating the development of collateral vessels, and/or restructuring new vessels after coronary artery occlusion. The model is based on Guo, et al. (1999) Proc Natl Acad. Sci USA. 96:11507-11512 (incorporated herein for these teachings) demonstrating that a robust infarct-sparing effect occurs during the early and the late phases of preconditioning in the mouse and that the quantitative aspects of this effect are consistent with previous experience in other species. The model is useful to elucidate the molecular basis of ischemic preconditioning by making it possible to apply molecular biology techniques to intact animal preparations to dissect the precise role of a specific LP during ischemic events.

### Example 32

### Effect of an LP Polypeptide in a Rat Corneal Wound Healing Model

This animal model examines effects of an LP polypeptide (or fragment thereof) for angiogenic or anti-angiogenic activity on the normally avascular cornea. Briefly, the protocol comprises making a 1-1.5 mm long incision from the center of the corneal epithelium of an anesthetized mouse (e.g., a C57BL mouse strain) into the stromal layer then inserting a spatula below the lip of the incision facing the outer corner of the eye to make a pocket (whose base is 1-1.5 mm form the edge of the eye). Next, a pellet comprising an LP polypeptide or fragment thereof, (in a dosage range of about 50 ng-5ug) is positioned within the pocket (being immobilized in a slow release form, e.g., in an inert hydron pellet of approximately 1-2 ml volume). Alternatively, treatment with an LP polypeptide (or fragment thereof) can also be applied topically to the corneal wound in a dosage range of 20 mg-500 mg (daily treatment for five days). Over a 5 to 7 day post-operative period any angiogenic effect (e.g., stimulating the in growth of vessels from the adjacent vascularized corneal limbus) is determined. A photographic record is created by slit lamp photography. The appearance, density and extent of these vessels are evaluated and scored. In some instances, the time course of the progression is followed in anesthetized animals, before sacrifice. Vessels are evaluated for length, density and the radial surface of the limbus from which they emanate (expressed as clock-faced hours). Corneal wound healing is also assessed using any other art known technique.

### Example 33

### Effect of an LP Polypeptide in a Diabetic Mouse and Glucocorticoid-Impaired Wound Healing Models

Diabetic Mouse (db+/db+) as a Model

A genetically-induced diabetic mouse is used to examine the effect of an LP polypeptide (or fragment thereof) on wound healing. Mutant diabetic (db+/db+) mice have a single autosomal recessive mutation on chromosome 4 (db+) are used (Coleman et al. (1982) Proc. Natl. Acad. Sci. USA 72283-293). Typically, homozygous (db−/db−) mice are obese in comparison to their normal heterozygous (db+/db+) littermates. The mutant mice (db+/db+) have unique behavioral characteristics (such as, e.g., polyphagia, polydipsia, and polyuria); characteristic physiology (e.g., elevated blood glucose, increased or normal insulin levels, and suppressed cell-mediated immunity); and specific pathologies (such as, e.g., peripheral neuropathy, myocardial complications, and microvascular lesions, basement membrane thickening, and glomerular filtration abnormalities (see, e.g., Mandel, et al. (1978) J. Immunol. 120: 1375; Debray-Sachs, et al. (1983) Clin. Exp. Immunol. 51 (1):1-7; Leiter, et al. (1985) Am. J. of Pathol. 114:46-55; Norido, et al. (1984) Exp. Neural. 83(2):221-232; Robertson, et al. (1980) Diabetes 29(1):60-67; Giacomelli, et al. (1979) Lab Invest. 40(4):460-473; Coleman, (1982) Diabetes 31 (Suppl): 1-6). These homozygous diabetic mice also develop a form of insulin-resistant hyperglycemia that is analogous to human type II diabetes (Mandel, et al. (1978) J. Immunol. 120: 1375-1377). All things considered, healing in the db+/db+mouse may model the healing observed in humans with diabetes (see, Greenhalgh, et al. (1990) Am. J. of Pathol. 136:1235-1246). Thus, full-thickness, wound-healing using the db+/db+mouse is a useful well-characterized, clinically relevant, and reproducible model of impaired wound healing in humans. Generally, it is agreed that healing of the diabetic wound is dependent on formation of granulation tissue and re-epithelialization rather than simply by contraction (see, e.g., Gartner, et al. (1992) J. Surg. Res. 52:389; Greenhalgh, et al (1990) Am. J. Pathol. 136:1235). Moreover, the diabetic db+/db+animals have many of the characteristic features observed in Type II diabetes mellitus. Therefore, the genetically-induced db+/db+diabetic mouse is useful to examine the effect of an LP polypeptide (or fragment thereof) on wound healing according to the following method. Genetically, diabetic female C57BWKsJ mice and their non-diabetic heterozygous littermates are purchased at 6 weeks of age (Jackson Laboratories) and are 8 weeks old at the start of testing. Animals are individually housed and received food and water ad libitum. All manipulations are performed using standard aseptic techniques. The wounding protocol is performed generally according to the method of Tsuboi & Rifkin, (1990) Exp. Med. 172:245-251.

Steroid Impaired Rat Model

The following method is designed to investigate the effect of a topical treatment of varying concentrations of an LP polypeptide (or fragment thereof) on the wound of a healing-impaired rat (methylprednisolone impairment of a full thickness excisional skin wound). The inhibition of wound healing by steroids (such as, e.g., the glucocorticoid methylprednisolone) is well documented both in vitro and in vivo (see, e.g., Wahl, (1989) Glucocorticoids and Wound healing. In: Anti-Inflammatory Steroid Action: Basic and Clinical Aspects pp. 280-302; Wahlet, al. (1975) J.lmmunol. 115: 476-481; and Werb, et al. (1978) J. Exp. Med. 147:1684-1694). Glucocorticoids (such as methylprednisolone) are believed to retard wound healing by inhibiting angiogenesis, decreasing vascular permeability, fibroblast proliferation, collagen synthesis, and by transiently reducing the level of circulating monocytes. Furthermore, the systemic administration of steroids (such as glucocorticoids) to impair wound healing is a well established method used in rodents, such as, e.g., the rat (see, e.g., Ebert, et al. (1952) An. Intern. Med. 37:701-705; Beck, et al. (1991) Growth Factors. 5: 295-304; Haynes, et al. (1978) J. Clin. Invest. 61: 703-797; Haynes, et al. (1978) J. Clin. Invest. 61: 703-797; and Wahl, (1989), supra); and Pierce, et al. (1989) Proc. Natl. Acad. Sci. USA 86: 2229-2233). Thus, such a model is useful in assessing the effect of an LP polypeptide (or fragment thereof) of the invention on wound healing.

The assays, methods, or examples described herein test the activity of an LP polynucleotide sequence or an LP polypeptide (or fragment thereof). However, an ordinarily skilled artisan could easily modify (without undue experimentation) any exemplar taught herein using a different composition and/or concentration (such as, e.g., an agonist and/or an antagonist of an LP polynucleotide sequence or an LP polypeptide (or fragment thereof) of the invention. It will be clear that the invention may be practiced otherwise than as specifically described in the foregoing description and examples. Numerous modifications and variations of the present invention are possible in light of the above teachings and, therefore, are within the scope of the appended claims. The entire disclosure of each document cited (including patents, patent applications, journal articles, abstracts, laboratory manuals, books, or other disclosures) in the Background of the Invention, Detailed Description, and Examples is hereby incorporated herein by reference for the teachings they were intended to convey. Moreover, the hard copy of the sequence listing submitted herewith and the corresponding computer readable form are both incorporated herein by reference in their entireties, including without reservation, all corresponding drawings, pictures, graphs, diagrams, figures, figure legends, and http sites (including all corresponding information contained therein). The foregoing written specification is considered sufficient to enable a person of ordinary skill in the art to practice the invention. Indeed, various modifications of the invention in addition to those shown and described herein will become apparent from the foregoing description and these modifications also fall within the scope of the appended claims. All references cited herein are incorporated herein by reference to the same extent as if each individual publication or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the fill scope of equivalents to which such claims are entitled.

### Sequence Listing

SEQ ID NO: 1 is primate LP231 nucleic acid sequence.

SEQ ID NO: 2 is primate LP231 amino acid sequence.

SEQ ID NO: 3 is primate LP285 nucleic acid sequence.

SEQ ID NO: 4 is primate LP285 amino acid sequence.

SEQ ID NO: 5 is primate LP272 nucleic acid sequence.

SEQ ID NO: 6 is primate LP272 amino acid sequence.

SEQ ID NO: 7 is primate LP357 nucleic acid sequence.

SEQ ID NO: 8 is primate LP357 amino acid sequence.

SEQ ID NO: 9 is a DNA primer

SEQ ID NO: 10 is a DNA primer

SEQ ID NO: 11 is a DNA primer

SEQ ID NO: 12 is a DNA primer

SEQ ID NO: 13 is a DNA primer

SEQ ID NO: 14 is a DNA primer

SEQ ID NO: 15 is a DNA primer

SEQ ID NO: 16 is a DNA primer

SEQ ID NO: 17 is a DNA primer

What is claimed is:

1. An isolated or recombinant polynucleotide comprising sequence encoding an antigenic polypeptide comprising at least 17 contiguous amino acids from a mature coding portion of SEQ ID NO: Y (LP231, LP272, LP285, or LP357).

2. The polynucleotide of claim 1, encoding:
a) a full length polypeptide of SEQ ID NO: Y or Table 1, 2, 3, or 4;
b) a mature polypeptide of SEQ ID NO: Y or Table 1, 2, 3, or 4;
c) an antigenic fragments at least 12 contiguous amino acid residues in length of SEQ ID NO: Y from an LP of Table 1, 2, 3, or 4;
d) at least two fragments of SEQ ID NO: Y from an LP of Table 1, 2, 3, or 4, wherein said fragments do not overlap;
e) a plurality of fragments of SEQ ID NO: Y from an LP of Table 1, 2, 3, or 4, wherein said fragments do not overlap; or
f) a mature polypeptide of SEQ ID NO:Y with less than five amino acid substitutions.

3. The polynucleotide of claim 1, which hybridizes at 55° C., less than 500 mM salt, to:
a) the mature coding portion of SEQ ID NO: 1;
b) the mature coding portion of SEQ ID NO: 3;
c) the mature coding portion of SEQ ID NO: 5; or
d) the mature coding portion of SEQ ID NO: 7.

4. The polynucleotide of claim 3, wherein said temperature is at least 65° C., and said salt is less than 300 mM.

5. The polypeptide of claim 3, comprising at least 30, 32, 34, 36, 38, 39, 40, 42, 44, 46, 48, 49, 50, 52, 54, 56, 58, 59, 75, or at least about 150 contiguous nucleotides of a nucleotide sequence of:
a) the mature coding portion of SEQ ID NO: 1;
b) the mature coding portion of SEQ ID NO: 3;
c) the mature coding portion of SEQ ID NO: 5; or
d) the mature coding portion of SEQ ID NO: 7.

6. An expression vector comprising a polynucleotide of claim 1, wherein said temperature is at least 65° C., and said salt is less than 300 mM.

7. The expression vector of claim 6, which further comprises a plurality of nucleotide segments with identity to the coding portion of SEQ ID NO: X.

8. A host cell containing the expression vector of claim 6, including a eukaryotic cell.

9. A method of making an antigenic polypeptide comprising expressing a recombinant polynucleotide of claim 1.

10. A method for detecting a polynucleotide of claim 1, comprising contacting said polynucleotide with a probe that hybridizes, under stringent conditions, to at least 25 contiguous nucleotides of:
a) the mature coding portion of SEQ ID NO: 1;
b) the mature coding portion of SEQ ID NO: 3;
c) the mature coding portion of SEQ ID NO: 5; or
d) the mature coding portion of SEQ ID NO: 7;
to form a duplex, wherein detection of said duplex indicates the presence of said polynucleotide.

11. A kit for the detection of a polynucleotide of claim 1, comprising a compartment containing a probe that hybridizes, under stringent hybridization conditions, to at least 34 contiguous nucleotides of a polynucleotide of claim 1 to form a duplex.

12. The kit of claim 11, wherein said probe is detectably labeled.

13. A binding compound comprising an antibody which specifically binds to at least a 17 contiguous amino acid antigen binding site region of:
a) primate LP231 (SEQ ID NO: 2);
b) primate LP272 (SEQ ID NO: 4);
c) primate LP285 (SEQ ID NO: 6); or
d) primate LP357 (SEQ ID NO: 8).

14. The binding compound of claim 13, wherein:
a) said antibody binding site is:
i) specifically immunoreactive with a polypeptide of SEQ ID NO: Y;
ii) specifically immunoreactive with a polypeptide of SEQ ID NO: 2;
iii) specifically immunoreactive with a polypeptide of SEQ ID NO: 4;
iv) specifically immunoreactive with a polypeptide of SEQ ID NO: 6;
v) specifically immunoreactive with a polypeptide of SEQ ID NO: 8;
vi) raised against a purified or recombinantly produced human LP protein selected from: LP231, LP272, LP285, or LP357;
vii) in a monoclonal antibody, Fab, or F(ab)2; or
b) said binding compound is:
i) an antibody molecule;
ii) a polyclonal antiserum;
iii) detectably labeled;
iv) sterile; or
v) in a buffered composition.

15. A method using the binding compound of claim 13, comprising contacting said binding compound with a biological sample comprising an antigen, thereby forming an LP binding compound:antigen complex.

16. The method of claim 15, wherein said biological sample is from a human, and wherein said binding compound is an antibody.

17. A detection kit comprising said binding compound of claim 14, and:
a) instructional material for the use of said binding compound for said detection; or
b) a compartment providing segregation of said binding compound.

18. A substantially pure or isolated antigenic polypeptide, which binds to said binding composition of claim 13, and further comprises at least 25 contiguous amino acids from:
a) primate LP231 (SEQ ID NO: 2);
b) primate LP272 (SEQ ID NO: 4);
c) primate LP285 (SEQ ID NO: 6); or
d) primate LP357 (SEQ ID NO: 8).

19. The polypeptide of claim 18, which:
a) comprises at least a fragment of at least 29 contiguous amino acid residues from a primate LP protein selected from: LP231, LP272, LP285, or LP357;
b) is a soluble polypeptide;
c) is detectably labeled;
d) is in a sterile composition;
e) is in a buffered composition;
f) is recombinantly produced, or
g) has a naturally occurring polypeptide sequence.

20. The binding compound of claim 14, where said compound is an antibody that:
a) is raised against a peptide sequence of a mature polypeptide of Table 1, 2, 3, or 4;
b) is produced in a mammal, or a plant;
c) is immunoselected; or
d) binds to a denatured polypeptide of Table 1, 2, 3, or 4.
